(19) United States
(12) Patent Application Publication
(10) Pub. No.: US 2005/0152944 A1
(43) Pub. Date: Jul. 14, 2005

(54) Soft tissue implants and anti-scarring agents

(75) Inventors: William L. Hunter, Vancouver (CA); David M. Gravett, Vancouver (CA); Philip M. Toleikis, Vancouver (CA); Arpita Maiti, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 11/006,881

(22) Filed: Dec. 7, 2004

Related U.S. Application Data
(63) Continuation of application No. 10/996,353, filed on Nov. 22, 2004.
Application No. 10/996,353 is a continuation-in-part of application No. 10/986,231, filed on Nov. 10, 2004.
Application No. 10/996,353 is a continuation-in-part of application No. 10/986,230, filed on Nov. 10, 2004.
(60) Provisional application No. 60/586,861, filed on Jul. 9, 2004.
(60) Provisional application No. 60/578,471, filed on Jun. 9, 2004.
(60) Provisional application No. 60/526,541, filed on Dec. 3, 2003.
(60) Provisional application No. 60/525,226, filed on Nov. 24, 2003.
(60) Provisional application No. 60/523,908, filed on Nov. 20, 2003.
(60) Provisional application No. 60/524,023, filed on Nov. 20, 2003.

(51) Int. Cl. A61F 2/12

(52) U.S. Cl. 424/423; 623/23.72; 623/11.11

(57) ABSTRACT

Soft tissue implants (e.g., breast, pectoral, chin, facial, lip, and nasal implants) are used in combination with an anti-scarring agent in order to inhibit scarring that may otherwise occur when the implant is placed within an animal.

### CROSS-REFERENCES TO RELATED APPLICATIONS

This application is a Continuation of co-pending U.S. Utility application Ser. No. 10/996,353, filed Nov. 22, 2004; which application is a Continuation-in-part of U.S. application Ser. No. 10/986,231, filed Nov. 10, 2004; and Ser. No. 10/986,230, filed Nov. 10, 2004; which application also claims the benefit under 35 U.S.C. 119(e) of U.S. Provisional Application Ser. Nos. 60/586,861, filed Jul. 9, 2004; No. 60/578,471, filed Jun. 9, 2004; 60/526,541, filed Dec. 3, 2003; 60/525,226, filed Nov. 24, 2003; 60/523,908, filed Nov. 20, 2003; and 60/524,023, filed Nov. 20, 2003, which applications are incorporated herein by reference in their entireties.

### BACKGROUND OF THE INVENTION

1. Field of the Invention

The present invention relates generally to soft tissue implants for use in cosmetic or reconstructive surgery, and more specifically, to compositions and methods for preparing and using such medical implants to make them resistant to overgrowth by inflammatory, fibrous scar tissue.

2. Description of the Related Art

The use of soft tissue implants for cosmetic applications (aesthetic and reconstructive) is common in breast augmentation, breast reconstruction after cancer surgery, craniofacial procedures, reconstruction after trauma, congenital craniofacial reconstruction and oculoplastic surgical procedures to name a few.

The clinical function of a soft tissue implant depends upon the implant being able to effectively maintain its shape over time. In many instances, for example, when these devices are implanted in the body, they are subject to a “foreign body” response from the surrounding host tissues. The body recognizes the implanted device as foreign, which triggers an inflammatory response followed by encapsulation of the implant with fibrous connective tissue. Encapsulation of surgical implants complicates a variety of reconstructive and cosmetic surgeries, and is particularly problematic in the case of breast reconstruction surgery where the breast implant becomes encapsulated by a fibrous connective tissue capsule that alters the anatomy and function. Scar capsules that harden and contract (known as “capsular contractures”) are the most common complication of breast implant or reconstructive surgery. Capsular (fibrous) contractures can result in hardening of the breast, loss of the normal anatomy and contour of the breast, discomfort, weakening and rupture of the implant shell, asymmetry, infection, and patient dissatisfaction. Further, fibrous encapsulation of any soft tissue implant can occur even after a successful implantation if the device is manipulated or Irritated by the daily activities of the patient.

Scarring and fibrous encapsulation can also result from a variety of other factors associated with implantation of a soft tissue implant. For example, unwanted scarring can result from surgical trauma to the anatomical structures and tissue surrounding the implant during the implantation of the device. Bleeding in and around the implant can also trigger a biological cascade that ultimately leads to excess scar tissue formation. Similarly, if the implant initiates a foreign body response, the surrounding tissue can be Inadvertently damaged from the resulting inflammation, leading to loss of function, tissue damage and/or tissue necrosis. Furthermore, certain types of implantable prostheses (such as breast implants) include gel fillers (e.g., silicone) that tend to leak through the membrane envelope of the Implant and can potentially cause a chronic inflammatory response in the surrounding tissue (which augments tissue encapsulation and contracture formation). When scarring occurs around the implanted device, the characteristics of the implant-tissue interface degrade, the subcutaneous tissue can harden and contract and the device can become disfigured. The effects of unwanted scarring in the vicinity of the implant are the leading cause of additional surgeries to correct defects, break down scar tissue, or remove the implant.

### BRIEF SUMMARY OF THE INVENTION

Briefly stated, the present invention discloses pharmaceutical agents that inhibit one or more aspects of the production of excessive fibrous (scar) tissue. In one aspect, the present invention provides compositions for delivery of selected therapeutic agents via medical implants, as well as methods for making and using these implants and devices. Compositions and methods are described for coating soft tissue implants with drug-delivery compositions such that the pharmaceutical agent is delivered in therapeutic levels over a period sufficient to prevent the implant from being encapsulated in fibrous tissue and to allow normal function of the implant to occur. Alternatively, locally administered compositions (e.g., topicals, injectables, liquids, gels, sprays, microspheres, pastes, wafers) containing an inhibitor of fibrosis are described that can be applied to the tissue adjacent to the soft tissue implant, such that the pharmaceutical agent is delivered in therapeutic levels over a period sufficient to prevent the implant from being encapsulated in fibrous tissue. And finally, numerous specific soft tissue implants are described that produce superior clinical results as a result of being coated with agents that reduce excessive scarring and fibrous tissue accumulation as well as other related advantages.

Within one aspect of the invention, drug-coated or drug-impregnated soft tissue implants are provided which reduce fibrosis in the tissue surrounding the implant, or inhibit scar development on the implant surface, thus enhancing the efficacy of the procedure. Within various embodiments, fibrosis is inhibited by local or systemic release of specific pharmacological agents that become localized to the adjacent tissue.

The repair of tissues following a mechanical or surgical intervention, such as the implantation of a soft tissue implant, involves two distinct processes: (1) regeneration (the replacement of injured cells by cells of the same type and (2) fibrosis (the replacement of injured cells by connective tissue). There are five general components to the process of fibrosis (or scarring) including: infiltration and activation of inflammatory cells (inflammation), migration and proliferation of connective tissue cells (such as fibroblasts or smooth muscle cells), the formation of new blood vessels (angiogenesis), deposition of extracellular matrix (ECM), and remodeling (maturation and organization of the fibrous tissue). As utilized herein, “inhibits (reduces) fibrosis” should be understood to refer to agents or compositions which decrease or limit the formation of fibrous or scar tissue (i.e., by reducing or inhibiting one or more of the processes of inflammation, connective tissue cell migration or proliferation, angiogenesis, ECM production, and/or remodeling). In addition, numerous therapeutic agents described in this invention will have the additional benefit of also reducing tissue regeneration where appropriate.

Within one embodiment of the invention, a soft tissue implant is adapted to release an agent that inhibits fibrosis through one or more of the mechanisms cited herein.

Within related aspects of the present invention, medical devices are provided comprising a soft tissue implant, wherein the implant or device releases an agent that inhibits fibrosis in vivo. “Release of an agent” refers to any statistically significant presence of the agent, or a subcomponent thereof, which has disassociated from the implant/device and/or remains active on the surface of (or within) the device/implant. Within yet other aspects of the present invention, methods are provided for manufacturing a medical device or implant, comprising the step of coating (e.g., spraying, dipping, wrapping, or administering drug through) a soft tissue implant. Additionally, the implant or medical device can be constructed so that the device itself is comprised of materials that inhibit fibrosis in or around the implant. A wide variety of soft tissue implants may be utilized within the context of the present invention, depending on the site and nature of treatment desired.

Within various embodiments of the invention, the soft tissue implant is further coated with a composition or compound, which delays the onset of activity of the fibrosis-inhibiting agent for a period of time after implantation.

Representative examples of such agents include heparin, PLGA/MePEG, PLA, and polyethylene glycol. Within further embodiments, the fibrosis-inhibiting implant or device is activated before, during, or after deployment (e.g., an inactive agent on the device is first activated to one that reduces or inhibits an in vivo fibrotic reaction).

Within various embodiments of the invention, the tissue surrounding the implant or device is treated with a composition or compound that contains an inhibitor of fibrosis. Locally administered compositions (e.g., topicals, injectables, liquids, gels, sprays, microspheres, pastes, wafers) or compounds containing an inhibitor of fibrosis are described that can be applied to the surface of, or infiltrated into, the tissue adjacent to the device, such that the pharmaceutical agent is delivered in therapeutic levels over a period sufficient to prevent the soft tissue implant from being encapsulated in fibrous tissue. This can be done in lieu of coating the implant with a fibrosis-inhibitor, or done in addition to coating the device or implant with a fibrosis-inhibitor. The local administration of the fibrosis-inhibiting agent can occur prior to, during, or after implantation of the soft tissue implant itself.

Within various embodiments of the invention, a soft tissue implant is coated in one aspect with a composition which inhibits fibrosis, as well as being coated with a composition or compound that promotes scarring on another aspect of the device (i.e., to affix the body of the device into a particular anatomical space). Representative examples of agents that promote fibrosis and scarring include silk, silica, bleomycin, neomycin, talcum powder, metallic beryllium, retinoic acid compounds, growth factors, and copper, as well as analogues and derivatives thereof.

Also provided by the present invention are methods for treating patients undergoing surgical, endoscopic or minimally invasive therapies where a soft tissue implant is placed as part of the procedure. As utilized herein, it should be understood that “inhibits fibrosis” refers to a statistically significant decrease in the amount of scar tissue in or around the device or an improvement in the interface between the device and the tissue and not to a permanent prohibition of any complications or failures of the device/implant.

The pharmaceutical agents and compositions are utilized to create novel drug-coated soft tissue implants that reduce the foreign body response to implantation and limit the growth of reactive tissue on the surface of, or around in the tissue surrounding the implant, such that performance is enhanced. Soft tissue implants coated with selected pharmaceutical agents designed to prevent scar tissue overgrowth, prevent encapsulation, improve function, reduce the need for repeat intervention, and enhance appearance and can offer significant clinical advantages over uncoated soft tissue implants.

For example, in one aspect the present invention is directed to medical devices that comprise a soft tissue implant and at least one of (i) an anti-scarring agent and (ii) a composition that comprises an anti-scarring agent. The agent is present so as to inhibit scarring that may otherwise occur when the implant is placed within an animal. In another aspect the present invention is directed to methods wherein both a soft tissue implant and at least one of (i) an anti-scarring agent and (ii) a composition that comprises an anti-scarring agent, are placed into an animal, and the agent inhibits scarring that may otherwise occur. These and other aspects of the invention are summarized below.

Thus, in various independent aspects, the present invention provides a device, comprising a soft tissue implant and an anti-scarring agent or a composition comprising an anti-scarring agent, wherein the agent inhibits scarring. These and other devices are described in more detail herein.

In each of the aforementioned devices, in separate aspects, the present invention provides that the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a p38 MAP kinase inhibitor. These and other agents are described in more detail herein.

In additional aspects, for each of the aforementioned soft tissue implants combined with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In addition to devices, the present invention also provides methods. For example, in additional aspects of the present invention, for each of the aforementioned devices, and for each of the aforementioned combinations of the soft tissue implants with the anti-scarring agents, the present invention provides methods whereby a specified soft tissue implant is implanted into an animal, and a specified agent associated with the implant inhibits scarring that may otherwise occur. Each of the soft tissue implants identified herein may be a “specified implant”, and each of the anti-scarring agents identified herein may be an “anti-scarring (or fibrosis-inhibiting) agent”, where the present invention provides, in independent embodiments, for each possible combination of the implant and the agent.

The agent may be associated with the soft tissue implant prior to, during and/or after placement of the soft tissue implant within the animal. For example, the agent (or composition comprising the agent) may be coated onto an implant, and the resulting device then placed within the animal. In addition, or alternatively, the agent may be independently placed within the animal in the vicinity of where the soft tissue implant is to be, is being, or has been placed within the animal. For example, the agent may be sprayed or otherwise placed onto, adjacent to, and/or within the tissue that will be contacting the medical implant or may otherwise undergo scarring. To this end, the present invention provides placing a soft tissue implant and an anti-scarring agent or a composition comprising an anti-scarring agent into an animal host, wherein the agent inhibits scarring.

In each of the aforementioned methods, in separate aspects, the present invention provides that: the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a p38 MAP kinase inhibitor. These and other agents that can inhibit fibrosis are described in more detail herein.

In additional aspects, for each of the aforementioned methods used in combination with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of soft tissue implant and agent described above, are set forth in greater detail herein.

These and other aspects of the present invention will become evident upon reference to the following detailed description and attached drawings. In addition, various references are set forth herein that describe in more detail certain procedures and/or compositions (e.g., polymers), and are therefore incorporated by reference in their entirety.

### BRIEF DESCRIPTION OF THE DRAWINGS

FIG. 1 is a diagram showing how a cell cycle inhibitor acts at one or more of the steps in the biological pathway.

FIG. 2 is a graph showing the results for the screening assay for assessing the effect of mitoxantrone on nitric oxide production by THP-1 macrophages.

FIG. 3 is a graph showing the results for the screening assay for assessing the effect of Bay 11-7082 on TNF-alpha production by THP-1 macrophages.

FIG. 4 is a graph showing the results for the screening assay for assessing the effect of rapamycin concentration for TNFα production by THP-1 macrophages.

FIG. 5 is graph showing the results of a screening assay for assessing the effect of mitoxantrone on proliferation of human fibroblasts.

FIG. 6 is graph showing the results of a screening assay for assessing the effect of rapamycin on proliferation of human fibroblasts.

FIG. 7 is graph showing the results of a screening assay for assessing the effect of paclitaxel on proliferation of human fibroblasts.

FIG. 8 is a picture that shows an uninjured carotid artery from a rat balloon injury model.

FIG. 9 is a picture that shows an injured carotid artery from a rat balloon injury model.

FIG. 10 is a picture that shows a paclitaxel/mesh treated carotid artery in a rat balloon injury model.

FIG. 11A schematically depicts the transcriptional regulation of matrix metalloproteinases.

FIG. 11B is a blot that demonstrates that IL-1 stimulates AP-1 transcriptional activity.

FIG. 11C is a graph that shows that IL-1 induced binding activity decreased in lysates from chondrocytes which were pretreated with paclitaxel.

FIG. 11D is a blot which shows that IL-1 induction increases collagenase and stromelysin in RNA levels in chondrocytes, and that this induction can be inhibited by pretreatment with paclitaxel.

FIGS. 12A-H are blots that show the effect of various anti-microtubule agents in inhibiting collagenase expression.

FIG. 13 is a graph showing the results of a screening assay for assessing the effect of paclitaxel on smooth muscle cell migration.

FIG. 14 is a graph showing the results of a screening assay for assessing the effect of geldanamycin on IL-13 production by THP-1 macrophages.

FIG. 15 is a graph showing the results of a screening assay for assessing the effect of geldanamycin on IL-8 production by THP-1 macrophages.

FIG. 16 is a graph showing the results of a screening assay for assessing the effect of geldanamycin on MCP-1 production by THP-1 macrophages.

FIG. 17 is graph showing the results of a screening assay for assessing the effect of paclitaxel on proliferation of smooth muscle cells.

FIG. 18 is graph showing the results of a screening assay for assessing the effect of paclitaxel for proliferation of the murine RAW 264.7 macrophage cell line.

FIG. 19 is a bar graph showing the area of granulation tissue in carotid arteries exposed to silk coated perivascular polyurethane (PU) films relative to arteries exposed to uncoated PU films.

FIG. 20 is a bar graph showing the area of granulation tissue in carotid arteries exposed to silk suture coated perivascular PU films relative to arteries exposed to uncoated PU films.

FIG. 21 is a bar graph showing the area of granulation tissue in carotid arteries exposed to natural and purified silk powder and wrapped with perivascular PU film relative to a control group in which arteries are wrapped with perivascular PU film only.

FIG. 22 is a bar graph showing the area of granulation tissue (at 1 month and 3 months) in carotid arteries sprinkled with talcum powder and wrapped with perivascular PU film relative to a control group in which arteries are wrapped with perivascular PU film only.

### DETAILED DESCRIPTION OF THE INVENTION

Definitions

Prior to setting forth the invention, it may be helpful to an understanding thereof to first set forth definitions of certain terms that is used hereinafter.

“Medical device,” “implant,” “device,” “medical device,” “medical implant,” “implant/device,” and the like are used synonymously to refer to any object that is designed to be placed partially or wholly within a patient's body for one or more therapeutic or prophylactic purposes such as for tissue augmentation, contouring, restoring physiological function, repairing or restoring tissues damaged by disease or trauma, and/or delivering therapeutic agents to normal, damaged or diseased organs and tissues. While medical devices are normally composed of biologically compatible synthetic materials (e.g., medical-grade stainless steel, titanium and other metals; exogenous polymers, such as polyurethane, silicon, PLA, PLGA), other materials may also be used in the construction of the medical implant. Specific medical devices and implants that are particularly useful for the practice of this invention include soft tissue implants for cosmetic and reconstructive surgery.

“Soft tissue implant” refers to a medical device or implant that includes a volume replacement material for augmentation or reconstruction to replace a whole or part of a living structure. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants include breast implants, chin implants, calf implants, cheek implants and other facial implants, buttocks implants, and nasal implants.

“Fibrosis” or “scarring” refers to the formation of fibrous (scar) tissue in response to injury or medical intervention. Therapeutic agents which inhibit fibrosis or scarring can do so through one or more mechanisms including inhibiting inflammation, inhibiting angiogenesis, inhibiting migration or proliferation of connective tissue cells (such as fibroblasts smooth muscle cells vascular smooth muscle cells), reducing ECM production or encouraging ECM breakdown, and/or inhibiting tissue remodeling. In addition, numerous therapeutic agents described in this invention will have the additional benefit of also reducing tissue regeneration (the replacement of injured cells by cells of the same type) when appropriate.

“Inhibit fibrosis,” “inhibit scar,” “reduce fibrosis,” “reduce scar,” “fibrosis-inhibitor,” “anti-scarring” and the like are used synonymously to refer to the action of agents or compositions which result in a statistically significant decrease in the formation, deposition and/or maturation of fibrous tissue that may be expected to occur in the absence of the agent or composition.

“Encapsulation” as used herein refers to the formation of a fibrous connective tissue capsule (containing fibroblasts, myofibroblasts, inflammatory cells, relatively few blood vessels and a collagenous extracellular matrix) encloses and isolates an implanted prosthesis or biomaterial from the surrounding body tissue. This fibrous tissue capsule, which is the result of unwanted scarring in response to an implanted prosthesis or biomaterial, has a tendency to progressively contract, thereby tightening around the implant/biomaterial and causing it to become very firm and disfigured. Further implications of encapsulation and associated contracture include tenderness of the tissue, pain, erosion of the adjacent tissue as well as other complications.

“Contracture” as used herein refers to permanent or non-permanent scar tissue formation in response to an implanted prosthesis or biomaterial. In general, the condition of contracture involves a fibrotic response that may involve inflammatory components, both acute and chronic. Unwanted scarring in response to an implanted prosthesis or biomaterial can form a fibrous tissue capsule around the area or implantable prosthesis or biomaterial that encloses and isolates it from the surrounding body tissue (as described for encapsulation). Contracture occurs when fibrous tissue capsule matures and starts to shrink (contract) forming a tight, hard capsule around the implant/biomaterial that can alter the anatomy, texture, shape and movement of the implant. In some cases, contracture also draws the overlying skin in towards the implant and leads to dimpling of the skin and disfuguration. Contracture and chronic inflammation can also contribute to tenderness around the implant, pain, and erosion of the adjacent tissue. Fibrotic contractures related to implantation of soft tissue implant/biomaterials may be caused by a variety of factors including surgical trauma and complications, revisions or repeat procedures (the incidence is higher if implantation is being attempted where contractures have occurred previously), inadequate hemostasis (bleeding control) during surgery, aggressive healing processes, underlying or pre-existent conditions, genetic factors (people prone to hypertrohic scar or keloid formation), and immobilization.

“Host,” “person,” “subject,” “patient,” and the like are used synonymously to refer to the living being (human or animal) into which a soft tissue implant of the present invention is implanted.

“Implanted” refers to having completely or partially placed a device within a host. A device is partially implanted when some of the device reaches, or extends to the outside of, a host.

“Release of an agent” refers to a statistically significant presence of the agent, or a subcomponent thereof, which has disassociated from the implant and/or remains active on the surface of (or within) the device/implant.

“Analogue” refers to a chemical compound that is structurally similar to a parent compound but differs slightly in composition (e.g., one atom or functional group is different, added, or removed). An analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

“Derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

“Inhibitor” refers to an agent that prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent that prevents a biological process from occurring or slows the rate or degree of occurrence of a biological process. While the process may be a general one, typically this refers to a drug mechanism by which the drug competes with a molecule for an active molecular site or prevents a molecule from interacting with the molecular site. In these situations, the effect is that the molecular process is inhibited.

“Agonist” refers to an agent that stimulates a biological process or rate or degree of occurrence of a biological process. The process may be a general one such as scarring or refer to a specific biological action such as, for example, a molecular process resulting in release of a cytokine.

“Anti-microtubule agent” should be understood to include any protein, peptide, chemical, or other molecule that impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett. 79(2): 213-219, 1994) and Mooberry et al., (Cancer Lett. 96(2): 261-266, 1995).

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to both one polymer or a mixture comprising two or more polymers. As used herein, the term “about” means±15%.

As discussed above, the present invention provides compositions, methods and devices relating to cosmetic and reconstructive devices and implants, which greatly increase their ability to inhibit the formation of reactive scar tissue on, or around, the surface of the implant. In one aspect, the present invention provides for the combination of an anti-scarring agent and a soft tissue implant for use in cosmetic or reconstructive surgery. In yet another aspect, soft tissue implants are provided that can reduce the development of surrounding scar capsules that harden and contract (also referred to herein as capsular or fibrous contracture), discomfort, leakage of fluid from the implant, infection, asymmetry, and patient dissatisfaction. Described in more detail below are methods for constructing soft tissue implants, compositions and methods for generating medical implants that inhibit fibrosis, and methods for utilizing such medical implants.

A. Clinical Applications of Soft Tissue Implants Which Include and Release a Fibrosis-Inhibiting Agent

There are numerous types of soft tissue implants where the occurrence of a fibrotic reaction will adversely affect the functioning or appearance of the implant or the tissue surrounding the implant. Typically, fibrotic encapsulation of the soft tissue implant (or the growth of fibrous tissue between the implant and the surrounding tissue) can result in fibrous contracture and other problems that can lead to suboptimal appearance and patient comfort. Accordingly, the present invention provides for soft tissue implants that include an agent that inhibits the formation of scar tissue to minimize or prevent encapsulation (and associated fibrous contracture) of the soft tissue implant.

Soft tissue implants are used in a variety of cosmetic, plastic, and reconstructive surgical procedures and may be delivered to many different parts of the body, including, without limitation, the face, nose, jaw, breast, chin, buttocks, chest, lip, and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants that can be coated with, or otherwise constructed to contain and/or release fibrosis-inhibiting agents provided herein, include, e.g., saline breast implants, silicone breast implants, triglyceride-filled breast implants, chin and mandibular implants, nasal implants, cheek implants, lip implants, and other facial implants, pectoral and chest implants, malar and submalar implants, and buttocks implants.

Soft tissue implants have numerous constructions and may be formed of a variety of materials, such as to conform to the surrounding anatomical structures and characteristics. In one aspect, soft tissue implants suitable for combining with a fibrosis-inhibitor are formed from a polymer such as silicone, poly(tetrafluoroethylene), polyethylene, polyurethane, polymethylmethacrylate, polyester, polyamide and polypropylene. Soft tissue implants may be in the form shell (or envelope) that is filled with a fluid material such as saline.

In one aspect, soft tissue implants include or are formed from silicone or dimethylsiloxane. Silicone implants can be solid, yet flexible and very durable and stable. They are manufactured in different durometers (degrees of hardness) to be soft or quite hard, which is determined by the extent of polymerization. Short polymer chains result in liquid silicone with less viscosity, while lengthening the chains produces gel-type substances, and cross-linking of the polymer chains results in high-viscosity silicone rubber. Silicone may also be mixed as a particulate with water and a hydrogel carrier to allow for fibrous tissue ingrowth. These implants are designed to enhance soft tissue areas rather than the underlying bone structure. In certain aspects, silicone-based implants (e.g., chin implants) may be affixed to the underlying bone by way of one or several titanium screws. Silicone implants can be used to augment tissue in a variety of locations in the body, including, for example, breast, nasal, chin, malar (e.g., cheek), and chest/pectoral area. Silicone gel with low viscosity has been primarily used for filling breast implants, while high viscosity silicone is used for tissue expanders and outer shells of both saline-filled and silicone-filled breast implants. For example, breast implants are manufactured by both Inamed Corporation (Santa Barbara, Calif.) and Mentor Corporation (Santa Barbara, Calif.).

In another aspect, soft tissue implants include or are formed from poly(tetrafluoroethylene) (PTFE). In certain aspects, the poly(tetrafluoroethylene) is expanded polytetrafluoroethylene (ePTFE). PTFE used for soft tissue implants may be formed of an expanded polymer of solid PTFE nodes with interconnecting, thin PTFE fibrils that form a grid pattern, resulting in a pliable, durable, biocompatible material. Soft tissue implants made of PTFE are often available in sheets that may be easily contoured and stacked to a desired thickness, as well as solid blocks. These implants are porous and can become integrated into the surrounding tissue that aids in maintaining the implant in its appropriate anatomical location. PTFE implants generally are not as firm as silicone implants. Further, there is less bone resorption underneath ePTFE implants as opposed to silicone implants. Soft tissue implants composed of PTFE may be used to augment tissue in a variety of locations in the body, including, for example, facial, chest, lip, nasal, and chin, as well as the mandibular and malar region and for the treatment of nasolabial and glabellar creases. For example, GORE-TEX (W.L. Gore & Associates, Inc., Newark, Del.) is an expanded synthetic PTFE that may be used to form facial implants for augmentation purposes.

In yet another aspect, soft tissue implants include or are formed from polyethylene. Polyethylene implants are frequently used, for example in chin augmentation. Polyethylene implants can be porous, such that they may become integrated into the surrounding tissue, which provides an alternative to using titanium screws for stability. Polyethylene implants may be available with varying biochemical properties, including chemical resistance, tensile strength, and hardness. Polyethylene implants may be used for facial reconstruction, including malar, chin, nasal, and cranial implants. For example, Porex Surgical Products Group (Newnan, Ga.) makes MEDPOR, which is a high-density, porous polyethylene implant that is used in facial reconstruction. The porosity allows for vascular and soft tissue ingrowth for incorporation of the implant.

In yet another aspect, soft tissue implants include or are formed from polypropylene. Polypropylene implants are a loosely woven, high density polymer having similar properties to polyethylene. These implants have good tensile strength and are available as a woven mesh, such as PROLENE (Ethicon, Inc., Sommerville, N.J.) or MARLEX (C.R. Bard, Inc., Billerica, Mass.). Polypropylene implants may be used, for example, as chest implants.

In yet another aspect, soft tissue implants include or are formed from polyamide. Polyamide is a nylon compound that is woven into a mesh that may be implanted for use in facial reconstruction and augmentation. These implants are easily shaped and sutured and undergo resorption over time. SUPRAMID and SUPRAMESH(S. Jackson, Inc., Minneapolis, Minn.) are nylon-based products that may be used for augmentation; however, because of their resorptive properties, their application is limited.

In yet another aspect, soft tissue implants include or are formed from polyester. Nonbiodegradable polyesters, such as MERSILENE Mesh (Ethicon, Inc.) and DACRON (available from Invista, Wichita, Kans.), may be suitable as implants for applications that require both tensile strength and stability, such as chest, chin and nasal augmentation.

In yet another aspect, soft tissue implants include or are formed from polymethylmethacrylate. These implants have a high molecular weight and have compressive strength and rigidity even though they have extensive porosity. Polymethylmethacrylate, such as Hard Tissue Replacement (HTR) polymer made by U.S. Surgical Corporation (Norwalk, Conn.), may be used for chin and malar augmentation as well as craniomaxillofacial reconstruction.

In yet another aspect, soft tissue implants include or are formed from polyurethane. Polyurethane may be used as a foam to cover breast implants. This polymer promotes tissue ingrowth resulting in low capsular contracture rate in breast implants.

Examples of commercially available polymeric soft tissue implants suitable for use in combination with a fibrosis-inhibitor include silicone implants from Surgiform Technology, Ltd. (Columbia Station, Ohio); ImplantTech Associates (Ventura, Calif.); Inamed Corporation (Santa Barbara, Calif.; see M766A Spectrum Catalog); Mentor Corporation (Santa Barbara, Calif.); and Allied Biomedical (Ventura, Calif.). Saline filled breast implants are made by both Inamed and Mentor and may also benefit from implantation in combination with a fibrosis inhibitor. Commercially available poly(tetrafluoroethylene) soft tissue implants suitable for use in combination with a fibrosis-inhibitor include poly(tetrafluoroethylene) cheek, chin, and nasal implants from W. L. Gore & Associates, Inc. (Newark, Del.). Commercially available polyethylene soft tissue implants suitable for use in combination with a fibrosis-inhibitor include polyethylene implants from Porex Surgical Inc. (Fairburn, Ga.) sold under the trade name MEDPOR Biomaterial. MEDPOR Biomaterial is composed of porous, high-density polyethylene material with an omni-directional latticework of interconnecting pores, which allows for integration into host tissues.

Upon implantation, excessive scar tissue growth can occur around the all or parts of the implant, which can lead to a reduction in the performance of these devices (as described previously). Soft tissue implants that release a therapeutic agent for reducing scarring at the implant-tissue interface can be used to enhance the appearance, increase the longevity, reduce the need for corrective surgery or repeat procedures, decrease the incidence of pain and other symptoms, and improve the clinical function of implant. Accordingly, the present invention provides soft tissue implants that are coated or otherwise incorporate an anti-scarring agent or a composition that includes an anti-scarring agent.

For greater clarity, several specific soft tissue implants and treatments will be described in greater detail including breast implants and other cosmetic implants.

B. Breast Implants

In one aspect, the soft tissue implant suitable for use in combination with a fibrosis-inhibitor is a breast implant. Breast implant placement for augmentation or breast reconstruction after mastectomy is one of the most frequently performed cosmetic surgery procedures. For example, in 2002 alone, over 300,000 women had breast implant surgery. Of these women, approximately 80,000 had breast reconstructions following a mastectomy due to cancer. An increased number of breast implant surgeries is highly likely given the incidence of breast cancer and current trends in cosmetic surgery.

In general, breast augmentation or reconstructive surgery involves the placement of a commercially available breast implant, which consists of a capsule filled with either saline or silicone, into the tissues underneath the mammary gland. Four different incision sites have historically been used for breast implantation: axillary (armpit), periareolar (around the underside of the nipple), inframamary (at the base of the breast where it meets the chest wall) and transumbilical (around the belly button). The tissue is dissected away through the small incision, often with the aid of an endoscope (particularly for axillary and transumbilical procedures where tunneling from the incision site to the breast is required). A pocket for placement of the breast implant is created in either the subglandular or the subpectorial region. For subglandular implants, the tissue is dissected to create a space between the glandular tissue and the pectoralis major muscle that extends down to the inframammary crease. For subpectoral implants, the fibres of the pectoralis major muscle are carefully dissected to create a space beneath the pectoralis major muscle and superficial to the rib cage. Careful hemostasis is essential (since it can contribute to complications such as capsular contractures), so much so that minimally invasive procedures (axillary, transumbilical approaches) must be converted to more open procedures (such as periareolar) if bleeding control is inadequate. Depending upon the type of surgical approach selected, the breast implant is often deflated and rolled up for placement in the patient. After accurate positioning is achieved, the implant can then be filled or expanded to the desired size.

Although many patients are satisfied with the initial procedure, significant percentages suffer from complications that frequently require a repeat intervention to correct. Encapsulation of a breast prosthesis that creates a periprosthetic shell (called capsular contracture) is the most common complication reported after breast enlargement, with up to 50% of patients reporting some dissatisfaction. Calcification can occur within the fibrous capsule adding to its firmness and complicating the interpretation of mammograms. Multiple causes of capsular contracture have identified including: foreign body reaction, migration of silicone gel molecules across the capsule and into the tissue, autoimmune disorders, genetic predisposition, infection, hematoma, and the surface characteristics of the prosthesis. Although no specific etiology has been repeatedly identified, at the cellular level, abnormal fibroblast activity stimulated by a foreign body is a consistent finding. Periprosthetic capsular tissues contain macrophages and occasional T- and B-lymphocytes, suggesting an inflammatory component to the process. Implant surfaces have been made both smooth and textured in an attempt to reduce encapsulation, however, neither has been proven to produce consistently superior results. Animal models suggest that there is an increased tendency for increased capsular thickness and contracture with textured surfaces that encourage fibrous tissue ingrowth on the surface. Placement of the implant in the subpectoral location appears to decrease the rate of encapsulation in both smooth and textured implants.

From a patient's perspective, the biological processes described above lead to a series of commonly described complaints. Implant malposition, hardness and unfavorable shape are the most frequently sited complications and are most often attributed to capsular contracture. When the surrounding scar capsule begins to harden and contract, it results in discomfort, weakening of the shell, asymmetry, skin dimpling and malpositioning. True capsular contractures will occur in approximately 10% of patients after augmentation, and in 25% to 30% of reconstruction cases, with most patients reporting dissatisfaction with the aesthetic outcome. Scarring leading to asymmetries occurs in 10% of augmentations and 30% of reconstructions and is the leading cause of revision surgery. Skin wrinkling (due to the contracture pulling the skin in towards the implant) is a complication reported by 10% to 20% of patients. Scarring has even been implicated in implant deflation (1-6% of patients; saline leaking out of the implant and “deflating” it), when fibrous tissue ingrowth into the diaphragmatic valve (the access site used to inflate the implant) causes it to become incontinent and leak. In addition, over 15% of patients undergoing augmentation will suffer from chronic pain and many of these cases are ultimately attributable to scar tissue formation. Other complications of breast augmentation surgery include late leaks, hematoma (approximately 1-6% of patients), seroma (2.5%), hypertrophic scarring (2-5%) and infections (about 1-4% of cases).

Correction can involve several options including removal of the implant, capsulotomy (cutting or surgically releasing the capsule), capsulectomy (surgical removal of the fibrous capsule), or placing the implant in a different location (i.e., from subglandular to subpectoral). Ultimately, additional surgery (revisions, capsulotomy, removal, re-implantation) is required in over 20% of augmentation patients and in over 40% of reconstruction patients, with scar formation and capsular contracture being far and away the most common cause. Procedures to break down the scar may not be sufficient, and approximately 8% of augmentations and 25% of reconstructions ultimately have the implant surgically removed.

A fibrosis-inhibiting agent or composition delivered locally from the breast implant, administered locally into the tissue surrounding the breast implant, or administered systemically to reach the breast tissue, can minimize fibrous tissue formation, encapsulation and capsular contracture. For example, attempts have been made to administer steroids either from the breast implant, or infiltrated into the intended mammary pocket, but this resulted in soft tissue atrophy and deformity. An ideal fibrosis-inhibiting agent will target only the components of the fibrous capsule and not harm the surrounding soft tissues. Incorporation of a fibrosis-inhibiting agent onto a breast implant (e.g., as a coating applied to the outer surface of the implant and/or incorporated into, and released from, the outer polymeric membrane of the implant) or into a breast implant (e.g., the agent is incorporated into the saline, gel or silicone within the implant and passively diffuses across the capsule into the surrounding tissue) may minimize or prevent fibrous contracture in response to gel or saline-containing breast implants that are placed subpectorally or subglandularly. Infiltration of a fibrosis-inhibiting agent or composition into the tissue surrounding the breast implant, or into the surgical pocket where the implant will be placed, is another strategy for preventing the formation of scar and capsular contracture in breast augmentation and reconstructive surgery. Each of these approaches for reducing complications arising from capsular contraction in breast implants is described separately herein.

Numerous breast implants are suitable for use in the practice of this invention and can be used for cosmetic and reconstructive purposes. Breast implants may be composed of a flexible soft shell filled with a fluid, such as saline solution, polysiloxane, or silicone gel. For example, the breast implant may be composed of an outer polymeric shell having a cavity filled with a plurality of hollow bodies of elastically deformable material containing a liquid saline solution. See, e.g., U.S. Pat. No. 6,099,565. The breast implant may be composed of an envelope of vulcanized silicone rubber that forms a hollow sealed water impermeable shell containing an aqueous solution of polyethylene glycol. See, e.g., U.S. Pat. No. 6,312,466. The breast implant may be composed of an envelope made from a flexible non-absorbable material and a filler material that is a shortening composition (e.g., vegetable oil). See, e.g., U.S. Pat. No. 6,156,066. The breast implant may be composed of a soft, flexible outer membrane and a partially-deformable elastic filler material that is supported by a compartmental internal structure. See, e.g., U.S. Pat. No. 5,961,552. The breast implant may be composed of a non-biodegradable conical shell filled with layers of monofilament yarns formed into resiliently compressible fabric. See, e.g., U.S. Pat. No. 6,432,138. The breast implant may be composed of a shell containing sterile continuous filler material made of continuous yarn of polyolefin or polypropylene. See, e.g., U.S. Pat. No. 6,544,287. The breast implant may be composed of an envelope containing a keratin hydrogel. See, e.g., U.S. Pat. No. 6,371,984. The breast implant may be composed of a hollow, collapsible shell formed from a flexible, stretchable material having a base portion reinforced with a resilient, non-deformable member and a cohesive filler material contained within. See, e.g., U.S. Pat. No. 5,104,409. The breast implant may be composed of a smooth, non-porous, polymeric outer envelope with an affixed non-woven, porous outer layer made of extruded fibers of polycarbonate urethane polymer, which has a soft filler material contained within. See, e.g., U.S. Pat. No. 5,376,117. The breast implant may be configured to be surgically implanted under the pectoral muscle with a second prosthesis implanted between the pectoral muscle and the breast tissue. See, e.g., U.S. Pat. No. 6,464,726. The breast implant may be composed of a homogenous silicone elastomer flexible shell of unitary construction with an interior filling and a rough-textured external surface with randomly formed interconnected cells to promote tissue ingrowth to prevent capsular contracture. See, e.g., U.S. Pat. No. 5,674,285. The breast implant may be a plastic implant with a covering of heparin, which is bonded to the surface to prevent or treat capsule formation and/or shrinkage in a blood dry tissue cavity. See, e.g., U.S. Pat. No. 4,713,073. The breast implant may be a sealed, elastic polymer envelope having a microporous structure that is filled with a viscoelastic material (e.g., salt of chondroitin sulfate) to provide a predetermined shape. See, e.g., U.S. Pat. No. 5,344,451.

Commercially available breast implant implants include those from INAMED Corporation (Santa Barbara, Calif.) that sells both Saline-Filled and Silicone-Filled Breast Implants. INAMED's Saline-Filled Breast Implants include the Style 68 Saline Matrix and Style 363LF as well as others in a variety of models, contours, shapes and sizes. INAMED's Silicone-Filled Breast Implants include the Style 10, Style 20 and Style 40 as well as others in a variety of shapes, contours and sizes. INAMED also sells breast tissue expanders, such as the INAMED Style 133 V series tissue expanders, which are used to encourage rapid tissue adherence to maximize expander immobility. Mentor Corporation (Santa Barbara, Calif.) sells the saline-filled Contour Profile Style Breast Implant (available in a variety of models, shapes, contours and sizes) and the SPECTRUM Postoperatively Adjustable Breast Implant that allows adjustment of breast size by adding or removing saline with a simple office procedure for six months post-surgery. Mentor also produces the Contour Profile® Gel (silicone) breast implant in a variety of models, shapes, contours and sizes. Breast implants such as these may benefit from release of a therapeutic agent able to reduce scarring at the implant-tissue interface to minimize the incidence of fibrous contracture. In one aspect, the breast implant is combined with a fibrosis-inhibiting agent or composition containing a fibrosis-inhibiting agent. Ways that this can be accomplished include, but are not restricted to, incorporating a fibrosis-inhibiting agent into the polymer that composes the shell of the implant (e.g., the polymer that composes the capsule of the breast implant is loaded with an agent that is gradually released from the surface), surface-coating the breast implant with an anti-scarring agent or a composition that includes an anti-scarring agent, and/or incorporating the fibrosis-inhibiting agent into the implant filling material (for example, saline, gel, silicone) such that it can diffuse across the capsule into the surrounding tissue.

Methods for incorporating fibrosis-inhibiting compositions onto or into a breast implant include (a) directly affixing to, or coating, the surface of the breast implant with a fibrosis-inhibiting composition (e.g., by either a spraying process or dipping process, with or without a carrier); (b) directly incorporating the fibrosis-inhibiting composition into the polymer that composes the outer capsule of the breast implant (e.g., by either a spraying process or dipping process, with or without a carrier); (c) by coating the breast implant with a substance such as a hydrogel which will in turn absorb the fibrosis-inhibiting composition, (d) by inserting the breast implant into a sleeve or mesh which is comprised of, or coated with, a fibrosis-inhibiting composition, (e) constructing the breast implant itself (or a portion of the implant) with a fibrosis-inhibiting composition, or (f) by covalently binding the fibrosis-inhibiting agent directly to the breast implant surface or to a linker (small molecule or polymer) that is coated or attached to the implant surface. The coating process can be performed in such a manner as to: (a) coat a portion of the breast implant; or (b) coat the entire implant with the fibrosis-inhibiting agent or composition. Specific methods of coating breast implants are described herein.

In another embodiment, the fibrosis-inhibiting agent or composition can be incorporated into the central core of the implant. As described above, the most common design of a breast implant involves an outer capsule (in a variety of shapes and sizes), which is filled with an aqueous or gelatinous material. Most commercial devices employ either saline or silicone as the “filling” material. However, numerous materials have been described for this purpose including, but not restricted to, polysiloxane, polyethylene glycol, vegetable oil, triglycerides, monofilament yarns (e.g., polyolefin, polypropylene), keratin hydrogel and chondroitin sulfate. The fibrosis inhibiting agent or composition can be incorporated into the filler material and then can diffuse through, or be actively transported across, the capsular material to reach the surrounding tissues and prevent capsular contracture. Methods of incorporating the fibrosis-inhibiting agent or composition into the central core material of the breast implant include, but are not restricted to: (a) dissolving a water soluble fibrosis-inhibiting agent into an aqueous core material (e.g., saline) at the appropriate concentration and dose; (b) using a solubilizing agent or carrier (e.g., micelles, liposomes, EDTA, a surfactant etc.) to incorporate an insoluble fibrosis-inhibiting agent into an aqueous core material at the appropriate concentration and dose; (c) dissolving a water-insoluble fibrosis-inhibiting agent into an organic solvent core material (e.g., vegetable oil, polypropylene etc.) at the appropriate concentration and dose; (d) incorporating the fibrosis-inhibiting agent into the threads (polyolefin yarns, polypropylene yarns, etc.) contained in the breast implant core; (d) incorporating, or loading, the fibrosis-inhibiting agent or composition into the central gel material (e.g., silicone gel, keratin hydrogel, chondroitin sulfate, hydrogels, etc.) at the appropriate concentration and dose; (e) formulating the fibrosis-inhibiting agent or composition into solutions, microspheres, gels, pastes, films, and/or solid particles which are then incorporated into, or dispersed in, the breast implant filler material; (f) forming a suspension of an insoluble fibrosis-inhibiting agent with an aqueous filler material; (g) forming a suspension of a aqueous soluble fibrosis-inhibiting agent and an insoluble (organic solvent) filler material; and/or (h) combinations of the above. Each of these methods illustrates an approach for combining a breast implant with a fibrosis-inhibiting (also referred to herein as an anti-scarring) agent according to the present invention. Using these or other techniques, an implant may be prepared which has a coating, where the coating is, e.g., uniform, non-uniform, continuous, discontinuous, or patterned. The coating may directly contact the implant, or it may indirectly contact the implant when there is something, e.g., a polymer layer, that is interposed between the implant and the coating that contains the fibrosis-inhibiting agent. Sustained release formulations suitable for incorporation into the core of the breast implant are described herein.

As an alternative to, or in addition to, coating or filling the implant with a composition that contains a fibrosis-inhibiting agent, a composition that includes an anti-scarring agent can be infiltrated into the space (surgically created pocket) where the breast implant will be implanted. This can be accomplished by applying the fibrosis-inhibiting agent, with or without a polymeric, non-polymeric, or secondary carrier either directly (during an open procedure) or via an endoscope: (a) to the breast implant surface (e.g., as an injectable, paste, gel or mesh) during the implantation procedure; (b) to the surface of the tissue (e.g., as an injectable, paste, gel, in situ forming gel or mesh) of the implantation pocket immediately prior to, or during, implantation of the breast implant; (c) to the surface of the breast implant and/or the tissue surrounding the implant (e.g., as an injectable, paste, gel, in situ forming gel or mesh) immediately after to the implantation of the soft tissue implant; (d) by topical application of the anti-fibrosis agent into the anatomical space where the soft tissue implant will be placed (particularly useful for this embodiment is the use of polymeric carriers which release the fibrosis-inhibiting agent over a period ranging from several hours to several weeks-fluids, suspensions, emulsions, microemulsions, microspheres, pastes, gels, microparticulates, sprays, aerosols, solid implants and other formulations which release the agent and can be delivered into the region where the implant will be inserted); (e) via percutaneous injection into the tissue surrounding the implant as a solution, as an infusate, or as a sustained release preparation; and/or (f) by any combination of the aforementioned methods.

It should be noted that certain polymeric carriers themselves can help prevent the formation of fibrous tissue around the breast implant. These carriers (to be described below) are particularly useful for infiltration into the tissue surrounding the breast implant (as described in the previous paragraph), either alone, or in combination with a fibrosis inhibiting composition. Numerous carriers suitable for the practice of this embodiment are described herein, but the following implantables are particularly preferred for infiltration into the vicinity of the implant-tissue interface and include: (a) sprayable collagen-containing formulations such as COSTASIS and crosslinked derivatized poly(ethylene glycol) collagen compositions (described, e.g., in U.S. Pat. Nos. 5,874,500 and 5,565,519 and referred to herein as “CT3” (both from Angiotech Pharmaceuticals, Inc., Canada), either alone, or loaded with a fibrosis-inhibiting agent, applied to the breast implantation site (or the breast implant surface); (b) sprayable PEG-containing formulations such as COSEAL or ADHIBIT (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.), either alone, or loaded with a fibrosis-inhibiting agent, applied to the breast implantation site (or the breast implant surface); (c) fibrinogen-containing formulations such as FLOSEAL or TISSEAL (both from Baxter Healthcare Corporation, Fremont, Calif.), either alone, or loaded with a fibrosis-inhibiting agent, applied to the breast implantation site (or the breast implant surface); (d) hyaluronic acid-containing formulations such as RESTYLANE or PERLANE (both from Q-Med AB, Sweden), HYLAFORM (Inamed Corporation, Santa Barbara, Calif.), PERLANE, SYNVISC (Biomatrix, Inc., Ridgefield, N.J.), SEPRAFILM or, SEPRACOAT (both from Genzyme Corporation), loaded with a fibrosis-inhibiting agent applied to the breast implantation site (or the breast implant surface); (e) polymeric gels for surgical implantation such as REPEL (Life Medical Sciences, Inc., Princeton, N.J.) or FLOWGEL (Baxter Healthcare Corporation) loaded with a fibrosis-inhibiting agent applied to the breast implantation site (or the breast implant surface); (f) glycol (pentaerythritol poly(ethylene glycol)ether tetra-succinimidyl glutarate (4-armed NHS-PEG) in an acidic solution (e.g., pH about 2.5) co-applied with a basic buffer (e.g., pH about 9.5 alone, or loaded with a fibrosis-inhibiting agent applied to the breast implantation site (or the breast implant surface); (g) polysaccharide gels such as the ADCON series of gels (available from Gliatech, Inc., Cleveland, Ohio) either alone, or loaded with a fibrosis-inhibiting agent, applied to the breast implantation site (or the breast implant surface); (h) electrospun material (e.g., collagen and PLGA), alone or loaded with a fibrosis-inhibiting agent, that is applied to the surface of the implant or that is placed at the site of implantation between the breast implant and the adjacent tissue; and/or (i) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) alone, or loaded with a fibrosis-inhibiting agent applied to the implantation site (or the implant surface). All of the above have the advantage of also acting as a temporary (or permanent) barrier (particularly formulations containing PEG, hyaluronic acid, and polysaccharide gels) that can help prevent the formation of fibrous tissue around the breast implant. Several of the above agents (e.g., formulations containing PEG, collagen, or fibrinogen such as COSEAL, CT3, ADHIBIT, COSTASIS, FOCALSEAL, SPRAYGEL, DURASEAL, TISSEAL AND FLOSEAL) have the added benefit of being hemostats and vascular sealants, which given the suspected role of inadequate hemostasis in the development of capsular contracture, may also be of benefit in the practice of this invention.

A preferred polymeric matrix which can be used to help prevent the formation of fibrous tissue around the breast implant, either alone or in combination with a fibrosis inhibiting agent composition, is formed from reactants comprising either one or both of pentaerythritol poly(ethylene glycol)ether tetra-sulfhydryl](4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino](4-armed amino PEG, which includes structures having a linking group(s) between an amino group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue around the breast implant.

Within various embodiments of the invention, the breast implant is coated on one aspect with a composition which inhibits fibrosis, as well as being coated with a composition or compound which promotes scarring on another aspect of the device (i.e., to affix the breast implant into the subglandular or subpectoral space). As described above, implant malposition (movement or migration of the implant after placement) can lead to a variety of complications such as asymmetry and movement below the inframammary crease, and is a leading cause of patient dissatisfaction and revision surgery. In one embodiment the breast implant is coated on the inferior surface (i.e., the surface facing the pectoralis muscle for subglandular breast implants or the surface facing the chest wall for subpectoral breast implants) with a fibrosis-promoting agent or composition, and the coated on the other surfaces (i.e., the surfaces facing the mammary tissue for subglandular breast implants or the surfaces facing the pectoralis muscle for subpectoral breast implants) with an agent or composition that inhibits fibrosis. This embodiment has the advantage of encouraging fibrosis and fixation of the breast implant into the anatomical location into which it was placed (preventing implant migration), while preventing the complications associated with encapsulation on the superficial aspects of the breast implant. Representative examples of agents that promote fibrosis and are suitable for delivery from the inferior (deep) surface of the breast implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFβ, PDGF, VEGF, bFGF, TNFα, NGF, GM-CSF, IGF-1,IL1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester (N(omega-nitro-L-arginine methyl ester)), and all-trans retinoic acid (ATRA)); as well as analogues and derivatives thereof. As an alternative to, or in addition to, coating the inferior surface of the breast implant with a composition that contains a fibrosis-promoting agent, a composition that includes a fibrosis-inducing agent can be infiltrated into the space (the base of the surgically created pocket) where the breast implant will be apposed to the underlying tissue.

In certain embodiments, the breast implant may include a fibrosis-inhibiting agent and/or an anti-microbial agent. Evidence of infection, particularly from skin flora such as S. aureus and S. epidermidis, is a common histological finding in cases of capsular contracture. Overt implant infection (occurs in about 1-4% of cases) resulting from wound infections, contaminated saline in the implant, contamination of the breast implant at the time of surgical implantation and other causes necessitates the removal of the implant. Delivery of an anti-microbial agent (e.g., antibiotics, micocycline, rifamycin, 5-FU, methotrexate, mitoxantrone, doxorubicin) as a coating, from the capsule, from the implant filler, and/or delivered into the surrounding tissue at the time of implantation, may reduce the incidence of breast implant infections and help prevent the formation of infection-induced capsular contracture. Four of the above agents (i.e., 5-FU, methotrexate, mitoxantrone, doxorubicin), as well as analogues and derivatives thereof, have the added benefit of also preventing fibrosis (as described herein).

In summary, embodiments of the present invention will create a breast implant with improved clinical outcomes and a lower incidence of common complications of breast augmentation surgery. Administration of a fibrosis-inhibitor can reduce the incidence of capsular contracture, asymmetry, skin dimpling, hardness and repeat surgical interventions (e.g., capsulotomy, capsulectomy, revisions, and removal) and improve patient satisfaction with the procedure. Administration of a fibrosis-inducing agent can reduce the incidence of migration, asymmetry and repeat surgical interventions (e.g., revisions and removal) and improve patient satisfaction. And finally, administration of an anti-infective agent can reduce the incidence of infection and capsular contracture.

C. Other Cosmetic Implants

A variety of other soft tissue cosmetic implants may be used in the practice of the invention:

1) Facial Implants

In one aspect, the soft tissue implant is a facial implant, including implants for the malar-midface region or submalar region (e.g., cheek implant). Malar and submalar augmentation is often conducted when obvious changes have occurred associated with aging (e.g., hollowing of the cheeks and ptosis of the midfacial soft tissue), midface hypoplasia (a dish-face deformity), post-traumatic and post-tumor resection deformities, and mild hemifacial microsomia. Malar and submalar augmentation may also be conducted for cosmetic purposes to provide a dramatic high and sharp cheek contour. Placement of a malar-submalar implant often enhances the result of a rhytidectomy or rhinoplasty by further improving facial balance and harmony.

There are numerous facial implants that can be used for cosmetic and reconstructive purposes. For example, the facial implant may be a thin teardrop-shaped profile with a broad head and a tapered narrow tail for the mid-facial or submalar region of the face to restore and soften the fullness of the cheeks. See, e.g., U.S. Pat. No. 4,969,901. The facial implant may be composed of a flexible material having a generally concave-curved lower surface and a convex-curved upper surface, which is used to augment the submalar region. See, e.g., U.S. Pat. No. 5,421,831. The facial implant may be a modular prosthesis composed of a thin planar shell and shims that provide the desired contour to the overlying tissue. See, e.g., U.S. Pat. No. 5,514,179. The facial implant may be composed of moldable silicone having a grid of horizontal and vertical grooves on a concave bone-facing rear surface to facilitate tissue ingrowth. See, e.g., U.S. Pat. No. 5,876,447. The facial implant may be composed of a closed-cell, cross-linked, polyethylene foam that is formed into a shell and of a shape to closely conform to the face of a human. See, e.g., U.S. Pat. No. 4,920,580. The facial implant may be a means of harvesting a dermis plug from the skin of the donor after applying a laser beam for ablating the epidermal layer of the skin thereby exposing the dermis and then inserting this dermis plug at a site of facial skin depression. See, e.g., U.S. Pat. No. 5,817,090. The facial implant may be composed of silicone-elastomer with an open-cell structure whereby the silicone elastomer is applied to the surface as a solid before the layer is cured. See, e.g., U.S. Pat. No. 5,007,929. The facial implant may be a hollow perforate mandibular or maxillary dental implant composed of a trans osseous bolt receptor that is secured against the alveolar ridge by contiguous straps. See, e.g., U.S. Pat. No. 4,828,492.

Commercially available facial implants suitable for the practice of this invention include: Tissue Technologies, Inc. (San Francisco, Calif.) sells the ULTRASOFT-RC Facial Implant which is made of soft, pliable synthetic e-PTFE used for soft tissue augmentation of the face. Tissue Technologies, Inc. also sells the ULTRASOFT, which is made of tubular e-PTFE indicated for soft tissue augmentation of the facial area and is particularly well suited for use in the lip border and the nasolabial folds. A variety of facial implants are available from ImplanTech Associates including the BINDER SUBMALAR facial implant, the BINDER SUBMALAR II FACIAL IMPLANT, the TERINO MALAR SHELL, the COMBINED SUBMALAR SHELL, the FLOWERS TEAR TROUGH implant; solid silicone facial and malar implants from Allied Biomedical; the Subcutaneous Augmentation Material (S.A.M.), made from microporous ePTFE which supports rapid tissue incorporation and preformed TRIMENSIONAL 3-D Implants from W. L. Gore & Associates, Inc.

Facial implants such as these may benefit from release of a therapeutic agent able to reduce scarring at the implant-tissue interface to minimize the occurrence of fibrous contracture. Incorporation of a fibrosis-inhibiting agent into or onto a facial implant (e.g., as a coating applied to the surface, incorporated into the pores of a porous implant, incorporated into the implant, incorporated into the polymers that compose the outer capsule of the implant and/or incorporated into the polymers that compose the inner portions of the implant) may minimize or prevent fibrous contracture in response to facial implants that are placed in the face for cosmetic or reconstructive purposes. The fibrosis-inhibiting agent can reduce the incidence of capsular contracture, asymmetry, skin dimpling, hardness and repeat surgical interventions (e.g., capsulotomy, capsulectomy, revisions, and removal) and improve patient satisfaction with the procedure. As an alternative to this, or in addition to this, a composition that includes an anti-scarring agent can be infiltrated into the space where the implant will be surgically implanted.

Regardless of the specific design features, for a facial implant to be effective in cosmetic or reconstructive procedures, the implant must be accurately positioned within the body. Facial implants can migrate following surgery and it is important to achieve attachment of the implant to the underlying periosteum and bone tissue. Facial implants have been described that have a grid of horizontal and vertical grooves on a concave bone-facing rear surface to facilitate tissue ingrowth. Within various embodiments of the invention, the facial implant is coated on one aspect with a composition which inhibits fibrosis, as well as being coated with a composition or compound which promotes scarring on another aspect of the device (i.e., to affix the facial implant to the underlying bone). Facial implant malposition (movement or migration of the implant after placement) can lead to asymmetry and is a leading cause of patient dissatisfaction and revision surgery. In one embodiment the facial implant is coated on the inferior surface (i.e., the surface facing the periosteum and bone) with a fibrosis-inducing agent or composition, and coated on the other surfaces (i.e., the surfaces facing the skin and subcutaneous tissues) with an agent or composition that inhibits fibrosis. This embodiment has the advantage of encouraging fibrosis and fixation of the facial implant into the anatomical location into which it was placed (preventing implant migration), while preventing the complications associated with encapsulation on the superficial aspects of the implant. Representative examples of agents that promote fibrosis and are suitable for delivery from the inferior (deep) surface of the facial implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester) (L-NAME), and all-trans retinoic acid (ATRA)); as well as analogues and derivatives thereof. As an alternative to, or in addition to, coating the inferior surface of the facial implant with a composition that contains a fibrosis-promoting agent, a composition that includes a fibrosis-inducing agent can be infiltrated onto the surface or space (e.g., the surface of the periosteum) where the facial implant will be apposed to the underlying tissue.

In certain embodiments, the facial implant may include a fibrosis-inhibiting agent and/or an anti-microbial agent. Delivery of an anti-microbial agent (e.g., antibiotics, 5-FU, methotrexate, mitoxantrone, doxorubicin) as a coating, from the capsule, from the implant filler, and/or delivered into the surrounding tissue at the time of implantation, may reduce the incidence of implant infections. Four of the above agents (5-FU, methotrexate, mitoxantrone, doxorubicin) have the added benefit of also preventing fibrosis (as are described herein).

2) Chin and Mandibular Implants

In one aspect, the soft tissue implant is a chin or mandibular implant. Incorporation of a fibrosis-inhibiting agent into or onto the chin or mandibular implant, or infiltration of the agent into the tissue around a chin or mandibular implant, may minimize or prevent fibrous contracture in response to implants placed for cosmetic or reconstructive purposes.

Numerous chin and mandibular implants can be used for cosmetic and reconstructive purposes. For example, the chin implant may be a solid, crescent-shaped implant tapering bilaterally to form respective tails and having a curved projection surface positioned on the outer mandible surface to create a natural chin profile and form a build-up of the jaw. See, e.g., U.S. Pat. No. 4,344,191. The chin implant may be a solid crescent with an axis of symmetry of forty-five degrees, which has a softer, lower durometer material at the point of the chin to simulate the fat pad. See, e.g., U.S. Pat. No. 5,195,951. The chin implant may have a concave posterior surface to cooperate with the irregular bony surface of the mandible and convex anterior surface with a protuberance for augmenting and providing a natural chin contour. See, e.g., U.S. Pat. No. 4,990,160. The chin implant may have a porous convex surface made of polytetrafluoroethylene having void spaces of size adequate to allow soft tissue ingrowth, while the concave surface made of silicone is nonporous to substantially prevent ingrowth of bony tissue. See, e.g., U.S. Pat. No. 6,277,150.

Examples of commercially available chin or mandibular implants include: the TERINO EXTENDED ANATOMICAL chin implant, the GLASGOLD WAFER, the FLOWERS MANDIBULAR GLOVE, MITTELMAN PRE JOWL-CHIN, GLASGOLD WAFER implants, as well as other models from ImplantTech Associates; and the solid silicone chin implants from Allied Biomedical.

Chin or mandibular implants such as these may benefit from release of a therapeutic agent able to reduce scarring at the implant-tissue interface to minimize the occurrence of fibrous contracture. Incorporation of a fibrosis-inhibiting agent into or onto a chin or mandibular implant (mandibular implant (e.g., as a coating applied to the surface, incorporated into the pores of a porous implant, incorporated into the implant, incorporated into the polymers that compose the outer capsule of the implant and/or incorporated into the polymers that compose the inner portions of the implant) may minimize or prevent fibrous contracture in response to implants that are placed in the chin or mandible for cosmetic or reconstructive purposes. The fibrosis-inhibiting agent can reduce the incidence of capsular contracture, asymmetry, skin dimpling, hardness and repeat surgical interventions (e.g., capsulotomy, capsulectomy, revisions, and removal) and improve patient satisfaction with the procedure. As an alternative to this, or in addition to this, a composition that includes an anti-scarring agent can be infiltrated into the space where the implant will be implanted.

Regardless of the specific design features, for a chin or mandibular implant to be effective in cosmetic or reconstructive procedures, the implant must be accurately positioned on the face. Chin or mandibular implants can migrate following surgery and it is important to achieve attachment of the implant to the underlying periosteum and bone tissue. Chin or mandibular implant malposition (movement or migration of the implant after placement) can lead to asymmetry and is a leading cause of patient dissatisfaction and revision surgery. Within various embodiments of the invention, the chin or mandibular implant is coated on one aspect with a composition which inhibits fibrosis, as well as being coated with a composition or compound which promotes scarring (or fibrosis) on another aspect of the device (Le., to affix the implant to the underlying mandible). In one embodiment the chin or mandibular implant is coated on the inferior surface (i.e., the surface facing the periosteum and the mandible) with a fibrosis-inducing agent or composition, and coated on the other surfaces (i.e., the surfaces facing the skin and subcutaneous tissues) with an agent or composition that inhibits fibrosis. This embodiment has the advantage of encouraging fibrosis and fixation of the chin or mandibular implant to the underlying mandible (preventing implant migration), while preventing the complications associated with encapsulation on the superficial aspects of the implant. Representative examples of agents that promote fibrosis and are suitable for delivery from the inferior (deep) surface of the chin or mandibular implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFβ, PDGF, VEGF, bFGF, TNFβ, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester) (L-NAME), and all-trans retinoic acid (ATRA)); as well as analogues and derivatives thereof. As an alternative to, or in addition to, coating the inferior surface of the chin or mandibular implant with a composition that contains a fibrosis-inducing agent, a composition that includes a fibrosis-inducing agent can be infiltrated onto the surface or space (e.g., the surface of the periosteum) where the implant will be apposed to the underlying tissue.

In certain embodiments, the chin or mandibular implant may include a fibrosis-inhibiting agent and/or an anti-microbial agent. Delivery of an anti-microbial agent (e.g., antibiotics, minocycline, 5-FU, methotrexate, mitoxantrone, doxorubicin) as a coating, from the capsule, from the implant filler, and/or delivered into the surrounding tissue at the time of implantation, may reduce the incidence of implant infections. Four of the above agents (5-FU, methotrexate, mitoxantrone, doxorubicin) have the added benefit of also preventing fibrosis (as described herein).

3) Nasal Implants

In one aspect, the soft tissue implant for use in the practice of the invention is a nasal implant. Incorporation of a fibrosis-inhibiting agent into or onto the nasal implant, or infiltration of the agent into the tissue around a nasal implant, may minimize or prevent fibrous contracture in response to implants placed for cosmetic or reconstructive purposes.

Numerous nasal implants are suitable for the practice of this invention that can be used for cosmetic and reconstructive purposes. For example, the nasal implant may be elongated and contoured with a concave surface on a selected side to define a dorsal support end that is adapted to be positioned over the nasal dorsum to augment the frontal and profile views of the nose. See, e.g., U.S. Pat. No. 5,112,353. The nasal implant may be composed of substantially hard-grade silicone configured in the form of an hourglass with soft silicone at the tip. See, e.g., U.S. Pat. No. 5,030,232. The nasal implant may be composed of essentially a principal component being an aryl acrylic hydrophobic monomer with the remainder of the material being a cross-linking monomer and optionally one or more additional components selected from the group consisting of UV-light absorbing compounds and blue-light absorbing compounds. See, e.g., U.S. Pat. No. 6,528,602. The nasal implant may be composed of a hydrophilic synthetic cartilaginous material with pores of controlled size randomly distributed throughout the body for replacement of fibrous tissue. See, e.g., U.S. Pat. No. 4,912,141.

Examples of commercially available nasal implants suitable for use in the practice of this invention include the FLOWERS DORSAL, RIZZO DORSAL, SHIRAKABE, and DORSAL COLUMELLA nasal implants from ImplantTech Associates and solid silicone nasal implants from Allied Biomedical.

Nasal implants such as these may benefit from release of a therapeutic agent able to reduce scarring at the implant-tissue interface to minimize the occurrence of fibrous contracture. Incorporation of a fibrosis-inhibiting agent into or onto a nasal implant (e.g., as a coating applied to the surface, incorporated into the pores of a porous implant, incorporated into the implant, incorporated into the polymers that compose the outer capsule of the implant and/or incorporated into the polymers that compose the inner portions of the implant) may minimize or prevent fibrous contracture in response to implants that are placed in the nose for cosmetic or reconstructive purposes. The fibrosis-inhibiting agent can reduce the incidence of capsular contracture, asymmetry, skin dimpling, hardness and repeat surgical interventions (e.g., capsulotomy, capsulectomy, revisions, and removal) and improve patient satisfaction with the procedure. As an alternative to this, or in addition to this, a composition that includes an anti-scarring agent can be infiltrated into the space where the implant will be implanted.

Regardless of the specific design features, for a nasal implant to be effective in cosmetic or reconstructive procedures, the implant must be accurately positioned on the face. Nasal implants can migrate following surgery and it is important to achieve attachment of the implant to the underlying cartilage and/or bone tissue in the nose. Nasal implant malposition (movement or migration of the implant after placement) can lead to asymmetry and is a leading cause of patient dissatisfaction and revision surgery. Within various embodiments of the invention, the nasal implant is coated on one aspect with a composition which inhibits fibrosis, as well as being coated with a composition or compound which promotes scarring on another aspect of the device (i.e., to affix the implant to the underlying cartilage or bone of the nose). In one embodiment the nasal implant is coated on the inferior surface (i.e., the surface facing the nasal cartilage and/or bone) with a fibrosis-inducing agent or composition, and coated on the other surfaces (i.e., the surfaces facing the skin and subcutaneous tissues) with an agent or composition that inhibits fibrosis. This embodiment has the advantage of encouraging fibrosis and fixation of the nasal implant to the underlying nasal cartilage or bone (preventing implant migration), while preventing the complications associated with encapsulation on the superficial aspects of the implant. Representative examples of agents that promote fibrosis and are suitable for delivery from the inferior (deep) surface of the nasal implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester) (L-NAME), and all-trans retinoic acid (ATRA)); as well as analogues and derivatives thereof. As an alternative to, or in addition to, coating the inferior surface of the nasal implant with a composition that contains a fibrosis-inducing agent, a composition that includes a fibrosis-inducing agent can be infiltrated onto the surface or space (e.g., the surface of the nasal cartilage or bone) where the implant will be apposed to the underlying tissue.

In certain embodiments, the nasal implant may include a fibrosis-inhibiting agent and/or an anti-microbial agent. Delivery of an anti-microbial agent (e.g., antibiotics, 5-FU, methotrexate, mitoxantrone, doxorubicin) as a coating, from the capsule, from the implant filler, and/or delivered into the surrounding tissue at the time of implantation, may reduce the incidence of implant infections. Four of the above agents (5-FU, methotrexate, mitoxantrone, doxorubicin) have the added benefit of also preventing fibrosis (as will be described herein).

4) Lip Implants

In one aspect, the soft tissue implant suitable for combining with a fibrosis-inhibiting agent is a lip implant. Incorporation of a fibrosis-inhibiting agent into or onto the lip implant, or infiltration of the agent into the tissue around a lip implant, may minimize or prevent fibrous contracture in response to implants placed for cosmetic or reconstructive purposes.

Numerous lip implants can be used for cosmetic and reconstructive purposes. For example, the lip implant may be composed of non-biodegradable expanded, fibrillated polytetrafluoroethylene having an interior cavity extending longitudinally whereby fibrous tissue ingrowth may occur to provide soft tissue augmentation. See, e.g., U.S. Pat. Nos. 5,941,910 and 5,607,477. The lip implant may comprise soft, malleable, elastic, non-resorbing prosthetic particles that have a rough, irregular surface texture, which are dispersed in a non-retentive compatible physiological vehicle. See, e.g., U.S. Pat. No. 5,571,182.

Commercially available lip implants suitable for use in the present invention include SOFTFORM from Tissue Technologies, Inc. (San Francisco, Calif.), which has a tube-shaped design made of synthetic ePTFE; ALLODERM sheets (Allograft Dermal Matrix Grafts), which are sold by LifeCell Corporation (Branchburg, N.J.) may also be used as an implant to augment the lip. ALLODERM sheets are very soft and easily augment the lip in a diffuse manner. W.L. Gore and Associates (Newark, Del.) sells solid implantable threads that may also be used for lip implants.

Lip implants such as these may benefit from release of a therapeutic agent able to reduce scarring at the implant-tissue interface to minimize the occurrence of fibrous contracture. Incorporation of a fibrosis-inhibiting agent into or onto a lip implant (e.g., as a coating applied to the surface, incorporated into the pores of a porous implant, incorporated into the implant, incorporated into the polymers that compose the outer capsule of the implant, incorporated into the threads or sheets that make up the lip implant and/or incorporated into the polymers that compose the inner portions of the implant) may minimize or prevent fibrous contracture in response to implants that are placed in the lips for cosmetic or reconstructive purposes. The fibrosis-inhibiting agent can reduce the incidence of asymmetry, skin dimpling, hardness and repeat interventions and improve patient satisfaction with the procedure. As an alternative to this, or in addition to this, a composition that includes an anti-scarring agent can be injected or infiltrated into the lips directly.

Within various embodiments of the invention, the lip implant is coated on one aspect with a composition that inhibits fibrosis, as well as being coated with a composition or compound that promotes fibrous tissue ingrowth on another aspect. This embodiment has the advantage of encouraging fibrosis and fixation of the lip implant to the adjacent tissues, while preventing the complications associated with fibrous encapsulation on the superficial aspects of the implant. Representative examples of agents that promote fibrosis and are suitable for delivery from the inferior (deep) surface of the lip implant include silk, wool, silica, bleomycin, neomycin, talcum powder, metallic beryllium, calcium phosphate, calcium sulfate, calcium carbonate, hydroxyapatite, copper, inflammatory cytokines (e.g., wherein the inflammatory cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester) (L-NAME), and all-trans retinoic acid (ATRA)); as well as analogues and derivatives thereof. As an alternative to, or in addition to, coating the inferior surface of the lip implant with a composition that contains a fibrosis-inducing agent, a composition that includes a fibrosis-inducing agent can be injected directly into the lip where the implant will be placed.

In certain embodiments, the lip implant may include a fibrosis-inhibiting agent and/or an anti-microbial agent. Delivery of an anti-microbial agent (e.g., antibiotics, 5-FU, methotrexate, mitoxantrone, doxorubicin) as a coating, from the surface, from the implant, and/or injected into the surrounding tissue at the time of implantation, may reduce the incidence of lip implant infections. Four of the above agents (5-FU, methotrexate, mitoxantrone, doxorubicin) have the added benefit of also preventing fibrosis (as will be described herein).

5) Pectoral Implants

In one aspect, the soft tissue implant suitable for combining with a fibrosis-inhibitor is a pectoral implant. Incorporation of a fibrosis-inhibiting agent into or onto the pectoral implant, or infiltration of the agent into the tissue around a lip implant, may minimize or prevent fibrous contracture in response to implants placed for cosmetic or reconstructive purposes.

There are numerous pectoral implants that can be combined with a fibrosis-inhibiting agent and used for cosmetic and reconstructive purposes. For example, the pectoral implant may be composed of a unitary rectangular body having a slightly concave cross-section that is divided by edges into sections. See, e.g., U.S. Pat. No. 5,112,352. The pectoral implant may be composed of a hollow shell formed of a flexible elastomeric envelope that is filled with a gel or viscous liquid containing polyacrylamide and derivatives of polyacrylamide. See, e.g., U.S. Pat. No. 5,658,329.

Commercially available pectoral implants suitable for use in the present invention include solid silicone implants from Allied Biomedical. Pectoral implants such as these may benefit from release of a therapeutic agent able to reduce scarring at the implant-tissue interface to minimize the incidence of fibrous contracture. In one aspect, the pectoral implant is combined with a fibrosis-inhibiting agent or composition containing a fibrosis-inhibiting agent. Ways that this can be accomplished include, but are not restricted to, incorporating a fibrosis-inhibiting agent into the polymer that composes the shell of the implant (e.g., the polymer that composes the capsule of the pectoral implant is loaded with an agent that is gradually released from the surface), surface-coating the pectoral implant with an anti-scarring agent or a composition that includes an anti-scarring agent, and/or incorporating the fibrosis-inhibiting agent into the implant filling material (saline, gel, silicone) such that it can diffuse across the capsule into the surrounding tissue. As an alternative to this, or in addition to this, a composition that includes an anti-scarring agent can be infiltrated into the space where the pectoral implant will be implanted.

Within various embodiments of the invention, the pectoral implant is coated on one aspect with a composition which inhibits fibrosis, as well as being coated with a composition or compound which promotes scarring on another aspect of the device (i.e., to affix the pectoral implant into the subpectoral space). As described previously, implant malposition (movement or migration of the implant after placement) can lead to a variety of complications such as asymmetry, and is a leading cause of patient dissatisfaction and revision surgery. In one embodiment the pectoral implant is coated on the inferior surface (i.e., the surface facing the chest wall) with a fibrosis-promoting agent or composition, and the coated on the other surfaces (i.e., the surfaces facing the pectoralis muscle) with an agent or composition that inhibits fibrosis. This embodiment has the advantage of encouraging fibrosis and fixation of the pectoral implant into the anatomical location into which it was placed (preventing implant migration), while preventing the complications associated with encapsulation on the superficial aspects of the pectoral implant. Representative examples of agents that promote fibrosis and are suitable for delivery from the inferior (deep) surface of the pectoral implant include silk, wool, silica, bleomycin, neomycin, talcum powder, metallic beryllium, calcium phosphate, calcium sulfate, calcium carbonate, hydroxyapatite, copper, cytokines (e.g., wherein the cytokine is selected from the group consisting of bone morphogenic proteins, demineralized bone matrix, TGFβ, PDGF, VEGF, bFGF, TNFα, NGF, GM-CSF, IGF-1, IL-1-β, IL-8, IL-6, and growth hormone), agents that stimulate cell proliferation (e.g., wherein the agent that stimulates cell proliferation is selected from the group consisting of dexamethasone, isotretinoin, 17-β-estradiol, estradiol, 1-α-25 dihydroxyvitamin D₃, diethylstibesterol, cyclosporine A, N(omega-nitro-L-arginine methyl ester) (L-NAME), and all-trans retinoic acid (ATRA)); as well as analogues and derivatives thereof. As an alternative to, or in addition to, coating the inferior surface of the pectoral implant with a composition that contains a fibrosis-promoting agent, a composition that includes a fibrosis-inducing agent can be infiltrated into the space (the base of the surgically created subpectoral pocket) where the pectoral implant will be apposed to the underlying tissue.

In certain embodiments, the pectoral implant may include a fibrosis-inhibiting agent and/or an anti-microbial agent. Delivery of an anti-microbial agent (e.g., antibiotics, 5-FU, methotrexate, mitoxantrone, doxorubicin) as a coating, from the capsule, from the implant filler, and/or delivered into the surrounding tissue at the time of implantation, may reduce the incidence of pectoral implant infections and help prevent the formation of infection-induced capsular contracture. Four of the above anti-infective agents (5-FU, methotrexate, mitoxantrone, doxorubicin), as well as analogues and derivatives thereof, have the added benefit of also preventing fibrosis (as will be described herein).

6) Autogenous Tissue Implants

In one aspect, the soft tissue implant suitable for use with a fibrosis-inhibitor is an autogenous tissue implant, which includes, without limitation, adipose tissue, autogenous fat implants, dermal implants, dermal or tissue plugs, muscular tissue flaps and cell extraction implants. Adipose tissue implants may also be known as autogenous fat implants, fat grafting, free fat transfer, autologous fat transfer/transplantation, dermal fat implants, liposculpture, lipostructure, volume restoration, micro-lipoinjection and fat injections.

Autogenous tissue implants have been used for decades for soft tissue augmentation in plastic and reconstructive surgery. Autogenous tissue implants may be used, for example, to enlarge a soft tissue site (e.g., breast or penile augmentation), to minimize facial scarring (e.g., acne scars), to improve facial volume in diseases (e.g., hemifacial atrophy), and to minimize facial aging, such as sunken cheeks and facial lines (e.g., wrinkles). These injectable autogenous tissue implants are biocompatible, versatile, stable, long-lasting and natural-appearing. Autogenous tissue implants involve a simple procedure of removing tissue or cells from one area of the body (e.g., surplus fat cells from abdomen or thighs) and then re-implanted them in another area of the body that requires reconstruction or augmentation. Autogenous tissue is soft and feels natural. Autogenous soft tissue implants may be composed of a variety of connective tissues, including, without limitation, adipose or fat, dermal tissue, fibroblast cells, muscular tissue or other connective tissues and associated cells. An autogenous tissue implant is introduced to correct a variety of deficiencies, it is not immunogenic, and it is readily available and inexpensive.

In one aspect, autogenous tissue implants may be composed of fat or adipose. The extraction and implantation procedure of adipose tissue involves the aspiration of fat from the subcutaneous layer, usually of the abdominal wall by means of a suction syringe, and then injected it into the subcutaneous tissues overlying a depression. Autologous fat is commonly used as filler for depressions of the body surface (e.g., for bodily defects or cosmetic purposes), or it may be used to protect other tissue (e.g., protection of the nerve root following surgery). Fat grafts may also be used for body prominences that require padding of soft tissue to prevent sensitivity to pressure. When fat padding is lacking, the overlying skin may be adherent to the bone, leading to discomfort and even pain, which occurs, for example, when a heel spur or bony projection occurs on the plantar region of the heel bone (also known as the calcaneous). In this case, fat grafting may provide the interposition of the necessary padding between the bone and the skin. U.S. Pat. No. 5,681,561 describes, for example, an autogenous fat graft that includes an anabolic hormone, amino acids, vitamins, and inorganic ions to improve the survival rate of the lipocytes once implanted into the body.

In another aspect, autogenous tissue implants may be composed of pedicle flaps that typically originate from the back (e.g., latissimus dorsi myocutaneous flap) or the abdomen (e.g., transverse rectus abdominus myocutaneous or TRAM flap). Pedicle flaps may also come from the buttocks, thigh or groin. These flaps are detached from the body and then transplanted by reattaching blood vessels using microsurgical procedures. These muscular tissue flaps are most frequently used for post-mastectomy closure and reconstruction. Some other common closure applications for muscular tissue flaps include coverage of defects in the head and neck area, especially defects created from major head and neck cancer resection; additional applications include coverage of chest wall defects other than mastectomy deformities. The latissimus dorsi may also be used as a reverse flap, based upon its lumbar perforators, to close congenital defects of the spine such as spina bifida or meningomyelocele. For example, U.S. Pat. No. 5,765,567 describes methodology of using an autogenous tissue implant in the form of a tissue flap having a cutaneous skin island that may be used for contour correction and enlargement for the reconstruction of breast tissue. The tissue flap may be a free flap or a flap attached via a native vascular pedicle.

In another aspect, the autogenous tissue implant may be a suspension of autologous dermal fibroblasts that may be used to provide cosmetic augmentation. See, e.g., U.S. Pat. Nos. 5,858,390; 5,665,372 and 5,591,444. These U.S. patents describes a method for correcting cosmetic and aesthetic defects in the skin by the injection of a suspension of autologous dermal fibroblasts into the dermis and subcutaneous tissue subadjacent to the defect. Typical defects that can be corrected by this method include rhytids, stretch marks, depressed scars, cutaneous depressions of non-traumatic origin, scaring from acne vulgaris, and hypoplasia of the lip. The fibroblasts that are injected are histocompatible with the subject and have been expanded by passage in a cell culture system for a period of time in protein free medium.

In another aspect, the autogenous tissue implant may be a dermis plug harvested from the skin of the donor after applying a laser beam for ablating the epidermal layer of the skin thereby exposing the dermis and then inserting this dermis plug at a site of facial skin depressions. See, e.g., U.S. Pat. No. 5,817,090. This autogenous tissue implant may be used to treat facial skin depressions, such as acne scar depression and rhytides. Dermal grafts have also been used for correction of cutaneous depressions where the epidermis is removed by dermabrasion.

As is the case for other types of synthetic implants (described above), autogenous tissue implants also have a tendency to migrate, extrude, become infected, or cause painful and deforming capsular contractures. Incorporation of a fibrosis-inhibiting agent into or onto an autogenous tissue implant may minimize or prevent fibrous contracture in response to autogenous tissue implants that are placed in the body for cosmetic or reconstructive purposes.

Autogenous tissue implants such as these may benefit from release of a therapeutic agent able to reducing scarring at the implant-tissue interface to minimize fibrous encapsulation. In one aspect, the implant includes, or is coated with, an anti-scarring agent or a composition that includes an anti-scarring agent. As an alternative to this, or in addition to this, a composition that includes an anti-scarring agent can be injected or infiltrated into the space where the implant will be implanted.

Although numerous soft tissue implants have been described above, all possess similar design features and cause similar unwanted tissue reactions following implantation. It should be obvious to one of skill in the art that commercial soft tissue implants not specifically cited above as well as next-generation and/or subsequently-developed commercial soft tissue implant products are to be anticipated and are suitable for use under the present invention. The cosmetic implant should be positioned in a very precise manner to ensure that augmentation is achieved correct anatomical location in the body. All, or parts, of a cosmetic implant can migrate following surgery, or excessive scar tissue growth can occur around the implant, which can lead to a reduction in the performance of these devices. Soft tissue implants that release a therapeutic agent for reducing scarring at the implant-tissue interface can be used to increase the efficacy and/or the duration of activity of the implant (particularly for fully-implanted, battery-powered devices). In one aspect, the present invention provides soft tissue implants that include an anti-scarring agent or a composition that includes an anti-scarring agent. Numerous polymeric and non-polymeric delivery systems for use in soft tissue implants have been described above. These compositions can further include one or more fibrosis-inhibiting agents such that the overgrowth of granulation or fibrous tissue is inhibited or reduced.

7) Combining Fibrosis-Inhibitors with Soft Tissue Implants

A variety of soft tissue implants including facial implants, chin and mandibular implants, nasal implants, lip implants, pectoral implants, autogenous tissue implants and breast implants are described herein for combining with a fibrosis-inhibitor. Although available in a plethora of shapes and sizes, the majority of soft tissue implants are made for the same materials and similar design features. Specifically, many soft tissue implants feature an outer capsule filled with saline, silicone or other gelatinous material.

In general, methods for incorporating fibrosis-inhibiting compositions onto or into these soft tissue implants include (a) directly affixing to, or coating, the surface of the soft tissue implant with a fibrosis-inhibiting composition (e.g., by either a spraying process or dipping process, with or without a carrier); (b) directly incorporating the fibrosis-inhibiting composition into the polymer that composes the outer capsule of the soft tissue implant (e.g., by either a spraying process or dipping process, with or without a carrier); (c) by coating the soft tissue implant with a substance such as a hydrogel which will in turn absorb the fibrosis-inhibiting composition, (d) by inserting the soft tissue implant into a sleeve or mesh which is comprised of, or coated with, a fibrosis-inhibiting composition, (e) constructing the soft tissue implant itself (or a portion of the implant) with a fibrosis-inhibiting composition, or (f) by covalently binding the fibrosis-inhibiting agent directly to the soft tissue implant surface or to a linker (small molecule or polymer) that is coated or attached to the implant surface. The coating process can be performed in such a manner as to: (a) coat a portion of the soft tissue implant; or (b) coat the entire implant with the fibrosis-inhibiting agent or composition.

In another embodiment, the fibrosis-inhibiting agent or composition can be incorporated into the central core of the implant. As described above, the most common design of a soft tissue implant involves an outer capsule (in a variety of shapes and sizes) that is filled with an aqueous or gelatinous material. Many commercial devices employ either saline or silicone as the “filling” material. However, numerous materials have been described for this purpose including, but not restricted to, polysiloxane, polyethylene glycol, vegetable oil, monofilament yarns (e.g., polyolefin, polypropylene), keratin hydrogel and chondroitin sulfate. The fibrosis inhibiting agent or composition can be incorporated into the filler material and then can diffuse through, or be actively transported across, the capsular material to reach the surrounding tissues and prevent capsular contracture. Methods of incorporating the fibrosis-inhibiting agent or composition into the central core material of the soft tissue implant include, but are not restricted to: (a) dissolving a water soluble fibrosis-inhibiting agent into an aqueous core material (e.g., saline) at the appropriate concentration and dose; (b) using a solubilizing agent or carrier (e.g., micelles, liposomes, EDTA, a surfactant etc.) to incorporate an insoluble fibrosis-inhibiting agent into an aqueous core material at the appropriate concentration and dose; (c) dissolving a water-insoluble fibrosis-inhibiting agent into an organic solvent core material (e.g., vegetable oil, polypropylene etc.) at the appropriate concentration and dose; (d) incorporating the fibrosis-inhibiting agent into the threads (PTFE, polyolefin yarns, polypropylene yarns, etc.) contained in the soft tissue implant core; (d) incorporating, or loading, the fibrosis-inhibiting agent or composition into the central gel material (e.g., silicone gel, keratin hydrogel, chondroitin sulfate, hydrogels, etc.) at the appropriate concentration and dose; (e) formulating the fibrosis-inhibiting agent or composition into solutions, microspheres, gels, pastes, films, and/or solid particles which are then incorporated into, or dispersed in, the soft tissue implant filler material; (f) forming a suspension of an insoluble fibrosis-inhibiting agent with an aqueous filler material; (g) forming a suspension of a aqueous soluble fibrosis-inhibiting agent and an insoluble (organic solvent) filler material; and/or (h) combinations of the above. Each of these methods illustrates an approach for combining a breast implant with a fibrosis-inhibiting (also referred to herein as an anti-scarring) agent according to the present invention. Using these or other techniques, an implant may be prepared that has a coating, where the coating is, e.g., uniform, non-uniform, continuous, discontinuous, or patterned. The coating may directly contact the implant, or it may indirectly contact the implant when there is something, e.g., a polymer layer, that is interposed between the implant and the coating that contains the fibrosis-inhibiting agent. Sustained release formulations suitable for incorporation into the core of the breast implant are described herein.

For porous implants, the fibrosis-inhibiting agent can be incorporated into a biodegradable polymer (e.g., PLGA, PLA, PCL, POLYACTIVE, tyrosine-based polycarbonates) that is then applied to the porous implant as a solution (sprayed or dipped) or in the molten state.

In yet another aspect, anti-scarring agent may be located within pores or voids of the soft tissue implant. For example, a soft tissue implant may be constructured to have cavities (e.g., divets or holes), grooves, lumen(s), pores, channels, and the like, which form voids or pores in the body of the implant. These voids may be filled (partially or completely) with a fibrosis-inhibiting agent or a composition that comprises a fibrosis-inhibiting agent.

In one aspect, a soft tissue implant may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot that can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

As an alternative to, or in addition to, coating or filling the soft tissue implant with a composition that contains a fibrosis-inhibiting agent, the active agent can be administered to the area via local or systemic drug-delivery techniques. A variety of drug-delivery technologies are available for systemic, regional and local delivery of therapeutic agents. Several of these techniques may be suitable to achieve preferentially elevated levels of fibrosis-inhibiting agents in the vicinity of the soft tissue implant, including: (a) using drug-delivery catheters for local, regional or systemic delivery of fibrosis-inhibiting agents to the tissue surrounding the implant. Typically, drug delivery catheters are advanced through the circulation or inserted directly into tissues under radiological guidance until they reach the desired anatomical location. The fibrosis inhibiting agent can then be released from the catheter lumen in high local concentrations in order to deliver therapeutic doses of the drug to the tissue surrounding the implant; (b) drug localization techniques such as magnetic, ultrasonic or MRI-guided drug delivery; (c) chemical modification of the fibrosis-inhibiting drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inhibiting drug or formulation designed to localize the drug to areas of bleeding or disrupted vasculature; and/or (e) direct injection of the fibrosis-inhibiting agent, for example, under endoscopic vision.

As an alternative to, or in addition to, the above methods of administering a fibrosis-inhibiting agent, a composition that includes an anti-scarring agent can be infiltrated into the space (surgically created pocket) where the soft tissue implant will be implanted. This can be accomplished by applying the fibrosis-inhibiting agent, with or without a polymeric, non-polymeric, or secondary carrier either directly (during an open procedure) or via an endoscope: (a) to the soft tissue implant surface (e.g., as an injectable, paste, gel or mesh) during the implantation procedure; (b) to the surface of the tissue (e.g., as an injectable, paste, gel, in situ forming gel or mesh) of the implantation pocket immediately prior to, or during, implantation of the soft tissue implant; (c) to the surface of the soft tissue implant and/or the tissue surrounding the implant (e.g., as an injectable, paste, gel, in situ forming gel or mesh) immediately after to the implantation of the soft tissue implant; (d) by topical application of the anti-fibrosis agent into the anatomical space where the soft tissue implant will be placed (particularly useful for this embodiment is the use of polymeric carriers which release the fibrosis-inhibiting agent over a period ranging from several hours to several weeks-fluids, suspensions, emulsions, microemulsions, microspheres, pastes, gels, microparticulates, sprays, aerosols, solid implants and other formulations which release the agent and can be delivered into the region where the implant will be inserted); (e) via percutaneous injection into the tissue surrounding the implant as a solution, as an infusate, or as a sustained release preparation; and/or (f) by any combination of the aforementioned methods.

It should be noted that certain polymeric carriers themselves can help prevent the formation of fibrous tissue around the soft tissue implant. These carriers (to be described below) are particularly useful for the practice of this embodiment, either alone, or in combination with a fibrosis-inhibiting composition. The following polymeric carriers can be infiltrated (as described previously) into the vicinity of the implant-tissue interface and include: (a) sprayable collagen-containing formulations such as COSTASIS or CT3 (Angiotech Pharmaceuticals, Inc., Canada), either alone, or loaded with a fibrosis-inhibiting agent, applied to the implantation site (or the soft tissue implant surface); (b) sprayable PEG-containing formulations such as COSEAL and ADHIBIT (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.), either alone, or loaded with a fibrosis-inhibiting agent, applied to the implantation site (or the soft tissue implant surface); (c) fibrinogen-containing formulations such as FLOSEAL or TISSEAL (both from Baxter Healthcare Corporation, Fremont, Calif.), either alone, or loaded with a fibrosis-inhibiting agent, applied to the implantation site (or the soft tissue implant surface); (d) hyaluronic acid-containing formulations such as RESTYLANE or PERLANE (both from Q-Med AB, Sweden), HYLAFORM (Inamed Corporation, Santa Barbara, Calif.), PERLANE, SYNVISC (Biomatrix, Inc., Ridgefield, N.J.), SEPRAFILM or, SEPRACOAT (both from Genzyme Corporation), loaded with a fibrosis-inhibiting agent applied to the implantation site (or the soft tissue implant surface), (e) polymeric gels for surgical implantation such as REPEL (Life Medical Sciences, Inc., Princeton, N.J.) or FLOWGEL (Baxter Healthcare Corporation) loaded with a fibrosis-inhibiting agent applied to the implantation site (or the soft tissue implant surface); (f) orthopedic “cements” used to hold prostheses and tissues in place loaded with a fibrosis-inhibiting agent applied to the implantation site (or the soft tissue implant surface), such as OSTEOBOND (Zimmer, Inc., Warsaw, Ind.), low viscosity cement (LVC) from Wright Medical Technology, Inc. (Arlington, Tenn.) SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.), either alone, or loaded with a fibrosis-inhibiting agent, applied to the implantation site (or the soft tissue implant surface); (h) other biocompatible tissue fillers loaded with a fibrosis-inhibiting agent, such as those made by BioCure, Inc. (Norcross, Ga.), 3M Company and Neomend, Inc. (Sunnyvale, Calif.), applied to the implantation site (or the soft tissue implant surface); (i) polysaccharide gels such as the ADCON series of gels (available from Gliatech, Inc., Cleveland, Ohio) either alone, or loaded with a fibrosis-inhibiting agent, applied to the implantation site (or the soft tissue implant surface); and/or (j) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) loaded with a fibrosis-inhibiting agent applied to the implantation site (or the soft tissue implant surface). Several of the above compositions have the added advantage of also acting as a temporary (or permanent) barrier (particularly formulations containing PEG, hyaluronic acid, and polysaccharide gels), that can help prevent the formation of fibrous tissue around the soft tissue implant. Several of the above agents (e.g., formulations containing PEG, collagen, or fibrinogen such as COSEAL, CT3, ADHIBIT, COSTASIS, FOCALSEAL, SPRAYGEL, DURASEAL, TISSEAL AND FLOSEAL) have the added benefit of being hemostats and vascular sealants, which given the suspected role of inadequate hemostasis in the development of fibrous encapsulation, may also be of benefit in the practice of this invention.

A preferred polymeric matrix which can be used to help prevent the formation of fibrous tissue around the soft tissue implant, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino](4-armed amino PEG, which includes structures having a linking group(s) between an amino group(s) and the terminus of the polyethylene glycol backbone) and pentaerythritol poly(ethylene glycol)ether tetra-succinimidyl glutarate](4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue around the soft tissue implant.

It should be apparent to one of skill in the art that potentially any anti-scarring agent described above may be utilized alone, or in combination, in the practice of this embodiment. As soft tissue implants are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the implant (i.e., as a coating or infiltrated into the surrounding tissue), the fibrosis-inhibiting agents, used alone or in combination, may be administered Under the following dosing guidelines:

Drugs and Dosage: The following preferred drugs and dosages of fibrosis-inhibitors are suitable for use with all of the above soft tissue implants including facial implants, chin and mandibular implants, nasal implants, lip implants, pectoral implants, autogenous tissue implants and breast implants. Therapeutic agents that may be used as fibrosis-inhibiting agents in the practice of this invention include, but are not limited to: antimicrotubule agents including taxanes (e.g., paclitaxel and docetaxel), other microtubule stabilizing and anti-microtubule agents, mycophenolic acid, sirolimus, tacrolimus, everolimus, ABT-578 and vinca alkaloids (e.g., vinblastine and vincristine sulfate) as well as analogues and derivatives thereof. Drugs are to be used at concentrations that range from several times more than a single systemic dose (e.g., the dose used in oral or i.v. administration) to a fraction of a single systemic dose (e.g., 50%, 10%, 5%, or even less than 1% of the concentration typically used in a single systemic dose application). In one aspect, the drug is released in effective concentrations for a period ranging from 1-90 days. Antimicrotubule agents including taxanes, such as paclitaxel and analogues and derivatives (e.g., docetaxel) thereof, and vinca alkaloids, including vinblastine and vincristine sulfate and analogues and derivatives thereof, should be used under the following parameters: total dose not to exceed 10 mg (range of 0.1 μg to 10 mg); preferred total dose 1 μg to 3 mg. Dose per unit area of the device of 0.05 μg-10 μg per mm²; preferred dose/unit area of 0.20 μg/mm²-5 μg/mm². Minimum concentration of 10⁻⁹-10⁻⁴ M of drug is to be maintained on the device surface. Immunomodulators including sirolimus (i.e., rapamycin, RAPAMUNE), everolimus, tacrolimus, pimecrolimus, ABT-578, should be used under the following parameters: total dose not to exceed 10 mg (range of 0.1 μg to 10 mg); preferred 1 μg to 5 mg. The dose per unit area of 0.1 μg-100 μg per mm²; preferred dose of 0.25 μg/mm²-10 μg/mm². Minimum concentration of 10⁻⁸-10⁻⁴ M is to be maintained on the device surface. Inosine monophosphate dehydrogenase inhibitors (e.g., mycophenolic acid, 1-alpha-25 dihydroxy vitamin D₃) and analogues and derivatives thereof should be used under the following parameters: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface.

D. Therapeutic Agents for Use with Soft Tissue Implants

As described previously, numerous therapeutic agents are potentially suitable to prevent fibrous tissue accumulation around soft tissue implants. These therapeutic agents can be used alone, or in combination, to prevent scar tissue build-up in the vicinity of the implant-tissue interface in order to improve the clinical performance and longevity of these implants. Suitable fibrosis-inhibiting agents may be readily identified based upon in vitro and in vivo (animal) models, such as those provided in Examples 19-32. Agents that inhibit fibrosis can also be identified through in vivo models including inhibition of intimal hyperplasia development in the rat balloon carotid artery model (Examples 24 and 32). The assays set forth in Examples 23 and 31 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. The assay set forth in Example 27 may be used to determine whether an agent may inhibit migration of fibroblasts and/or smooth muscle cells. In one aspect of the invention, the agent has an IC₅₀ for inhibition of cell migration within a range of about 10⁻⁵ to about 10⁻⁹M. Assays set forth herein may be used to determine whether an agent is able to inhibit inflammatory processes, including nitric oxide production in macrophages (Example 19), and/or TNF-alpha production by macrophages (Example 20), and/or IL-1 beta production by macrophages (Example 28), and/or IL-8 production by macrophages (Example 29), and/or inhibition of MCP-1 by macrophages (Example 30). In one aspect of the invention, the agent has an IC₅₀ for inhibition of any one of these inflammatory processes within a range of about 10⁻⁶ to about 10⁻¹⁰M. The assay set forth in Example 25 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 26 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents that reduce the formation of surgical adhesions may be identified through in vivo models including the rabbit surgical adhesions model (Example 22) and the rat caecal sidewall model (Example 21).

These pharmacologically active agents (described herein) can be delivered at appropriate dosages (described herein) into to the tissue either alone, or via carriers (formulations are described herein), to treat the clinical problems described previously (described herein). Numerous therapeutic compounds have been identified that are of utility in the present invention including:

1) Angiogenesis Inhibitors

In one embodiment, the pharmacologically active compound is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulphate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo(4.5)decane-2-propanamine, N,N-diethyl-8,8-dipropyl, dimaleate), ATN-224, CHIR-258, combretastatin A-4 (phenol, 2-methoxy-5-(2-(3,4,5-trimethoxyphenyl)ethenyl)-, (Z)-), GCS-100LE, or an analogue or derivative thereof).

2) 5-Lipoxygenase Inhibitors and Antagonists

In another embodiment, the pharmacologically active compound is a 5-lipoxygenase inhibitor or antagonist (e.g., Wy-50295 (2-naphthaleneacetic acid, alpha-methyl-6-(2-quinolinylmethoxy)-, (S)-), ONO-LP-269 (2,11,14-eicosatrienamide, N-(4-hydroxy-2-(1H-tetrazol-5-yl)-8-quinolinyl)-, (E,Z,Z)-), licofelone (1H-pyrrolizine-5-acetic acid, 6-(4-chlorophenyl)-2,3-dihydro-2,2-dimethyl-7-phenyl-), CM I-568 (urea, N-butyl-N-hydroxy-N′-(4-(3-(methylsulfonyl)-2-propoxy-5-(tetrahydro-5-(3,4,5-trimethoxyphenyl)-2-furanyl)phenoxy)butyl)-,trans-), IP-751 ((3R,4R)-(delta 6)-THC-DMH-11-oic acid), PF-5901 (benzenemethanol, alpha-pentyl-3-(2-quinolinylmethoxy)-), LY-293111 (benzoic acid, 2-(3-(3-((5-ethyl-4′-fluoro-2-hydroxy(1,1′-biphenyl)-4-yl)oxy)propoxy)-2-propylphenoxy)-), RG-5901-A (benzenemethanol, alpha-pentyl-3-(2-quinolinylmethoxy)-, hydrochloride), rilopirox (2(1H)-pyridinone, 6-((4-(4-chlorophenoxy)phenoxy)methyl)-1-hydroxy-4-methyl-), L-674636 (acetic acid, ((4-(4-chlorophenyl)-1-(4-(2-quinolinylmethoxy)phenyl)butyl)thio)-AS)), 7-((3-(4-methoxy-tetrahydro-2H-pyran-4-yl)phenyl)methoxy)-4-phenylnaphtho(2,3-c)furan-1 (3H)-one, MK-886 (1H-indole-2-propanoic acid, 1-((4-chlorophenyl)methyl)-3-((1,1-dimethylethyl)thio)-alpha, alpha-dimethyl-5-(1-methylethyl)-), quiflapon (1H-indole-2-propanoic acid, 1-((4-chlorophenyl)methyl)-3-((1,1-dimethylethyl)thio)-alpha, alpha-dimethyl-5-(2-quinolinylmethoxy)-), quiflapon (1H-Indole-2-propanoic acid, 1-((4-chlorophenyl)methyl)-3-((1,1-dimethylethyl)thio)-alpha, alpha-dimethyl-5-(2-quinolinylmethoxy)-), docebenone (2,5-cyclohexadiene-1,4-dione, 2-(12-hydroxy-5,10-dodecadiynyl)-3,5,6-trimethyl-), zileuton (urea, N-(1-benzo(b)thien-2-ylethyl)-N-hydroxy-), or an analogue or derivative thereof).

3) Chemokine Receptor Antagonists CCR (1,3, and 5)

In another embodiment, the pharmacologically active compound is a chemokine receptor antagonist which inhibits one or more subtypes of CCR (13, and 5) (e.g., ONO-4128 (1,4,9-triazaspiro(5.5)undecane-2,5-dione, 1-butyl-3-(cyclohexylmethyl)-9-((2,3-dihydro-1,4-benzodioxin-6-yl)methyl-), L-381, CT-112 (L-arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-dimethyl-N-(4-(2-(4-methylphenyl)-6,7-dihydro-5H-benzocyclohepten-8-ylcarboxamido)benyl)tetrahydro-2H-pyran-4-aminium chloride), TAK-220, KRH-1120), GSK766994, SSR-150106, or an analogue or derivative thereof). Other examples of chemokine receptor antagonists include a-Immunokine-NNS03, BX-471, CCX-282, Sch-350634; Sch-351125; Sch-417690; SCH-C, and analogues and derivatives thereof.

4) Cell Cycle Inhibitors

In another embodiment, the pharmacologically active compound is a cell cycle inhibitor. Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl Cancer Inst. 83(4): 288-291, 1991; Pazdur et al., Cancer Treat Rev. 19(40): 351-386, 1993), etanidazole, nimorazole (B. A. Chabner and D. L. Longo. Cancer Chemotherapy and Biotherapy-Principles and Practice. Lippincott-Raven Publishers, New York, 1996, p. 554), perfluorochemicals with hyperbaric oxygen, transfusion, erythropoietin, BW12C, nicotinamide, hydralazine, BSO, WR-2721, ludR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int J. Radiat Biol. Relat. Stud. Phys., Chem. Med. 40(2): 153-61, 1981), SR-2508 (Brown et al., Int. J. Radiat. Oncol., Biol. Phys. 7(6): 695-703, 1981), 2H-isoindolediones (J. A. Myers, 2H-Isoindolediones, the synthesis and use as radiosensitizers. U.S. Pat. No. 4,494,547, Jan. 22, 1985), chiral (((2-bromoethyl)-amino)methyl)-nitro-1H-imidazole-1-ethanol (V. G. Beylin, et al., Process for preparing chiral (((2-bromoethyl)-amino)methyl)-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and the use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, December 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and the use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, June 1 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T. Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12): 3156-3174, 1996), camptothecin (Ewend M. G. et al. Local delivery of chemotherapy and concurrent external beam radiotherapy prolongs survival in metastatic brain tumor models. Cancer Research 56(22): 5217-5223, 1996) and paclitaxel (Tishler R. B. et al. Taxol: a novel radiation sensitizer. International Journal of Radiation Oncology and Biological Physics 22(3): 613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, fluorouracil, epirubicin, doxorubicin, vindesine and etoposide. Analogues and derivatives include (CPA)₂Pt(DOLYM) and (DACH)Pt(DOLYM) cisplatin (Choi et al., Arch. Pharmacal Res. 22(2): 151-156, 1999), Cis-(PtCl₂(4,7-H-5-methyl-7-oxo)1,2,4(triazolo(1,5-a)pyrimidine)₂) (Navarro et al., J. Med. Chem. 41(3): 332-338, 1998), (Pt(cis-1,4-DACH)(trans-Cl₂)(CBDCA))•½ MeOH cisplatin (Shamsuddin et al., Inorg. Chem. 36(25): 5969-5971, 1997), 4-pyridoxate diammine hydroxy platinum (Tokunaga et al., Pharm. Sci. 3(7): 353-356, 1997), Pt(II) . . . Pt(II) (Pt₂(NHCHN(C(CH₂)(CH₃)))₄) (Navarro et al., Inorg. Chem. 35(26): 7829-7835, 1996), 254-S cisplatin analogue (Koga et al., Neurol. Res. 18(3): 244-247, 1996), o-phenylenediamine ligand bearing cisplatin analogues (Koeckerbauer & Bednarski, J. Inorg. Biochem. 62(4): 281-298, 1996), trans, cis-(Pt(OAc)₂I₂(en)) (Kratochwil et al., J. Med. Chem. 39(13): 2499-2507, 1996), estrogenic 1,2-diarylethylenediamine ligand (with sulfur-containing amino acids and glutathione) bearing cisplatin analogues (Bednarski, J. Inorg. Biochem. 62(1): 75, 1996), cis-1,4-diaminocyclohexane cisplatin analogues (Shamsuddin et al., J. Inorg. Biochem. 61(4): 291-301, 1996), 5′ orientational isomer of cis-(Pt(NH₃)(4-aminoTEMP-O){d(GpG)}) (Dunham & Lippard, J. Am. Chem. Soc. 117(43): 10702-12, 1995), chelating diamine-bearing cisplatin analogues (Koeckerbauer & Bednarski, J. Pharm. Sci. 84(7): 819-23, 1995), 1,2-diarylethyleneamine ligand-bearing cisplatin analogues (Otto et al., J. Cancer Res. Clin. Oncol. 121(1): 31-8, 1995), (ethylenediamine)platinum(II) complexes (Pasini et al., J. Chem. Soc., Dalton Trans. 4: 579-85, 1995) CI-973 cisplatin analogue (Yang et al., Int. J. Oncol. 5(3): 597-602, 1994), cis-diamminedichloroplatinum(II) and its analogues cis-1,1-cyclobutanedicarbosylato(2R)-2-methyl-1,4-butanediammineplatinum(II) and cis-diammine(glycolato)platinum (Claycamp & Zimbrick, J. Inorg. Biochem., 26(4): 257-67, 1986; Fan et al., Cancer Res. 48(11): 3135-9, 1988; Heiger-Bemays et al., Biochemistry 29(36): 8461-6, 1990; Kikkawa et al., J. Exp. Clin. Cancer Res. 12(4): 233-40, 1993; Murray et al., Biochemistry 31(47): 11812-17, 1992; Takahashi et al., Cancer Chemother. Pharmacol. 33(1): 31-5, 1993), cis-amine-cyclohexylamine-dichloroplatinum(II) (Yoshida et al., Biochem. Pharmacol. 48(4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23): 4479-85, 1992), cisplatin analogues containing a tethered dansyl group (Hartwig et al., J. Am. Chem. Soc. 114(21): 8292-3, 1992), platinum(II)polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2): 311-15, 1991), trans-diamminedichloroplatinum(II) and cis-(Pt(NH₃)₂(N₃-cytosine)Cl) (Bellon & Lippard, Biophys. Chem. 35(2-3): 179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1): 41-58, 1989), diaminocarboxylatoplatinum (EPA 296321), trans-(D,1)-1,2-diaminocyclohexane carrier ligand-bearing platinum analogues (Wynick & Chaney, J. Labelled Compd. Radiopharm. 25(4): 349-57, 1988), aminoalkylaminoanthraquinone-derived cisplatin analogues (Kitov et al., Eur. J. Med. Chem. 23(4): 381-3, 1988), spiroplatin, carboplatin, iproplatin and JM40 platinum analogues (Schroyen et al., Eur. J. Cancer Clin. Oncol. 24(8): 1309-12, 1988), bidentate tertiary diamine-containing cisplatinum derivatives (Orbell et al., Inorg. Chim. Acta 152(2): 125-34, 1988), platinum(II), platinum(IV) (Liu & Wang, Shandong Yike Daxue Xuebao 24(1): 35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6: 443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4): 259-67, 1985); 4-hydroperoxycylcophosphamide (Ballard et al., Cancer Chemother. Pharmacol. 26(6): 397-402, 1990), acyclouridine cyclophosphamide derivatives (Zakerinia et al., Helv. Chim. Acta 73(4): 912-15, 1990), 1,3,2-dioxa- and -oxazaphosphorinane cyclophosphamide analogues (Yang et al., Tetrahedron 44(20): 6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1): 31-7, 1989), phenylketophosphamide cyclophosphamide analogues (Ludeman et al., J. Med. Chem. 29(5): 716-27, 1986), ASTA Z-7557 cyclophosphamide analogues (Evans et al., Int. J. Cancer 34(6): 883-90, 1984), 3-(1-oxy-2,2,6,6-tetramethyl-4-piperidinyl)cyclophosphamide (Tsui et al., J. Med. Chem. 25(9): 1106-10, 1982), 2-oxobis(2-α-chloroethylamino)-4-,6-dimethyl-1,3,2-oxazaphosphorinane cyclophosphamide (Carpenter et al., Phosphorus Sulfur 12(3): 287-93, 1982), 5-fluoro- and 5-chlorocyclophosphamide (Foster et al., J. Med. Chem. 24(12): 1399-403, 1981), cis- and trans-4-phenylcyclophosphamide (Boyd et al., J. Med. Chem. 23(4): 372-5, 1980), 5-bromocyclophosphamide, 3,5-dehydrocyclophosphamide (Ludeman et al., J. Med. Chem. 22(2): 151-8, 1979), 4-ethoxycarbonyl cyclophosphamide analogues (Foster, J. Pharm. Sci. 67(5): 709-10, 1978), arylaminotetrahydro-2H-1,3,2-oxazaphosphorine 2-oxide cyclophosphamide analogues (Hamacher, Arch. Pharm. (Weinheim, Ger.) 310(5):J, 428-34, 1977), NSC-26271 cyclophosphamide analogues (Montgomery & Struck, Cancer Treat. Rep. 60(4): J381-93, 1976), benzo annulated cyclophosphamide analogues (Ludeman & Zon, J. Med. Chem. 18(12): J1251-3, 1975), 6-trifluoromethylcyclophosphamide (Farmer & Cox, J. Med. Chem. 18(11): J1106-10, 1975), 4-methylcyclophosphamide and 6-methycyclophosphamide analogues (Cox et al., Biochem. Pharmacol. 24(5): J599-606, 1975); FCE 23762 doxorubicin derivative (Quaglia et al., J. Liq. Chromatogr. 17(18): 3911-3923, 1994), annamycin (Zou et al., J. Pharm. Sci. 82(11): 1151-1154, 1993), ruboxyl (Rapoport et al., J. Controlled Release 58(2): 153-162, 1999), anthracycline disaccharide doxorubicin analogue (Pratesi et al., Clin. Cancer Res. 4(11): 2833-2839, 1998), N-(trifluoroacetyl)doxorubicin and 4′-O-acetyl-N-(trifluoroacetyl)doxorubicin (Berube & Lepage, Synth. Commun. 28(6): 11109-1116, 1998), 2-pyrrolinodoxorubicin (Nagy et al., Proc. Nat'l Acad. Sci. U.S.A. 95(4): 1794-1799, 1998), disaccharide doxorubicin analogues (Arcamone et al., J. Nat'l Cancer Inst 89(16): 1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)-adriamicinone doxorubicin disaccharide analogue (Monteagudo et al., Carbohydr. Res. 300(1): 11-16, 1997), 2-pyrrolinodoxorubicin (Nagy et al., Proc. Natl Acad. Sci. U.S.A. 94(2): 652-656, 1997), morpholinyl doxorubicin analogues (Duran et al., Cancer Chemother. Pharmacol. 38(3): 210-216, 1996), enaminomalonyl-β-alanine doxorubicin derivatives (Seitz et al., Tetrahedron Lett. 36(9): 1413-16, 1995), cephalosporin doxorubicin derivatives (Vrudhula et al., J. Med. Chem. 38(8): 1380-5, 1995), hydroxyrubicin (Solary et al., Int. J. Cancer 58(1): 85-94, 1994), methoxymorpholino doxorubicin derivative (Kuhl et al., Cancer Chemother. Pharmacol. 33(1): 10-16, 1993), (6-maleimidocaproyl)hydrazone doxorubicin derivative (Wiliner et al., Bioconjugate Chem. 4(6): 521-7, 1993), N-(5,5-diacetoxypent-1-yl) doxorubicin (Cherif & Farquhar, J. Med. Chem. 35(17): 3208-14, 1992), FCE 23762 methoxymorpholinyl doxorubicin derivative (Ripamonti et al., Br. J. Cancer 65(5): 703-7, 1992), N-hydroxysuccinimide ester doxorubicin derivatives (Demant et al., Biochim. Biophys. Acta 1118(1): 83-90, 1991), polydeoxynucleotide doxorubicin derivatives (Ruggiero et al., Biochim. Biophys. Acta 1129(3): 294-302, 1991), morpholinyl doxorubicin derivatives (EPA 434960), mitoxantrone doxorubicin analogue (Krapcho et al., J. Med. Chem. 34(8): 2373-80. 1991), AD198 doxorubicin analogue (Traganos et al., Cancer Res. 51(14): 3682-9, 1991), 4-demethoxy-3′-N-trifluoroacetyidoxorubicin (Horton et al., Drug Des. Delivery 6(2): 123-9, 1990), 4′-epidoxorubicin (Drzewoski et al., Pol. J. Pharmacol. Pharm. 40(2): 159-65, 1988; Weenen et al., Eur. J. Cancer Clin. Oncol. 20(7): 919-26, 1984), alkylating cyanomorpholino doxorubicin derivative (Scudder et al), J. Nat'l Cancer Inst. 80(16): 1294-8, 1988), deoxydihydroiodooxorubicin (EPA 275966), adriblastin (Kalishevskaya et al., Vestn. Mosk. Univ., 16(Biol. 1): 21-7, 1988), 4′-deoxydoxorubicin (Schoelzel et al., Leuk. Res. 10(12): 1455-9, 1986), 4-demethyoxy-4′-o-methyldoxorubicin (Giuliani et al., Proc. Int. Congr. Chemother. 16: 285-70-285-77, 1983), 3′-deamino-3′-hydroxydoxorubicin (Horton et al., J. Antibiot. 37(8): 853-8, 1984), 4-demethyoxy doxorubicin analogues (Barbieri et al., Drugs Exp. Clin. Res. 10(2): 85-90, 1984), N-L-leucyl doxorubicin derivatives (Trouet et al., Anthracyclines (Proc. Int Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int J. Cancer 27(1): 5-13, 1981), aglycone doxorubicin derivatives (Chan & Watson, J. Pharm. Sci. 67(12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6): 1337-44, 1992), azo and azoxy misonidazole derivatives (Gattavecchia & Tonelli, Int J. Radiat. Biol. Relat Stud. Phys., Chem. Med. 45(5): 469-77, 1984); RB90740 (Wardman et al., Br. J. Cancer, 74 Suppl. (27): S70-S74, 1996); 6-bromo and 6-chloro-2,3-dihydro-1,4-benzothiazines nitrosourea derivatives (Rai et al., Heterocycl. Commun. 2(6): 587-592, 1996), diamino acid nitrosourea derivatives (Dulude et al., Bioorg. Med. Chem. Lett. 4(22): 2697-700, 1994; Dulude et al., Bioorg. Med. Chem. 3(2): 151-60, 1995), amino acid nitrosourea derivatives (Zheleva et al., Pharmazie 50(1): 25-6, 1995), 3′,4′-didemethoxy-3′,4′-dioxo-4-deoxypodophyllotoxin nitrosourea derivatives (Miyahara et al., Heterocycles 39(1): 361-9, 1994), ACNU (Matsunaga et al., Immunopharmacology 23(3): 199-204, 1992), tertiary phosphine oxide nitrosourea derivatives (Guguva et al., Pharmazie 46(8): 603, 1991), sulfamerizine and sulfamethizole nitrosourea derivatives (Chiang et al., Zhonghua Yaozue Zazhi 43(5): 401-6, 1991), thymidine nitrosourea analogues (Zhang et al., Cancer Commun. 3(4): 119-26, 1991), 1,3-bis(2-chloroethyl)-1-nitrosourea (August et al., Cancer Res. 51(6): 1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9): 1311-16, 1989), pyrimidine (II) nitrosourea derivatives (Wei et al., Chung-hua Yao Hsuch Tsa Chih 41(1): 19-26, 1989), CGP 6809 (Schieweck et al., Cancer Chemother. Pharmacol. 23(6): 341-7, 1989), B-3839 (Prajda et al., In Vivo 2(2): 151-4, 1988), 5-halogenocytosine nitrosourea derivatives (Chiang & Tseng, T'ai-wan Yao Hsuch Tsa Chih 38(1): 37-43, 1986), 1-(2-chloroethyl)-3-isobutyl-3-(β-maltosyl)-1-nitrosourea (Fujimoto & Ogawa, J. Pharmacobio-Dyn. 10(7): 341-5, 1987), sulfur-containing nitrosoureas (Tang et al., Yaoxue Xuebao 21(7): 502-9, 1986), sucrose, 6-((((2-chloroethyl)nitrosoamino-)carbonyl)amino)-6-deoxysucrose (NS-1 C) and 6′-((((2-chloroethyl)nitrosoamino)carbonyl)amino)-6′-deoxysucrose (NS-1 D) nitrosourea derivatives (Tanoh et al., Chemotherapy (Tokyo) 33(11): 969-77, 1985), CNCC, RFCNU and chlorozotocin (Mena et al., Chemotherapy (Basel) 32(2): 131-7, 1986), CNUA (Edanami et al., Chemotherapy (Tokyo) 33(5): 455-61, 1985), 1-(2-chloroethyl)-3-isobutyl-3-(β-maltosyl)-1-nitrosourea (Fujimoto & Ogawa, Jpn. J. Cancer Res. (Gann) 76(7): 651-6, 1985), choline-like nitrosoalkylureas(Belyaev et al., Izv. Akad. NA UK SSSR, Ser. Khim. 3: 553-7, 1985), sucrose nitrosourea derivatives (JP 84219300), sulfa drug nitrosourea analogues (Chiang et al., Proc. Nat'l Sci. Counc., Repub. China, Part A 8(1): 18-22, 1984), DONU (Asanuma et al., J. Jpn. Soc. Cancer Ther. 17(8): 2035-43, 1982), N,N′-bis (N-(2-chloroethyl)-N-nitrosocarbamoyl)cystamine (CNCC) (Blazsek et al., Toxicol. Appl. Pharmacol. 74(2): 250-7, 1984), dimethylnitrosourea (Krutova et al., Izv. Akad. NAUK SSSR, Ser. Biol. 3: 439-45, 1984), GANU (Sava & Giraldi, Cancer Chemother. Pharmacol. 10(3): 167-9, 1983), CCNU (Capelli et al., Med., Biol., Environ. 11(1): 111-16, 1983), 5-aminomethyl-2′-deoxyuridine nitrosourea analogues (Shiau, Shih Ta Hsuch Pao (Taipei) 27: 681-9, 1982), TA-077 (Fujimoto & Ogawa, Cancer Chemother. Pharmacol. 9(3): 134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.): 165-74, 1981), thiocolchicine nitrosourea analogues (George, Shih Ta Hsuch Pao (Taipei) 25: 355-62, 1980), 2-chloroethyl-nitrosourea (Zeller & Eisenbrand, Oncology 38(1): 39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8): 1393-401, 1980), N-deacetylmethyl thiocolchicine nitrosourea analogues (Lin et al., J. Med. Chem. 23(12): 1440-2, 1980), pyridine and piperidine nitrosourea derivatives (Crider et al., J. Med. Chem. 23(8): 848-51, 1980), methyl-CCNU (Zimber & Perk, Refu. Vet. 35(1): 28, 1978), phensuzimide nitrosourea derivatives (Crider et al., J. Med., Chem. 23(3): 324-6, 1980), ergoline nitrosourea derivatives (Crider et al., J. Med. Chem. 22(1): 32-5, 1979), glucopyranose nitrosourea derivatives (JP 78 95917), 1-(2-chloroethyl)-3-cyclohexyl-1-nitrosourea (Farmer et al., J. Med. Chem. 21(6): 514-20, 1978), 4-(3-(2-chloroethyl)-3-nitrosoureid-o)-cis-cyclohexanecarboxylic acid (Drewinko et al., Cancer Treat. Rep. 61(8): J1513-18, 1977), RPCNU (ICIG 1163) (Lamicol et al., Biomedicine 26(3): J176-81, 1977), IOB-252 (Sorodoc et al., Rev. Roum. Med., Virol. 28(1): J 55-61, 1977), 1,3-bis(2-chloroethyl)-1-nitrosourea (BCNU) (Siebert & Eisenbrand, Mutat. Res. 42(1): J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10): 793-6, 1995), 6-mercaptopurine (6-MP) (Kashida et al., Biol. Pharm. Bull. 18(11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56(4): 249-64, 1994), methyl-D-glucopyranoside mercaptopurine derivatives (Da Silva et al., Eur. J. Med. Chem. 29(2): 149-52, 1994) and s-alkynyl mercaptopurine derivatives (Ratsino et al., Khim.-Farm. Zh. 15(8): 65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12): 2287-2293, 1996), benzoxazine or benzothiazine moiety-bearing methotrexate derivatives (Matsuoka et al., J. Med. Chem. 40(1): 105-111, 1997), 10-deazaminopterin analogues (DeGraw et al., J. Med. Chem. 40(3): 370-376, 1997), 5-deazaminopterin and 5,10-dideazaminopterin methotrexate analogues (Piper et al., J. Med. Chem. 40(3): 377-384, 1997), indoline moiety-bearing methotrexate derivatives (Matsuoka et al., Chem. Pharm. Bull. 44(7): 1332-1337, 1996), lipophilic amide methotrexate derivatives (Pignatello et al., World Meet. Pharm., Biopharm. Pharm. Technol., 563-4, 1995), L-threo-(2S, 4S)-4-fluoroglutamic acid and DL-3,3-difluoroglutamic acid-containing methotrexate analogues (Hart et al., J. Med. Chem. 39(1): 56-65, 1996), methotrexate tetrahydroquinazoline analogue (Gangjee, et al., J. Heterocycl. Chem. 32(1): 243-8, 1995), N-(α-aminoacyl)methotrexate derivatives (Cheung et al., Pteridines 3(1-2): 101-2, 1992), biotin methotrexate derivatives (Fan et al., Pteridines 3(1-2): 131-2, 1992), D-glutamic acid or D-erythrou, threo-4-fluoroglutamic acid methotrexate analogues (McGuire et al., Biochem. Pharmacol. 42(12): 2400-3, 1991), β, γ-methano methotrexate analogues (Rosowsky et al., Pteridines 2(3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int Symp. Pteridines Folic Acid Deriv., 1154-7, 1989), N-(L-α-aminoacyl)methotrexate derivatives (Cheung et al., Heterocycles 28(2): 751-8, 1989), meta and ortho isomers of aminopterin (Rosowsky et al., J. Med. Chem. 32(12): 2582, 1989), hydroxymethylmethotrexate (DE 267495), γ-fluoromethotrexate (McGuire et al., Cancer Res. 49(16): 4517-25, 1989), polyglutamyl methotrexate derivatives (Kumar et al., Cancer Res. 46(10): 5020-3, 1986), gem-diphosphonate methotrexate analogues (WO 88/06158), α- and γ-substituted methotrexate analogues (Tsushima et al., Tetrahedron 44(17): 5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7): 1332-7, 1988), 8-deaza methotrexate analogues (Kuehl et al., Cancer Res. 48(6): 1481-8, 1988), acivicin methotrexate analogue (Rosowsky et al., J. Med. Chem. 30(8): 1463-9, 1987), polymeric platinol methotrexate derivative (Carraher et al., Polym. Sci. Technol. (Plenum), 35(Adv. Biomed. Polym.): 311-24, 1987), methotrexate-γ-dimyristoylphophatidylethanolamine (Kinsky et al., Biochim. Biophys. Acta 917(2): 211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem. Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15): 2607-13, 1986), polyglutamate methotrexate derivatives (Kamen & Winick, Methods Enzymol. 122 (Vitam. Coenzymes, Pt. G): 339-46, 1986), 5-methyl-5-deaza analogues (Piper et al., J. Med. Chem. 29(6): 1080-7, 1986), quinazoline methotrexate analogue (Mastropaolo et al., J. Med. Chem. 29(1): 155-8, 1986), pyrazine methotrexate analogue (Lever & Vestal, J. Heterocycl. Chem. 22(1): 5-6, 1985), cysteic acid and homocysteic acid methotrexate analogues (U.S. Pat. No. 4,490,529), γ-tert-butyl methotrexate esters (Rosowsky et al., J. Med. Chem. 28(5): 660-7, 1985), fluorinated methotrexate analogues (Tsushima et al., Heterocycles 23(1): 45-9, 1985), folate methotrexate analogue (Trombe, J. Bacteriol. 160(3): 849-53, 1984), phosphonoglutamic acid analogues (Sturtz & Guillamot, Eur. J. Med. Chem.—Chim. Ther. 19(3): 267-73, 1984), poly(L-lysine)methotrexate conjugates (Rosowsky et al., J. Med. Chem. 27(7): 888-93, 1984), dilysine and trilysine methotrexate derivates (Forsch & Rosowsky, J. Org. Chem. 49(7): 1305-9, 1984), 7-hydroxymethotrexate (Fabre et al., Cancer Res. 43(10): 4648-52, 1983), poly-γ-glutamyl methotrexate analogues (Piper & Montgomery, Adv. Exp. Med. Biol., 163(Folyl Antifolyl Polyglutamates): 95-100, 1983), 3′,5′-dichloromethotrexate (Rosowsky & Yu, J. Med. Chem. 26(10): 1448-52, 1983), diazoketone and chloromethylketone methotrexate analogues (Gangjee et al., J. Pharm. Sci. 71(6): 717-19, 1982), 10-propargylaminopterin and alkyl methotrexate homologs (Piper et al., J. Med. Chem. 25(7): 877-80, 1982), lectin derivatives of methotrexate (Lin et al., JNCI 66(3): 523-8, 1981), polyglutamate methotrexate derivatives (Galivan, Mol. Pharmacol. 17(1): 105-10, 1980), halogentated methotrexate derivatives (Fox, JNCI 58(4): J955-8, 1977), 8-alkyl-7,8-dihydro analogues (Chaykovsky et al., J. Med. Chem. 20(10): J1323-7, 1977), 7-methyl methotrexate derivatives and dichloromethotrexate (Rosowsky & Chen, J. Med. Chem. 17(12): J1308-11, 1974), lipophilic methotrexate derivatives and 3′,5′-dichloromethotrexate (Rosowsky, J. Med. Chem. 16(10): J1190-3, 1973), deaza amethopterin analogues (Montgomery et al., Ann. N.Y. Acad. Sci. 186: J227-34, 1971), MX068 (Pharma Japan, 1658: 18, 1999) and cysteic acid and homocysteic acid methotrexate analogues (EPA 0142220); N3-alkylated analogues of 5-fluorouracil (Kozai et al., J. Chem. Soc., Perkin Trans. 1(19): 3145-3146, 1998), 5-fluorouracil derivatives with 1,4-oxaheteroepane moieties (Gomez et al., Tetrahedron 54(43): 13295-13312, 1998), 5-fluorouracil and nucleoside analogues (Li, Anticancer Res. 17(1A): 21-27, 1997), cis- and trans-5-fluoro-5,6-dihydro-6-alkoxyuracil (Van der Wilt et al., Br. J. Cancer 68(4): 702-7, 1993), cyclopentane 5-fluorouracil analogues (Hronowski & Szarek, Can. J. Chem. 70(4): 1162-9, 1992), A-OT-fluorouracil (Zhang et al., Zongguo Yiyao Gongye Zazhi 20(11): 513-15, 1989), N4-trimethoxybenzoyl-5′-deoxy-5-fluorocytidine and 5′-deoxy-5-fluorouridine (Miwa et al., Chem. Pharm. Bull. 38(4): 998-1003, 1990), 1-hexylcarbamoyl-5-fluorouracil (Hoshi et al., J. Pharmacobio-Dun. 3(9): 478-81, 1980; Maehara et al., Chemotherapy (Basel) 34(6): 484-9, 1988), B-3839 (Prajda et al., In Vivo 2(2): 151-4, 1988), uracil-1-(2-tetrahydrofuryl)-5-fluorouracil (Anai et al., Oncology 45(3): 144-7, 1988), 1-(2′-deoxy-2′-fluoro-β-D-arabinofuranosyl)-5-fluorouracil (Suzuko et al., Mol. Pharmacol. 31(3): 301-6, 1987), doxifluridine (Matuura et al., Oyo Yakuri 29(5): 803-31, 1985), 5′-deoxy-5-fluorouridine (Bollag & Hartmann, Eur. J. Cancer 16(4): 427-32, 1980), 1-acetyl-3-O-toluyl-5-fluorouracil (Okada, Hiroshima J. Med. Sci. 28(1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4): 391-400, 1979); and Cu(II)-VP-16 (etoposide) complex (Tawa et al., Bioorg. Med. Chem. 6(7): 1003-1008, 1998), pyrrolecarboxamidino-bearing etoposide analogues (Ji et al., Bioorg. Med. Chem. Lett. 7(5): 607-612, 1997), 4β-amino etoposide analogues (Hu, University of North Carolina Dissertation, 1992), γ-lactone ring-modified arylamino etoposide analogues (Zhou et al., J. Med. Chem. 37(2): 287-92, 1994), N-glucosyl etoposide analogue (Allevi et al., Tetrahedron Lett. 34(45): 7313-16, 1993), etoposide A-ring analogues-(Kadow et al., Bioorg. Med. Chem. Left. 2(1): 17-22, 1992), 4′-deshydroxy-4′-methyl etoposide (Saulnier et al., Bioorg. Med. Chem. Left. 2(10): 1213-18, 1992), pendulum ring etoposide analogues (Sinha et al., Eur. J. Cancer 26(5): 590-3, 1990) and E-ring desoxy etoposide analogues (Saulnier et al., J. Med. Chem. 32(7): 1418-20, 1989).

Within one embodiment of the invention, the cell cycle inhibitor is paclitaxel, a compound that disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93: 2325, 1971), which has been obtained from the harvested and dried bark of Taxus brevifolia (Pacific Yew) and Taxomyces Andreanae and Endophytic Fungus of the Pacific Yew (Stierle et al., Science 60: 214-216, 1993). “Paclitaxel” (which may be understood herein to include formulations, prodrugs, analogues and derivatives such as, for example, TAXOL (Bristol Myers Squibb, New York, N.Y., TAXOTERE (Aventis Pharmaceuticals, France), docetaxel, 10-desacetyl analogues of paclitaxel and 3′N-desbenzoyl-3′N-t-butoxy carbonyl analogues of paclitakel) may be readily prepared utilizing techniques known to those skilled in the art (see, e.g., Schiff et al., Nature 277: 665-667, 1979; Long and Fairchild, Cancer Research 54: 4355-4361, 1994; Ringel and Horwitz, J. Natl Cancer Inst. 83(4): 288-291, 1991; Pazdur et al., Cancer Treat Rev. 19(4): 351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52): 9709-9712, 1994; J. Med. Chem. 35: 4230-4237, 1992; J. Med. Chem. 34: 992-998, 1991; J. Natural Prod. 57(10): 1404-1410, 1994; J. Natural Prod. 57(11): 1580-1583, 1994; J. Am. Chem. Soc. 110: 6558-6560, 1988), or obtained from a variety of commercial sources, including for example, Sigma Chemical Co., St. Louis, Mo. (T7402-from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′, 7-dibenzoyl taxol derivatives, other prod rugs (2′-acetyltaxol; 2′,7-diacetyltaxol; 2′-succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarbbxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin II, debenzoyl-2-acyl paclitaxel derivatives, benzoate paclitaxel derivatives, phosphonooxy and carbonate paclitaxel derivatives, sulfbnated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (C1): 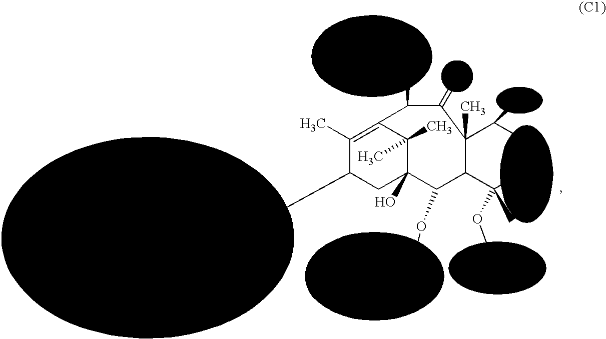
where the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2): 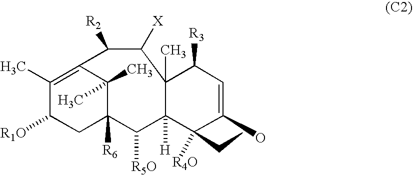
wherein X may be oxygen (paclitaxel), hydrogen (9-deoxy derivatives), thioacyl, or dihydroxylprecursors; R₁ is selected from paclitaxel or TAXOTERE side chains or alkanoyl of the formula (C3) 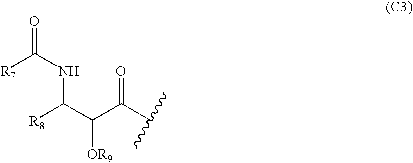
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as cell cycle inhibitors are disclosed in PCT International Patent Application No. WO 93/10076. As disclosed in this publication, the analogue or derivative may have a side chain attached to the taxane nucleus at C₁₃, as shown in the structure below (formula C4), in order to confer antitumor activity to the taxane.

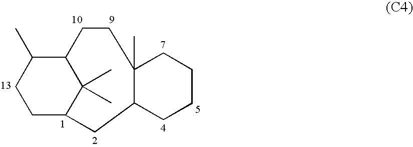

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the taxane-based cell cycle inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a cell cycle inhibitor by acting as an anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the anti-microtuble agent (microtubule inhibitor) is albendazole (carbamic acid, (5-(propylthio)-1H-benzimidazol-2-yl)-, methyl ester), LY-355703 (1,4-dioxa-8,11-diazacyclohexadec-13-ene-2,5,9,12-tetrone, 10-((3-chloro-4-methoxyphenyl)methyly)-6,6-dimethyl-3-(2-methylpropyl)-16-((1 S)-1-((2S,3R)-3-phenyloxiranyl)ethyl)-, (3S,10R,13E,16S)-), vindesine (vincaleukoblastine, 3-(aminocarbonyl)-O4-deacetyl-3-de(methoxycarbonyl)-), or WAY-174286.

In another aspect, the cell cycle inhibitor is a vinca alkaloid. Vinca alkaloids have the following general structure. They are indole-dihydroindole dimers.

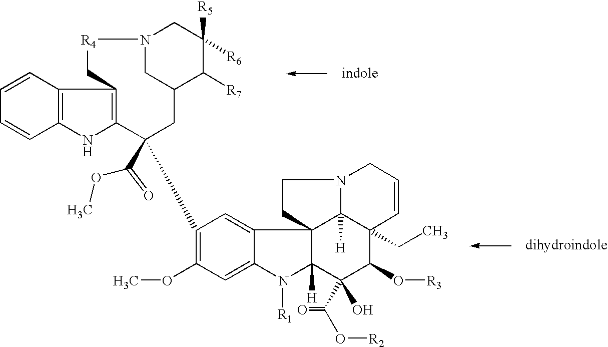

As disclosed in U.S. Pat. Nos. 4,841,045 and 5,030,620, R₁ can be a formyl or methyl group or alternately H. R₁ can also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group, such as maleoyl amino acid. R₃ can also be substituted to form an alkyl ester, which may be further substituted. R₄ may be —CH₂— or a single bond. R₅ and R₇ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

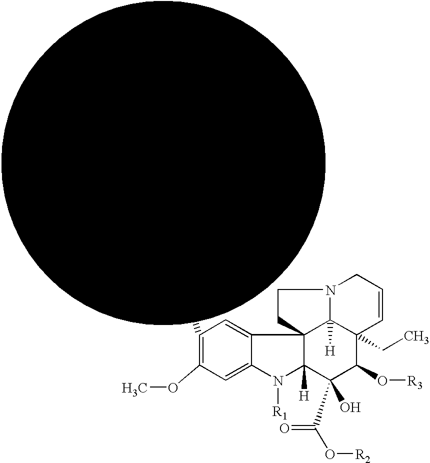

| | R₁ | R₂ | R₃ | R₄ | R₅ |
|---|---|---|---|---|---|
| Visblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogues typically require the side group (shaded area) in order to have activity. These compounds are thought to act as cell cycle inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the cell cycle inhibitor is a camptothecin, or an analog or derivative thereof. Camptothecins have the following general structure.

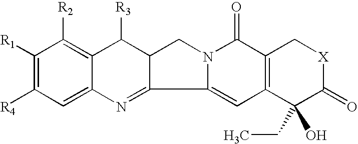

In this structure, X is typically 0, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocarhptothecin, 10-hydroxycamptothecin. Exemplary compounds have the structures:

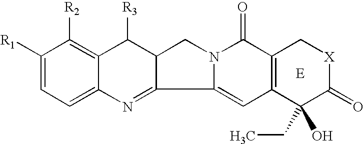

| | R₁ | R₂ | R₃ |
|---|---|---|---|
| Comptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as cell cycle inhibitors, where they can function as topoisomerase I inhibitors and/or DNA cleavage agents. They have been shown useful in the treatment of proliferative disorders, including, for example, NSC lung; small cell lung; and cervical cancers.

In another aspect, the cell cycle inhibitor is a podophyllotoxin, or a derivative or an analogue thereof. Exemplary compounds of this type are etoposide or teniposide, which have the following structures:

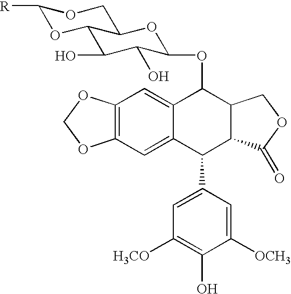

| | R |
|---|---|
| Etoposide | CH₃ |
| Teniposide | 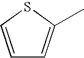 |

These compounds are thought to function as cell cycle inhibitors by being topoisomerase II inhibitors and/or by DNA cleaving agents. They have been shown useful as antiproliferative agents in, e.g., small cell lung, prostate, and brain cancers, and in retinoblastoma.

Another example of a DNA topoisomerase inhibitor is lurtotecan dihydrochloride (11H-1,4-dioxino(2,3-g)pyrano(3′,4′: 6,7)indolizino(1,2-b)quinoline-9,12(8H,14H)-dione, 8-ethyl-2,3-dihydro-8-hydroxy-15-((4-methyl-1-piperazinyl)methyl)-, dihydrochloride, (S)-).

In another aspect, the cell cycle inhibitor is an anthracycline. Anthracyclines have the following general structure, where the R groups may be a variety of organic groups:

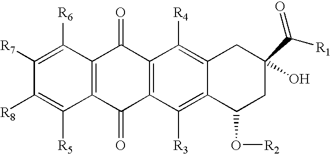

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 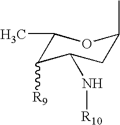
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which. R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

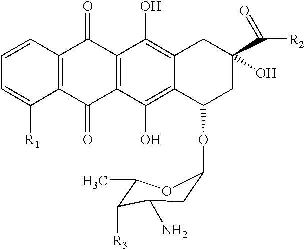

| | | |
|---|---|---|
| R₁ | R₂ | R₃ |

| | | | |
|---|---|---|---|
| Doxorubicin: | OCH₃ | CH₂OH | OH out of ring plane |
| Epirubicin: | OCH₃ | CH₂OH | OH in ring plane |
| (4′ epimer | | | |
| of doxo- | | | |
| rubicin) | | | |
| Dauno- | OCH₃ | CH₃ | OH out of ring plane |
| rubicin: | | | |
| Idarubicin: | H | CH₃ | OH out of ring plane |
| Pinrubicin | OCH₃ | OH | A |
| Zorubicin | OCH₃ | ═N—NHC(O)C₂H₅ | B |
| Carubicin: | OH | CH₃ | B |

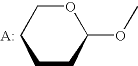

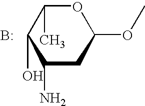

Other suitable anthracyclines are anthramycin, mitoxantrone, menogaril, nogalamycin, aclacinomycin A, olivomycin A, chromomycin A₃, and plicamycin having the structures:

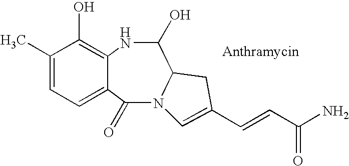

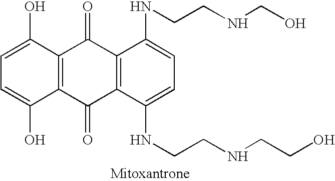

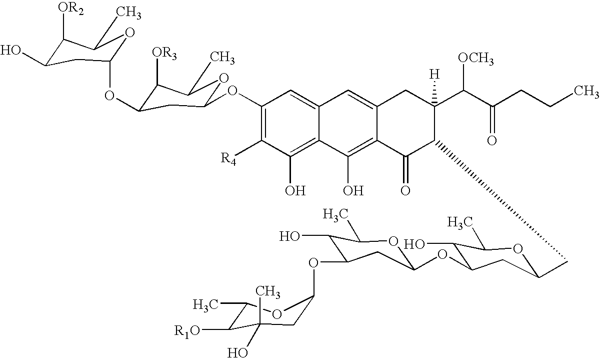

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CXH₃ | COCH₃ | CH₃ |
| Plicamycin | H | H | H | CH₃ |

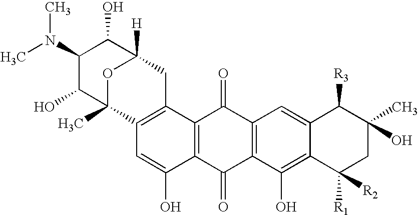

| | R₁ | R₂ | R₃ |
|---|---|---|---|
| Menogaril | H | OCH₃ | H |
| Nogalamycin | O-sugar | H | COOCH₃ |

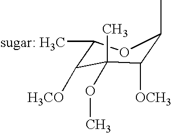

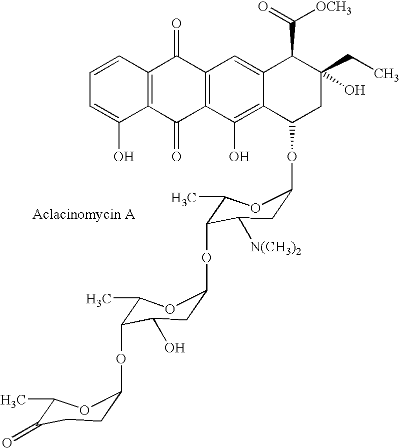

These compounds are thought to function as cell cycle inhibitors by being topoisomerase inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the cell cycle inhibitor is a platinum compound. In general, suitable platinum complexes may be of Pt(II) or Pt(IV) and have this basic structure: 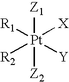
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

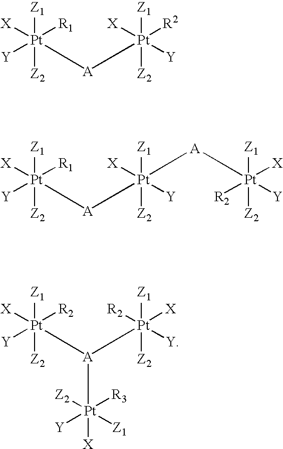

Exemplary platinum compounds are cisplatin, carboplatin, oxaliplatin, and miboplatin having the structures:

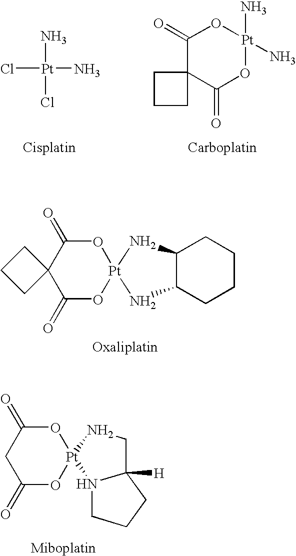

These compounds are thought to function as cell cycle inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the cell cycle inhibitor is a nitrosourea. Nitrosoureas have the following general structure (C5), where typical R groups are shown below.

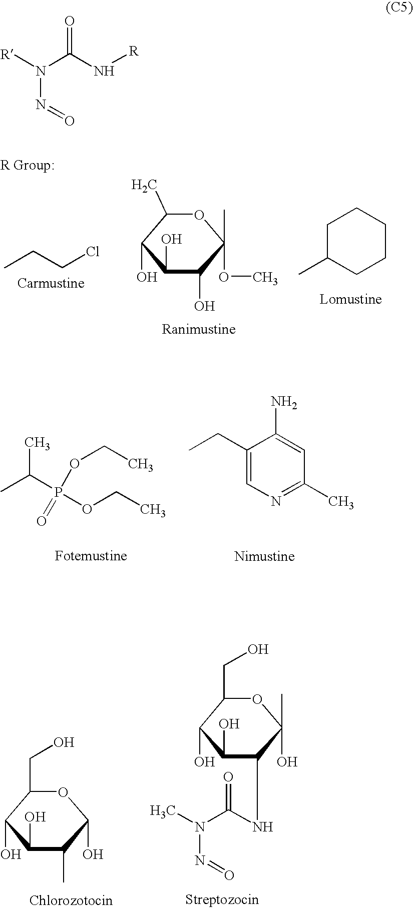

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N-R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, aEide, carboxylic acid, ether, thioether and alcohol groups. As disclosed in U.S. Pat. No. 4,472,379, R of formula (C5) may be an amide bond and a pyranose structure (e.g., methyl 2′-(N-(N-(2-chloroethyl)-N-nitroso-carbamoyl)-glycyl)amnio-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

Exemplary nitrosoureas are BCNU (carmustine), methyl-CCNU (semustine), CCNU (lomustine), ranimustine, nimustine, chlorozotocin, fotemustine, and streptozocin, having the structures:

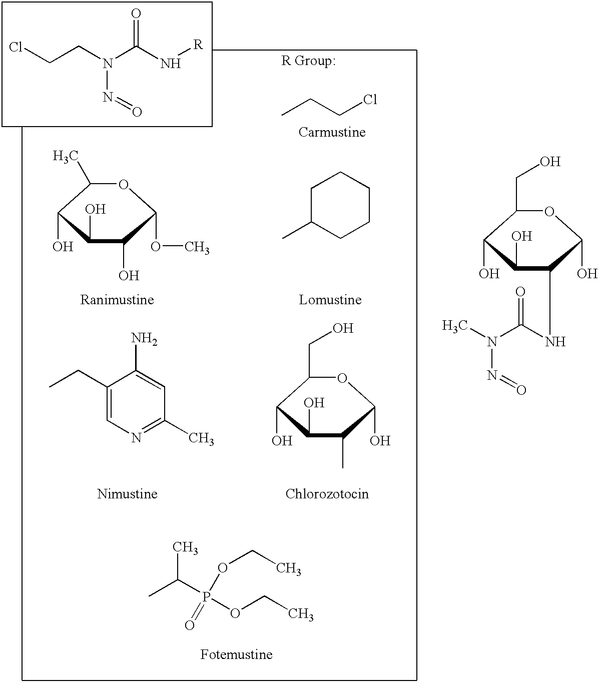

These nitrosourea compounds are thought to function as cell cycle inhibitors by binding to DNA, that is, by functioning as DNA alkylating agents. These cell cycle inhibitors have been shown useful in treating cell proliferative disorders such as, for example, islet cell; small cell lung; melanoma; and brain cancers.

In another aspect, the cell cycle inhibitor is a nitroimidazole, where exemplary nitroimidazoles are metronidazole, benznidazole, etanidazole, and misonidazole, having the structures:

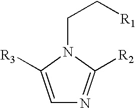

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the cell cycle inhibitor is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

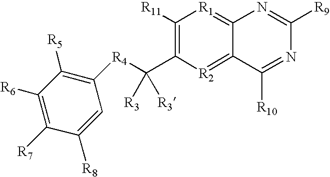

The identity of the R group may be selected from organic groups, particularly those groups setforth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 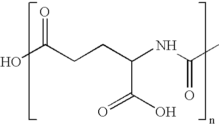
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

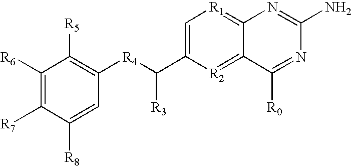

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | N(CH₂CH₂) | H | H | A (n = 1) | H |
| Trimetrexate | NH₂ | N | C(CH₃) | H | NH | H | OCH₃ | OCH₃ | OCH₃ |
| Pteropterin | NH₂ | N | N | H | N(CH₃) | H | H | A (n = 3) | H |
| Denopterin | OH | N | N | CH₃ | N(CH₃) | H | H | A (n = 1) | H |
| Pirtrexim | NH₂ | N | C(CH₃)H | single | OCH₃ | H | H | OCH₃ | H |
| | | | | bond | | | | | |

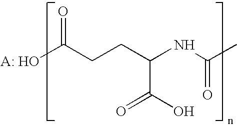

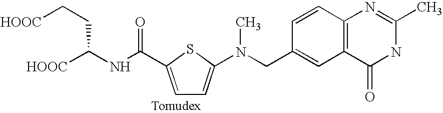

These compounds are thought to function as cell cycle inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the cell cycle inhibitor is a cytidine analogue, such as cytarabine or derivatives or analogues thereof, including enocitabine, FMdC ((E(−2′-deoxy-2′-(fluoromethylene)cytidine), gemcitabine, 5-azacitidine, ancitabine, and 6-azauridine. Exemplary compounds have the structures:

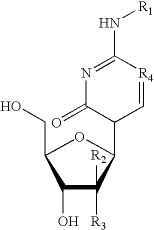

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

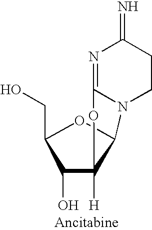

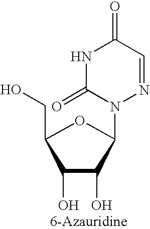

These compounds are thought to function as cell cycle inhibitors as acting as antimetabolites of pyrimidine. These compounds have been shown useful in the treatment of cell proliferative disorders including, for example, pancreatic, breast, cervical, NSC lung, and bile duct cancers.

In another aspect, the cell cycle inhibitor is a pyrimidine analogue. In one aspect, the pyrimidine analogues have the general structure: 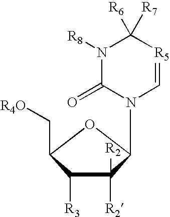
wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2°amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆═—NH—R₁ and R₇═H. R₈ is H or R₇ and R₈ together can form a double bond or R₈ can be X, where X is:

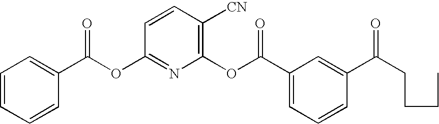

Specific pyrimidine analogues are disclosed in U.S. Pat. No. 3,894,000, (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the cell cycle inhibitor is a fluoropyrimidine analogue, such as 5-fluorouracil, or an analogue or derivative thereof, including carmofur, doxifluridine, emitefur, tegafur, and floxuridine. Exemplary compounds have the structures:

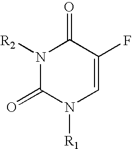

| | R₁ | R₂ |
|---|---|---|
| 5-Fluorouracil | H | H |
| Camofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | | H |

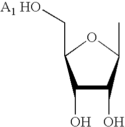

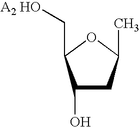

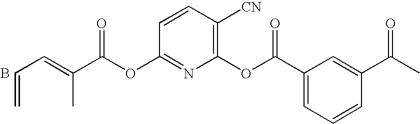

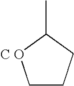

Other suitable fluoropyrimidine analogues include 5-FudR (5-fluoro-deoxyuridine), or an analogue or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Exemplary compounds have the structures:

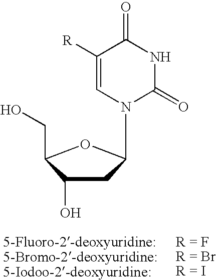

These compounds are thought to function as cell cycle inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the cell cycle inhibitor is a purine analogue. Purine analogues have the following general structure 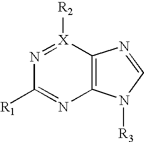
wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R₂ is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the formula 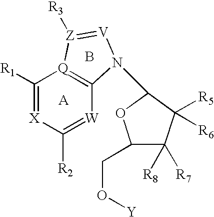
wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono-di or tri phosphate.

Exemplary suitable purine analogues include 6-mercaptopurine, thiguanosine, thiamiprine, cladribine, fludaribine, tubercidin, puromycin, pentoxyfilline; where these compounds may optionally be phosphorylated. Exemplary compounds have the structures:

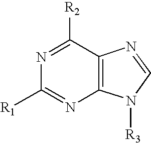

| | R₁ | R₂ | R₃ |
|---|---|---|---|
| 6-Mercaptopurine | H | SH | H |
| Thioguanosine | NH₂ | SH | B₁ |
| Thiamiprine | NH₂ | A | H |
| Cladribine | Cl | NH₂ | B₂ |
| Fludarabine | F | NH₂ | Bp₃ |
| Puromycin | H | N(CH₃)₂ | B₄ |
| Tubercidin | H | NH₂ | B₁ |

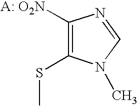

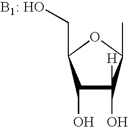

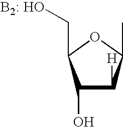

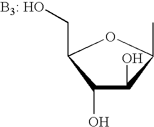

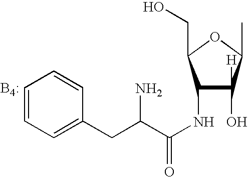

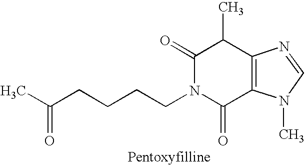

These compounds are thought to function as cell cycle inhibitors by serving as antimetabolites of purine.

In another aspect, the cell cycle inhibitor is a nitro gen mustard. Many suitable nitrogen mustards are known and are suitably used as a cell cycle inhibitor in the present invention. Suitable nitrogen mustards are also known as cyclophosohamides.

A preferred nitrogen mustard has the general structure: 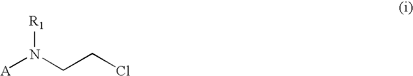
Where A is: 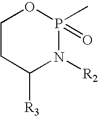
or —CH₃ or other alkane, or chloronated alkane, typically CH₂CH(CH₃)Cl, or a polycyclic group such as B, or a substituted phenyl such as C or a heterocyclic group such as D.

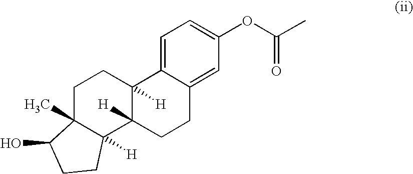

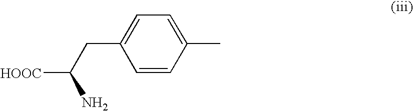

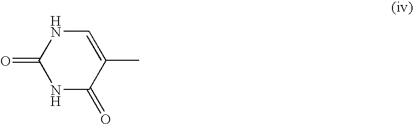

Examples of suitable nitrogen mustards are disclosed in U.S. Pat. No. 3,808,297, wherein A is:

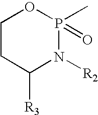

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure: 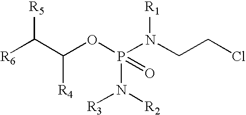
wherein R₁ is H or CH₂CH₂Cl, and R₂₋₆ are various substituent groups.

Exemplary nitrogen mustards include methylchloroethamine, and analogues or derivatives thereof, including methylchloroethamine oxide hydrohchloride, novembichin, and mannomustine (a halogenated sugar). Exemplary compounds have the structures:

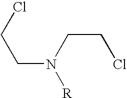

| | R |
|---|---|
| Mechlorethanime | CH₃ |
| Novembichin | CH₂CH(CH₃)Cl |

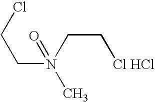

| | R |
|---|---|
| Mechlorethanime Oxide | HCl |

The nitrogen mustard may be cyclophosphamide, ifosfamide, perfosfamide, or torofosfamide, where these compounds have the structures:

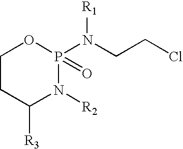

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The nitrogen mustard may be estramustine, or an analogue or derivative thereof, including phenesterine, prednimustine, and estramustine PO₄. Thus, suitable nitrogen mustard type cell cycle inhibitors of the present invention have the structures:

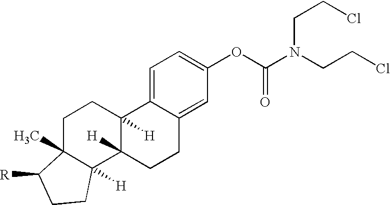

| | R |
|---|---|
| Estramustine | OH |
| Phenesterine | C(CH₃)(CH₂)₃CH(CH₃)₂ |

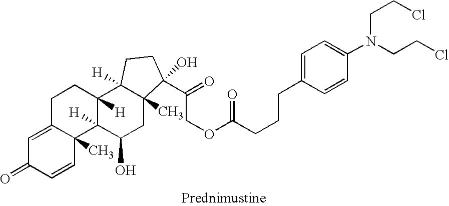

The nitrogen mustard may be chlorambucil, or an analogue or derivative thereof, including melphalan and chlormaphazine. Thus, suitable nitrogen mustard type cell cycle inhibitors of the present invention have the structures:

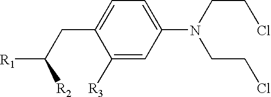

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The nitrogen mustard may be uracil mustard, which has the structure:

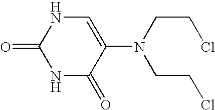

The nitrogen mustards are thought to function as cell cycle inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The cell cycle inhibitor of the present invention may be a hydroxyurea. Hydroxyureas have the following general structure:

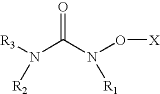

Suitable hydroxyureas are disclosed in, for example, U.S. Pat. No. 6,080,874, wherein R₁ is: 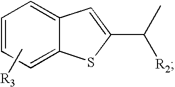
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 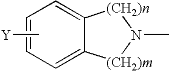
wherein m is 1 or 2, n is 0-2 and Y is an alkyl group.

In one aspect, the hydroxy urea has the structure:

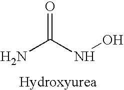

Hydroxyureas are thought to function as cell cycle inhibitors by serving to inhibit DNA synthesis.

In another aspect, the cell cycle inhibitor is a mytomicin, such as mitomycin C, or an analogue or derivative thereof, such as porphyromycin. Exemplary compounds have the structures:

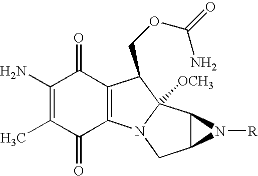

| | R |
|---|---|
| Mitomycin C | H |
| Porphyromycin | CH₃ |
| (N-methyl Mitomycin C) | |

These compounds are thought to function as cell cycle inhibitors by serving as DNA alkylating agents. Mitomycins have been shown useful in the treatment of cell proliferative disorders such as, for example, esophageal, liver, bladder, and breast cancers.

In another aspect, the cell cycle inhibitor is an alkyl sulfonate, such as busulfan, or an analogue or derivative thereof, such as treosulfan, improsulfan, piposulfan, and pipobroman. Exemplary compounds have the structures:

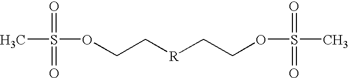

| | R |
|---|---|
| Busulfan | single band |
| Improsulfan | —CH₂—NH—CH₂— |
| Piposulfan | 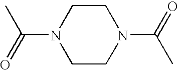 |

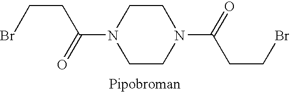

These compounds are thought to function as cell cycle inhibitors by serving as DNA alkylating agents.

In another aspect, the cell cycle inhibitor is a benzamide. In yet another aspect, the cell cycle inhibitor is a nicotinamide. These compounds have the basic structure: 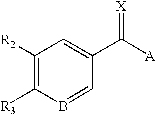
wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-ethyl (see, e.g., U.S. Pat. No. 5,215,738).

Suitable benzamide compounds have the structures: 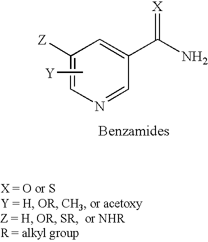
where additional compounds are disclosed in U.S. Pat. No. 5,215,738, (listing some 32 compounds).

Suitable nicotinamide compounds have the structures:

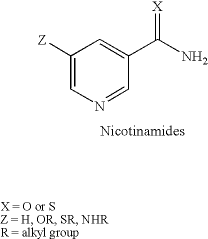

where additional compounds are disclosed in U.S. Pat. No. 5,215,738.

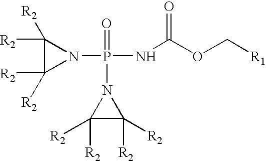

| | R₁ | R₂ |
|---|---|---|
| Benzodepa | phenyl | H |
| Meturedepa | CH₃ | CH₃ |
| Uredepa | CH₃ | H |

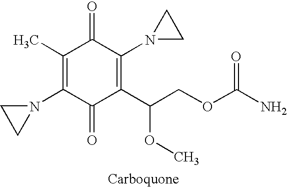

In another aspect, the cell cycle inhibitor is a halogenated sugar, such as mitolactol, or an analogue or derivative thereof, including mitobronitol and mannomustine. Exemplary compounds have the structures:

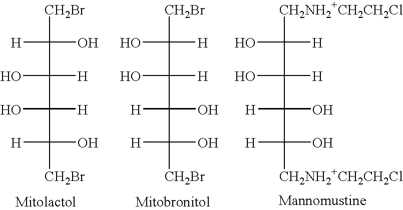

In another aspect, the cell cycle inhibitor is a diazo compound, such as azaserine, or an analogue or derivative thereof, including 6-diazo-5-oxo-L-norleucine and 5-diazouracil (also a pyrimidine analog). Exemplary compounds have the structures:

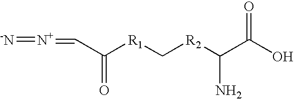

| | R₁ | R₂ |
|---|---|---|
| Azaserine | O | single bond |
| 6-diazo-5-oxo-L-norleucine | single bond | CH₂ |

Other compounds that may serve as cell cycle inhibitors according to the present invention are pazelliptine; wortmannin; metoclopramide; RSU; buthionine sulfoxime; tumeric; curcumin; AG337-, a thymidylate synthase inhibitor; levamisole; lentinan, a polysaccharide; razoxane, an EDTA analogue; indomethacin; chlorpromazine; α and β interferon; MnBOPP; gadolinium texaphyrin; 4-amino-1,8-naphthalimide; staurosporine derivative of CGP; and SR-2508.

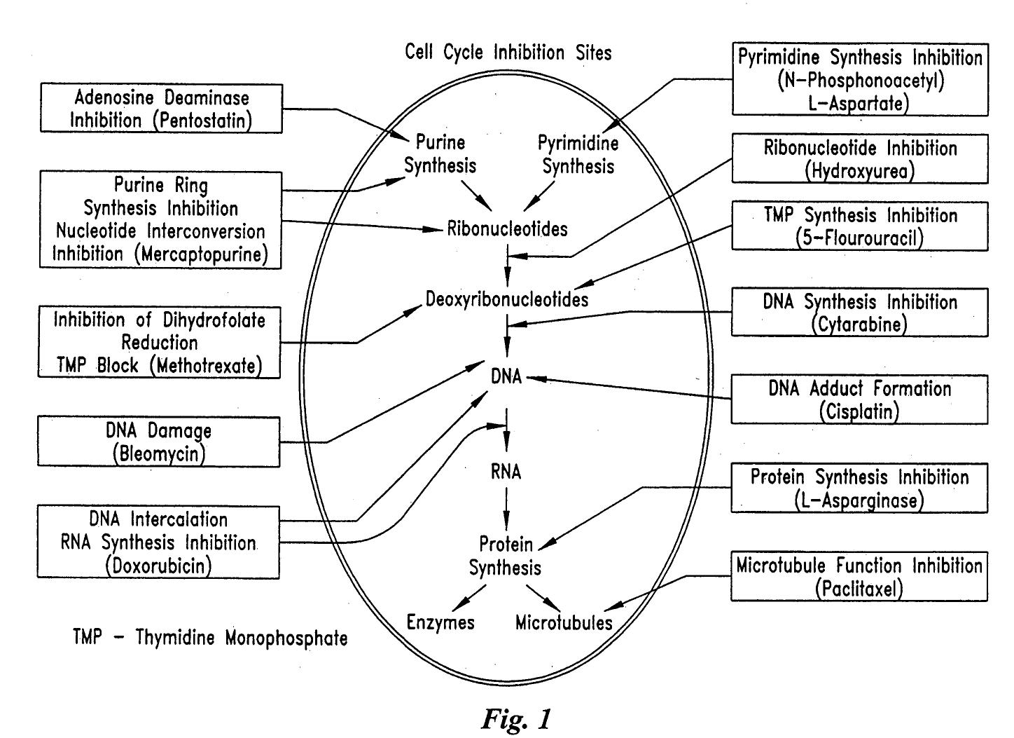

Thus, in one aspect, the cell cycle inhibitor is a DNA alylating agent. In another aspect, the cell cycle inhibitor is an anti-microtubule agent. In another aspect, the cell cycle inhibitor is a topoisomerase inhibitor. In another aspect, the cell cycle inhibitor is a DNA cleaving agent. In another aspect, the cell cycle inhibitor is an antimetabolite. In another aspect, the cell cycle inhibitor functions by inhibiting adenosine deaminase (e.g., as a purine analogue). In another aspect, the cell cycle inhibitor functions by inhibiting purine ring synthesis and/or as a nucleotide interconversion inhibitor (e.g., as a purine analogue such as mercaptopurine). In another aspect, the cell cycle inhibitor functions by inhibiting dihydrofolate reduction and/or as a thymidine monophosphate block (e.g., methotrexate). In another aspect, the cell cycle inhibitor functions by causing DNA damage (e.g., bleomycin). In another aspect, the cell cycle inhibitor functions as a DNA intercalation agent and/or RNA synthesis inhibition (e.g., doxorubicin, aclarubicin, or detorubicin (acetic acid, diethoxy-, 2-(4-((3-amino-2,3,6-trideoxy-alpha-L-lyxo-hexopyranosyl)oxy)-1,2,3,4,6,11-hexahydro-2,5,12-trihydroxy-7-methoxy-6,11-dioxo-2-naphthacenyl)-2-oxoethyl ester, (2S-cis)-)). In another aspect, the cell cycle inhibitor functions by inhibiting pyrimidine synthesis (e.g., N-phosphonoacetyl-L-aspartate). In another aspect, the cell cycle inhibitor functions by inhibiting ribonucleotides (e.g., hydroxyurea). In another aspect, the cell cycle inhibitor functions by inhibiting thymidine monophosphate (e.g., 5-fluorouracil). In another aspect, the cell cycle inhibitor functions by inhibiting DNA synthesis (e.g., cytarabine). In another aspect, the cell cycle inhibitor functions by causing DNA adduct formation (e.g., platinum compounds). In another aspect, the cell cycle inhibitor functions by inhibiting protein synthesis (e.g., L-asparginase). In another aspect, the cell cycle inhibitor functions by inhibiting microtubule function (e.g., taxanes). In another aspect, the cell cycle inhibitor acts at one or more of the steps in the biological pathway shown in FIG. 1.

Additional cell cycle inhibitor s useful in the present invention, as well as a discussion of the mechanisms of action, may be found in Hardman J. G., Limbird L E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In another embodiment, the cell-cycle inhibitor is camptothecin, mitoxantrone, etoposide, 5-fluorouracil, doxorubicin, methotrexate, peloruside A, mitomycin C, or a CDK-2 inhibitor or an analogue or derivative of any member of the class of listed compounds.

In another embodiment, the cell-cycle inhibitor is HTI-286, plicamycin; or mithramycin, or an analogue or derivative thereof.

Other examples of cell cycle inhibitors also include, e.g., 7-hexanoyltaxol (QP-2), cytochalasin A, lantrunculin D, actinomycin-D, Ro-31-7453 (3-(6-nitro-1-methyl-3-indolyl)-4-(1-methyl-3-indolyl)pyrrole-2,5-dione), PNU-151807, brostallicin, C2-ceramide, cytarabine ocfosfate (2(1H)-pyrimidinone, 4-amino-1-(5-O-(hydroxy(octadecyloxy)phosphinyly)-β-D-arabinofuranosyl)-, monosodium salt), paclitaxel (5β,20-epoxy-1,2 alpha,4,7β,10β,13 alpha-hexahydroxytax-11-en-9-one-4,10-diacetate-2-benzoate-13-(alpha-phenylhippurate)), doxorubicin (5,12-naphthacenedione, 10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-, (8S)-cis-), daunorubicin (5,12-naphthacenedione, 8-acetyl-10-((3-amino-2,3,6-trideoxy-alpha-L-lyxo-hexopyranosyl)oxy)-7,8,9,10-tetrahydro-6,8,1-trihydroxy-1-methoxy-, (8S-cis)-), gemcitabine hydrochloride (cytidine, 2′-deoxy-2′,2′-difluoro-,monohydrochloride), nitacrine (1,3-propanediamine, N,N-dimethyl-N′-(1-nitro-9-acridinyly), carboplatin (platinum, diammine(1,1-cyclobutanedicarboxylato(2-))-, (SP-4-2)-), altretamine (1,3,5-triazine-2,4,6-triamine, N,N,N′,N′,N″,N″-hexamethyl-), teniposide (furo(3′,4′:6,7)naphtho(2,3-d)-1,3-dioxol-6(5aH)-one, 5,8,8a,9-tetrahydro-5-(4-hydroxy-3,5-dimethoxyphenyl)-9-((4,6-O-(2-thienylmethylene)-β-D-glucopyranosyl)oxy)-, (5R-(5alpha,5aβ,8aAlpha,9β(R*)))-), eptaplatin (platinum, ((4R,5R)-2-(1-methylethyl)-1,3-dioxolane-4,5-dimethanamine-kappa N4,kappa N5)(propanedioato(2-)-kappa O1, kappa O3)-, (SP-4-2)-), amrubicin hydrochloride (5,12-naphthacenedione, 9-acetyl-9-amino-7-((2-deoxy-β-D-erythro-pentopyranosyl)oxy)-7,8,9,10-tetrahydro-6,11-dihydroxy-, hydrochloride, (7S-cis)-), ifosfamide (2H-1,3,2-oxazaphosphorin-2-amine, N,3-bis(2-chloroethyl)tetrahydro-,2-oxide), cladribine (adenosine, 2-chloro-2′-deoxy-), mitobronitol (b-mannitol, 1,6-dibromo-1,6-dideoxy-), fludaribine phosphate (9H-purin-6-amine, 2-fluoro-9-(5-O-phosphono-β-D-arabinofuranosyl)-), enocitabine (docosanamide, N-(1-β-D-arabinofuranosyl-1,2-dihydro-2-oxo-4-pyrimidinyl)-), vindesine (vincaleukoblastine, 3-(aminocarbonyl)-O4-deacetyl-3-de(methoxycarbonyl)-), idarubicin (5,12-naphthacenedione, 9-acetyl-7-((3-amino-2,3,6-trideoxy-alpha-L-lyxo-hexopyranosyl)oxy)-7,8,9,10-tetrahydro-6,9,11-trihydroxy-, (7S-cis)-), zinostatin (neocarzinostatin), vincristine (vincaleukoblastine, 22-oxo-), tegafur (2,4(1H,3H)-pyrimidinedione, 5-fluoro-1-(tetrahydro-2-furanyl)-), razoxane (2,6-piperazinedione, 4,4′-(1-methyl-1,2-ethanediyl)bis-), methotrexate (L-glutamic acid, N-(4-(((2,4-diamino-6-pteridinyl)methyl)methylamino)benzoyl)-), raltitrexed (L-glutamic acid, N-((5-(((1,4-dihydro-2-methyl-4-oxo-6-quinazolinyl)methyl)methylamino)-2-thienyl)carbonyl)-), oxaliplatin (platinum, (1,2-cyclohexanediamine-N,N′)(ethanedioato(2-)-O,O′)-, (SP-4-2-(1R-trans))-), doxifluridine (uridine, 5′-deoxy-5-fluoro-), mitolactol (galactitol, 1,6-dibromo-1,6-dideoxy-), piraubicin (5,12-naphthacenedione 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,11-trihydroxy-8-(hydroxyacetyl)-1-methoxy-, (8S-(8 alpha, 10 alpha(S*)))-), docetaxel ((2R,3S)-N-carboxy-3-phenylisoserine, N-tert-butyl ester, 13-ester with 5β,20-epoxy-1,2 alpha,4,7β,10β, 13 alpha-hexahydroxytax-11-en-9-one 4-acetate 2-benzoate-), capecitabine (cytidine, 5-deoxy-5-fluoro-N-((pentyloxy)carbonyl)-), cytarabine (2(1H)-pyrimidone, 4-amino-1-β-D-arabino furanosyl-), valrubicin (pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-alpha-L-lyxo-hexopyranosyl)oxy)-2-naphthacenyl)-2-oxoethyl ester (2S-cis)-), trofosfamide (3-2-(chloroethyl)-2-(bis(2-chloroethyl)amino)tetrahydro-2H-1,3,2-oxazaphosphorin 2-oxide), prednimustine (pregna-1,4-diene-3,20-dione, 21-(4-(4-(bis(2-chloroethyl)amino)phenyl)-1-oxobutoxy)-11,17-dihydroxy-, (11β)-), lomustine (Urea, N-(2-chloroethyl)-N′-cyclohexyl-N-nitroso-), epirubicin (5,12-naphthacenedione, 10-((3-amino-2,3,6-trideoxy-alpha-L-arabino-hexopyranosyl)oxy)-7,8,9,10-tetrahydro-6,8,1-trihydroxy-8-(hydroxyacetyl)-1-methoxy-, (8S-cis)-), or an analogue or derivative thereof).

5) Cyclin Dependent Protein Kinase Inhibitors

In another embodiment, the pharmacologically active compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-), SU-9516, AG-12275, PD-0166285, CGP-79807, fascaplysin, GW-8510 (benzenesulfonamide, 4-(((Z)-(6,7-dihydro-7-oxo-8H-pyrrolo(2,3-g)benzothiazol-8-ylidene)methyl)amino)-N-(3-hydroxy-2,2-dimethylpropyl)-), GW-491619, Indirubin 3′ monoxime, GW8510, AZD-5438, ZK-CDK or an analogue or derivative thereof.

6) EGF (Epidermal Growth Factor) Receptor Kinase Inhibitors

In another embodiment, the pharmacologically active compound is an EGF (epidermal growth factor) kinase inhibitor (e.g., erlotinib (4-quinazolinamine, N-(3-ethynylphenyl)-6,7-bis(2-methoxyethoxy)-, monohydrochloride), erbstatin, BIBX-1382, gefitinib (4-quinazolinamine, N-(3-chloro-4-fluorophenyl)-7-methoxy-6-(3-(4-morpholinyl)propoxy)), or an analogue or derivative thereof).

7) Elastase Inhibitors

In another embodiment, the pharmacologically active compound is an elastase inhibitor (e.g., ONO-6818, sivelestat sodium hydrate (glycine, N-(2-(((4-(2,2-dimethyl-1-oxopropoxy)phenyl)sulfonyl)amino)benzoyl)-), erdosteine (acetic acid, ((2-oxo-2-((tetrahydro-2-oxo-3-thienyl)amino)ethyl)thio)-), MDL-100948A, MDL-104238 (N-(4-(4-morpholinylcarbonyl)benzoyl)-L-valyl-N′-(3,3,4,4,4-pentafluoro-1-(1-methylethyl)-2-oxobutyl)-L-2-azetamide), MDL-27324 (L-prolinamide, N-((5-(dimethylamino)-1-naphthalenyl)sulfonyl)-L-alanyl-L-alanyl-N-(3,3,3-trifluoro-1-(1-methylethyl)-2-oxopropyl)-, (S)-), SR-26831 (thieno(3,2-c)pyridinium, 5-((2-chlorophenyl)methyl)-2-(2,2-dimethyl-1-oxopropoxy)-4,5,6,7-tetrahydro-5-hydroxy-), Win-68794, Win-63110, SSR-69071 (2-(9(2-piperidinoethoxy)-4-oxo-4H-pyrido(1,2-a)pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (N alpha-(1-adamantanesulphonyl)-N-(4-carboxybenzoyl)-L-lysyl-alanyl-L-valinal), R-665, FCE-28204, ((6R,7R)-2-(benzoyloxy)-7-methoxy-3-methyl-4-pivaloyl-3-cephem 1,1-dioxide), 1,2-benzisothiazol-3(2H)-one, 2-(2,4-dinitrophehyl)-, 1,1-dioxide, L-658758 (L-proline, 1-((3-((acetyloxy)methyl)-7-methoxy-8-oxo-5-thia-1-azabicyclo(4.2.0)oct-2-en-2-yl)carbonyl)-, S,S-dioxide, (6R-cis)-), L-659286 (pyrrolidine, 1-((7-methoxy-8-oxo-3-(((1,2,5,6-tetrahydro-2-methyl-5,6-dioxo-1,2,4-triazin-3-yl)thio)methyl)-5-thia-1-azabicyclo(4.2.0)oct-2-en-2-yl)carbonyl)-, S, S-dioxide, (6R-cis)-), L-680833 (benzeneacetic acid, 4-((3,3-diethyl-1-(((1-(4-methylphenyl)butyl)amino)carbonyl)-4-oxo-2-azetidinyl)oxy)-, (S-(R*,S*))-), FK-706 (L-prolinamide, N-(4-(((carboiymethyl)amino)carbonyl)benzoyl)-L-valyl-N-(3,3,3-trifluoro-1-(1-niethylethyl)-2-oxopropyl)-, monosodium salt), Roche R-665, or an analogue or derivative thereof).

8) Factor Xa Inhibitors

In another embodiment, the pharmacologically active compound is a factor Xa inhibitor (e.g., CY-222, fondaparinux sodium (alpha-D-glucopyranoside, methyl O-2-deoxy-6-O-sulfo-2-(sulfoamino)-alpha-D-glucopyranosyl-(1-4)-O-β-D-glucopyranuronosyl-(1-4)-O-2-deoxy-3,6-di-O-sulfo-2-(sulfoamino)-alpha-D-glucopyranosyl-(1-4)-O-β-O-sulfo-alpha-L-idopyranuronosyl-(1-4)-2-deoxy-2-(sulfoamino)-, 6-(hydrogen sulfate)), danaparoid sodium, or an analogue or derivative thereof).

9) Farnesyltransferase Inhibitors

In another embodiment, the pharmacologically active compound is a farnesyltransferase inhibitor (e.g., dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, perillyl alcohol (1-cyclohexene-1-methanol, 4-(1-methylethenyl)-, RPR-114334, lonafarnib (1-piperidinecarboxamide, 4-(2-(4-((11 R)-3,10-dibromo-8-chloro-6,11-dihydro-5H-benzo(5,6)cyclohepta(1,2-b)pyridin-11-yl)-1-piperidinyl)-2-oxoethyl)-), Sch-48755, Sch-226374, (7,8-dichloro-5H-dibenzo(b,e)(1,4)diazepin-11-y1)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))-), L-744832 (butanoic acid, 2-((2-((2-((2-amino-3-mercaptopropyl)amino)-3-methylpentyl)oxy)-1-oxo-3-phenylpropyl)aminb)-4-(methylsulfonyl)-, 1-methylethyl ester, (2S-(1(R*(R*)),2R*(S*),3R*))-), L-745631 (1-piperazinepropanethiol, β-amino-2-(2-methoxyethyl)-4-(1-naphthalenylcarbonyl)-, (βR,2S)-), N-acetyl-N-naphthylmethyl-2(S)-((1-(4-cyanobenzyl)-1H-imidazol-5-yl)acetyl)amino-3(S)-methylpentamine, (2alpha)-2-hydroxy-24,25-dihydroxylanost-8-en-3-one, BMS-316810, UCF-1-C (2,4-decadienamide, N-(5-hydroxy-5-(7-((2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1 alpha,3(2E,4E,6S*),5 alpha, 5(1E,3E,5E), 6 alpha))-), UCF-116-B, ARGLABIN (3H-oxireno(8,8a)azuleno(4,5-b)furan-8(4aH)-one, 5,6,6a,7,9a,9b-hexahydro-1,4a-dimethyl-7-methylene-, (3aR,4aS,6aS,9aS,9bR)-) from ARGLABIN-Paracure, Inc. (Virginia Beach, Va.), or an analogue or derivative thereof).

10) Fibrinogen Antagonists

In another embodiment, the pharmacologically active compound is a fibrinogen antagonist (e.g., 2(S)-((p-toluenesulfonyl)amino)-3-(((5,6,7,8,-tetrahydro-4-oxo-5-(2-(piperidin-4-yl)ethyl)-4H-pyrazolo-(1,5-a)(1,4)diazepin-2-yl)carbonyl)-amino)propionic acid, streptokinase (kinase (enzyme-activating), strepto-), urokinase (kinase (enzyme-activating), uro-), plasminogen activator, pamiteplase, monteplase, heberkinase, anistreplase, alteplase, pro-urokinase, picotamide (1,3-benzenedicarboxamide, 4-methoxy-N,N′-bis(3-pyridinylmethyl)-), or an analogue or derivative thereof).

11) Guanylate Cyclase Stimulants

In another embodiment, the pharmacologically active compound is a guanylate cyclase stimulant (e.g., isosorbide-5-mononitrate (D-glucitol, 1,4:3,6-dianhydro-, 5-nitrate), or an analogue or derivative thereof).

12) Heat Shock Protein 90 Antagonists

In another embodiment, the pharmacologically active compound is a heat shock protein 90 antagonist (e.g., geldanamycin; NSC-33050 (17-allylaminogeldanamycin), rifabutin (rifamycin XIV, 1′,4-didehydro-1-deoxy-1,4-dihydro-5′-(2-methylpropyl)-1-oxo-), 17AAG, or an analogue or derivative thereof).

13) HMGCoA Reductase Inhibitors

In another embodiment, the pharmacologically active compound is an HMGCoA reductase inhibitor (e.g., BCP-671, BB-476, fluvastatin (6-heptenoic acid, 7-(3-(4-fluorophenyl)-1-(1-methylethyl)-1H-indol-2-yl)-3,5-dihydroxy-, monosodium salt, (R*,S*-(E))-(±)-), dalvastatin (2H-pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4alpha,6β(E))-(+/−)-), glenvastatin (2H-pyran-2-one, 6-(2-(4-(4-fluorophenyl)-2-(1-methylethyl)-6-phenyl-3-pyridinyl)ethenyl)tetrahydro-4-hydroxy-, (4R-(4alpha,6β(E)))-), S-2468, N-(1-oxododecyl)-4Alpha,10-dimethyl-8-aza-trans-decal-3β-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl)-, calcium salt (R—(R*,R*))-), CP-83101 (6,8-nonadienoic acid, 3,5-dihydroxy-9,9-diphenyl-, methyl ester, (R*,S*-(E))-(+/−)-), pravastatin (1-naphthaleneheptanoic acid, 1,2,6,7,8,8a-hexahydro-β,delta,6-trihydroxy-2-methyl-8-(2-methyl-1-oxobutoxy)-, monosodium salt, (1S-(1 alpha(βS*,deltaS*),2 alpha,6 alpha,8β(R*),8a alpha))-), U-20685, pitavastatin (6-heptenoic acid, 7-(2-cyclopropyl-4-(4-fluorophenyl)-3-quinolinyl)-3,5-dihydroxy-, calcium salt (2:1), (S—(R*,S*-(E)))-), N-((1-methylpropyl)carbonyl)-8-(2-(tetrahydro-4-hydroxy-6-oxo-2H-pyran-2-yl)ethyl)-perhydro-isoquinoline, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha, 4a alpha,7β,8β(2S*,4S*),8aβ))-), HBS-107, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha,4a alpha,7β,8β(2S*,4S*),8aβ))-), L-669262 (butanoic acid, 2,2-dimethyl-, 1,2,6,7,8,8a-hexahydro-3,7-dim ethyl-6-oxo-8-(2-(tetrahydro-4-hydroxy-6-oxo-2H-pyran-2-yl)ethyl)-1-naphthalenyl(1 S-(1 Alpha,7β,8β(2S*,4S*),8aβ))-), simvastatin (butanoic acid, 2,2-dimethyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1alpha, 3alpha,7β,8β(2S*,4S*),8aβ))-), rosuvastatin calcium (6-heptenoic acid, 7-(4-(4-fluorophenyl)-6-(1-methylethyl)-2-(methyl(methylsulfonyl)amino)-5-pyrimdinyl)-3,5-dihydroxy-calcium salt (2:1) (S—(R*,S*-(E)))), meglutol (2-hydroxy-2-methyl-1,3-propandicarboxylic acid), lovastatin (butanoic acid, 2-methyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha.(R*),3 alpha,7β,8β(1(2S*,4S*),8aβ))-), or an analogue or derivative thereof).

14) Hydroorotate Dehydrogenase Inhibitors

In another embodiment, the pharmacologically active compound is a hydroorotate dehydrogenase inhibitor (e.g., leflunomide (4-isoxazolecarboxamide, 5-methyl-N-(4-(trifluoromethyl)phenyl)-), laflunimus (2-propenamide, 2-cyano-3-cyclopropyl-3-hydroxy-N-(3-methyl-4(trifluoromethyl)phenyl)-, (Z)-), or atovaquone (1,4-naphthalenedione, 2-(4-(4-chlorophenyl)cyclohexyl)-3-hydroxy-, trans-, or an analogue or derivative thereof.

15) IKK2 Inhibitors

In another embodiment, the pharmacologically active compound is an IKK2 inhibitor (e.g., MLN-120B, SPC-839, or an analogue or derivative thereof).

16) IL-1 ICE and IRAK Antagonists

In another embodiment, the pharmacologically active compound is an IL-1, ICE or an IRAK antagonist (e.g., E-5090 (2-propenoic acid, 3-(5-ethyl-4-hydroxy-3-methoxy-1-naphthalenyl)-2-methyl-, (Z)-), CH-164, CH-172, CH-490, AMG-719, iguratimod (N-(3-(fommylamino)-4-oxo-6-phenoxy-4H-chromen-7-yl) methanesulfonamide), AV94-88, pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), (2S-cis)-5-(benzyloxycarbonylamino-1,2,4,5,6,7-hexahydro-4-(oxoazepino(3,2,1-hi)indole-2-carbonyl)-amino)-4-oxobutanoic acid, AVE-9488, esonarimod (benzenebutanoic acid, alpha-((acetylthio)methyl)-4-methyl-gamma-oxo-), pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), tranexamic acid (cyclohexanecarboxylic acid, 4-(aminomethyl)-, trans-), Win-72052, romazarit (Ro-31-3948) (propanoic acid, 2-((2-(4-chlorophenyl)-4-methyl-5-oxazolyl)methoxy)-2-methyl-), PD-163594, SDZ-224-015 (L-alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-), L-709049 (L-alaninamide, N-acetyl-L-tyrosyl-L-valyl-N-(2-carboxy-1-formylethyl)-, (S)-), TA-383 (1H-imidazole, 2-(4-chlorophenyl)-4,5-dihydro-4,5-diphenyl-, monohydrochloride, cis-), EI-1507-1 (6a,12a-epoxybenz(a)anthracen-1,12(2H,7H)-dione, 3,4-dihydro-3,7-dihydroxy-8-methoxy-3-methyl-), ethyl 4-(3,4-dimethoxyphenyl)-6,7-dimethoxy-2-(1,2,4-triazol-1-yl methyl)quinoline-3-carboxylate, EI-1941-1, TJ-114, anakinra (interleukin 1 receptor antagonist (human isoform x reduced), N2-L-methionyl-), IX-207-887 (acetic acid, (10-methoxy-4H-benzo(4,5)cyclohepta(1,2-b)thien-4-ylidene)-), K-832, or an analogue or derivative thereof).

17) IL-4 Agonists

In another embodiment, the pharmacologically active compound is an IL-4 agonist (e.g., glatiramir acetate (L-glutamic acid, polymer with L-alanine, L-lysine and L-tyrosine, acetate (salt)), or an analogue or derivative thereof).

18) Immunomodulatory Agents

In another embodiment, the pharmacologically active compound is an immunomodulatory agent (e.g., biolimus, ABT-578, methylsulfamic acid 3-(2-methoxyphenoxy)-2-(((methylamino)sulfonyl)oxy)propyl ester, sirolimus (also referred to as rapamycin or RAPAMUNE (American Home Products, Inc., Madison, N.J.)), CCI-779 (rapamycin 42-(3-hydroxy-2-(hydroxymethyl)-2-methylpropanoate)), LF-15-0195, NPC15669 (L-leucine, N-(((2,7-dimethyl-9H-fluoren-9-yl)methoxy)carbonyl)-), NPC-15670 (L-leucine, N-(((4,5-dimethyl-9H-fluoren-9-yl)methoxy)carbonyl)-), NPC-16570 (4-(2-(fluoren-9-yl)ethyloxy-carbonyl)aminobenzoic acid), sufosfamide (ethanol, 2-((3-(2-chloroethyl)tetrahydro-2H-1,3,2-oxazaphosphorin-2-yl)amino)-, methanesulfonate (ester), P-oxide), tresperimus (2-(N-(4-(3-aminopropylamino)butyl)carbamoyloxy)-N-(6-guanidinohexyl)acetamide), 4-(2-(fluoren-9-yl)ethoxycarbonylamino)-benzo-hydroxamic acid, iaquinimod, PBI-1411, azathioprine (6-((1-Methyl-4-nitro-1H-imidazol-5-yl)thio)-1H-purine), PBI0032, beclometasone, MDL-28842 (9H-purin-6-amine, 9-(5-deoxy-5-fluoro-β-D-threo-pent-4-enofuranosyl)-, (Z)-), FK-788, AVE-1726, ZK-90695, ZK-90695, Ro-54864, didemnin-B, Illinois (didemnin A, N-(1-(2-hydroxy-1-oxopropyl)-L-prolyl)-, (S)-), SDZ-62-826 (ethanaminium, 2-((hydroxy((1′-((octadecyloxy)carbonyl)-3-piperidinyl)methoxy)phosphinyl)oxy)-N,N,N-trimethyl-, inner salt), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo(21.2.1)-hexacosa-1(25),23(26)-diene-2,5,8,11,14,17,20-heptaone), everolimus (rapamycin, 42-O-(2-hydroxyethyl)-), SAR-943, L-687795, 6-((4-chlorophenyl)sulfinyl)-2,3-dihydro-2-(4-methoxy-phenyl)-5-methyl-3-oxo-4-pyridazinecarbonitrile, 91Y78 (1H-imidazo[4,5-c)pyridin-4-amine, 1-β-D-ribofuranosyl-), auranofin (gold, (1-thio-β-D-glucopyranose 2,3,4,6-tetraacetato-S)(triethylphosphine)-), 27-O-demethylrapamycin, tipredane (androsta-1,4-dien-3-one, 17-(ethylthio)-9-fluoro-11-hydroxy-17-(methylthio)-, (11β,17 alpha)-), AI-402, LY-178002 (4-thiazolidinone, 5-((3,5-bis(1,1-dimethylethyl)-4-hydroxyphenyl)methylene)-), SM-8849 (2-thiazolamine, 4-(1-(2-fluoro(1,1′-biphenyl)-4-yl)ethyl)-N-methyl-), piceatannol, resveratrol, triamcinolone acetonide (pregna-1,4-diene-3,20-dione, 9-fluoro-11,21-dihydroxy-16,17-((1-methylethylidene)bis(oxy))-, (11β,16 alpha)-), ciclosporin (cyclosporin A), tacrolimus (15,19-epoxy-3H-pyrido(2,1-c)(1,4)oxaazacyclotricosine-1,7,20,21(4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1S*,3S*,4S*)),4S*,5R*,8S*,9E,12R*,14R*,15S*,16R*,18S*,19S*,26aR*))-), gusperimus (heptanamide, 7-((aminoiminomethyl)amino)-N-(2-((4-((3-aminopropyl)amino)butyl)amino)-1-hydroxy-2-oxoethyl)-, (+/−)-), tixocortol pivalate (pregn-4-ene-3,20-dione, 21-((2,2-dimethyl-1-oxopropyl)thio)-11,17-dihydroxy-, (11β)-), alefacept (1-92 LFA-3 (antigen) (human) fusion protein with immunoglobulin G1 (human hinge-CH2—CH3 gamma1-chain), dimer), halobetasol propionate (pregna-1,4-diene-3,20-dione, 21-chloro-6,9-difluoro-11-hydroxy-16-methyl-17-(1-oxopropoxy)-, (6Alpha,11β,16β)-), iloprost trometamol (pentanoic acid, 5-(hexahydro-5-hydroxy-4-(3-hydroxy-4-methyl-1-octen-6-ynyl)-2(1H)-pentalenylidene)-), beraprost (1H-cyclopenta(b)benzofuran-5-butanoic acid, 2,3,3a,8b-tetrahydro-2-hydroxy-1-(3-hydroxy-4-methyl-1-octen-6-ynyl)-), rimexolone (androsta-1,4-dien-3-one, 11-hydroxy-16,17-dimethyl-17-(1-oxopropyl)-, (11 β,16Alpha,17β)-), dexamethasone (pregna-1,4-diene-3,20-dione,9-fluoro-11,17,21-trihydroxy-16-methyl-, (111,16alpha)-), sulindac (cis-5-fluoro-2-methyl-1-((p-methylsulfinyl)benzylidene)indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-), alclometasone dipropionate (pregna-1,4-diene-3,20-dione, 7-chloro-11-hydroxy-16-methyl-17,21-bis(1-oxopropoxy)-, (7alpha, 11β,16alpha)-), pimecrolimus (15,19-epoxy-3H-pyrido(2,1-c)(1,4)oxaazacyclotricosine-1,7,20,21 (4H,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*))-), hydrocortisone-17-butyrate (pregn-4-ene-3,20-dione, 11,21-dihydroxy-17-(1-oxobutoxy)-, (11β)-), mitoxantrone (9,10-anthracenedione, 1,4-dihydroxy-5,8-bis((2-((2-hydroxyethyl)amino)ethyl)amino)-), mizoribine (1H-imidazole-4-carboxamide, 5-hydroxy-1-β-D-ribofuranosyl-), prednicarbate (pregna-1,4-diene-3,20-dione, 17-((ethoxycarbonyl)oxy)-1]-hydroxy-21-(1-oxopropoxy), (11β)-), iobenzarit (benzoic acid, 2-((2-carboxyphenyl)amino)-4-chloro-), glucametacin (D-glucose, 2-(((1-(4-chlorobenzoyl)-5-methoxy-2-methyl-1H-indol-3-yl)acetyl)amino)-2-deoxy-), fluocortolone monohydrate ((6 alpha)-fluoro-16alpha-methylpregna-1,4-dien-11β,21-diol-3,20-dione), fluocortin butyl (pregna-1,4-dien-21-oic acid, 6-fluoro-11-hydroxy-16-methyl-3,20-dioxo-, butyl ester, (6alpha, 11β,16alpha)-), difluprednate (pregna-1,4-diene-3,20-dione, 21-(acetyloxy)-6,9-difluoro-11-hydroxy-17-(1-oxobutoxy)-, (6 alpha, 11β)-), diflorasone diacetate (pregna-1,4-diene-3,20-dione, 17,21-bis(acetyloxy)-6,9-difluoro-11-hydroxy-16-methyl-, (6Alpha,11β,16β)-), dexamethasone valerate (pregna-1,4-diene-3,20-dione, 9-fluoro-11,21-dihydroxy-16-methyl-17-((1-oxopentyl)oxy)-, (11β,16Alpha)-), methylprednisolone, deprodone propionate (pregna-1,4-diene-3,20-dione, 11-hydroxy-17-(1-oxopropoxy)-, (11.beta.)-), bucillamine (L-cysteine, N-(2-mercapto-2-methyl-1-oxopropyl)-), amcinonide (benzeneacetic acid, 2-amino-3-benzoyl-, monosodium salt, monohydrate), acemetacin (1H-indole-3-acetic acid, 1-(4-chlorobenzoyl)-5-methoxy-2-methyl-, carboxymethyl ester), or an analogue or derivative thereof).

Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| RAPAMUNE | NSC-226080 | | |
| Rapamycin | | | |
| Tacrolimus | FK506 | Fujusawa | See below |

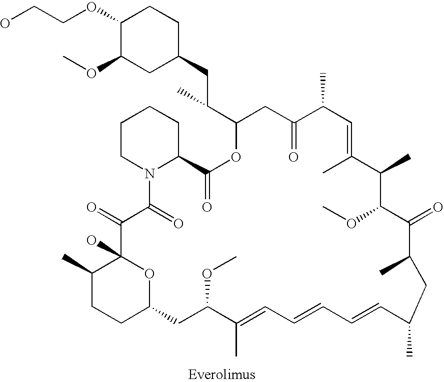

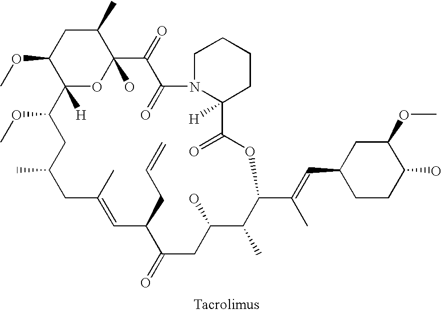

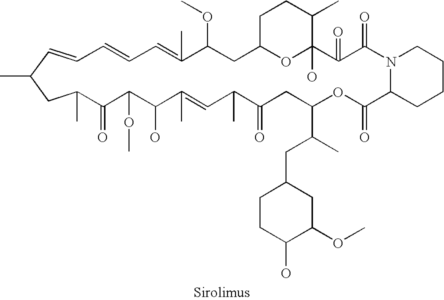

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502, WO 96/41807, WO 96/35423, WO 96/03430, WO 9600282, WO 95/16691, WO 9515328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

In one aspect, the fibrosis-inhibiting agent may be, e.g., rapamycin (sirolimus), everolimus, biolimus, tresperimus, auranofin, 27-O-demethylrapamycin, tacrolimus, gusperimus, pimecrolimus, or ABT-578.

19) Inosine Monophosphate Dehydrogenase Inhibitors

In another embodiment, the pharmacologically active compound is an inosine monophosphate dehydrogenase (IMPDH) inhibitor (e.g., mycophenolic acid, mycophenolaite mofetil (4-hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-triazole-3-carboxamide, 1-β-D-ribofuranosyl-), tiazofurin (4-thiazolecarboxamide, 2-β-D-ribofuranosyl-), viramidine, aminothiadiazole, thiophenfurin, tiazofurin) or an analogue or derivative thereof. Additional representative examples are included in U.S. Pat. Nos. 5,536,747, 5,807,876, 5,932,600, 6,054,472, 6,128,582, 6,344,465, 6,395,763, 6,399,773, 6,420,403, 6,479,628, 6,498,178,6,514,979, 6,518,291, 6,541,496, 6,596,747, 6,617,323, 6,624,184, Patent Application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201 A1, 2003/0181497A1, 2003/0186974A1, 2003/0186989A1, 2003/0195202A1, and PCT Publication Nos. WO 0024725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 2051814A1, WO 2057287A2, WO2057425A2, WO 2060875A1, WO 2060896A1, WO 2060898A1, WO 2068058A2, WO 3020298A1, WO 3037349A1, WO 3039548A1, WO 3045901A2, WO 3047512A2, WO 3053958A1, WO 3055447A2, WO 3059269A2, WO 3063573A2, WO 3087071 A1, WO 90/01545A1, WO 97/40028A1, WO 97/41211A1, WO 98/40381A1, and WO 99/55663A1).

20) Leukotriene Inhibitors

In another embodiment, the pharmacologically active compound is a leukotreine inhibitor (e.g., ONO-4057(benzenepropanoic acid, 2-(4-carboxybutoxy)-6-((6-(4-methoxyphenyl)-5-hexenyl)oxy)-, (E)-), ONO-LB-448, pirodomast 1,8-naphthyridin-2(1H)-one, 4-hydroxy-1-phenyl-3-(1-pyrrolidinyl)-, Sch-40120 (benzo(b)(1,8)naphthyridin-5(7H)-one, 10-(3-chlorophenyl)-6,8,9,10-tetrahydro-), L-656224 (4-benzofuranol, 7-chloro-2-((4-methoxyphenyl)methyl)-3-methyl-5-propyl-), MAFP (methyl arachidonyl fluorophosphonate), ontazolast (2-benzoxazolamine, N-(2-cyclohexyl-1-(2-pyridinyl)ethyl)-5-methyl-, (S)-), amelubant (carbamic acid, ((4-((3-((4-(1-(4-hydroxyphenyl)-1-methylethyl)phenoxy)methyl)phenyl)methoxy)phenyl)iminomethyl)-ethyl ester), SB-201993 (benzoic acid, 3-((((6-((1E)-2-carboxyethenyl)-5-((8-(4-methoxyphenyl)octyl)oxy)-2-pyridinyl)methyl)thio)methyl)-), LY-203647 (ethanone, 1-(2-hydroxy-3-propyl-4-(4-(2-(4-(1H-tetrazol-5-yl)butyl)-2H-tetrazol-5-yl)butoxy)phenyl)-), LY-210073, LY-223982 (benzenepropanoic acid, 5-(3-carboxybenzoyl)-2-((6-(4-methoxyphenyl)-5-hexenyl)oxy)-, (E)-), LY-293111 (benzoic acid, 2-(3-(3-((5-ethyl-4′-fluoro-2-hydroxy(1,1′-biphenyl)-4-yl)oxy)propoxy)-2-propylphenoxy)-), SM-9064 (pyrrolidine, 1-(4,11-dihydroxy-13-(4-methoxyphenyl)-1-oxo-5,7,9-tridecatrienyl)-, (E,E,E)-), T-0757 (2,6-octadienamide, N-(4-hydroxy-3,5-dimethylphenyl)-3,7-dimethyl-, (2E)-), or an analogue or derivative thereof.

21) MCP-1 Antagonists

In another embodiment, the pharmacologically active compound is a MCP-1 antagonist (e.g., nitronaproxen (2-napthaleneacetic acid, 6-methoxy-alpha-methyl 4-(nitrooxy)butyl ester (alpha S)-), bindarit (2-(1-benzylindazol-3-ylmethoxy)-2-methylpropanoic acid), 1-alpha-25 dihydroxy vitamin D₃, or an analogue or derivative thereof).

22) MMP Inhibitors

In another embodiment, the pharmacologically active compound is a matrix metalloproteinase (MMP) inhibitor (e.g., D-9120, doxycycline (2-naphthacenecarboxamide, 4-(dimethylamino)-1,4,4a,5,5a,6,11,12a-octahydro-3,5,10,12,12a-pentahydroxy-6-methyl-1,11-dioxo-(4S-(4 alpha, 4a alpha, 5 alpha, 5a alpha, 6 alpha, 12a alpha)O), BB-2827, BB-1101 (2S-allyl-N-1-hydroxy-3R-isobutyl-N-4-(1S-methylcarbamoyl-2-phenylethyl)-succinamide), BB-2983, solimastat (N′-(2,2-dimethyl-[(S)-(N-(2-pyridyl)carbamoyl)propyl)-N-4-hydroxy-2(R)-isobutyl-3(S)-methoxysuccinamide), batimastat (butanediamide, N4-hydroxy-N1-(2-(methylamino)-2-oxo-1-(phenylmethyl)ethyl)-2-(2-methylpropyl)-3-((2-thienylthio)methyl)-, (2R—(1(S*),2R*,3S*))-), CH-138, CH-5902, D-1927, D-5410, EF-13 (gamma-linolenic acid lithium salt), CMT-3 (2-naphthacenecarboxamide, 1,4,4a,5,5a,6,11,12a-octahydro-3,10,12,12a-tetrahydroxy-1,1]-dioxo-, (4aS,5aR,12aS)), marimastat (N-(2,2-dimethyl-1 (S)-(N-methylcarbamoyl)propyl)-N,3(S)-dihydroxy-2(R)-isobutylsuccinamide), TIMP'S, ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-), PS-508, CH-715, nimesulide (methanesulfonamide, N-(4-nitro-2-phenoxyphenyl)-), hexahydro-2-(2(R)-(1(RS)-(hydroxycarbamoyl)-4-phenylbutyl)nonanoyl)-N-(2,2,6,6-etramethyl-4-piperidinyl)-3(S)-pyridazine carboxamide, Rs-113-080, Ro-1130830, cipemastat (1-piperidinebutanamide, β-(cyclopentylmethyl)-N-hydroxy-gamma-oxo-alpha-((3,4,4-trimethyl-2,5-dioxo-1-imidazolidinyl)methyl)-,(alpha R,βR)-), 5-(4′-biphenyl)-5-(N-(4-nitrophenyl)piperazinyl)barbituric acid, 6-methoxy-1,2,3,4-tetrahydro-norharman-1-carboxylic acid, Ro-31-4724 (L-alanine, N-(2-(2-(hydroxyamino)-2-oxoethyl)-4-methyl-1-oxopentyl)-L-leucyl-, ethyl ester), prinomastat (3-thiomorpholinecarboxamide, N-hydroxy-2,2-dimethyl-4-((4-(4-pyridinyloxy) phenyl)sulfonyl)-, (3R)-), AG-3433 (1H-pyrrole-3-propanic acid, 1-(4′-cyano(1,1′-biphenyl)-4-yl)-b-((((3S)-tetrahydro-4,4-dimethyl-2-oxo-3-furanyl)amino)carbonyl)-, phenylmethyl ester, (bS)-), PNU-142769 (2H-Isoindole-2-butanamide, 1,3-dihydro-N-hydroxy-alpha-((3S)-3-(2-methylpropyl)-2-oxo-1-(2-phenylethyl)-3-pyrrolidinyl)-1,3-dioxo-, (alpha R)-), (S)-1-(2-((((4,5-dihydro-5-thioxo-1,3,4-thiadiazol-2-yl)amino)-carbonyl)amino)-1-oxo-3-(pentafluorophenyl)propyl)-4-(2-pyridinyl)piperazine, SU-5402 (1H-pyrrole-3-propanoic acid, 2-((1,2-dihydro-2-oxo-3H-indol-3-ylidene)methyl)-4-methyl-), SC-77964, PNU-171829, CGS-27023A, N-hydroxy-2(R)-((4-methoxybenzene-sulfonyl)(4-picolyl)amino)-2-(2-tetrahydrofuranyl)-acetamide, L-758354 ((1,1′-biphenyl)-4-hexanoic acid, alpha-butyl-gamma-(((2,2-dimethyl-1-((methylamino)carbonyl)propyl)amino)carbonyl)-4′-fluoro-, (alpha S-(alpha R*, gammaS*(R*)))-, GI-155704A, CPA-926, TMI-005, XL-754, or an analogue or derivative thereof). Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795;5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359.

3) NF Kappa B Inhibitors

In another embodiment, the pharmacologically active compound is a NF kappa B (NFKB) inhibitor (e.g., AVE-0545, Oxi-104 (benzamide, 4-amino-3-chloro-N-(2-(diethylamino)ethyl)-), dexlipotam, R-flurbiprofen ((1,1′-biphenyl)-4-acetic acid, 2-fluoro-alpha-methyl), SP100030 (2-chloro-N-(3,5-di(trifluoromethyl)phenyl)-4-(trifluoromethyl)pyrimidine-5-carboxamide), AVE-0545, Viatris, AVE-0547, Bay 11-7082, Bay 11-7085, 15 deoxy-prostaylandin J2, bortezomib (boronic acid, ((1R)-3-methyl-1-(((2S)-1-oxo-3-phenyl-2-((pyrazinylcarbonyl)amino)propyl)amino)butyl)-, benzamide an d nicotinamide derivatives that inhibit NF-kappaB, such as those described in U.S. Pat. Nos. 5,561,161 and 5,340,565 (OxiGene), PG490-88Na, or an analogue or derivative thereof).

24) NO Antagonists

In another embodiment, the pharmacologically active compound is a NO antagonist (e.g., NCX-4016 (benzoic acid, 2-(acetyloxy)-, 3-((nitrooxy)methyl)phenyl ester, NCX-2216, L-arginine or an analogue or derivative thereof).

25) P38 MAP Kinase Inhibitors

In another embodiment, the pharmacologically active compound is a p38 MAP kinase inhibitor (e.g., GW-2286, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, PD-98059 (4H-1-benzopyran-4-one, 2-(2-amino-3-methoxyphenyl)-), CGH-2466, doramapimod, SB-203580 (pyridine, 4-(5-(4-fluorophenyl)-2-(4-(methylsulfinyl)phenyl)-1H-imidazol-4-yl)-), SB-220025 ((5-(2-amino-4-pyrimidinyl)-4-(4-fluorophenyl)-1-(4-piperidinyl)imidazole), SB-281832, PD169316, SB202190, GSK-681323, EO-1606, GSK-681323, or an analogue or derivative thereof). Additional representative examples are included in U.S. Pat. Nos. 6,300,347; 6,316,464; 6,316,466; 6,376,527; 6,444,696; 6,479,507; 6,509,361; 6,579,874; 6,630,485, U.S. Patent Application Publication Nos. 2001/0044538A1; 2002/0013354A1; 2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 20030139462A1; 2003/0149031A1; 2003/0166647A1; 2003/0181411A1; and PCT Publication Nos. WO 00/63204A2; WO 01/21591A1; WO 01/35959A1; WO 01/74811A2; WO 02/18379A2; WO 2064594A2; WO 2083622A2; WO 2094842A2; WO 2096426A1; WO 2101015A2; WO 2103000A2; WO 3008413A1; WO 3016248A2; WO 0.3020715A1; WO 3024899A2; WO 3031431 A1; WO3040103A1; WO 3053940A1; WO 3053941A2; WO 3063799A2; WO 3079986A2; WO 3080024A2; WO 3082287A1; WO 97/44467A1; WO 99/01449A1; and WO 99/58523A1.

26) Phosphodiesterase Inhibitors

In another embodiment, the pharmacologically active compound is a phosphodiesterase inhibitor (e.g., CDP-840 (pyridine, 4-((2R)-2-(3-(cyclopentyloxy)-4-methoxyphenyl)-2-phenylethyl)-), CH-3697, CT-2820, D-22888 (imidazo[1,5-a)pyrido(3,2-e)pyrazin-6(5H)-one, 9-ethyl-2-methoxy-7-methyl-5-propyl-), D-4418 (8-methoxyquinoline-5-(N-(2,5-dichloropyridin-3-yl))carboxamide), 1-(3-cyclopentyloxy-4-methoxyphenyl)-2-(2,6-dichloro-4-pyridyl)ethanone oxime, D-4396, ONO-6126, CDC-998, CDC-801, V-11294A (3-(3-(cyclopentyloxy)-4-methoxybenzyl)-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahyrochloride, rolipram (2-pyrrolidinone, 4-(3-(cyclopentyloxy)-4-methoxyphenyl)-), CP-293121, CP-353164 (5-(3-cyclopentyloxy-4-methoxyphenyl)pyridine-2-carboxamide), oxagrelate (6-phthalazinecarboxylic acid, 3,4-dihydro-1-(hydroxymethyl)-5,7-dimethyl-4-oxo-, ethyl ester), PD-168787, ibudilast (1-propanone, 2-methyl-1-(2-(1-methylethyl)pyrazolo(1,5-a)pyridin-3-yl)-), oxagrelate (6-phthalazinecarboxylic acid, 3,4-dihydro-1-(hydroxymethyl)-5,7-dimethyl-4-oxo-, ethyl ester), griseolic acid (alpha-L-talo-oct-4-enofuranuronic acid, 1-(6-amino-9H-purin-9-yl)-3,6-anhydro-6-C-carboxy-1,5-dideoxy-), KW-4490, KS-506, T-440, roflumilast (benzamide, 3-(cyclopropylmethoxy)-N-(3,5-dichloro-4-pyridinyl)-4-(difluoromethoxy)-), rolipram, milrinone, triflusinal (benzoic acid, 2-(acetyloxy)-4-(trifluoromethyl)-), anagrelide hydrochloride (imidazo[2,1-b)quinazolin-2(3H)-one, 6,7-dichloro-1,5-dihydro-, monohydrochloride), cilostazol (2(1H)-quinolinone, 6-(4-(1-cyclohexyl-1H-tetrazol-5-yl)butoxy)-3,4-dihydro-), propentofylline (1H-purine-2,6-dione, 3,7-dihydro-3-methyl-1-(5-oxohexyl)-7-propyl-), sildenafil citrate (piperazine, 1-((3-(4,7-dihydro-1-methyl-7-oxo-3-propyl-1H-pyrazolo(4,3-d)pyrimidin-5-yl)-4-ethoxyphenyl)sulfonyl)-4-methyl, 2-hydroxy-1,2,3-propanetricarboxylate-(1:1)), tadalafil (pyrazino(1′,2′: 1,6)pyrido(3,4-b)indolel1,4-dione, 6-(1,3-benzodioxol-5-yl)-2,3,6,7,12,12a-hexahydro-2-methyl-, (6R-trans)), vardenafil (piperazine, 1-(3-(1,4-dihydro-5-methyl(−4-oxo-7-propylimidazo(5,1-f)(1,2,4)-triazin-2-yl)-4-ethoxyphenyl)sulfonyl)-4-ethyl-), milrinone ((3,4′-bipyridine)-5-carbonitrile, 1,6-dihydro-2-methyl-6-oxo-), enoximone (2H-imidazol-2-one, 1,3-dihydro-4-methyl-5-(4-(methylthio)benzoyl)-), theophylline (1H-purine-2,6-dione, 3,7-dihydro-1,3-dimethyl-), ibudilast (1-propanone, 2-methyl-1-(2-(1-methylethyl)pyrazolo(1,5-a)pyridin-3-yl)-), aminophylline (1H-purine-2,6-dione, 3,7-dihydro-1,3-dimethyl-, compound with 1,2-ethanediamine (2:1)-), acebrophylline (7H-purine-7-acetic acid, 1,2,3,6-tetrahydro-1,3-dimethyl-2,6-dioxo-,compd. with trans-4-(((2-amino-3,5-dibromophenyl)methyl)amino)cyclohexanol (1:1)), plafibride (propanamide, 2-(4-chlorophenoxy)-2-methyl-N-(((4-morpholinylmethyl)amino)carbonyl)-), ioprinone hydrochloride (3-pyridinecarbonitrile, 1,2-dihydro-5-imidazo(1,2-a)pyridin-6-yl-6-methyl-2-oxo-, monohydrochloride-), fosfosal (benzoic acid, 2-(phosphonooxy)-), amrinone ((3,4′-bipyridin)-6(1H)-one, 5-amino-, or an analogue or derivative thereof).

Other examples of phosphodiesterase inhibitors include denbufylline (1H-purine-2,6-dione, 1,3-dibutyl-3,7-dihydro-7-(2-oxopropyl)-), propentofylline (1H-purine-2,6-dione, 3,7-dihydro-3-methyl-1-(5-oxohexyl)-7-propyl-) and pelrinone (5-pyrimidinecarbonitrile, 1,4-dihydro-2-methyl-4-oxo-6-((3-pyridinylmethyl)amino)-).

Other examples of phosphodiesterase III inhibitors include enoximone (2H-imidazol-2-one, 1,3-dihydro-4-methyl-5-(4-(methylthio)benzoyl)-), and saterinone (3-pyridinecarbonitrile, 1,2-dihydro-5-(4-(2-hydroxy-3-(4-(2-methoxyphenyl)-1′-piperazinyl)propoxy)phenyl)-6-methyl-2-oxo-).

Other examples of phosphodiesterase IV inhibitors include AWD-12-281, 3-auinolinecarboxylic acid, 1-ethyl-6-fluoro-1,4-dihydro-7-(4-methyl-1-piperazinyl)-4-oxo-), tadalafil (pyrazino(1′,2′:1,6)pyrido(3,4-b)indolel,4-dione, 6-(1,3-benzodioxol-5-yl)-2,3,6,7,12,12a-hexahydro-2-methyl-, (6R-trans)), and filaminast (ethanone, 1-(3-(cyclopentyloxy)-4-methoxyphenyl)-, 0-(aminocarbonyl)oxime, (1E)-)

Another example of a phosphodiesterase V inhibitor is vardenafil (piperazine, 1-(3-(1,4-dihydro-5-methyl(−4-oxo-7-propylimidazo(5,1-f)(1,2,4)-triazin-2-yl)-4-ethoxyphenyl)sulfonyl)-4-ethyl-).

27) TGF beta Inhibitors

In another embodiment, the pharmacologically active compound is a TGF beta Inhibitor (e.g., mannose-6-phosphate, LF-984, tamoxifen (ethanamine, 2-(4-(1,2-diphenyl-1-butenyl)phenoxy)-N,N-dimethyl-, (Z)-), tranilast, or an analogue or derivative thereof).

28) Thromboxane A2 Antagonists

In another embodiment, the pharmacologically active compound is a thromboxane A2 antagonist (e.g., CGS-22652 (3-pyridineheptanoic acid, γ-(4-(((4-chlorophenyl)sulfonyl)amino)butyl)-, (.+−.)-), ozagrel (2-propenoic acid, 3-(4-(1H-imidazol-1-ylmethyl)phenyl)-, (E)-), argatroban (2-piperidinecarboxylic acid, 1-(5-((aminoiminomethyl)amino)-1-oxo-2-(((1,2,3,4-tetrahydro-3-methyl-8-quinolinyl)sulfonyl)amino)pentyl)-4-methyl-), ramatroban (9H-carbazole-9-propanoic acid, 3-(((4-fluorophenyl)sulfonyl)amino)-1,2,3,4-tetrahydro-, (R)-), torasemide (3-pyridinesulfonamide, N-(((1-methylethyl)amino)carbonyl)-4-((3-methylphenyl)amino)-), gamma linoleic acid ((Z,Z,Z)-6,9,12-octadecatrienoic acid), seratrodast (benzeneheptanoic acid, zeta-(2,4,5-trimethyl-3,6-dioxo-1,4-cyclohexadien-1-yl)-, (+/−)-, or an analogue or derivative thereof).

29) TNFα Antagonists and TACE Inhibitors

In another embodiment, the pharmacologically active compound is a TNFα antagonist or TACE inhibitor (e.g., E-5531 (2-deoxy-6-0-(2-deoxy-3-0-(3(R)-(5(Z)-dodecenoyloxy)-decyl)-6-0-methyl-2-(3-oxotetradecanamido)-4-O -phosphono-β-D-glucopyranosyl)-3-0-(3(R)-hydroxydecyl)-2-(3-oxotetradecanamido)-alpha-D-glucopyranose-1-O-phosphate), AZD-4717, glycophosphopeptical, UR-12715 (B=benzoic acid, 2-hydroxy-5-((4-(3-(4-(2-methyl-1H-imidazol(4,5-c)pyridin-1-yl)methyl)-1-piperidinyl)-3-oxo-1-phenyl-1-propenyl)phenyl)azo) (Z)), PMS-601, AM-87, xyloadenosine (9H-purin-6-amine, 9-β-D-xylofuranosyl-), RDP-58, RDP-59, BB2275, benzydamine, E-3330 (undecanoic acid, 2-((4,5-dimethoxy-2-methyl-3,6-dioxo-1,4-cyclohexadien-1-yl)methylene)-, (E)-), N-(D,L-2-(hydroxyaminocarbonyl)methyl-4-methylpentanoyl)-L-3-(2′-naphthyl)alanyl-L-alanine, 2-aminoethyl amide, CP-564959, MLN-608, SPC-839, ENMD-0997, Sch-23863 ((2-(10,11-dihydro-5-ethoxy-5H-dibenzo (a,d) cyclohepten-S-yl)-N,N-dimethyl-ethanamine), SH-636, PKF-241-466, PKF-242-484, TNF-484A, cilomilast (cis-4-cyano-4-(3-(cyclopentyloxy)-4-methoxyphenyl)cyclohexane-1-carboxylic acid), GW-3333, GW-4459, BMS-561392, AM-87, cloricromene (acetic acid, ((8-chloro-3-(2-(diethylamino)ethyl)-4-methyl-2-oxo-2H-1-benzopyran-7-yl)oxy)-, ethyl ester), thalidomide (1H-Isoindole-1,3(2H)-dione, 2-(2,6-dioxo-3-piperidinyl)-), vesnarinone (piperazine, 1-(3,4-dimethoxybenzoyl)-4-(1,2,3,4-tetrahydro-2-oxo-6-quinolinyl)-), infliximab, lentinan, etanercept (1-235-tumor necrosis factor receptor (human) fusion protein with 236-467-immunoglobulin G1 (human gamma1-chain Fc fragment)), diacerein (2-anthracenecarboxylic acid, 4,5-bis(acetyloxy)-9,10-dihydro-9,10-dioxo-, or an analogue or derivative thereof).

30) Tyrosine Kinase Inhibitors

In another embodiment, the pharmacologically active compound is a tyrosine kinase inhibitor (e.g., SKI-606, ER-068224, SD-208, N-(6-benzothiazolyl)-4-(2-(1-piperazinyl)pyrid-5-yl)-2-pyrimidineamine, celastrol (24,25,26-trinoroleana-1(10),3,5,7-tetraen-29-oic acid, 3-hydroxy-9,13-dimethyl-2-oxo-, (9 beta.,13alpha,14β,20 alpha)-), CP-127374 (geldanamycin, 17-demethoxy-17-(2-propenylamino)-), CP-564959, PD-171026, CGP-52411(1H-Isoindole-1,3(2H)-dione, 4,5-bis(phenylamino)-), CGP-53716 (benzamide, N-(4-methyl-3-((4-(3-pyridinyl)-2-pyrimidinyl)amino)phenyl)-), imatinib (4-((methyl-1-piperazinyl)methyl)-N-(4-methyl-3-((4-(3-pyridinyl)-2-pyrimidinyl)amino)-phenyl)benzamide methanesulfonate), NVP-MK980-NX, KF-250706 (13-chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-(3-(3-methoxy-4-(2-((E)-2-phenylethenyl)-4-nyl)-4-omxazolylmethyl)-2,4-oxazolidinedione, genistein, NV-06, or an analogue or derivative thereof).

31) Vitronectin Inhibitors

In another embodiment, the pharmacologically active compound is a vitronectin inhibitor (e.g., O-(9,10-dimethoxy-1,2,3,4,5,6-hexahydro-4-((1,4,5,6-tetrahydro-2-pyrimidinyl)hydrazono)-8-benz(e)azulenyl)-N-((phenylmethoxy)carbonyl)-DL-homoserine 2,3-dihydroxypropyl ester, (2S)-benzoylcarbonylamino-3-(2-((4S)-(3-(4,5-dihydro-1H-imidazol-2-ylamino)-propyl)-2,5-dioxo-imidazolidin-1-yl)-acetylamino)-propionate, Sch-221153, S-836, SC-68448 (β-((2-2-(((3-((aminoiminomethyl)amino)-phenyl)carbonyl)amino)acetyl)amino)-3,5-dichlorobenzenepropanoic acid), SD-7784, S-247, or an analogue or derivative thereof.

32) Fibroblast Growth Factor Inhibitors

In another embodiment, the pharmacologically active compound is a fibroblast growth factor inhibitor (e.g., CT-052923 (((2H-benzo(d)1,3-dioxalan-5-methyl)amino)(4-(6,7-dimethoxyquinazolin-4-yl)piperazinyl)methane-1-thione), or an analogue or derivative thereof).

33) Protein Kinase Inhibitors

In another embodiment, the pharmacologically active compound is a protein kinase inhibitor (e.g., KP-0201448, NPC15437 (hexanamide, 2,6-diamino-N-((1-(1-oxotridecyi)-2-piperidinyl)methyl)-), fasudil (1H-1,4-diazepine, hexahydro-1-(5-isoquinolinylsulfonyl)-), midostaurin (benzamide, N-(2,3,10,11,12,13-hexahydro-10-methoxy-9-methyl-1-oxo-9,13-epoxy-1H,9H-diindolo(1,2,3-gh:3′,2′, 1′-Im)pyrrolo(3,4-j)(1,7)benzodiazonin-11-yl)-N-methyl-, (9Alpha,10β,11β,13Alpha)-),fasudil (1H-1,4-diazepine, hexahydro-1-(5-isoquinolinylsulfonyl)-, dexniguldipine (3,5-pyridinedicarboxylic acid, 1,4-dihydro-2,6-dimethyl-4-(3-nitrophenyl)-, 3-(4,4-diphenyl-1-piperidinyl)propyl methyl ester, monohydrochloride, (R)-), LY-317615 (1H-pyrole-2,5-dione, 3-(1-methyl-1H-indol-3-yl)-4-(1-(1-(2-pyridinylmethyl)-4-piperidinyl)-1H-indol-3-yl)-, monohydrochloride), perifosine (piperidinium, 4-((hydroxy(octadecyloxy)phosphinyl)oxy)-1,1-dimethyl-, inner salt), LY-333531 (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)-), Kynac; SPC-100270 (1,3-octadecanediol, 2-amino-, (S—(R*,R*))-), Kynacyte, or an analogue or derivative thereof).

34) PDGF Receptor Kinase Inhibitors

In another embodiment, the pharmacologically active compound is a PDGF receptor kinase inhibitor (e.g., RPR-127963E, or an analogue or derivative thereof).

35) Endothelial Growth Factor Receptor Kinase Inhibitors

In another embodiment, the pharmacologically active compound is an endothelial growth factor receptor kinase inhibitor (e.g., CEP-7055, SU-0879 ((E)-3-(3,5-di-tert-butyl-4-hydroxyphenyl)-2-(aminothiocarbonyl)acrylonitile), BIBF-1000, AG-013736 (CP-868596), AMG-706, AVE-0005, NM-3 (3-(2-methylcarboxymethyl)-6-methoxy-8-hydroxy-isocoumarin), Bay-43-9006, SU-011248, or an analogue or derivative thereo).

36) Retinoic Acid Receptor Antagonists

In another embodiment, the pharmacologically active compound is a retinoic acid receptor antagonist (e.g., etarotene (Ro-15-1570) (naphthalene, 6-(2-(4-(ethylsulfonyl)phenyl)-1-methylethenyl)-1,2,3,4-tetrahydro-1,1,4,4-tetramethyl-, (E)-), (2E,4E)-3-methyl-5-(2-((E)-2-(2,6,6-trimethyl-1-cyclohexen-1-yl)ethenyl)-1-cyclohexen-1-yl)-2,4-pentadienoic acid, tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(±)), aliretinoin (retinoic acid, cis-9, trans-13-), bexarotene (benzoic acid, 4-(1-(5,6,7,8-tetrahydro-3,5,5,8,8-pentamethyl-2-naphthalenyl)ethenyl)-), tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(±)-, or an analogue or derivative thereof).

37) Platelet Derived Growth Factor Receptor Kinase Inhibitors

In another embodiment, the pharmacologically active compound is a platelet derived growth factor receptor kinase inhibitor (e.g., leflunomide (4-isoxazolecarboxamide, 5-methyl-N-(4-(trifluoromethyl)phenyl)-, or an analogue or derivative thereof).

38) Fibronogin Antagonists

In another embodiment, the pharmacologically active compound is a fibrinogin antagonist (e.g., picotamide (1,3-benzenedicarboxamide, 4-methoxy-N,N′-bis(3-pyridinylmethyl)-, or an analogue or derivative thereof).

39) Antimycotic Agents

In another embodiment, the pharmacologically active compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl)methoxy)phenyl)-4-(1-methylethyl)-, cis-), isoconazole (1-(2-(2-6-dichlorobenzyloxy)-2-(2-,4-dichlorophenyl)ethyl)), griseofulvin (spiro(benzofuran-2(3H), ¹′-(2)cyclohexane)-3,4′-dione, 7-chloro-2′,4,6-trimeth-oxy-6′methyl-, (1′S-trans)-), bifonazole (1H-imidazole, 1-((1,1′-biphenyl)-4-ylphenylmethyl)-), econazole nitrate (1-(2-((4-chlorophenyl)methoxy)-2-(2,4-dichlorophenyl)ethyl)-1H-imidazole nitrate), croconazole (1H-imidazole, 1-(1-(2-((3-chlorophenyl)methoxy)phenyl)ethenyl)-), sertaconazole (1H-imidazole, 1-(2-((7-chlorobenzo(b)thien-3-yl)methoxy)-2-(2,4-dichlorophenyl)ethyl)-), omoconazole (1H-imidazole, 1-(2-(2-(4-chlorophenoxy)ethoxy)-2-(2,4-dichlorophenyl)-1-methylethenyl)-, (Z)-), flutrimazole (1H-imidazole, 1-((2-fluorophenyl)(4-fluorophenyl)phenylmethyl)-), fluconazole (1H-1,2,4-triazole-1-ethanol, alpha-(2,4-difluorophenyl)-alpha-(1H-1,2,4-triazol-1-ylmethyl)-), neticonazole (1H-Imidazole, 1-(2-(methylthio)-1-(2-(pentyloxy)phenyl)ethenyl)-, monohydrochloride, (E)-), butoconazole (1H-imidazole, 1-(4-(4-chlorophenyl)-2-((2,6-dichlorophenyl)thio)butyl)-, (+/−)-), clotrimazole (1-((2-chlorophenyl)diphenylmethyl)-1H-imidazole, or an analogue or derivative thereof).

40) Bisphosphonates

In another embodiment, the pharmacologically active compound is a bisphosphonate (e.g., clodronate, alendronate, pamidronate, zoledronate, or an analogue or derivative thereof).

41) Phospholipase A1 Inhibitors

In another embodiment, the pharmacologically active compound is a phospholipase A1 inhibitor (e.g., ioteprednol etabonate (androsta-1,4-diene-17-carboxylic acid, 17-((ethoxycarbonyl)oxy)-11-hydroxy-3-oxo-, chloromethyl ester, (11β, 17 alpha)-, or an analogue or derivative thereof).

42) Histamine H1/H2/H3 Receptor Antagonists

In another embodiment, the pharmacologically active compound is a histamine H1, H2, or H3 receptor antagonist (e.g., ranitidine (1,1-ethenediamine, N-(2-(((5-((dimethylamino)methyl)-2-furanyl)methyl)thio)ethyl)-N′-methyl-2-nitro-), niperotidine (N-(2-((5-((dimethylamino)methyl)furfuryl)thio)ethyl)-2-nitro-N′-piperonyl-1,1-ethenediamine), famotidine (propanimidamide, 3-(((2-((aminoiminomethyl)amino)-4-thiazolyl)methyl)thio)-N-(aminosulfonyl)-), roxitadine acetate HCl (acetamide, 2-(acetyloxy)-N-(3-(3-(1-piperidinylmethyl)phenoxy)propyl)-, monohydrochloride), lafutidine (acetamide, 2-((2-furanylmethyl)sulfinyl)-N-(4-((4-(1-piperidinylmethyl)-2-pyridinyl)oxy)-2-butenyl)-, (Z)-), nizatadine (1,1-ethenediamine, N-(2-(((2-((dimethylamino)methyl)-4-thiazolyl)methyl)thio)ethyl)-N′-methyl-2-nitro-), ebrotidine (benzenesulfonamide, N-(((2-(((2-((aminoiminomethyl)amino)-4-thiazoly)methyl)thio)ethyl)amino)methylene)-4-bromo-), rupatadine (5H-benzo(5,6)cyclohepta(1,2-b)pyridine, 8-chloro-6,11-dihydro-11-(1-((5-methyl-3-pyridinyl)methyl)-4-piperidinylidene)-, trihydrochloride-), fexofenadine HCl (benzeneacetic acid, 4-(1-hydroxy-4-(4(hydroxydiphenylmethyl)-1-piperidinyl)butyl)-alpha, alpha-dimethyl-, hydrochloride, or an analogue or derivative thereof).

43) Macrolide Antibiotics

In another embodiment, the pharmacologically active compound is a macrolide antibiotic (e.g., dirithromycin (erythromycin, 9-deoxo-11-deoxy-9,11-(imino(2-(2-methoxyethoxy)ethylidene)oxy)-, (9S(R))-), flurithromycin ethylsuccinate (erythromycin, 8-fluoro-mono(ethyl butanedioate) (ester)-), erythromycin stinoprate (erythromycin, 2′-propanoate, compound with N-acetyl-L-cysteine (1:1)), clarithromycin (erythromycin, 6-O-methyl-), azithromycin (9-deoxo-9a-aza-9a-methyl-9a-homoerythromycin-A), telithromycin (3-de((2,6-dideoxy-3-C-methyl-3-O-methyl-alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-), roxithromycin (erythromycin, 9-(O-((2-methoxyethoxy)methyl)oxime)), rokitamycin (leucomycin V, 4B-butanoate 3B-propanoate), RV-11 (erythromycin monopropionate mercaptosuccinate), midecamycin acetate (leucomycin V, 3B,9-diacetate 3,4B-dipropanoate), midecamycin (leucomycin V, 3,4B-dipropanoate), josamycin (leucomycin V, 3-acetate 4B-(3-methylbutanoate), or an analogue or derivative thereof).

44) GPIIb IIIa Receptor Antagonists

In another embodiment, the pharmacologically active compound is a GPIIb IIa receptor antagonist (e.g., tirofiban hydrochloride (L-tyrosine, N-(butylsulfonyl)-O-(4-(4-piperidinyl)butyl)-, monohydrochloride-), eptifibatide (L-cysteinamide, N6-(aminoiminomethyl)-N-2-(3-mercapto-1-oxopropyl)L-lysylglycyl-L-alpha-aspartyl-L-tryptophyl-L-prolyl-, cyclic(1->6)-disulfide), xemilofiban hydrochloride, or an analogue or derivative thereof).

45) Endothelin Receptor Antagonists

In another embodiment, the pharmacologically active compound is an endothelin receptor antagonist (e.g., bosentan (benzenesulfonamide, 4-(1,1-dimethylethyl)-N-(6-(2-hydroxyethoxy)-5-(2-methoxyphenoxy)(2,2′-bipyrimidin)-4-yl)-, or an analogue or derivative thereof).

46) Peroxisome Proliferator-Activated Receptor Agonists

In another embodiment, the pharmacologically active compound is a peroxisome proliferator-activated receptor agonist (e.g., gemfibrozil (pentanoic acid, 5-(2,5-dimethylphenoxy)-2,2-dimethyl-), fenofibrate (propanoic acid, 2-(4-(4-chlorobenzoyl)phenoxy)-2-methyl-, 1-methylethyl ester), ciprofibrate (propanoic acid, 2-(4-(2,2-dichlorocyclopropyl)phenoxy)-2-methyl-), rosiglitazone maleate (2,4-thiazolidinedione, 5-((4-(2-(methyl-2-pyridinylamino)ethoxy)phenyl)methyl)-, (Z)-2-butenedioate (1:1)), pioglitazone hydrochloride (2,4-thiazolidinedione, 5-((4-(2-(5-ethyl-2-pyridinyl)ethoxy)phenyl)methyl)-, monohydrochloride (+/−)-), etofylline clofibrate (propanoic acid, 2-(4-chlorophenoxy)-2-methyl-, 2-(1,2,3,6-tetrahydro-1,3-dimethyl-2,6-dioxo-7H-purin-7-yl)ethyl ester), etofibrate (3-pyridinecarboxylic acid, 2-(2-(4-chlorophenoxy)-2-methyl-1-oxopropoxy)ethyl ester), clinofibrate (butanoic acid, 2,2′-(cyclohexylidenebis(4,1-phenyleneoxy))bis(2-methyl-)), bezafibrate (propanoic acid, 2-(4-(2-((4-chlorobenzoyl)amino)ethyl)phenoxy)-2-methyl-), binifibrate (3-pyridinecarboxylic acid, 2-(2-(4-chlorophenoxy)-2-methyl-1-oxopropoxy)-1,3-propanediyl ester), or an analogue or derivative thereof).

In one aspect, the pharmacologically active compound is a peroxisome proliferator-activated receptor alpha agonist, such as GW-590735, GSK-677954, GSK501516, pioglitazone hydrochloride (2,4-thiazolidinedione, 5-((4-(2-(5-ethyl-2-pyridinyl)ethoxy)phenyl)methyl)-, monohydrochloride (+/−)-, or an analogue or derivative thereof).

47) Estrogen Receptor Agents

In another embodiment, the pharmacologically active compound is an estrogen receptor agent (e.g., estradiol, 17-α-estradiol, or an analogue or derivative thereof).

48) Somatostatin Analogues

In another embodiment, the pharmacologically active compound is a somatostatin analogue (e.g., angiopeptin, or an analogue or derivative thereof).

49) Neurokinin 1 Antagonists

In another embodiment, the pharmacologically active compound is a neurokinin 1 antagonist (e.g., GW-597599, lanepitant ((1,4′-bipiperidine)-1′-acetamide, N-(2-(acetyl((2-methoxyphenyl)methyl)amino)-1-(1H-indol-3-ylmethyl)ethyl)-(R)-), nolpitantium chloride (1-azoniabicyclo(2.2.2)octane, 1-(2-(3-(3,4-dichlorophenyl)-1-((3-(1-methylethoxy)phenyl)acetyl)-3-piperidinyl)ethyl)-4-phenyl-, chloride, (S)-), or saredutant (benzamide, N-(4-(4-(acetylamino)-4-phenyl-1-piperidinyl)-2-(3,4-dichlorophenyl)butyl)-N-methyl-, (S)-), or vofopitant (3-piperidinamine, N-((2-methoxy-5-(5-(trifluoromethyl)-1H-tetrazol-1-yl)phenyl)methyl)-2-phenyl-, (2S,3S)-, or an analogue or derivative thereof).

50) Neurokinin 3 Antagonist

In another embodiment, the pharmacologically active compound is a neurokinin 3 antagonist (e.g., talnetant (4-quinolinecarboxamide, 3-hydroxy-2-phenyl-N-((1 S)-1-phenylpropyl)-, or an analogue or derivative thereof).

51) Neurokinin Antagonist

In another embodiment, the pharmacologically active compound is a neurokinin antagonist (e.g., GSK-679769, GSK-823296, SR-489686 (benzamide, N-(4-(4-(acetylamino)-4-phenyl-1-piperidinyl)-2-(3,4-dichlorophenyl)butyl)-N-methyl-, (S)-3, SB-223412; SB-235375 (4-quinolinecarboxamide, 3-hydroxy-2-phenyl-N-((1S)-1-phenylpropyl)-), UK-226471, or an analogue or derivative thereof).

52) VLA-4 Antagonist

In another embodiment, the pharmacologically active compound is a LA-4 antagonist (e.g., GSK683699, or an analogue or derivative thereof).

53) Osteoclast Inhibitor

In another embodiment, the pharmacologically active compound is a osteoclast inhibitor (e.g., ibandronic acid (phosphonic acid, (1-hydroxy-3-(methylpentylamino)propylidene)bis-), alendronate sodium, or an analogue or derivative thereof).

54) DNA topoisomerase ATP Hydrolysing Inhibitor

In another embodiment, the pharmacologically active compound is a DNA topoisomerase ATP hydrolysing inhibitor (e.g., enoxacin (1,8-naphthyridine-3-carboxylic acid, 1-ethyl-6-fluoro-1,4-dihydro-4-oxo-7-(1-piperazinyl)-), levofloxacin (7H-Pyrido(1,2,3-de)-1,4-benzoxazine-6-carboxylic acid, 9-fluoro-2,3-dihydro-3-methyl-10-(4-methyl-1-piperazinyl)-7-oxo-, (S)-), ofloxacin (7H-pyrido(1,2,3-de)-1,4-benzoxazine-6-carboxylic acid, 9-fluoro-2,3-dihydro-3-methyl-10-(4-methyl-1-piperazinyl)-7-oxo-, (+/−)-), pefloxacin (3-quinolinecarboxylic acid, 1-ethyl-6-fluoro-1,4-dihydro-7-(4-methyl-1-piperazinyl)-4-oxo-), pipemidic acid (pyrido(2,3-d)pyrimidine-6-carboxylic acid, 8-ethyl-5,8-dihydro-5-oxo-2-(1-piperazinyl)-), pirarubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,1-trihydroxy-8-(hydroxyacetyl)-1-methoxy-, (8S-(8 alpha,10 alpha(S*)))-), sparfloxacin (3-quinolinecarboxylic acid, 5-amino-1-cyclopropyl-7-(3,5-dimethyl-1-piperazinyl)-6,8-difluoro-1,4-dihydro-4-oxo-, cis-), AVE-6971, cinoxacin ((1,3)dioxolo(4,5-g)cinnoline-3-carboxylic acid, 1-ethyl-1,4-dihydro-4-oxo-), or an analogue or derivative thereof).

55) Angiotensin I Converting Enzyme Inhibitor

In another embodiment, the pharmacologically active compound is an angiotensin I converting enzyme inhibitor (e.g., ramipril (cyclopenta(b)pyrrole-2-carboxylic acid, 1-(2-((1-(ethoxycarbonyl)-3-phenylpropyl)amino)-1-oxopropyl)octahydro-, (2S-(1 (R*(R*)),2 alpha, 3aβ, 6aβ))-), trandolapril (1H-indole-2-carboxylic acid, 1-(2-((1-carboxy-3-phenylpropyl)amino)-1-oxopropyl)octahydro-, (2S-(1(R*(R*)),2 alpha,3a alpha,7aβ))-), fasidotril (L-alanine, N-((2S)-3-(acetylthio)-2-(1,3-benzodioxol-5-ylmethyl)-1-oxopropyl)-, phenylmethyl ester), cilazapril (6H-pyridazino(1,2-a)(1,2)diazepin-1-carboxylic acid, 9-((1-(ethoxycarbonyl)-3-phenylpropyl)amino)octahydro-10-oxo-, (1S-(1 alpha, 9 alpha(R*)))-), ramipril (cyclopenta(b)pyrrole-2-carboxylic acid, 1-(2-((1-(ethoxycarbonyl)-3-phenylpropyl)amino)-1-oxopropyl)octahydro-, (2S-(1(R*(R*)), 2 alpha,3aβ,6aβ))-, or an analogue or derivative thereof).

56) Angiotensin II Antagonist

In another embodiment, the pharmacologically active compound is an angiotensin II antagonist (e.g., HR-720 (1H-imidazole-5-carboxylic acid, 2-butyl-4-(methylthio)-1-((2′-((((propylamino)carbonyl)amino)sulfonyl)(1,1′-biphenyl)-4-yl)methyl)-, dipotassium salt, or an analogue or derivative thereof).

57) Enkephalinase Inhibitor

In another embodiment, the pharmacologically active compound is an enkephalinase inhibitor (e.g., Aventis 100240 (pyrido(2,1-a)(2)benzazepine-4-carboxylic acid, 7-((2-(acetylthio)-1-oxo-3-phenylpropyl)amino)-1,2,3,4,6,7,8,12b-octahydro-6-oxo-, (4S-(4 alpha, 7 alpha(R*),12bβ))-), AVE-7688, or an analogue or derivative thereof).

58) Peroxisome Proliferator-Activated Receptor Gamma Agonist Insulin Sensitizer

In another embodiment, the pharmacologically active compound is peroxisome proliferator-activated receptor gamma agonist insulin sensitizer (e.g., rosiglitazone maleate (2,4-thiazolidinedione, 5-((4-(2-(methyl-2-pyridinylamino)ethoxy)phenyl)methyl)-, (Z)-2-butenedioate (1:1), farglitazar (GI-262570, GW-2570, GW-3995, GW-5393, GW-9765), LY-929, LY-519818, LY-674, or LSN-862), or an analogue or derivative thereof).

59) Protein Kinase C Inhibitor

In another embodiment, the pharmacologically active compound is a protein kinase C inhibitor, such as ruboxistaurin mesylate (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)-), safingol (1,3-octadecanediol, 2-amino-, (S—(R*,R*))-), or enzastaurin hydrochloride (1H-pyrole-2,5-dione, 3-(1-methyl-1H-indol-3-yl)-4-(1-(1-(2-pyridinylmethyl)-4-piperidinyl)-1H-indol-3-yl)-, monohydrochloride), or an analogue or derivative thereof.

60) ROCK (rho-Associated Kinase) Inhibitors

In another embodiment, the pharmacologically active compound is a ROCK (rho-associated kinase) inhibitor, such as Y-27632, HA-1077, H-1152 and 4-1-(aminoalkyl)-N-(4-pyridyl)cyclohexanecarboxamide or an analogue or derivative thereof.

61) CXCR3 Inhibitors

In another embodiment, the pharmacologically active compound is a CXCR3 inhibitor such as T-487, T0906487 or analogue or derivative thereof.

62) Itk Inhibitors

In another embodiment, the pharmacologically active compound is an Itk inhibitor such as BMS-509744 or an analogue or derivative thereof.

63) Cytosolic Phospholipase A₂-Alpha Inhibitors

In another embodiment, the pharmacologically active compound is a cytosolic phospholipase A₂-alpha inhibitor such as efipladib (PLA-902) or analogue or derivative thereof.

64) PPAR Agonist

In another embodiment, the pharmacologically active compound is a PPAR agonist (e.g., Metabolex ((−)-benzeneacetic acid, 4-chloro-alpha-(3-(trifluoromethyl)-phenoxy)-, 2-(acetylamino)ethyl ester), balaglitazone (5-(4-(3-methyl-4-oxo-3,4-dihydro-quinazolin-2-yl-methoxy)-benzyl)-thiazolidine-2,4-dione), ciglitazone (2,4-thiazolidinedione, 5-((4-((1-methylcyclohexyl)methoxy)phenyl)methyl)-), DRF-10945, farglitazar, GSK-677954, GW-409544, GW-501516, GW-590735, GW-590735, K-111, KRP-101, LSN-862, LY-519818, LY-674, LY-929, muraglitazar; BMS-298585 (Glycine, N-((4-methoxyphenoxy)carbonyl)-N-((4-(2-(5-methyl-2-phenyl-4-oxazolyl)ethoxy)phenyl)methyl)-), netoglitazone; isaglitazone (2,4-thiazolidinedione, 5-((6-((2-fluorophenyl)methoxy)-2-naphthalenyl)methyl)-), Actos AD-4833; U-72107A (2,4-thiazolidinedione, 5-(4-(2-(5-ethyl-2-pyridinyl)ethoxy)phenyl)methyl)-, monohydrochloride (+/−)-), JTT-501; PNU-182716 (3,5-Isoxazolidinedione, 4-((4-(2-(5-methyl-2-phenyl-4-oxazolyl)ethoxy)phenyl)methyl), AVANDIA (from SB Pharmco Puerto Rico, Inc. (Puerto Rico); BRL-48482; BRL-49653; BRL-49653c; NYRACTA and Venvia(both from (SmithKline Beecham (United Kingdom)); tesaglitazar ((2S)-2-ethoxy-3-(4-(2-(4-((methylsulfonyl)oxy)phenyl)ethoxy)phenyl)propanoic acid), troglitazone (2,4-Thiazolidinedione, 5-((4-((3,4-dihydro-6-hydroxy-2,5,7,8-tetramethyl-2H-1-benzopyran-2-yl)methoxy)phenyl)methyl)-), and analogues and derivatives thereof).

65) Immunosuppressants

In another embodiment, the pharmacologically active compound is an immunosuppressant (e.g., batebulast (cyclohexanecarboxylic acid, 4-(((aminoiminomethyl)amino)methyl)-, 4-(1,1-dimethylethyl)phenyl ester, trans-), cyclomunine, exalamide (benzamide, 2-(hexyloxy)-), LYN-001, CCI-779 (rapamycin 42-(3-hydroxy-2-(hydroxymethyl)-2-methylpropanoate)), 1726; 1726-D; AVE-1726, or an analogue or derivative thereof).

66) Erb Inhibitor

In another embodiment, the pharmacologically active compound is an Erb inhibitor (e.g., canertinib dihydrochloride (N-(4-(3-(chloro-4-fluoro-phenylamino)-7-(3-morpholin-4-yl-propoxy)-quinazolin-6-yl)-acrylamide dihydrochloride), CP-724714, or an analogue or derivative thereof).

67) Apoptosis Agonist

In another embodiment, the pharmacologically active compound is an apoptosis agonist (e.g., CEFLATONIN (CGX-635) (from Chemgenex Therapeutics, Inc., Menlo Park, Calif.), CHML, LBH-589, metoclopramide (benzamide, 4-amino-5-chloro-N-(2-(diethylamino)ethyl)-2-methoxy-), patupilone (4,17-dioxabicyclo(14.1.0)heptadecane-5,9-dione, 7,11-dihydroxy-8,8,10,12,16-pentamethyl-3-(1-methyl-2-(2-methyl-4-thiazolyl)ethenyl, (1R,3S,7S,10R,11S,12S,16R)), AN-9; pivanex (butanoic acid, (2,2-dimethyl-1-oxopropoxy)methyl ester), SL-100; SL-102; SL-11093; SL-11098; SL-11099; SL-93; SL-98; SL-99, or an analogue or derivative thereof).

68) Lipocortin Agonist

In another embodiment, the pharmacologically active compound is an lipocortin agonist (e.g., CGP-13774 (9Alpha-chloro-6Alpha-fluoro-11β,17alpha-dihydroxy-16Alpha-methyl-3-oxo-1,4-androstadiene-17β-carboxylic acid-methylester-17-propionate), or analogue or derivative thereof).

69) VCAM-1 Antagonist

In another embodiment, the pharmacologically active compound is a VCAM-1 antagonist (e.g., DW-908e, or an analogue or derivative thereof.

70) Collagen Antagonist

In another embodiment, the pharmacologically active compound is a collagen antagonist (e.g., E-5050 (Benzenepropanamide, 4-(2,6-dimethylheptyl)-N-(2-hydroxyethyl)-β-methyl-), lufironil (2,4-Pyridinedicarboxamide, N,N′-bis(2-methoxyethyl)-), or an analogue or derivative thereof.

71) Alpha 2 Integrin Antagonist

In another embodiment, the pharmacologically active compound is an alpha 2 integrin antagonist (e.g., E-7820, or an analogue or derivative thereof.

72) TNF Alpha Inhibitor

In another embodiment, the pharmacologically active compound is a TNF alpha inhibitor (e.g., ethyl pyruvate, Genz-29155, lentinan (Ajinomoto Co., Inc. (Japan)), linomide (3-quinolinecarboxamide, 1,2-dihydro-4-hydroxy-N, 1-dimethyl-2-oxo-N-phenyl-), UR-1505, or an analogue or derivative thereof).

73) Nitric Oxide Inhibitor

In another embodiment, the pharmacologically active compound is a nitric oxide inhibitor (e.g., guanidioethyldisulfide, or an analogue or derivative thereof).

74) Cathepsin Inhibitor

In another embodiment, the pharmacologically active compound is a cathepsin inhibitor (e.g., SB-462795 or an analogue or derivative thereof).

Combination Therapies

In addition to incorporation of a fibrosis-inhibiting agent, one or more other pharmaceutically active agents can be incorporated into the present compositions to improve or enhance efficacy. In one aspect, the composition may further include a compound that acts to have an inhibitory effect on pathological processes in or around the treatment site. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

In one aspect, the present invention also provides for the combination of a soft tissue implant (as well as compositions and methods for making soft tissue implants) that includes an anti-fibrosing agent and an anti-infective agent, which reduces the likelihood of infections.

Infection is a common complication of the implantation of foreign bodies such as, for example, medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based the assays provided in Example 37. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

a) Anthracyclines

Anthracyclines have the following general structure, where the R groups may be a variety of organic groups:

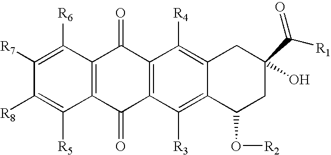

According to U.S. Pat. No. 5,594,158, suitable R groups are as follows: R₁, is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 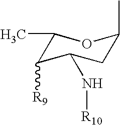
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

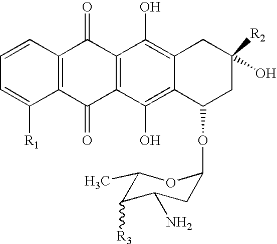

| | R₁ | R₂ | R₃ |
|---|---|---|---|
| Doxorubicin: | OCH₃ | C(O)CH₂OH | OH out of ring plane |
| Epirubicin: | OCH₃ | C(O)CH₂OH | OH in ring plane |
| (4′ epimer of | | | |
| doxorubicin) | | | |
| Daunorubicin: | OCH₃ | C(O)CH₃ | OH out of ring plane |
| Idarubicin: | H | C(O)CH₃ | OH out of ring plane |
| Pirarubicin: | OCH₃ | C(O)CH₂OH | 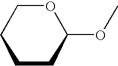 |
| Zorubicin: | OCH₃ | C(CH₃)(═N)NHC(O) | OH |
| | | C₆H₅ | |
| Carubicin: | OH | C(O)CH₃ | OH out of ring plane |

Other suitable anthracyclines are anthramycin, mitoxantrone, menogaril, nogalamycin, aclacinomycin A, olivomycin A, chromomycin A₃, and plicamycin having the structures:

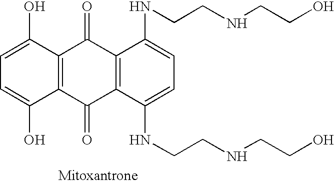

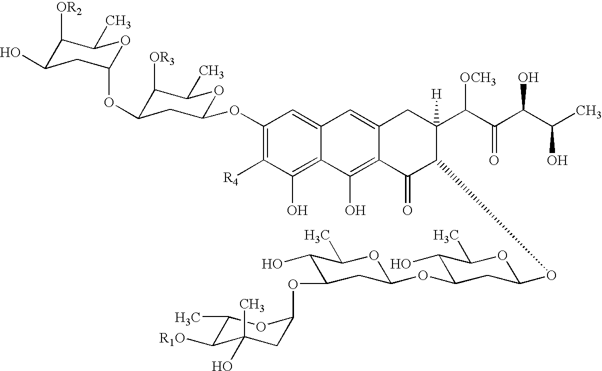

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

| | R₁ | R₂ | R₃ |
|---|---|---|---|
| Menogaril | H | OCH₃ | H |
| Nogalamycin | O-sugar | H | COOCH₃ |

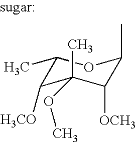

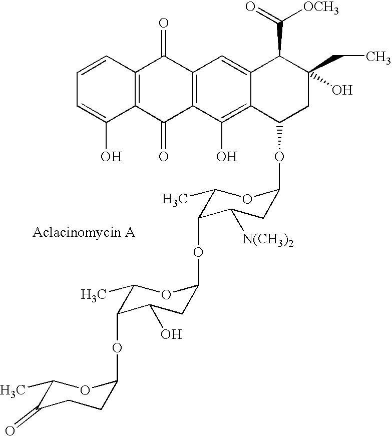

Other representative anthracyclines include, FCE 23762, a doxorubicin derivative (Quaglia et al., J. Liq. Chromatogr. 17(18): 3911-3923, 1994), annamycin (Zou et al., J. Pharm. Sci. 82(11): 1151-1154, 1993), ruboxyl (Rapoport et al., J. Controlled Release 58(2).153-162, 1999), anthracycline disaccharide doxorubicin analogue (Pratesi et al., Clin. Cancer Res. 4(11): 2833-2839, 1998), N-(trifluoroacetyl)doxorubicin and 4′-O-acetyl-N-(trifluoroacetyl)doxorubicin (Berube & Lepage, Synth. Commun. 28(6): 1109-1116, 1998), 2-pyrrolinodoxorubicin (Nagy et al., Proc. Nat'l Acad. Sci. U.S.A. 95(4): 1794-1799, 1998), disaccharide doxorubicin analogues (Arcamone et al., J. Nat'l Cancer Inst. 89(16): 1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)-adriamicinone doxorubicin disaccharide analogue (Monteagudo et al., Carbohydr. Res. 300(1): 11-16, 1997), 2-pyrrolinodoxorubicin (Nagy et al., Proc. Nat'l Acad. Sci. U.S.A. 94(2): 652-656, 1997), morpholinyl doxorubicin analogues (Duran et al., Cancer Chemother. Pharmacol. 38(3): 210-216, 1996), enaminomalonyl-β-alanine doxorubicin derivatives (Seitz et al., Tetrahedron Lett. 36(9): 1413-16, 1995), cephalosporin doxorubicin derivatives (Vrudhula et al., J. Med. Chem. 38(8): 1380-5, 1995), hydroxyrubicin (Solary et al., Int J. Cancer 58(1): 85-94, 1994), methoxymorpholino doxorubicin derivative (Kuhl et al., Cancer Chemother. Pharmacol. 33(1): 10-16, 1993), (6-maleimidocaproyl)hydrazone doxorubicin derivative (Wiliner et al., Bioconjugate Chem. 4(6): 521-7, 1993), N-(5,5-diacetoxypent-1-yl) doxorubicin (Cherif & Farquhar, J. Med. Chem. 35(17): 3208-14, 1992), FCE 23762 methoxymorpholinyl doxorubicin derivative (Ripamonti et al., Br. J. Cancer 65(5): 703-7, 1992), N-hydroxysuccinimide ester doxorubicin derivatives (Demant et al., Biochim. Biophys. Acta 1118(1): 83-90, 1991), polydeoxynucleotide doxorubicin derivatives (Ruggiero et al., Biochim. Biophys. Acta 1129(3): 294-302, 1991), morpholinyl doxorubicin derivatives (EPA 434960), mitoxantrone doxorubicin analogue (Krapcho et al., J. Med. Chem. 34(8): 2373-80. 1991), AD198 doxorubicin analogue (Traganos et al., Cancer Res. 51(14): 3682-9, 1991), 4-demethoxy-3′-N-trifluoroacetyldoxorubicin (Horton et al., Drug Des. Delivery 6(2): 123-9, 1990), 4′-epidoxorubicin (Drzewoski et al., Pol. J. Pharmacol. Pharm. 40(2): 159-65, 1988; Weenen et al., Eur. J. Cancer Clin. Oncol. 20(7): 919-26, 1984), alkylating cyanomorpholino doxorubicin derivative (Scudder et al., J. Nat'l Cancer Inst. 80(16): 1294-8, 1988), deoxydihydroiodooxorubicin (EPA 275966), adriblastin (Kalishevskaya et al., Vestn. Mosk. Univ., 16(Biol. 1): 21-7, 1988), 4′-deoxydoxorubicin (Schoeizel et al., Leuk. Res. 10(12): 1455-9, 1986), 4-demethyoxy-4′-o-methyldoxorubicin (Giuliani et al., Proc. Int. Congr. Chemother. 16: 285-70-285-77, 1983), 3′-deamino-3′-hydroxydoxorubicin (Horton et al., J Antibiot. 37(8): 853-8, 1984), 4-demethyoxy doxorubicin analogues (Barbieri et al., Drugs Exp. Clin. Res. 10(2): 85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int J. Cancer 27(1): 5-13, 1981), aglycone doxorubicin derivatives (Chan & Watson, J. Pharm. Sci. 67(12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

b) Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

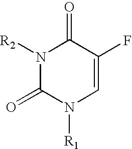

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

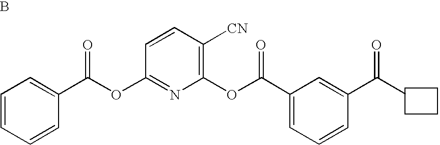

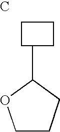

Other suitable fluoropyrimidine analogues include 5-FudR (5-fluoro-deoxyuridine), or an analogue or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Exemplary compounds have the structures:

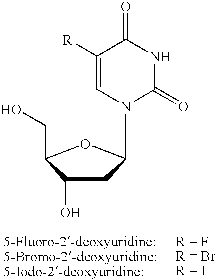

Other representative examples of fluoropyrimidine analogues include N3-alkylated analogues of 5-fluorouracil (Kozai et al., J. Chem. Soc., Perkin Trans. 1(19): 3145-3146, 1998), 5-fluorouracil derivatives with 1,4-oxaheteroepane moieties (Gomez et al., Tetrahedron 54(43): 13295-13312, 1998), 5-fluorouracil and nucleoside analogues (Li, Anticancer Res. 17(1A): 21-27, 1997), cis- and trans-5-fluoro-5,6-dihydro-6-alkoxyuracil (Van der Wilt et al., Br. J. Cancer 68(4): 702-7, 1993), cyclopentane 5-fluorouracil analogues (Hronowski & Szarek, Can. J. Chem. 70(4): 1162-9, 1992), A-OT-fluorouracil (Zhang et al., Zongguo Yiyao Gongye Zazhi 20(11): 513-15, 1989), N4-trimethoxybenzoyl-5′-deoxy-5-fluorocytidine and 5′-deoxy-5-fluorouridine (Miwa et al., Chem. Pharm. Bull. 38(4): 998-1003, 1990), 1-hexylcarbamoyl-5-fluorouracil (Hoshi et al., J. Pharmacobio-Dun. 3(9): 478-81, 1980; Maehara et al., Chemotherapy (Basel) 34(6): 484-9, 1988), B-3839 (Prajda et al., In Vivo 2(2): 151-4, 1988), uracil-1-(2-tetrahydrofuryl)-5-fluorouracil (Anai et al., Oncology 45(3): 144-7, 1988), 1-(2′-deoxy-2′-fluoro-β-D-arabinofuranosyl)-5-fluorouracil (Suzuko et al., Mol. Pharmacol. 31(3): 301-6, 1987), doxifluridine (Matuura et al., Oyo Yakuri 29(5): 803-31, 1985), 5′-deoxy-5-fluorouridine (Bollag & Hartmann, Eur. J. Cancer 16(4): 427-32, 1980), 1-acetyl-3-O-toluyl-5-fluorouracil (Okada, Hiroshima J. Med. Sci. 28(1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

c) Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

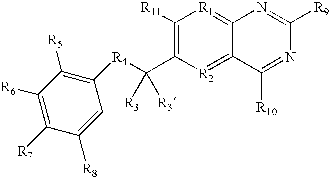

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 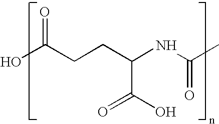
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

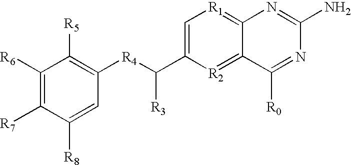

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | N(CH₂CH₃) | H | H | A(n = 1) | H |
| Trimetrexate | NH₂ | N | C(CH₃) | H | NH | H | OCH₃ | OCH₃ | OCH₃ |
| Pteropterin | OH | N | N | H | NH | H | H | A(n = 3) | H |
| Denopterin | OH | N | N | CH₃ | N(CH₃) | H | H | A(n = 1) | H |
| Peritrexim | NH₂ | N | C(CH₃) | H | single | OCH₃ | H | H | OCH₃ |
| | | | | bond | | | | | |

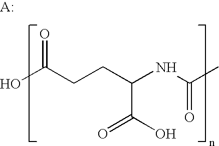

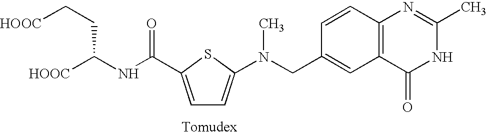

Other representative examples include 6-S-aminoacyloxymethyl mercaptopurine derivatives (Harada et al., Chem. Pharm. Bull. 43(10): 793-6, 1995), 6-mercaptopurine (6-MP) (Kashida et al, Biol. Pharm. Bull. 18(11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56(4): 249-64, 1994), methyl-D-glucopyranoside mercaptopurine derivatives (Da Silva et al., Eur. J. Med. Chem. 29(2): 149-52, 1994) and s-alkynyl mercaptopurine derivatives (Ratsino et al., Khim.-Farm. Zh. 15(8): 65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12): 2287-2293, 1996), benzoxazine or benzothiazine moiety-bearing methotrexate derivatives (Matsuoka et al., J. Med. Chem. 40(1): 105-111, 1997), 10-deazaminopterin analogues (DeGraw et al., J. Med. Chem. 40(3): 370-376, 1997), 5-deazaminopterin and 5,10-dideazaminopterin methotrexate analogues (Piper et al., J. Med. Chem. 40(3): 377-384, 1997), indoline moiety-bearing methotrexate derivatives (Matsuoka et al., Chem. Pharm. Bull. 44(7): 1332-1337, 1996), lipophilic amide methotrexate derivatives (Pignatello et al., World Meet. Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1): 56-65, 1996), methotrexate tetrahydroquinazoline analogue (Gangjee, et al., J. Heterocycl. Chem. 32(1): 243-8, 1995), N-(α-aminoacyl)methotrexate derivatives (Cheung et al., Pteridines 3(1-2): 101-2, 1992), biotin methotrexate derivatives (Fan et al., Pteridines 3(1-2): 131-2, 1992), D-glutamic acid or D-erythrou, threo-4-fluoroglutamic acid methotrexate analogues (McGuire et al., Biochem. Pharmacol. 42(12): 2400-3, 1991), β,γ-methano methotrexate analogues (Rosowsky et al., Pteridines 2(3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-a-aminoacyl) methotrexate derivatives (Cheung et al., Heterocycles 28(2): 751-8, 1989), meta and ortho isomers of aminopterin (Rosowsky et al., J. Med. Chem. 32(12): 2582, 1989), hydroxymethylmethotrexate (DE 267495), γ-fluoromethotrexate (McGuire et al., Cancer Res. 49(16): 4517-25, 1989), polyglutamyl methotrexate derivatives (Kumar et al., Cancer Res. 46(10): 5020-3, 1986), gem-diphosphonate methotrexate analogues (WO 88/06158), α- and γ-substituted methotrexate analogues (Tsushima et al., Tetrahedron 44(17): 5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7): 1332-7, 1988), 8-deaza methotrexate analogues (Kuehl et al., Cancer Res. 48(6): 1481-8, 1988), acivicin methotrexate analogue (Rosowsky et al., J. Med. Chem. 30(8): 1463-9, 1987), polymeric platinol methotrexate derivative (Carraher et al., Polym. Sci. Technol. (Plenum), 35(Adv. Biomed. Polym.): 311-24, 1987), methotrexate-γ-dimyristoylphophatidylethanolamine (Kinsky et al., Biochim. Biophys. Acta 917(2): 211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-y-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines FQlic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15): 2607-13, 1986), polyglutamate methotrexate derivatives (Kamen & Winick, Methods Enzymol. 122(Vitam. Coenzymes, Pt. G): 339-46, 1986), 5-methyl-5-deaza analogues (Piper et al., J. Med. Chem. 29(6): 1080-7, 1986), quinazoline methotrexate analogue (Mastropaolo et al., J. Med. Chem. 29(1): 155-8, 1986), pyrazine methotrexate analogue (Lever & Vestal, J. Heterocycl. Chem. 22(1): 5-6, 1985), cysteic acid and homocysteic acid methotrexate analogues (U.S. Pat. No. 4,490,529), γ-tert-butyl methotrexate esters (Rosowsky et al., J. Med. Chem. 28(5): 660-7, 1985), fluorinated methotrexate analogues (Tsushima et al., Heterocycles 23(1): 45-9, 1985), folate methotrexate analogue (Trombe, J. Bacteriol. 160(3): 849-53, 1984), phosphbnoglutamic acid analogues (Sturtz & Guillamot, Eur. J. Med. Chem.—Chim. Ther. 19(3): 267-73,1984), poly(L-lysine)methotrexate conjugates (Rosowsky et al., J. Med. Chem. 27(7): 888-93, 1984), dilysine and trilysine methotrexate derivates (Forsch & Rosowsky, J. Org. Chem. 49(7): 1305-9, 1984), 7-hydroxyrnethotrexate (Fabre et al., Cancer Res. 43(10): 4648-52, 1983), poly-γ-glutamyl methotrexate analogues (Piper & Montgomery, Adv. Exp. Med. Biol., 163(Folyl Antifolyl Polyglutamates): 95-100, 1983), 3′,5′-dichloromethotrexate (Rosowsky & Yu, J. Med. Chem. 26(10): 1448-52, 1983), diazoketone and chloromethylketone methotrexate analogues (Gangjee et al., J. Pharm. Sci. 71(6): 717-19, 1982), 10-propargylaminopterin and alkyl methotrexate homologs (Piper et al., J. Med. Chem. 25(7): 877-80, 1982), lectin derivatives of methotrexate (Lin et al., JNCI 66(3): 523-8, 1981), polyglutamate methotrexate derivatives (Galivan, Mol. Pharmacol. 17(1): 105-10, 1980), halogentated methotrexate derivatives (Fox, JNCI 58(4): J955-8, 1977), 8-alkyl-7,8-dihydro analogues (Chaykovsky et al., J. Med. Chem. 20(10): J1323-7, 1977), 7-methyl methotrexate derivatives and dichloromethotrexate (Rosowsky & Chen, J. Med. Chem. 17(12): J1308-11, 1974), lipophilic methotrexate derivatives and 3′,5′-dichloromethotrexate (Rosowsky, J. Med. Chem. 16(10): J1190-3, 1973), deaza amethopterin analogues (Montgomery et al., Ann. N.Y. Acad. Sci. 186: J227-34, 1971), MX068 (Pharma Japan, 1658: 18, 1999) and cysteic acid and homocysteic acid methotrexate analogues (EPA 0142220);

These compounds are believed to act as antimetabolites of folic acid.

d) Podophyllotoxins

In another aspect, the therapeutic agent is a podophyllotoxin, or a derivative or an analogue thereof. Exemplary compounds of this type are etoposide or teniposide, which have the following structures:

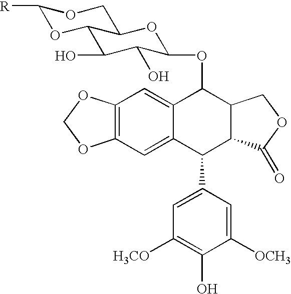

| | R |
|---|---|
| Etoposide | CH₃ |
| Teniposide | 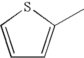 |

Other representative examples of podophyllotoxins include Cu(II)-VP-16 (etoposide) complex (Tawa et al., Bioorg. Med. Chem. 6(7): 1003-1008, 1998), pyrrolecarboxamidino-bearing etoposide analogues (Ji et al., Bioorg. Med. Chem. Left. 7(5): 607-612, 1997), 4β-amino etoposide analogues (Hu, University of North Carolina Dissertation, 1992), γ-lactone ring-modified arylamino etoposide analogues (Zhou et al., J. Med. Chem. 37(2): 287-92, 1994), N-glucosyl etoposide analogue (Allevi et al., Tetrahedron Lett. 34(45): 7313-16, 1993), etoposide A-ring analogues (Kadow et al., Bioorg. Med. Chem. Lett. 2(1): 17-22, 1992), 4′-deshydroxy-4′-methyl etoposide (Saulnier et al., Bioorg. Med. Chem. Left. 2(10): 1213-18, 1992), pendulum ring etoposide analogues (Sinha et al., Eur. J. Cancer 26(5): 590-3, 1990) and E-ring desoxy etoposide analogues (Saulnier et al., J. Med. Chem. 32(7): 1418-20, 1989).

These compounds are believed to act as topoisomerase 11 inhibitors and/or DNA cleaving agents.

e) Camptothecins

In another aspect, the therapeutic agent is camptothecin, or an analogue or derivative thereof. Camptothecins have the following general structure.

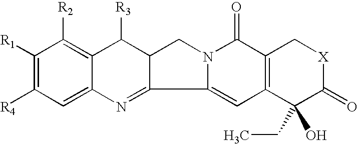

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

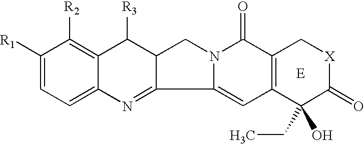

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

f) Hydroxyureas

The therapeutic agent of the present invention may be a hydroxyurea. Hydroxyureas have the following general structure:

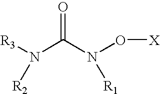

Suitable hydroxyureas are disclosed in, for example, U.S. Pat. No. 6,080,874, wherein R₁ is: 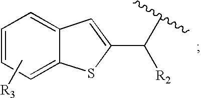
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 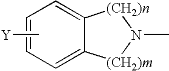
wherein m is 1 or 2, n is 0-2 and Y is an alkyl group.

In one aspect, the hydroxyurea has the structure:

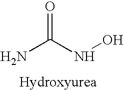

These compounds are thought to function by inhibiting DNA synthesis.

g) Platinum Complexes

In another aspect, the therapeutic agent is a platinum compound. In general, suitable platinum complexes may be of Pt(II) or Pt(IV) and have this basic structure: 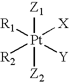
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

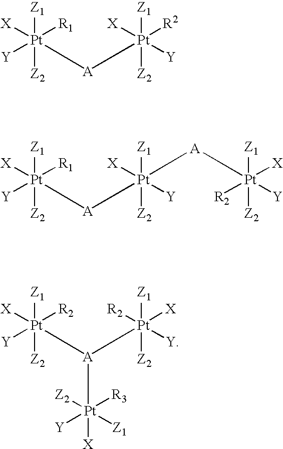

Exemplary platinum compounds are cisplatin, carboplatin, oxaliplatin, and miboplatin having the structures:

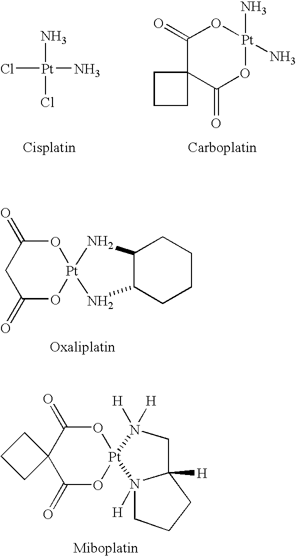

Other representative platinum compounds include (CPA)₂Pt(DOLYM) and (DACH)Pt(DOLYM)cisplatin (Choi et al., Arch. Pharmacal Res. 22(2): 151-156, 1999), Cis-(PtCl₂(4,7-H-5-methyl-7-oxo)1,2,4(triazolo(1,5-a)pyrimidine)₂) (Navarro et al., J. Med. Chem. 41(3): 332-338, 1998), (Pt(cis-1,4-DACH)(trans-Cl₂)(CBDCA))•½MeOH cisplatin (Shamsuddin et al., Inorg. Chem. 36(25): 5969-5971, 1997), 4-pyridoxate diammine hydroxy platinum (Tokunaga et al., Pharm. Sci. 3(7): 353-356, 1997), Pt(II) . . . Pt(II) (Pt₂(NHCHN(C(CH₂)(CH₃)))₄) (Navarro et al., Inorg. Chem. 35(26): 7829-7835, 1996), 254-S cisplatin analogue (Koga et al., Neurol. Res. 18(3): 244-247, 1996), o-phenylenediamine ligand bearing cisplatin analogues (Koeckerbauer & Bednarski, J. Inorg. Biochem. 62(4): 281-298, 1996), trans, cis-(Pt(OAc)₂I₂(en)) (Kratochwil et al., J. Med. Chem. 39(13): 2499-2507, 1996), estrogenic 1,2-diarylethylenediamine ligand (with sulfur-containing amino acids and glutathione) bearing cisplatin analogues (Bednarski, J. Inorg. Biochem. 62(1): 75, 1996), cis-1,4-diaminocyclohexane cisplatin analogues (Shamsuddin et al., J. Inorg. Biochem. 61(4): 291-301, 1996), 5′ orientational isomer of cis-(Pt(NH₃)(4-amino TEMP-O){d(GpG)}) (Dunham & Lippard, J. Am. Chem. Soc. 117(43): 10702-12, 1995), chelating diamine-bearing cisplatin analogues (Koeckerbauer& Bednarski, J. Pharm. Sci. 84(7): 819-23, 1995), 1,2-diarylethyleneamine ligand-bearing cisplatin analogues (Otto et al., J. Cancer Res. Clin. Oncol. 121(1): 31-8, 1995), (ethylenediamine)platinum(II) complexes (Pasini et al., J. Chem. Soc., Dalton Trans. 4: 579-85, 1995), Cl-973 cisplatin analogue (Yang et al., Int. J. Oncol. 5(3): 597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4): 257-67, 1986; Fan et al., Cancer Res. 48(11): 3135-9, 1988; Heiger-Bemays et al., Biochemistry 29(36): 8461-6, 1990; Kikkawa et al., J. Exp. Clin. Cancer Res. 12(4): 233-40, 1993; Murray et al., Biochemistry 31(47): 11812-17, 1992; Takahashi et al., Cancer Chemother. Pharmacol. 33(1): 31-5, 1993), cis-amine-cyclohexylamine-dichloroplatinum(II). (Yoshida et al., Biochem. Pharmacol. 48(4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23): 4479-85, 1992), cisplatin analogues containing a tethered dansyl group (Hartwig et al., J. Am. Chem. Soc. 114(21): 8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2): 311-15, 1991), trans-diamminedichloroplatinum(II) and cis-(Pt(NH₃)₂(N₃-cytosine)Cl) (Bellon & Lippard, Biophys. Chem. 35(2-3): 179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1): 41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4): 349-57, 1988), aminoalkylaminoanthraquinone-derived cisplatin analogues (Kitov et al., Eur. J. Med. Chem. 23(4): 381-3, 1988), spiroplatin, carboplatin, iproplatin and JM40 platinum analogues (Schroyen et aL, Eur. J. Cancer Clin. Oncol. 24(8): 1309-12, 1988), bidentate tertiary diamine-containing cisplatinum derivatives (Orbell et al., Inorg. Chim. Acta 152(2): 125-34, 1988), platinum(II), platinum(IV) (Liu & Wang, Shandong Yike Daxue Xuebao 24(1): 35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)₂((PtCL4).cis-(PtCl2-(NH2Me)2)) (Brammer et al., J. Chem. Soc., Chem. Commun. 6: 443-5, 1987), aliphatic tricarboxylic acid platinum complexes (EPA 185225), and cis-dichloro(amino acid)(tert-butylamine)platinum(II) complexes (Pasini & Bersanetti, Inorg. Chim. Acta 107(4): 259-67, 1985). These compounds are thought to function by binding to DNA, i.e., acting as alkylating agents of DNA.

As medical implants are made in a variety of configurations and sizes, the exact dose administered may vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the cardiac implant, the anticancer agents, used alone or in combination, may be administered under the following dosing guidelines:

(a) Anthracyclines. Utilizing the anthracycline doxorubicin as an example, whether applied as a polymer coating, incorporated into the polymers that make up the implant components, or applied without a carrier polymer, the total dose of doxorubicin applied to the implant should not exceed 25 mg (range of. 0.1 μg to 25 mg). In one embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In one embodiment, doxorubicin should be applied to the implant surface at a dose of 0.1 μg/mm²-10 μg/mm². As different polymer and non-polymer coatings may release doxorubicin at differing rates, the above dosing parameters should be utilized in combination with the release rate of the drug from the implant surface such that a minimum concentration of 10⁻⁸-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In one embodiment, doxorubicin is released from the surface of the implant such that anti-infective activity is maintained for a period ranging from several hours to several months. In one embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers that make up the implant, or applied without a carrier polymer, the total dose of mitoxantrone applied should not exceed 5 mg (range of 0.01 μg to 5 mg). In one embodiment, the total amount of drug applied should be in the range of 0.1 μg to 3 mg. The dose per unit area (i.e., the amount of drug as a function of the surface area of the portion of the implant to which drug is applied and/or incorporated) should fall within the range of 0.01 μg-20 μg per mm² of surface area. In one embodiment, mitoxantrone should be applied to the implant surface at a dose of 0.05 μg/mm²-5 μg/mm². As different polymer and non-polymer coatings will release mitoxantrone at differing rates, the above dosing parameters should be utilized in combination with the release rate of the drug from the implant surface such that a minimum concentration of 10⁻⁴-10⁻⁸M of mitoxantrone is maintained. It is necessary to insure that drug concentrations on the implant surface exceed concentrations of mitoxantrohe known to be lethal to multiple species of bacteria and fungi (i.e., are in excess of 10⁻⁵ M; although for some embodiments lower drug levels will be sufficient). In one embodiment, mitoxantrone is released from the surface of the implant such that anti-infective activity is maintained for a period ranging from several hours to several months. In one embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines. Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In one embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.05 μg-200 μg per mm² of surface area. In one embodiment, 5-fluorouracil should be applied to the implant surface at a dose of 0.5 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In one embodiment, 5-fluorouracil is released from the implant surface such that anti-infective activity is maintained for a period ranging from several hours to several months. In one embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins. Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In one embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In one embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁴-10⁻⁷ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In one embodiment, etoposide is released from the surface of the implant such that anti-infective activity is maintained for a period ranging from several hours to several months. In one embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It may be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the composition.

In another aspect, an anti-infective agent (e.g., anthracyclines (e.g., doxorubicin or mitoxantrone), fluoropyrimidines (e.g., 5-fluorouracil), folic acid antagonists. (e.g., methotrexate and/or podophylotoxins (e.g., etoposide)) can be combined with traditional antibiotic and/or antifungal agents to enhance efficacy. The anti-infective agent may be further combined with anti-thrombotic and/or antiplatelet agents (for example, heparin, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

In addition to incorporation of the above-mentioned therapeutic agents (i.e., anti-infective agents or fibrosis-inhibiting agents), one or more other pharmaceutically active agents can be incorporated into the present compositions and devices to improve or enhance efficacy. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

Soft tissue implants and compositions for use with soft tissue implants may further include an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound that prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

Compositions for use with soft tissue implants may be or include a hydrophilic polymer gel that itself has anti-thrombogenic properties. For example, the composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Soft tissue implants and compositions for use with soft tissue implants may further include a compound that acts to have an inhibitory effect on pathological processes in or around the treatment site. In certain aspects, the agent may be selected from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331 A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071 A1, WO 99/001545A1, WO 97/40028A1, WO 97/41211A1, WO 98/40381 A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/018141A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be combined With soft tissue implants according to the invention include tyrosine kinase inhibitors, such as imantinib, ZK-222584, CGP-52411, CGP-53716, NVP-AAK980-NX, CP-127374, CP-564959, PD-171026, PD-173956, PD-180970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and dexlipotam; p38 MAP kinase inhibitors such as include CGH-2466 and PD-98-59; immunosuppressants such as argyrin B, macrocyclic lactone, ADZ-62-826, CCl-779, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo(b,f)oxepin); and caspase inhibitors (e.g., PF-5901 (benzenemethanol, alpha-pentyl-3-(2-quinolinylmethoxy)-), and JNK inhibitor (e.g., AS-602801).

In another aspect, the soft tissue implants may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

In certain aspects, a polymeric composition comprising a fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., paclitaxel, rapamycin, everolimus) and a CYP inhibitor, which may be combined (e.g., coated) with any of the devices described herein. Representative examples of CYP inhibitors include flavones, azole antifungals, macrolide antibiotics, HIV protease inhibitors, and anti-sense oligomers. Devices comprising a combination of a fibrosis-inhibiting agent and a CYP inhibitor may be used to treat a variety of proliferative conditions that can lead to undesired scarring of tissue, including intimal hyperplasia, surgical adhesions, and tumor growth.

Within various embodiments of the invention, a device incorporates or is coated on one aspect, portion or surface with a composition which inhibits fibrosis (and/or restenosis), as Well as with a composition or compound which promotes or stimulates fibrosis on another aspect, portion or surface of the device. Compounds that promote or stimulate fibrosis can be identified by, for example, the in vivo (animal) models provided in Examples 33-36. Representative examples of agents that promote fibrosis include silk and other irritants (e.g., talc, wool (including animal wool, wood wool, and synthetic wool), talcum powder, copper, metallic beryllium (or its oxides), quartz dust, silica, crystalline silicates), polymers (e.g., polylysine, polyurethanes, poly(ethylene terephthalate), PTFE, poly(alkylcyanoacrylates), and poly(ethylene-co-vinylacetate); vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-s (TGF-β-1, TGF-β-2, TGF-β-3, platelet-derived growth factor (PDGF), fibroblast growth factor (acidic-aFGF; and basic-bFGF), fibroblast stimulating factor-1, activins, vascular endothelial growth factor (including VEGF-2, VEGF-3, VEGF-A, VEGF-B, VEGF-C, placental growth factor-PIGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNF-α), nerve growth factor (NGF), interferon-α, interferon-β, histamine, endothelin-1, angiotensin II, growth hormone (GH), and synthetic peptides, analogues or derivatives of these factors are also suitable for release from specific implants and devices to be described later. Other examples include CTGF (connective tissue growth factor); inflammatory microcrystals (e.g., crystalline minerals such as crystalline silicates); bromocriptine, methylsergide, methotrexate, chitosan, N-carboxybutyl chitosan, carbon tetrachloride, thioacetamide, fibrosin, ethanol, bleomycin, naturally occurring or synthetic peptides containing the Arg-Gly-Asp (RGD) sequence, generally at one or both termini (see, e.g., U.S. Pat. No. 5,997,895), and tissue adhesives, such as cyanoacrylate and crosslinked poly(ethylene glycol)-methylated collagen compositions. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885): 1528-1534.

Other representative examples of fibrosis-inducing agents include components of extracellular matrix (e.g., fibronectin, fibrin, fibrinogen, collagen (e.g., bovine collagen), including fibrillar and non-fibrillar collagen, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as TIMPs (tissue inhibitors of matrix metalloproteinases) and synthetic TIMPs, such as, e.g., marimistat, batimistat, doxycycline, tetracycline, minocycline, TROCADE, Ro-1130830, CGS 27023A, and BMS-275291 and analogues and derivatives thereof.

Although the above therapeutic agents have been provided for the purposes of illustration, it may be understood that the present invention is not so limited. For example, although agents are specifically referred to above, the present invention may be understood to include analogues, derivatives and conjugates of such agents. As an illustration, paclitaxel may be understood to refer to not only the common chemically available form of paclitaxel, but analogues (e.g., TAXOTERE, as noted above) and paclitaxel conjugates (e.g., paclitaxel-PEG, paclitaxel-dextran, or paclitaxel-xylos). In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

E. Dosages

Since soft tissue implants, such as facial implants, chin and mandibular implants, nasal implants, lip implants, pectoral implants, autogenous tissue implants and breast implants, are made in a variety of configurations and sizes, the exact dose administered will vary with device size, surface area and design. However, certain principles can be applied in the application of this art. Drug dose can be calculated as a function of dose (i.e., amount) per unit area of the portion of the device being coated. Surface area can be measured or determined by methods known to one of ordinary skill in the art. Total drug dose administered can be measured and appropriate surface concentrations of active drug can be determined. Drugs are to be used at concentrations that range from several times more than to 50%, 10%, 5%, or even less than 1% of the concentration typically used in a single chemotherapeutic systemic dose application. In one aspect, the drug is released in effective concentrations for a period ranging from 1-90 days. Regardless of the method of application of the drug to the device, the fibrosis-inhibiting agents, used alone or in combination, may be administered under the following dosing guidelines:

As described above, soft tissue implants may be used in combination with a composition that includes an anti-scarring agent. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-25001g/mm².

It may be apparent to one of skill in the art that potentially any anti-scarring agent described above may be utilized alone, or in combination, in the practice of this embodiment. Within one embodiment of the invention, soft tissue implants may be adapted to release an agent that inhibits one or more of the five general components of the process of fibrosis (or scarring), including: inflammation, migration and proliferation of connective tissue cells (such as fibroblasts or smooth muscle cells), formation of new blood vessels (angiogenesis), deposition of extracellular matrix (ECM), and remodeling (maturation and organization of the fibrous tissue). By inhibiting one or more of the components of fibrosis, the overgrowth of scar tissue may be inhibited or reduced.

In various aspects, the present invention provides a soft tissue implant containing an angiogenesis inhibitor in a dosage as set forth above. In various aspects, the present invention provides a soft tissue implant containing a 5-lipoxygenase inhibitor or antagonist in a dosage as set forth above. In various aspects, the present invention provides a soft tissue implant containing a chemokine receptor antagonist in a dosage as set forth above. In various aspects, the present invention provides a soft tissue implant containing a cell cycle inhibitor in a dosage as set forth above. In various aspects, the present invention provides a soft tissue implant containing an anthracycline (e.g., doxorubicin and mitoxantrone) in a dosage as set forth above. In various aspects, the present invention provides a soft tissue implant containing a taxane (e.g., paclitaxel or an analogue or derivative of paclitaxel) in a dosage as set forth above. In various aspects, the present invention provides a soft tissue implant containing a podophyllotoxin (e.g., etoposide) in a dosage as set forth above. In various aspects, the present invention provides a soft tissue implant containing a vinca alkaloid in a dosage as set forth above. In various aspects, the present invention provides a soft tissue implant containing a camptothecin or an analogue or derivative thereof in a dosage as set forth above. In various aspects, the present invention provides a soft tissue implant containing a platinum compound in a dosage as set forth above. In various aspects, the present invention provides a soft tissue implant containing a nitrosourea in a dosage as set forth above. In various aspects, the present invention provides a soft tissue implant containing a nitroimidazole in a dosage as set forth above. In various aspects, the present invention provides a soft tissue implant containing a folic acid antagonist in a dosage as set forth above. In various aspects, the present invention provides a soft tissue implant containing a cytidine analogue in a dosage as set forth above. In various aspects, the present invention provides a soft tissue implant containing a pyrimidine analogue in a dosage as set forth above. In various aspects, the present invention provides a soft tissue implant containing a fluoropyrimidine analogue in a dosage as set forth above. In various aspects, the present invention provides a soft tissue implant containing a purine analogue in a dosage as set forth above. In various aspects, the present invention provides a soft tissue implant containing a nitrogen mustard in a dosage as set forth above. In various aspects, the present invention provides a soft tissue implant containing a hydroxyurea in a dosage as set forth above. In various aspects, the present invention provides a soft tissue implant containing a mytomicin in a dosage as set forth above. In various aspects, the present invention provides a soft tissue implant containing an alkyl sulfonate in a dosage as set forth above. In various aspects, the present invention provides a soft tissue implant containing a benzamide in a dosage as set forth above. In various aspects, the present invention provides a soft tissue implant containing a nicotinamide in a dosage as set forth above. In various aspects, the present invention provides a soft tissue implant containing a halogenated sugar in a dosage as set forth above. In various aspects, the present invention provides a soft tissue implant containing a DNA alkylating agent in a dosage as set forth above. In various aspects, the present invention provides a soft tissue implant containing an anti-microtubule agent in a dosage as set forth above. In various aspects, the present invention provides a soft tissue implant containing a topoisomerase inhibitor in a dosage as set forth above. In various aspects, the present invention provides a soft tissue implant containing a DNA cleaving agent in a dosage as set forth above. In various aspects, the present invention provides a soft tissue implant containing an antimetabolite in a dosage as set forth above. In various aspects, the present invention provides a soft tissue implant containing an agent that inhibits adenosine deaminase in a dosage as set forth above. In various aspects, the present invention provides a soft tissue implant containing an agent that inhibits purine ring synthesis in a dosage as set forth above. In various aspects, the present invention provides a soft tissue implant containing a nucleotide interconversion inhibitor in a dosage as set forth above. In various aspects, the present invention provides a soft tissue implant containing an agent that inhibits dihydrofolate reduction in a dosage as set forth above. In various aspects, the present invention provides a soft tissue implant containing an agent that blocks thymidine monophosphate functioning a dosage as set forth above. In various aspects, the present invention provides a soft tissue implant containing an agent that causes DNA damage in a dosage as set forth above. In various aspects, the present invention provides a soft tissue implant containing a DNA intercalation agent in a dosage as set forth above. In various aspects, the present invention provides a soft tissue implant containing an agent that is a RNA synthesis inhibitor in a dosage as set forth above. In various aspects, the present invention provides a soft tissue implant containing an agent that is a pyrimidine synthesis inhibitor in a dosage as set forth above. In various aspects, the present invention provides a soft tissue implant containing an agent that inhibits ribonucleotide synthesis in a dosage as set forth above. In various aspects, the present invention provides a soft tissue implant containing an agent that inhibits thymidine monophosphate synthesis in a dosage as set forth above. In various aspects, the present invention provides a soft tissue implant containing an agent that inhibits DNA synthesis in a dosage as set forth above. In various aspects, the present invention provides a soft tissue implant containing an agent that causes DNA adduct formation in a dosage as set forth above. In various aspects, the present invention provides a soft tissue implant containing an agent that inhibits protein synthesis in a dosage as set forth above. In various aspects, the present invention provides a soft tissue implant containing an agent that inhibits microtubule function in a dosage as set forth above. In various aspects, the present invention provides a soft tissue implant containing an immunomodulatory agent (e.g., sirolimus, everolimus, tacrolimus, or an analogue or derivative thereof) in a dosage as set forth above. In various aspects, the present invention provides a soft tissue implant containing a heat shock protein 90 antagonist (e.g., geldanamycin) in a dosage as set forth above. In various aspects, the present invention provides a soft tissue implant containing an HMGCoA reductase inhibitor (e.g., simvastatin) in a dosage as set forth above. In various aspects, the present invention provides a soft tissue implant containing an inosine monophosphate dehydrogenase inhibitor (e.g., mycophenolic acid, 1-alpha-25 dihydroxy vitamin D₃) in a dosage as set forth above. In various aspects, the present invention provides a soft tissue implant containing an NF kappa B inhibitor (e.g., Bay 11-7082) in a dosage as set forth above. In various aspects, the present invention provides a soft tissue implant containing an antimycotic agent (e.g., sulconizole) in a dosage as set forth above. In various aspects, the present invention provides a soft tissue implant containing a p38 MAP kinase inhibitor (e.g., SB202190) in a dosage as set forth above. In various aspects, the present invention provides a soft tissue implant containing a cyclin dependent protein kinase inhibitor in a dosage as set forth above. In various aspects, the present invention provides a soft tissue implant containing an epidermal growth factor kinase inhibitor in a dosage as set forth above. In various aspects, the present invention provides a soft tissue implant containing an elastase inhibitor in a dosage as set forth above. In various aspects, the present invention provides a soft tissue implant containing a factor Xa inhibitor in a dosage as set forth above. In various aspects, the present invention provides a soft tissue implant containing a farnesyltransferase inhibitor in a dosage as set forth above. In various aspects, the present invention provides a soft tissue implant containing a fibrinogen antagonist in a dosage as set forth above. In various aspects, the present invention provides a soft tissue implant containing a guanylate cyclase stimulant in a dosage as set forth above. In various aspects, the present invention provides a soft tissue implant containing a hydroorotate dehydrogenase inhibitor in a dosage as set forth above. In various aspects, the present invention provides a soft tissue implant containing an IKK2 inhibitor in a dosage as set forth above. In various aspects, the present invention provides a soft tissue implant containing an IL-1 antagonist in a dosage as set forth above. In various aspects, the present invention provides a soft-tissue implant containing an ICE antagonist in a dosage as set forth above. In various aspects, the present invention provides a soft tissue implant containing an IRAK antagonist in a dosage as set forth above. In various aspects, the present invention provides a soft tissue implant containing an IL-4 agonist in a dosage as set forth above. In various aspects, the present invention provides a soft tissue implant containing a leukotriene inhibitor in a dosage as set forth above. In various aspects, the present invention provides a soft tissue implant containing an MCP-1 antagonist in a dosage as set forth above. In various aspects, the present invention provides a soft tissue implant containing a MMP inhibitor in a dosage as set forth above. In various aspects, the present invention provides a soft tissue implant containing an NO antagonist in a dosage as set forth above. In various aspects, the present invention provides a soft tissue implant containing a phosphodiesterase inhibitor in a dosage as set forth above. In various aspects, the present invention provides a soft tissue implant containing a TGF beta inhibitor in a dosage as set forth above. In various aspects, the present invention provides a soft tissue implant containing a thromboxane A2 antagonist in a dosage as set forth above. In various aspects, the present invention provides a soft tissue implant containing a TNFα antagonist in a dosage as set forth above. In various aspects, the present invention provides a soft tissue implant containing a TACE inhibitor in a dosage as set forth above. In various aspects, the present invention provides a soft tissue implant containing a tyrosine kinase inhibitor in a dosage as set forth above. In various aspects, the present invention provides a soft tissue implant containing a vitronectin inhibitor in a dosage as set forth above. In various aspects, the present invention provides a soft tissue implant containing a fibroblast growth factor inhibitor in a dosage as set forth above. In various aspects, the present invention provides a soft tissue implant containing a protein kinase inhibitor in a dosage as set forth above. In various aspects, the present invention provides a soft tissue implant containing a PDGF receptor kinase inhibitor in a dosage as set forth above. In various aspects, the present invention provides a soft tissue implant containing an endothelial growth factor receptor kinase inhibitor in a dosage as set forth above. In various aspects, the present invention provides a soft tissue implant containing a retinoic acid receptor antagonist in a dosage as set forth above. In various aspects, the present invention provides a soft tissue implant containing a platelet derived growth factor receptor kinase inhibitor in a dosage as set forth above. In various aspects, the present invention provides a soft tissue implant containing a fibronogin antagonist in a dosage as set forth above. In various aspects, the present invention provides a soft tissue implant containing a bisphosphonate in a dosage as set forth above. In various aspects, the present invention provides a soft tissue implant containing a phospholipase A1 inhibitor in a dosage as set forth above. In various aspects, the present invention provides a soft tissue implant containing a histamine H1/H2/H3 receptor antagonist in a dosage as set forth above. In various aspects, the present invention provides a soft tissue implant containing a macrolide antibiotic in a dosage as set forth above. In various aspects, the present invention provides a soft tissue implant containing a GPIIb IIIa receptor antagonist in a dosage as set forth above. In various aspects, the present invention provides a soft tissue implant containing an endothelin receptor antagonist in a dosage as set forth above. In various aspects, the present invention provides a soft tissue implant containing a peroxisome proliferator-activated receptor agonist in a dosage as set forth above. In various aspects, the present invention provides a soft tissue implant containing an estrogen receptor agent in a dosage as set forth above. In various aspects, the present invention provides a soft tissue implant containing a somastostatin analogue in a dosage as set forth above. In various aspects, the present invention provides a soft tissue implant containing a neurokinin 1 antagonist in a dosage as set forth above. In various aspects, the present invention provides a soft tissue implant containing a neurokinin 3 antagonist in a dosage as set forth above. In various aspects, the present invention provides a soft tissue implant containing a VLA-4 antagonist in a dosage as set forth above. In various aspects, the present invention provides a soft tissue implant containing an osteoclast inhibitor in a dosage as set forth above. In various aspects, the present invention provides a soft tissue implant containing a DNA topoisomerase ATP hydrolyzing inhibitor in a dosage as set forth above. In various aspects, the present invention provides a soft tissue implant containing an angiotensin I converting enzyme inhibitor in a dosage as set forth above. In various aspects, the present invention provides a soft tissue implant containing an angiotensin II antagonist in a dosage as set forth above. In various aspects, the present invention provides a soft tissue implant containing an enkephalinase inhibitor in a dosage as set forth above. In various aspects, the present invention provides a soft tissue implant containing a peroxisome proliferator-activated receptor gamma agonist insulin sensitizer in a dosage as set forth above. In various aspects, the present invention provides a soft tissue implant containing a protein kinase C inhibitor in a dosage as set forth above. In various aspects, the present invention provides soft tissue implants containing a ROCK (rho-associated kinase) inhibitor in a dosage as set forth above. In various aspects, the present invention provides soft tissue implants containing a CXCR3 inhibitor in a dosage as set forth above. In various aspects, the present invention provides soft tissue implants containing a Itk inhibitor in a dosage as set forth above. In various aspects, the present invention provides soft tissue implants containing a cytosolic phospholipase A₂-alpha inhibitor in a dosage as set forth above. In various aspects, the present invention provides soft tissue implants containing a PPAR agonist in a dosage as set forth above. In various aspects, the present invention provides soft tissue implants containing an Immunosuppressant in a dosage as set forth above. In various aspects, the present invention provides soft tissue implants containing an Erb inhibitor in a dosage as set forth above. In various aspects, the present invention provides soft tissue implants containing an apoptosis agonist in a dosage as set forth above. In various aspects, the present invention provides soft tissue implants containing a lipocortin agonist in a dosage as set forth above. In various aspects, the present invention provides soft tissue implants containing a VCAM-1 antagonist in a dosage as set forth above. In various aspects, the present invention provides soft tissue implants containing a collagen antagonist in a dosage as set forth above. In various aspects, the present invention provides soft tissue implants containing an alpha 2 integrin antagonist in a dosage as set forth above. In various aspects, the present invention provides soft tissue implants containing a TNF alpha inhibitor in a dosage as set forth above. In various aspects, the present invention provides soft tissue implants containing a nitric oxide inhibitor in a dosage as set forth above. In various aspects, the present invention provides soft tissue implants containing a cathepsin inhibitor in a dosage as set forth above.

Provided below are exemplary dosage ranges for a variety of anti-scarring agents which can be used in conjunction with soft tissue implants in accordance with the invention. (A) Cell cycle inhibitors including doxorubicin and mitoxantrone. Doxorubicin analogues and derivatives thereof: total dose not to exceed 25 mg (range of 0.1 μg to 25 mg); preferred 1 μg to 3 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 3 mg. The dose per unit area of the device of 0.01 μg-20 μg per mm²; preferred dose of 0.05 μg/mM²-5 μg/mm². Minimum concentration of 10⁻⁸-10⁻⁴ M of mitoxantrone is to be maintained on the device surface. (B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.05 μg-10 μg per mm²; preferred dose of 0.20 μg/mm²-51 g/mm². Minimum concentration of 10⁻⁹-10⁻⁴ M of paclitaxel is to be maintained on the device surface. (C) Cell cycle inhibitors such as podophyllotoxins (e.g., etoposide): total dose not to exceed 25 mg (range of 0.1 μg to 25 mg); preferred 1 μg to 5 mg. The dose per unit area of the device of 0.1 μg-100 μg per mm²; preferred dose of 0.1 μg/mm²-10 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 5 mg. The dose per unit area of 0.1 μg-100 μg per mm²; preferred dose of 0.25 μg/mm²-10 μg/mM². Minimum concentration of 10⁻⁸-10⁻⁴ M is to be maintained on the device surface. Everolimus and derivatives and analogues thereof: Total dose may not exceed 10 mg (range of 0.1 μg to 10 mg); preferred 10 μg to 5 mg. The dose per unit area of 0.1 μg-100 μg per mm² of surface area; preferred dose of 0.25 μg/mm²-10 μg/mM². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCOA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosage ranges for use with soft tissue implants include the following: (A) Biolimus and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of auranofin is to be maintained on the device surface. (D) 27-O-Demethylrapamycin and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of 27-O-Demethylrapamycin is to be maintained on the device surface. (E) Gusperimus and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁷ M of gusperimus is to be maintained on the device surface. (F) Pimecrolimus and derivatives and analogues thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 5 mg. The dose per unit area of 0.1 μg-100 μg per mm² of surface area; preferred dose of 0.1 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosage ranges for use with ear ventilation devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.2 μg/mm²-5 μg/mm². Minimum concentration of 10⁻⁸-10⁻⁴ M of drug is to be maintained on the device surface.

F. Methods for Generating Soft Tissue Implants Which Include and Release a Fibrosis-Inhibiting Agent

In the practice of this invention, drug-coated or drug-impregnated soft tissue implants are provided which inhibit fibrosis in and around the soft tissue implant. Within various embodiments, fibrosis is inhibited by local, regional or systemic release of specific pharmacological agents that become localized to the tissue adjacent to the implant. There are numerous soft tissue implants where the occurrence of a fibrotic reaction will adversely affect the functioning or aesthetic appearance of the implant. Typically, fibrotic encapsulation of the soft tissue implant (or the growth of fibrous tissue between the implant and the surrounding tissue) can result in fibrous contracture of tissue surrounding the implant. This can cause the implant to become displaced, disfigured, asymmetric, dimple the overlying skin, harden, cause patient dissatisfaction and require repeat surgical intervention (capsulectomy, capsulotomy, implant revision, or implant removal). For many soft tissue implants, the fibrosis-inhibiting agent may be delivered via a carrier system to optimize dosage and allow sustained release of the agent into the target tissue for a period of time after implantation surgery. There are numerous methods available for optimizing delivery of the fibrosis-inhibiting agent to the site of the intervention and several of these are described below.

1) Sustained-Release Preparations of Fibrosis-Inhibiting Agents

As described previously, desired fibrosis-inhibiting agents may be admixed with, blended with, conjugated to, or, otherwise modified to contain a polymer composition (which may be either biodegradable or non-biodegradable), or a non-polymeric composition, in order to release the therapeutic agent over a prolonged period of time. For many of the aforementioned embodiments, localized delivery as well as localized sustained delivery of the fibrosis-inhibiting agent may be required. For example, a desired fibrosis-inhibiting agent may be admixed with, blended with, conjugated to, or otherwise modified to contain a polymeric composition (which may be either biodegradable or non-biodegradable), or non-polymeric composition, in order to release the fibrosis-inhibiting agent over a period of time. In certain aspects, the polymer composition may include a bioerodible or biodegradable polymer. Representative examples of biodegradable polymer compositions suitable for the delivery of fibrosis-inhibiting agents include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), polyesters where the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, poly(ethylene terephthalate), poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ, where X is a polyalkylene oxide and Y is a polyester, where the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLA, PCL, polydioxanone and copolymers thereof), R is a multifunctional initiator and their copolymers as well as blends thereof. (see generally, Illum, L., Davids, S. S. (eds.) “Polymers in Controlled Drug Delivery” Wright, Bristol, 1987; Arshady, J. Controlled Release 17: 1-22, 1991; Pitt, Int J. Phar. 59: 173-196, 1990; Holland et al., J. Controlled Release 4: 155-0180, 1986).

Representative examples of non-degradable polymers suitable for the delivery of fibrosis-inhibiting agents include poly(ethylene-co-vinyl acetate) (“EVA”) copolymers, silicone rubber, acrylic polymers (polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate)poly(octylcyanoacrylate)), polyethylene, polypropylene, polyamides (nylon 6,6), polyurethanes (e.g., CHRONOFLEX AL and CHRONOFLEX AR (both from CardioTech International, Inc., Woburn, Mass.) and BIONATE (Polymer Technology Group, Inc., Emeryville, Calif.)), poly(ester urethanes), poly(ether urethanes), poly(ester-urea), polyethers (poly(ethylene oxide), poly(propylene oxide), block copolymers based on ethylene oxide and propylene oxide (i.e., copolymers of ethylene oxide and propylene oxide polymers), such as the family of PLURONIC polymers available from BASF Corporation (Mount Olive, N.J.), and poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof (see generally, Dunn et al., J. Applied Polymer Sci. 50: 353-365,1993; Cascone et al., J. Materials Sci.: Materials in Medicine 5: 770-774,1994; Shiraishi et al., Biol. Pharm. Bull. 16(11): 1164-1168, 1993;Thacharodi and Rao, Int'l J. Pharm. 120: 115-118, 1995; Miyazaki et al., Int'l J. Pharm. 118: 257-263,1995).

Examples of preferred polymeric carriers include poly(ethylene-co-vinyl acetate), polyurethanes (e.g., CHRONOFLEX AL and CHRONOFLEX AR (both from CardioTech International, Inc., Woburn, Mass.) and BIONATE (Polymer Technology Group, Inc., Emeryville, Calif.)), poly(D,L-lactic acid) oligomers and polymers, poly(L-lactic acid) oligomers and polymers, poly(glycolic acid), copolymers of lactic acid and glycolic acid, poly(caprolactone), poly (valerolactone), polyanhydrides, copolymers of poly(caprolactone) or poly(lactic acid) with a polyethylene glycol (e.g., MePEG), silicone rubbers, poly(styrene)block-poly(isobutylene)-block-poly(styrene), poly(acrylate)polymers and blends, admixtures, or co-polymers of any of the above. Other examples of polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of fibrosis-inhibiting agents include carboxylic polymers, polyacetates, polyacrylamides, polycarbonates, polyethers, polyesters, polyethylenes, polyvinylbutyrals, polysilanes, polyureas, polyurethanes (e.g., CHRONOFLEX AL and CHRONOFLEX AR (both from CardioTech International, Inc., Woburn, Mass.) and BIONATE (Polymer Technology Group, Inc., Emeryville, Calif.)), polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, nitrocellulose and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555,5,997,517, 6,007,833, 6,071,447, 6,090,995, 6,106,473, 6,110,483,6,121,027, 6,156,345, 6,214,901, 6,368,611 6,630,155, 6,528,080, RE37,950, 6,46,1631,6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Patent Application Publication Nos. 2003/0068377, 2002/0192286, 2002/0076441, and 2002/0090398.

It may be obvious to one of skill in the art that the polymers as described herein can also be blended or copolymerized in various compositions as required to deliver therapeutic doses of fibrosis-inhibiting agents.

Polymeric carriers for fibrosis-inhibiting agents can be fashioned in a variety of forms, with desired release characteristics and/or with specific properties depending upon the device, composition or implant being utilized. For example, polymeric carriers may be fashioned to release a fibrosis-inhibiting agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48: 343-354,1993; Dong et al., J. Controlled Release 19: 171-178, 1992; Dong and Hoffman, J. Controlled Release 15: 141-152, 1991; Kim et al., J. Controlled Release 28: 143-152,1994; Comejo-Bravo et al., J. Controlled Release 33: 223-229,1995; Wu and Lee, Pharm. Res. 10(10): 1544-1547, 1993; Serres et al., Pharm. Res. 13(2): 196-201, 1996; Peppas, “Fundamentals of pH- and Temperature-Sensitive Delivery Systems,” in Gumy et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide Imonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inhibiting agents can be delivered via polymeric carriers which are temperature sensitive (see, e.g., Chen et al., “Novel Hydrogels of a Temperature-Sensitive PLURONIC Grafted to a Bioadhesive Polyacrylic Acid. Backbone for Vaginal Drug Delivery,” in Proceed. Intern. Symp. Control. Rel. Bioact Mater. 22: 167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22: 111-112, Controlled Release Society, Inc., 1995; Johnston et al., Pharm. Res. 9(3): 425-433, 1992; Tung, Int'l J. Pharm. 107: 85-90, 1994; Harsh and Gehrke, J. Controlled Release 17: 175-186,1991; Bae et al., Pharm. Res. 8(4): 531-537,1991; Dinarvand and D'Emanuele, J. Controlled Release 36: 221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3): 283-290, 1992; Bae et al., Pharm. Res. 8(5): 624-628, 1991; Kono et al., J. Controlled Release 30: 69-75, 1994; Yoshida et al., J. Controlled Release 32: 97-102, 1994; Okano et al., J. Controlled Release 36: 125-133,1995; Chun and Kim, J. Controlled Release 38: 39-47,1996; D'Emanuele and Dinarvand, Intl J. Pharm. 118: 237-242, 1995; Katono et al., J. Controlled Release 16: 215-228,1991; Hoffman, “Thermally-Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27,1987, pp. 297-305; Gutowska et al., J. Controlled Release 22: 95-104, 1992; Palasis and Gehrke, J. Controlled Release 18: 1-12, 1992; Paavola et al., Pharm. Res. 12(12): 1997-2002, 1995).

Representative examples of thermogelling polymers, and their gelatin temperature (LCST (OC)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isoprbpylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y, R—(Y—X)ₙ, R—(X—Y)ₙ and X—Y—X where X in a polyalkylene oxide and Y is a biodegradable polyester, where the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator and PLURONICs such as F-127, 10-15° C.; L-122, 19° C.; L-92, 26° C.; L-81, 20° C.; and L-61, 24° C.

Representative examples of patents relating to thermally gelling polymers and their preparation include U.S. Pat. Nos. 6,451,346; 6,201,072; 6,117,949; 6,004,573; 5,702,717; and 5,484,610 and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inhibiting agents may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films and sprays.

Within certain aspects of the present invention, therapeutic compositions may be fashioned into particles having any size ranging from 50 nm to 500 μm, depending upon the particular use. These compositions can be in the form of microspheres, microparticles and/or nanoparticles. These compositions can be formed by spray-drying methods, milling methods, coacervation methods, W/O emulsion methods, W/O/W, emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of paste or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inhibiting agents are particularly useful for application to the surface of tissues that will be in contact with the implant or device.

Within yet other aspects of the invention, the therapeutic compositions of the present invention may be formed as a film or tube. These films or tubes can be porous or non-porous. Such films or tubes are generally less than 5, 4, 3, 2, or 1 mm thick, or less than 0.75 mm, or less than 0.5 mm, or less than 0.25 mm, or, less than 0.10 mm thick. Films or tubes can also be generated of thicknesses less than 50 μm, 25 μm or 10 μm. Such films may be flexible with a good tensile strength (e.g., greater than 50, or greater than 100, or greater than 150 or 200 N/cm²), good adhesive properties (i.e., adheres to moist or wet surfaces), and have controlled permeability. Fibrosis-inhibiting agents contained in polymeric films are particularly useful for application to the surface of a device or implant as well as to the surface of tissue, cavity or an organ.

Within further aspects of the present invention, polymeric carriers are provided which are adapted to contain and release a hydrophobic fibrosis-inhibiting compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix that contains the hydrophobic fibrosis-inhibiting compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan, hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inhibiting agents described herein include: hydroxypropyl cyclodextrin (Cserhati and Hollo, Int. J. Pharm. 108: 69-75, 1994), liposomes (see, e.g., Sharma et al., Cancer Res. 53: 5877-5881, 1993; Sharma and Straubinger, Pharm. Res. 11(60): 889-896, 1994; WO 93/18751; U.S. Pat. No. 5,242,073), liposome/gel (WO 94/26254), nanocapsules (Bartoli et al., J. Microencapsulation 7(2): 191-197, 1990), micelles (Alkan-Onyuksel et al., Pharm. Res. 11(2): 206-212,1994), implants (Jampel et al., Invest. Ophthalm. Vis. Science 34(11): 3076-3083, 1993; Walter et al., Cancer Res. 54: 22017-2212, 1994), nanoparticles (Violante and Lanzafame PAACR), nanoparticles-modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bloact. Mater. 22,1995; Kwon et al., Pharm Res. 12(2): 192-195; Kwon et al., Pharm Res. 10(7): 970-974; Yokoyama et al., J. Contr. Rel. 32: 269-277, 1994; Gref et al., Science 263: 1600-1603, 1994; Bazile et al., J. Pharm. Sci. 84: 493-498, 1994) and implants (U.S. Pat. No. 4,882,168).

As mentioned elsewhere herein, the present invention provides for polymeric crosslinked matrices, and polymeric carriers, that may be used to assist in the prevention of the formation or growth of fibrous connective tissue. The composition may contain and deliver fibrosis-inhibiting agents in the vicinity of the implanted device. The following compositions are particularly useful when it is desired to infiltrate around the device, with or without a fibrosis-inhibiting agent. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a delivery device (e.g., a syringe) in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifunctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include:
(i) synthetic polypeptides that have been synthesized to contain two or more primary amino groups or thiol groups; and (ii) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000.

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetra methylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), di(2-aminoethyl)amine (HN—(CH₂—CH₂—NH₂)₂), and tris(2-aminoethyl)amine (N—(CH₂—CH₂—NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers that are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

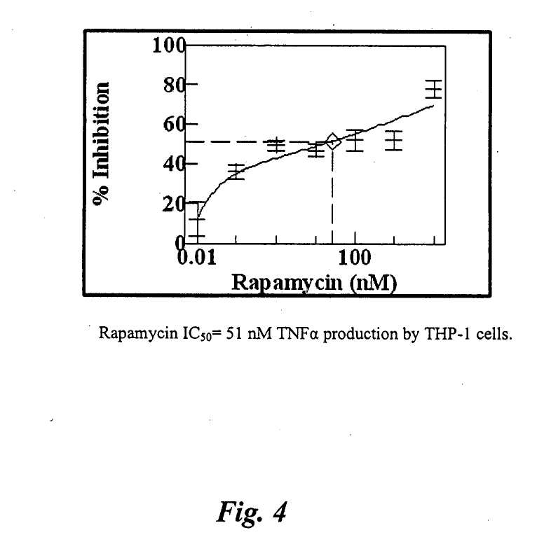

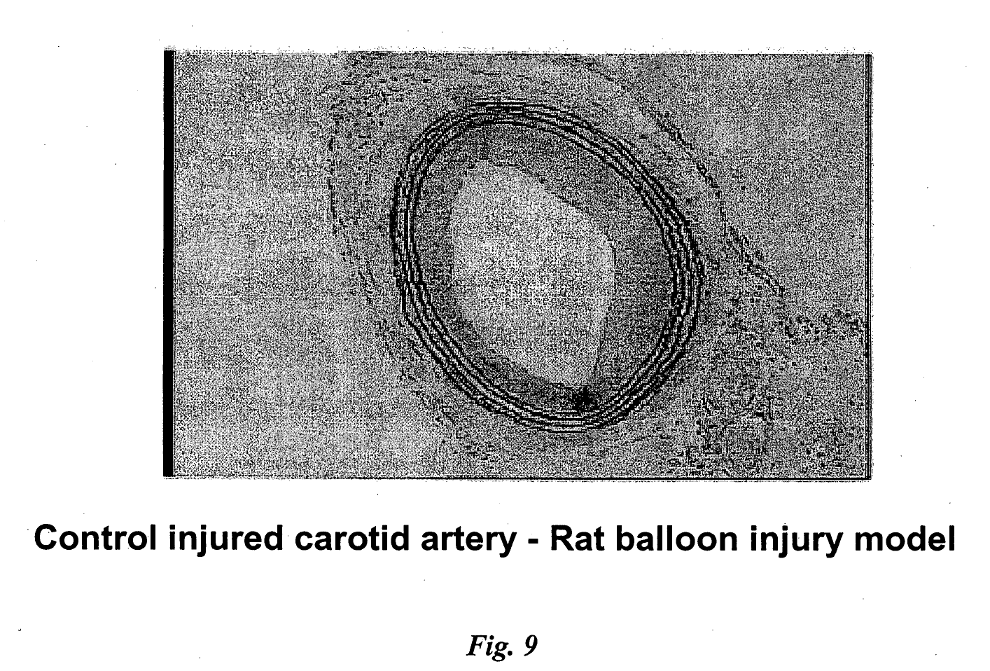

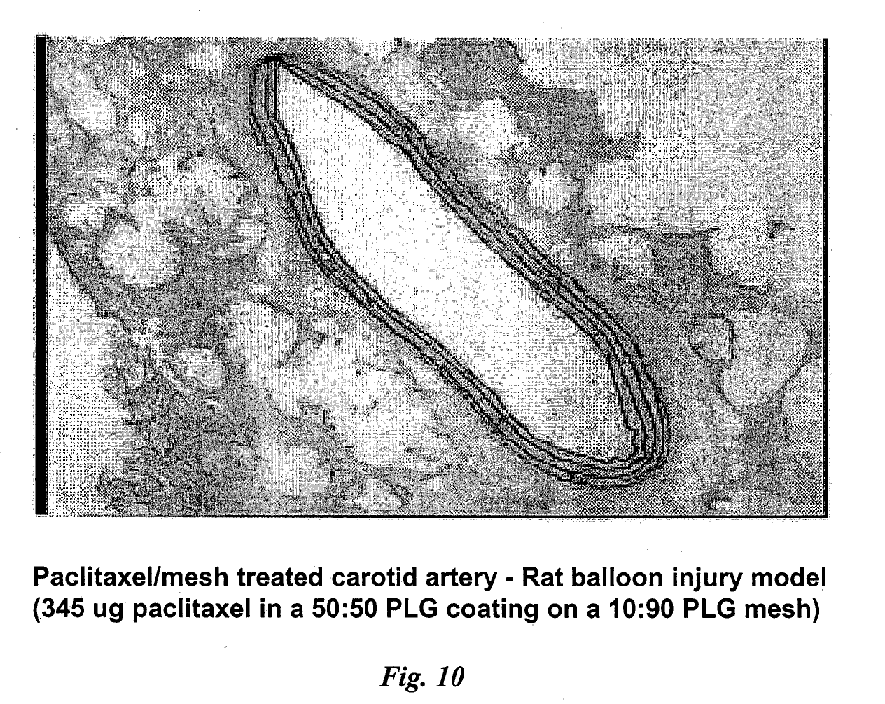

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. No. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl] (4-armed thiol PEG) and pentaerythritol poly(ethylene glycol)ether tetra-succinimidyl glutarate](4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (1-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers that contain a relatively small proportion of oxygen or nitrogen atoms.

Hydrophobic polymers which already contain two or more succinimidyl groups include, without limitation, disuccinimidyl suberate (DSS), bis(sulfdsuccinimidyl) suberate (BS3), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The above-referenced polymers are commercially available from Pierce (Rockford, Ill.), undercatalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis (2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:
Polymer-Xₘ+Polymer-Yₙ→Polymer-Z-Polymer
wherein m≦2, n≦2, and m+n≦5;
where exemplary X groups include —NH₂, —SH, —OH, —PH₂, CO—NH—NH₂, etc., where the X groups may be the same or different in polymer-Xₘ;
where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and
where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂O)ₙ— or —(CH(CH₃)CH₂O)ₙ— or —(CH₂—CH₂—O)ₙ—(CH(CH₃)CH₂—O)ₙ—. In these cases the synthetic polymer would be difunctional.

The required functional group X or Y is commonly coupled to the polymer backbone by a linking group (represented below as “Q”), many of which are known or possible. There are many ways to prepare the various functionalized polymers, some of which are listed below:
Polymer-Q₁-X+Polymer-Q₂-Y→Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ₙ—; and —O—R₂—CO—NH—, which provide synthetic polymers of the partial structures: polymer-O—(CH2)ₙ—(X or Y); polymer-S—(CH₂)ₙ—(X or Y); polymer-NH—(CH₂)ₙ—(X or Y); polymer-O₂C—NH—(CH₂)ₙ—(X or Y); polymer-O₂C—(CH₂)ₙ—(X or Y); polymer-O₂C—(CR¹H)ₙ—(X or Y); and polymer-O—R₂—CO—NH—(X or Y), respectively. In these structures, n=1-10, R¹=Hor alkyl (i.e., CH₃, C₂H₅, etc.); R²═CH₂, or CO—NH—CH₂CH₂; and Q₁ and Q₂ may be the same or different.

For example, when Q₂=OCH₂CH₂ (there is no Q₁ in this case); Y═—CO₂—N(COCH₂)₂; and X═—NH₂, —SH, or —OH, the resulting reactions and Z groups would be as follows:
Polymer-NH₂+Polymer-O—CH₂—CH₂—CO₂—N(COCH₂)₂→Polymer-NH—CO—CH₂—CH₂—O-Polymer;
Polymer-SH+Polymer-O—CH₂—CH₂—CO₂—N(COCH₂)₂→Polymer-S—COCH₂CH₂—O-Polymer; and
Polymer-OH+Polymer-O—CH₂—CH₂—CO₂—N(COCH₂)₂→Polymer-O—COCH₂CH₂—O-Polymer.

An additional group, represented below as “D”, can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.
Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), E-caprolactone, and amino acids. The polymers may be referred to as polylactide, polyglycolide, poly(co-lactide-glycolide); poly-e-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) would contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis that typically occurs upon exposure of such electrophilic groups to aqueous media. Processes for preparing synthetic hydrophilic polymers containing multiple electrophylic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In one aspect, a composition comprising naturally-occurring protein and both of the first and second synthetic polymer as described above is used to form the crosslinked matrix according to the present invention. In one aspect, a composition comprising collagen and both of the first and second synthetic polymer as described above is used to form the crosslinked matrix according to the present invention. In one aspect, a composition comprising methylated collagen and both of the first and second synthetic polymer as described above is used to form the crosslinked matrix according to the present invention. In one aspect, a composition comprising fibrinogen and both of the first and second synthetic polymer as described above is used to form the crosslinked matrix according to the present invention. In one aspect, a composition comprising thrombin and both of the first and second synthetic polymer as described above is used to form the crosslinked matrix according to the present invention. In one aspect, a composition comprising albumin and both of the first and second synthetic polymer as described above is used to form the crosslinked matrix according to the present invention. In one aspect, a composition comprising fibrin and both of the first and second synthetic polymer as described above is used to form the crosslinked matrix according to the present invention. In one aspect, a composition comprising naturally occurring polysaccharide and both of the first and second synthetic polymer as described above is used to form the crosslinked matrix according to the present invention. In one aspect, a composition comprising glycosaminoglycan and both of the first and second synthetic polymer as described above is used to form the crosslinked matrix according to the present invention. In one aspect, a composition comprising deacetylated glycosaminoglycan and both of the first and second synthetic polymer as described above is used to form the crosslinked matrix according to the present invention. In one aspect, a composition comprising desulfated glycosaminoglycan and both of the first and second synthetic polymer as described above is used to form the crosslinked matrix according to the present invention.

In one aspect, a composition comprising naturally-occurring protein and the first synthetic polymer as described above is used to form the crosslinked matrix according to the present invention. In one aspect, a composition comprising collagen and the first synthetic polymer as described above is used to form the crosslinked matrix according to the present invention. In one aspect, a composition comprising methylated collagen and the first synthetic polymer as described above is used to form the crosslinked matrix according to the present invention. In one aspect, a composition comprising fibrinogen and the first synthetic polymer as described above is used to form the crosslinked matrix according to the present invention. In one aspect, a composition comprising thrombin and the first synthetic polymer as described above is used to form the crosslinked matrix according to the present invention. In one aspect, a composition comprising albumin and the first synthetic polymer as described above is used to form the crosslinked matrix according to the present invention. In one aspect, a composition comprising fibrin and the first synthetic polymer as described above is used to form the crosslinked matrix according to the present invention. In one aspect, a composition comprising naturally occurring polysaccharide and the first synthetic polymer as described above is used to form the crosslinked matrix according to the present invention. In one aspect, a composition comprising glycosaminoglycan and the first synthetic polymer as described above is used to form the crosslinked matrix according to the present invention. In one aspect, a composition comprising deacetylated glycosaminoglycan and the first synthetic polymer as described above is used to form the crosslinked matrix according to the present invention. In one aspect, a composition comprising desulfated glycosaminoglycan and the first synthetic polymer as described above is used to form the crosslinked matrix according to the present invention.

In one aspect, a composition comprising naturally-occurring protein and the second synthetic polymer as described above is used to form the crosslinked matrix according to the present invention. In one aspect, a composition comprising collagen and the second synthetic polymer as described above is used to form the crosslinked matrix according to the present invention. In one aspect, a composition comprising methylated collagen and the second synthetic polymer as described above is used to form the crosslinked matrix according to the present invention. In one aspect, a composition comprising fibrinogen and the second synthetic polymer as described above is used to form the crosslinked matrix according to the present invention. In one aspect, a composition comprising thrombin and the second synthetic polymer as described above is used to form the crosslinked matrix according to the present invention. In one aspect, a composition comprising albumin and the second synthetic polymer as described above is used to form the crosslinked matrix according to the present invention. In one aspect, a composition comprising fibrin and the second synthetic polymer as described above is used to form the crosslinked matrix according to the present invention. In one aspect, a composition comprising naturally occurring polysaccharide and the second synthetic polymer as described above is used to form the crosslinked matrix according to the present invention. In one aspect, a composition comprising glycosaminoglycan and the second synthetic polymer as described above is used to form the crosslinked matrix according to the present invention. In one aspect, a composition comprising deacetylated glycosaminoglycan and the second synthetic polymer as described above is used to form the crosslinked matrix according to the present invention. In one aspect, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In one aspect, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In one aspect, the polymer may be collagen or a collagen derivative, for example methylated collagen. An example of an in situ forming composition uses pentaerythritol poly(ethylene glycol)ether tetra-sulfhydryl](4-armed thiol PEG), pentaerythritol poly(ethylene glycol)ether tetra-succinimidyl glutarate](4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage that commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing soft tissue augmentation in delicate facial tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

Hydrophilic Polymer + Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitiri sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillat in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

Them nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae

| | | |
|---|---|---|
| (I) | R¹(—[Q¹]_{q}—X)ₘ | (component A), |
| (II) | R²(—[Q²]ᵣ—Y)ₙ | (component B), and |
| (III) | R³(—[Q³]ₛ—Fn)ₚ | (optional component C), |

wherein:
R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;
X represents one of the m nucleophilic groups of component A, and the various X moieties on A may be the same or different;
Y represents one of the n electrophilic groups of component B, and the various Y moieties on A may be the same or different;
Fn represents a functional group on optional component C;
Q¹, Q² and Q³ are linking groups;
m≧2, n≧2, m+n is 24, q, and r are independently zero or 1, and when optional component C is present, p>1, and s is independently zero or 1.

Reactive Groups:

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra in Section E.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted Or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups is particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE

| REPRESENTATIVE | | |
|---|---|---|
| NUCLEOPHILIC | | |
| COMPONENT | REPRESENTATIVE | |
| (A, optional | ELECTROPHILIC | |
| component C | COMPONENT | |
| element FN_{NU}) | (B, FN_{EL}) | RESULTING LINKAGE |
| R¹-NH₂ | R²-O—(CO)—O—N(COCH₂) | R¹-NH—(CO)—O-R² |
| | (succinimidyl carbonate | |
| | terminus) | |
| R¹-SH | R²-O—(CO)—O—N(COCH₂) | R¹-S—(CO)—O-R² |
| R¹-OH | R²-O—(CO)—O—N(COCH₂) | R¹-O—(CO)-R² |
| R¹-NH₂ | R²-O(CXO)—CH═CH₂ | R¹-NH—CH₂CH₂—(CO)—O-R² |
| R¹-SH | R²-O—(CO)—CH═CH₂ | R¹-S—CH₂CH₂—(CO)—O-R² |
| R¹-OH | R²-O—(CO)—CH═CH₂ | R¹-O—CH₂CH₂—(CO)—O-R² |
| R¹-NH₂ | R²-O(CO)—(CH₂)₃—CO₂—N(COCH₂) | R¹-NH—(CO)—(CH₂)₃—(CO)—OR² |
| | (succinimidyl glutarate | |
| | terminus) | |
| R¹-SH | R²-O(CO)—(CH₂)₃—CO₂—N(COCH₂) | R¹-S—(CO)—(CH₂)₃—(CO)—OR² |
| R¹-OH | R²-O(CO)—(CH₂)₃—CO₂—N(COCH₂) | R¹-O—(CO)—(CH₂)₃—(CO)—OR² |
| R¹-NH₂ | R²-O—CH₂—CO₂—N(COCH₂) | R¹-NH—(CO)—CH₂—OR² |
| | (succinimidyl acetate | |
| | terminus) | |
| R¹-SH | R²-O—CH₂—CO₂—N(COCH₂) | R¹-S—(CO)—CH₂—OR² |
| R¹-OH | R²-O—CH₂—CO₂—N(COCH₂) | R¹-O—(CO)—CH₂—OR² |
| R¹-NH₂ | R²-O—NH(CO)—(CH₂)₂—CO₂—N(COCH₂) | R¹-NH—(CO)—(CH₂)₂—(CO)—NH—OR² |
| | (succinimidyl succinamide | |
| | terminus) | |
| R¹-SH | R²-O—NH(CO)—(CH₂)₂—CO₂—N(COCH₂) | R¹-S—(CO)—(CH₂)₂—(CO)—NH—OR² |
| R¹-OH | R²-O—NH(CO)—(CH₂)₂—CO₂—N(COCH₂) | R¹-O—(CO)—(CH₂)₂—(CO)—NH—OR² |
| R¹-NH₂ | R²-O—(CH₂)₂—CHO | R¹NH—(CO)—(CH₂)₂—OR² |
| | (propionaldehyde | |
| | terminus) | |
| R¹-NH₂ | 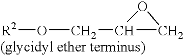 | R¹-NH—CH₂—CH(OH)—CH₂—OR² and R¹-N[CH₂—CH(OH)—CH₂—OR²]₂ |
| R¹-NH₂ | R²-O—(CH₂)₂—N═C═O | R¹—NH—(CO)—NH—CH₂—OR² |
| | (isocyanate terminus) | |
| R¹-NH₂ | R²-SO₂—CH═CH₂ | R¹-NH—CH₂CH₂—SO₂—R² |
| | (vinyl sulfone terminus) | |
| R¹-SH | R²-SO₂+113 CH═CH₂ | R¹-S—CH₂CH₂—SO₂—R² |

Linking Groups:

The functional groups X and Y and FN on optional component C may be directly attached to the compound core (R¹, R² or R³ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages; ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in the following Table:

TABLE

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹(-[Q¹]_{q}—X)ₘ, for component A, (II) R²(-[Q²]ᵣ—Y)ₙ for component B, and (III)

R³(-[Q³]ₛ-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components

In one aspect, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides. A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6: 315), peptide chemistry (see Mutter et al., The Peptides, Academic: New York, N.Y. 2: 285-332; and Zalipsky et al., Int. J. Peptide Protein Res. (1987) 30: 740), and the synthesis of polymeric drugs (see Zalipsky et al., Eur. Polym. J. (1983) 19: 1177; and Ouchi et al., J. Macromol. Sci. Chem. (1987) A24: 1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998).

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC. Polyols such as trimethylblpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. No. 6,454,786 to Holm et al.; U.S. Pat. No. 6,461,325 to Delmotte et al.; U.S. Pat. No. 5,585,007 to Antanavich et al.; U.S. Pat. No. 5,116,315 to Capozzi et al.; and U.S. Pat. No. 4,631,055 to Redl et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in Water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen + Fibrinogen and/or Thrombin (e.g. Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient to whom the composition is to be delivered. The plasma can be used “as is” after standard preparation that includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition, which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NlHu, most usually about 200 to about 500 NlHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NlHu to 10,000 NlHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Patent Nos. RE38158; 4,839,345; 5,514,379, 5,583,114; 6,458,147; 6,371,975; 5,290,552; 6,096,309; U.S. Patent Application Publication Nos. 2002/0161399; 2001/0018598 and PCT Publication Nos. WO 03/090683; WO 01/45761; WO 99/66964 and WO 96/03159).

Self-Reactive Compounds

In one aspect, the therapeutic agent is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

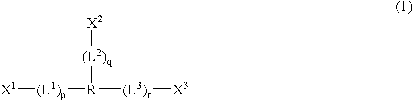

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl that is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):
[X′-(L⁴)ₐ—Y-(L⁵)_{b}]_{c}—R′
where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II): 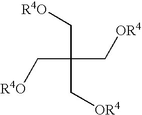
where R⁴ has the formula:

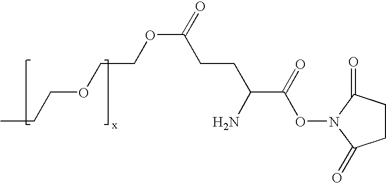

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

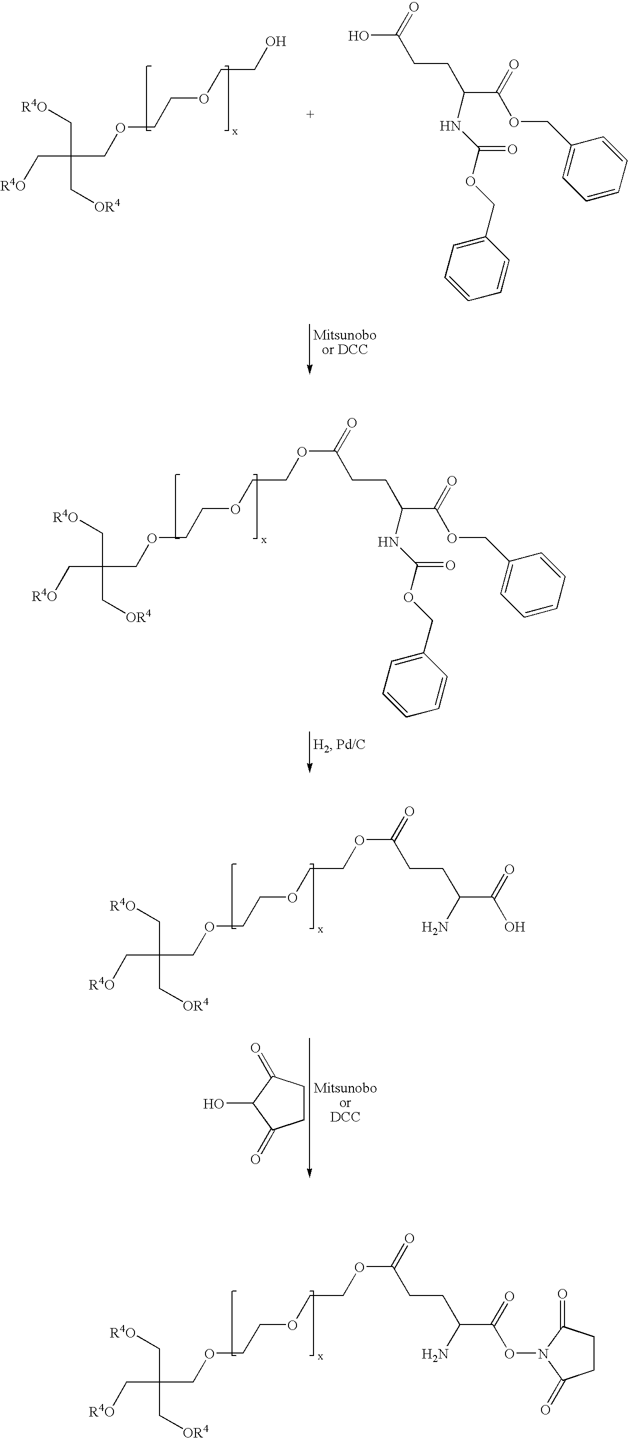

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix. Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

A. Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups are approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

1. Electrophilic and Nucleophilic Reactive Groups

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also, referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (Z_{EL}). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂, —PHR¹, —P(R¹)₂, —NH—NH₂, —CO—NH—NH₂, —C₅H₄N, etc. wherein R¹ is a hydrocarbyl group and each R1 may be the same or different. R¹ is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═NH, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C═O); (7) thioisocyanato group (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH2), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table:

TABLE

| Representative | | |
|---|---|---|
| Nucleophilic | Representative Electrophilic | |
| Group (X, Z_{NU}) | Group (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH₂—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)+113 (CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S—(CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂—N(COCH₂)₂ | —NH—(CO)—(CH₂)₂—(CO)—NH—O— |
| | succinimidyl succinamide | |
| | terminus | |
| —SH | —O—NH(CO)—(CH₂)₂—CO₂—N(COCH₂)₂ | —S—(CO)—(CH₂)₂—(CO)—NH—O— |
| —OH | —O—NH(CO)—(CH₂)₂—CO₂—N(COCH₂)₂ | —O—(CO)—(CH₂)₂—(CO)—NH—O— |
| —NH₂ | —O—(CH₂)₂—CHO | —NH—(CO)—(CH₂)₂—O— |
| | propionaldehyde terminus | |
| —NH₂ | 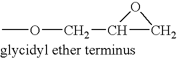 | —NH—CH₂—CH(OH)—CH₂—O— and —N[CH₂—CH(OH)—CH₂—O—]₂ |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| | vinyl sulfone terminus | |
| —SH | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH 9.5.

In another embodiment the modification of a dry initial environment comprises dissolving the self-reactive compound in a first buffer solution having a pH within the range of about 1.0 to 5.5 to form a homogeneous solution, and (ii) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution, is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7 g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

2. Redox Reactive Groups

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

0.3. Oxidative Coupling Reactive Groups

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

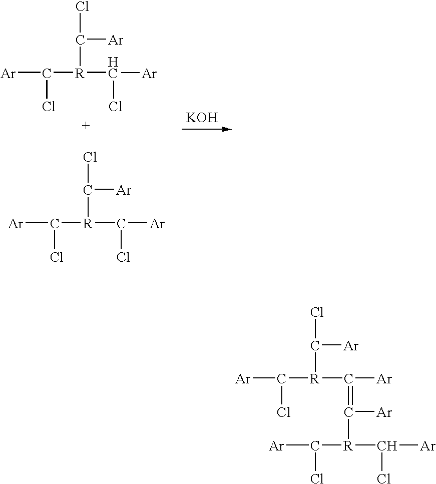

For self-reactive compounds containing oxidative coupling reactive groups, the initial environment typically can be can be dry and sterile, or a non-basic medium. The modification of the initial environment will typically comprise the addition of a base.

4. Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

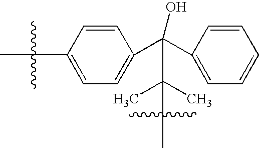

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

5. Temperature-sensitive Reactive Groups

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

B. Linking Groups

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; α-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance.

By way of example, particular linking groups and corresponding formulas are indicated in the following Table:

| Linking group | Component structure |
|---|---|
| —O—(CH₂)ₓ— | —O—(CH₂)ₓ—X |
| | —O—(CH₂)ₓ—Y |
| | —O—(CH₂)ₓ—Z |
| —S—(CH₂)ₓ— | —S—(CH₂)ₓ—X |
| | —S—(CH₂)ₓ—Y |
| | —S—(CH₂)ₓ—Z |
| —NH—(CH₂)ₓ— | —NH—(CH₂)ₓ—X |
| | —NH—(CH₂)ₓ—Y |
| | —NH—(CH₂)ₓ—Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ—Z |
| —NH—(CO)—O—(CH₂)ₓ— | —NH—(CO)—O—(CH₂)ₓ—X |
| | —NH—(CO)—O—(CH₂)ₓ—Y |
| | —NH—(CO)—O—(CH₂)ₓ—Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ—Z |
| —(CO)—O—(CH₂)ₓ— | —(CO)—O—(CH₂)ₓ—X |
| | —(CO)—O—(CH₂)ₓ—Y |
| | —(CO)—O—(CH₂)ₓ—Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ—Z |
| —O—(CO)—CHR²— | —O—(CO)—CHR²—X |
| | —O—(CO)—CHR²—Y |
| | —O—(CO)—CHR²—Z |
| —O—R³—(CO)—NH— | —O—R³—(CO)—NH—X |
| | —O—R³—(CO)—NH—Y |
| | —O—R³—(CO)—NH—Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

C. The Core

The “core” of each self-reactive compound is comprised of the molecular structure to which the reactive groups are bound. The molecular core can a polymer, which includes synthetic polymers and naturally occurring polymers. In one embodiment, the core is a polymer containing repeating monomer units. The polymers can be hydrophilic, hydrophobic, or amphiphilic. The molecular core can also be a low molecular weight components such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

1. Hydrophilic Polymers

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), poly(N-isopropyl-acrylamide), and copolymers thereof; poly(olefinic alcohols) such as poly(vinyl alcohols) and copolymers thereof; poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows. Non-transgenic, recombinant collagen expression in yeast and other cell lines) is described in U.S. Pat. No. 6,413,742 to Olsen et al., 6,428,978 to Olsen et al., and 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

2. Hydrophobic Polymers

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

3. Amphiphilic Polymers

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains), or a hydrophobic polymer that has been modified with hydrophilic moieties (e.g., “PEGylated” phospholipids such as polyethylene glycolated phospholipids).

4. Low Molecular Weight Components

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)-based tetracarboxylic acid, heptanedioic acid, octanedioic acid (suberic acid), and hexadecanedioic acid (thapsic acid).

Low molecular weight di- and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

D. Preparation

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al. (1990) Crit. Rev. Therap. Drug Carrier Syst. 6: 315), peptide chemistry (see Mutter et al., The Peptides, Academic: New York, N.Y. 2: 285-332; and Zalipsky et al. (1987) Int. J. Peptide Protein Res. 30: 740), and the synthesis of polymeric drugs (see Zalipsky et al. (1983) Eur. Polym. J. 19: 1177; and Ouchi et al. (1987) J. Macromol. Sci. Chem. A24: 1011).

Functionalized forms of PEG, including multi-functionalized PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, CaliforEia, formerly Shearwater Polymers, Huntsville, Ala.) and from Huntsman's Performance Chemicals Group (Houston, Tex.) under the name Jeffaminee polyoxyalkyleneamines. Multi-amino PEGs useful in the present invention: include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol)ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

Other in situ Crosslinking Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., 6,632,457). The in situ forming material may be a multi-component hydrogel system (see, e.g., U.S. Pat. No. 6,379, 373). The in situ forming material may be a multi-arm block copolymer that includes a central core molecule, such as a residue of a polyol, and at least three copolymer arms covalently attached to the central core molecule, each copolymer arm, comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water soluble macromers (see, e.g., U.S. Pat. No. 5,410,016). The in situ forming material may be formed from a two component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene)glycol polymer solution whereby upon mixing the solution cross-links to form a mechanical non-liquid covering structure which acts as a sealant. See, e.g., U.S. Pat. Nos. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See, e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See, e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See, e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol). See, e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See, e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See, e.g., U.S. Pat. No. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See, e.g., U.S. Patent Application No. 2004/0063613A1 and PCT Publication Nos. WO 01/45761 and WO 03/090683. The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See, e.g., U.S. Patent Application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See, e.g., PCT Publication No. WO 99/66964. The in situ forming material may be composed of collagen and a peroxidase and hydrogen peroxide, such that the collagen is crosslinked to from a semi-solid gel that seals a wound. See, e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See, e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See, e.g., U.S. Pat. No. 5,206,331.

Within another aspect of the present invention, polymeric carriers can be materials that are formed in situ from precursor molecules including the following: In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible light, UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 0.00/64977, U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975, and U.S. Patent Application Publication Nos. 2002/012796A1, 2002/0127266A1, 2002/0151650A1, 2003/0104032A1, 2002/0091229A1, and 2003/0059906A1.

In another embodiment, the reagents can undergo an electrophilic-nucleophilic reaction to produce a crosslinked matrix. For example, a 4-armed thiol derivatized polyethylene glycol (pentaerythritol poly(ethylene glycol)ether tetra-succinimidyl glutarate (4-armed NHS PEG)) can be reacted with a 4 armed NHS-derivatized polyethylene glycol (pentaerythritol poly(ethylene glycol)ether tetra-sulfhydryl (4-armed thiol PEG)) under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; and PCT Application Publication Nos. WO 04/060405 and WO 04/060346.

Other examples of in situ forming materials that can be used include those based on the crosslinking of proteins (described in U.S. Patent Nos. RE38158; 4,839,345; 5,514,379, 5,583,114; 6,458,147; 6,371,975; U.S. Patent Application Publication Nos. 2002/0161399; 2001/0018598 and PCT Publication Nos. WO 03/090683; WO 01/45761; WO 99/66964 and WO 96/03159).

In another embodiment, the anti-fibrosing agent can be coated onto the entire device or a portion of the device. In certain embodiments, the agent is present as part of a coating on a surface of the soft tissue implant. The coating may partially cover or may completely cover the surface of the soft tissue implant. Further, the coating may directly or indirectly contact the soft tissue implant. For example, the soft tissue implant may be coated with a first coating and then coated with a second coating that includes the anti-scarring agent.

Soft tissue implants may be coated using a variety of coating methods, including by dipping, spraying, painting, by vacuum deposition, or by any other method known to those of ordinary skill in the art.

As described above, the anti-fibrosing agent can be coated onto the appropriate soft tissue implant using the polymeric coatings described above. In addition to the coating compositions and methods described above, there are various other coating compositions and methods that are known in the art. Representative examples of these coating compositions and methods are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,231; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Within another aspect of the invention, the biologically active agent can be delivered with non-polymeric agents. These non-polymeric agents can include sucrose derivatives (e.g., sucrose acetate isobutyrate, sucrose oleate), sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites, and combinations and mixtures thereof.

Representative examples of patents relating to non-polymeric delivery systems and their preparation include U.S. Pat. Nos. 5,736,152; 5,888,533; 6,120,789; 5,968,542; and 5,747,058.

The fibrosis-inhibiting agent may be delivered as a solution (e.g., in a saline filled implant). The fibrosis-inhibiting agent can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, CMC, and the like). In another aspect of the invention, the solution can include a biocompatible solvent, such as ethanol, DMSO, glycerol, PEG-200, PEG-300 or NMP.

Within another aspect of the invention, the fibrosis-inhibiting agent can further comprise a secondary carrier. The secondary carrier can be in the form of microspheres (e.g., PLGA, PLLA, PDLLA, PCL, gelatin, polydioxanone, poly(alkylcyanoacrylate), nanospheres (e.g., PLGA, PLLA, PDLLA, PCL, gelatin, polydioxanone, poly(alkylcyanoacrylate)), liposomes, emulsions, microemulsions, micelles (e.g., SDS, block copolymers of the form X—Y, X—Y—X or Y—X—Y, R—(Y—X)ₙ, R—(X—Y)ₙ where X is a poly(alkylene oxide) or alkyl ether thereof and Y is a polyester where the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLLA, PDLLA, PCL, polydioxanone) and R is a multifunctional initiator, zeolites or cyclodextrins.

Within another aspect of the invention, these fibrosis-inhibiting agent secondary carrier compositions can be (a) incorporated directly into, or onto, the soft tissue implant, (b) incorporated into a solution (e.g., the saline within a soft tissue implant), (c) incorporated into a gel or viscous solution (e.g., the silicone or gelatinous filler of a soft tissue implant), (d) incorporated into the composition used for coating the soft tissue implant, or (e) incorporated into, or onto, the soft tissue implant following coating of the implant with a coating composition.

For example, fibrosis-inhibiting agent loaded PLGA microspheres may be incorporated into a polyurethane coating solution, which is then coated onto the soft tissue implant.

In yet another example, the soft tissue implant can be coated with a polyurethane and then allowed to partially dry such that the surface is still tacky. A particulate form of the fibrosis-inhibiting agent or fibrosis-inhibiting agent/secondary carrier can then be applied to all or a portion of the tacky coating after which the device is dried.

In yet another example, the soft tissue implant can be coated with one of the coatings described above. A thermal treatment process can then be used to soften the coating, after which the fibrosis-inhibiting agent or the fibrosis-inhibiting agent/secondary carrier is applied to the entire implant or to a portion of the implant (e.g., outer surface).

Within another aspect of the invention, the coated soft tissue implant that inhibits or reduces an in vivo fibrotic reaction is further coated with a compound or compositions which delay the release of and/or activity of the fibrosis-inhibiting agent. Representative examples of such agents include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation).

For example, in one embodiment of the invention the active agent on the soft tissue implant is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the blood or body fluids, the MePEG will dissolve out of the PLGA, leaving channels through the PLGA to an underlying layer containing the fibrosis-inhibiting agent, which then can then diffuse into the tissue and initiate its biological activity.

In another embodiment of the invention, for example, a particulate form of the active agent may be coated onto the soft tissue implant using a polymer (e.g., PLG, PLA, polyurethane). A second polymer that dissolves slowly or degrades (e.g., MePEG-PLGA or PLG) and that does not contain the active agent may be coated over the first layer. Once the top layer dissolves or degrades, it exposes the under coating which allows the active agent to be exposed to the treatment site or to be released from the coating.

Within another aspect of the invention, the outer layer of the coating of a coated soft tissue implant that inhibits an in vivo fibrotic response is further treated to crosslink the outer layer of the coating. This can be accomplished by subjecting the coated implant to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the implant surface with an inert molecule that prevents access to the active site through steric hindrance, or by coating the surface with an inactive form of the fibrosis-inhibiting agent, which is later activated. For example, the implant can be coated with an enzyme, which causes either release of the fibrosis-inhibiting agent or activates the fibrosis-inhibiting agent.

Another example of a suitable soft tissue implant surface coating includes an anticoagulant such as heparin, which can be coated on top of the fibrosis-inhibiting agent. The presence of the anticoagulant delays coagulation. As the anticoagulant dissolves away, the anticoagulant activity may stop, and the newly exposed fibrosis-inhibiting agent may inhibit or reduce fibrosis from occurring in the adjacent tissue or coating the implant.

The soft tissue implant can be coated with an inactive form of the fibrosis-inhibiting agent, which is then activated once the device is deployed. Such activation can be achieved by injecting another material into the treatment area after the device (as described below) is deployed or after the fibrosis-inhibiting agent has been administered to the treatment area (via, e.g., injections, spray, wash, drug delivery catheters or balloons). For example, the soft tissue implant can be coated with an inactive form of the fibrosis-inhibiting agent. Once the implant is deployed, the activating substance is injected or applied into or onto the treatment site where the inactive form of the fibrosis-inhibiting agent has been applied. For example, a soft tissue implant can be coated with a biologically active fibrosis-inhibiting agent and a first substance having moieties that capable of forming an ester bond with another material. The coating can be covered with a second substance such as polyethylene glycol. The first and second substances can react to form an ester bond via, e.g., a condensation reaction. Prior to the deployment of the implant, an esterase is injected into the treatment site around the outside of the soft tissue implant, which can cleave the bond between the ester and the fibrosis-inhibiting agent, allowing the agent to initiate fibrosis-inhibition.

The devices and compositions of the invention may include one or more additional ingredients and/or therapeutic agents, such as surfactants (e.g., PLURONICS, such as F-127, L-122, L-101, L-92, L-81, and L-61), anti-inflammatory agents (e.g., dexamethasone or aspirin), anti-thrombotic agents (e.g., heparin, high activity heparin, heparin quaternary amine complexes (e.g., heparin benzalkonium chloride complex)), anti-infective agents (e.g., 5-fluorouracil (5-FU), triclosan, rifamycim, and silver compounds), preservatives, anti-oxidants and/or anti-platelet agents.

Within certain embodiments of the invention, the device or therapeutic composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, a composition may be echogenic or radiopaque (e.g., made With echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). For visualization under MRI, contrast agents (e.g., gadolinium (III) chelates or iron oxide compounds) may be incorporated into the composition. In some embodiments, a medical device may include radio-opaque or MRI visible markers (e.g., bands) that may be used to orient and guide the device during the implantation procedure.

The devices may, alternatively, or in addition, be visualized under visible light, using fluorescence, or by other spectroscopic means. Visualization agents that can be included for this purpose include dyes, pigments, and other colored agents. In one aspect, the composition may further include a colorant to improve visualization of the composition in vivo and/or ex vivo. Frequently, compositions can be difficult to visualize upon delivery into a host, especially at the margins of an implant or tissue. A coloring agent can be incorporated into a composition to reduce or eliminate the incidence or severity of this problem. The coloring agent provides a unique color, increased contrast, or unique fluorescence characteristics to the composition. In one aspect, a composition is provided that includes a colorant such that it is readily visible (under visible light or using a fluorescence technique) and easily differentiated from its implant site. In another aspect, a colorant can be included in a liquid or semi-solid composition. For example, a single component of a two-component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan.

In one aspect, the devices and compositions of the present invention include one or more coloring agents, also referred to as dyestuffs, which may be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the devices compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the devices and compositions of the present invention include one or more antioxidants, present in an effective amount. Examples of the antioxidant include sulfites, alpha-tocopherol and ascorbic acid.

Within certain aspects of the present invention, the therapeutic composition may be biocompatible, and release one or more fibrosis-inhibiting agents over a period of several hours, days, or, months. As described above, “release of an agent” refers to any statistically significant presence of the agent, or a subcomponent thereof, which has disassociated from the compositions and/or remains active on the surface of (or within) the composition. The compositions of the present invention may release the anti-scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to reduce or inhibit any one or more components of fibrosis (or scarring) (or gliosis), including: formation of new blood vessels (angiogenesis), migration and/or proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring agent at a time such that at least one of the components of fibrosis (or gliosis) is inhibited or reduced. Moreover, the predetermined release rate may reduce agent loading and/or concentration as well as potentially providing minimal drug washout and thus, increases efficiency of drug effect. Any one of the anti-scarring agents described herein may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, and substantially non-releasing.

The total amount of anti-scarring agent made available on, in or near the device may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The surface amount of anti-scarring agent on, in or near the device may be in an amount ranging from less than 0.01 μg to about 250 μg per mm² of device surface area. Generally, the anti-scarring agent may be in the amount ranging from less than 0.01 μg per mm²; or from 0.01 μg to about 10 μg per mm²; or from 10 μg to about 250 μg per mm².

The anti-scarring agent that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring agent released from the composition as a function of time may be determined based on the in vitro release characteristics Of the agent from the composition. The in vitro release rate may be determined by placing the anti-scarring agent within the composition or device in an appropriate buffer such as 0.1 M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from an amount ranging from about 0.01 μg (micrograms) to about 2500 mg (milligrams). Generally, the anti-scarring agent that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring agent is made available to the susceptible tissue site in a programmed, sustained, and/or controlled manner that results in increased efficiency and/or efficacy. Further, the release rates may vary during either or both of the initial and subsequent release phases. There may also be additional phase(s) for release of the same substance(s) and/or different substance(s).

Further, therapeutic compositions and devices of the present invention may have a stable shelf-life of at least several months and be capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In another aspect, the compositions and devices of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics. In another embodiment, a secondary package can be used for the product. In another embodiment, product can be in a sterile container that is placed in a box that is labeled to describe the contents of the box.

2) Coating of Soft Tissue Implants with Fibrosis-inhibiting Agents

As described above, a range of polymeric and non-polymeric materials can be used to incorporate the fibrosis-inhibiting agent onto or into a soft tissue implant. Coating the soft tissue implant with these fibrosis-inhibiting agent-containing compositions, or with the fibrosis-inhibiting agent only, is one process that can be used to incorporate the fibrosis-inhibiting agent into or onto the implant.

a) Dip coating

Dip coating is an example of coating process that can be used to associate the anti-scarring agent with the soft tissue implant. In one embodiment, the fibrosis-inhibiting agent is dissolved in a solvent for the fibrosis-inhibiting agent and is then coated onto the soft tissue implant.

Fibrosis-Inhibiting Agent with an Inert Solvent

In one embodiment, the solvent is an inert solvent for the soft tissue implant such that the solvent does not dissolve the medical implant to any great extent and is not absorbed by the implant to any great extent. The soft tissue implant can be immersed, either partially or completely, in the fibrosis-inhibiting agent/solvent solution for a specific period of time. The rate of immersion into the fibrosis-inhibiting agent/solvent solution can be altered (e.g., 0.001 cm per sec to 50 cm per sec). The implant can then be removed from the solution. The rate at which the implant is withdrawn from the solution can be altered (e.g., 0.001 cm per sec to 50 cm per sec). The coated implant can be air-dried. The dipping process can be repeated one or more times depending on the specific application, where higher repetitions generally increase the amount of agent that is coated onto the soft tissue implant. The implant can be dried under vacuum to reduce residual solvent levels. This process will result in the fibrosis-inhibiting agent being coated on the surface of the soft tissue implant.

Fibrosis-Inhibiting Agent with a Swelling Solvent

In one embodiment, the solvent is one that will not dissolve the soft tissue implant but will be absorbed by the implant. In certain cases, these solvents can swell the implant to some extent. The implant can be immersed, either partially or completely, in the fibrosis-inhibiting agent/solvent solution for a specific period of time (seconds to days). The rate of immersion into the fibrosis-inhibiting agent/solvent solution can be altered (e.g., 0.001 cm per sec to 50 cm per sec). The implant can then be removed from the solution. The rate at which the soft tissue implant is withdrawn from the solution can be altered (e.g., 0.001 cm per sec to 50 cm per sec). The coated implant can be air-dried. The dipping process can be repeated one or more times depending on the specific application. The implant can be dried under vacuum to reduce residual solvent levels. This process will result in the fibrosis-inhibiting agent being adsorbed into the soft tissue implant. The fibrosis-inhibiting agent may also be present on the surface of the implant. The amount of surface associated fibrosis-inhibiting agent may be reduced by dipping the coated implant into a solvent for the fibrosis-inhibiting agent, or by spraying the coated implant with a solvent for the fibrosis-inhibiting agent.

Fibrosis-Inhibiting Agent with a Solvent

In one embodiment, the solvent is one that will be absorbed by the soft tissue implant and that will not dissolve the implant. The implant can be immersed, either partially or completely, in the fibrosis-inhibiting agent/solvent solution for a specific period of time (seconds to hours). The rate of immersion into the fibrosis-inhibiting agent/solvent solution can be altered (e.g., 0.001 cm per sec to 50 cm per sec). The implant can then be removed from the solution. The rate at which the implant is withdrawn from the solution can be altered (e.g., 0.001 cm per sec to 50 cm per sec). The coated implant can be air-dried. The dipping process can be repeated one or more times depending on the specific application. The implant can be dried under vacuum to reduce residual solvent levels. This process will result in the fibrosis-inhibiting agent being adsorbed into the soft tissue implant as well as being surface associated. Preferably, the exposure time of the implant to the solvent does not incur significant permanent dimensional changes to the implant. The fibrosis-inhibiting agent may also be present on the surface of the implant. The amount of surface associated fibrosis-inhibiting agent may be reduced by dipping the coated implant into a solvent for the fibrosis-inhibiting agent or by spraying the coated implant with a solvent for the fibrosis-inhibiting agent.

In one embodiment, the fibrosis-inhibiting agent and a polymer are dissolved in a solvent, for both the polymer and the fibrosis-inhibiting agent, and are then coated onto the soft tissue implant.

In the above description the soft tissue implant can be one that has not been modified or one that has been further modified by coating with a polymer, surface treated by plasma treatment, flame treatment, corona treatment, surface oxidation or reduction, surface etching, mechanical smoothing or roughening, or grafting prior to the coating process.

In any one the above dip coating methods, the surface of the soft tissue implant can be treated with a plasma polymerization method prior to coating of the fibrosis-inhibiting agent or fibrosis-inhibiting agent-containing composition, such that a thin polymeric layer is deposited onto the implant surface. Examples of such methods include the use of various monomers such hydrocyclosiloxane monomers.

b) Spray coating Soft Tissue Implants

Spray coating is another coating process that can be used in the practice of this invention. In the spray coating process, a solution or suspension of the fibrosis-inhibiting agent, with or without a polymeric or non-polymeric carrier, is nebulized and directed to the soft tissue implant to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting agent is dissolved in a solvent for the fibrosis agent and is then sprayed onto the soft tissue implant.

Fibrosis-Inhibiting Agent with an Inert Solvent

In one embodiment, the solvent is an inert solvent for the soft tissue implant such that the solvent does not dissolve the medical implant to any great extent and is not absorbed to any great extent. The implant can be held in place or mounted onto a mandrel or rod that has the ability to move in an X, Y or Z plane or a combination of these planes. Using one of the above described spray devices, the soft tissue implant can be spray coated such that it is either partially or completely coated with the fibrosis-inhibiting agent/solvent solution. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. This process will result in the fibrosis-inhibiting agent being coated on the surface of the soft tissue implant.

Fibrosis-Inhibiting Agent with a Swelling solvent

In one embodiment, the solvent is one that will not dissolve the soft tissue implant but will be absorbed by it. These solvents can thus swell the implant to some extent. The soft tissue implant can be spray coated, either partially or completely, in the fibrosis-inhibiting agent/solvent solution. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. This process will result in the fibrosis-inhibiting agent being adsorbed into the soft tissue implant. The fibrosis-inhibiting agent may also be present on the surface of the implant. The amount of surface associated fibrosis-inhibiting agent may be reduced by dipping the coated implant into a solvent for the fibrosis-inhibiting agent, or by spraying the coated implant with a solvent for the fibrosis-inhibiting agent.

Fibrosis-Inhibiting Agent with a Solvent

In one embodiment, the solvent is one that will be absorbed by the soft tissue implant and that will dissolve it. The soft tissue implant can be spray coated, either partially or completely, in the fibrosis-inhibiting agent/solvent solution. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. This process will result in the fibrosis-inhibiting agent being adsorbed into the soft tissue implant as well as being surface associated. In the preferred embodiment, the exposure time of the implant to the solvent may not incur significant permanent dimensional changes to it. The fibrosis-inhibiting agent may also be present on the surface of the implant. The amount of surface associated fibrosis-inhibiting agent may be reduced by dipping the coated implant into a solvent for the fibrosis-inhibiting agent, or by spraying the coated implant with a solvent for the fibrosis-inhibiting agent.

In the above description the soft tissue implant can be one that has not been modified as well as one that has been further modified by coating with a polymer, surface treated by plasma treatment, flame treatment, corona treatment, surface oxidation or reduction, surface etching, mechanical smoothing or roughening, or grafting prior to the coating process.

In one embodiment, the fibrosis-inhibiting agent and a polymer are dissolved in a solvent, for both the polymer and the anti-fibrosing agent, and are then spray coated onto the soft tissue implant.

Fibrosis-inhibiting Agent/Polymer with an Inert Solvent

In one embodiment, the solvent is an inert solvent for the soft tissue implant such that the solvent does not dissolve it to any great extent and is not absorbed by it to any great extent. The soft tissue implant can be spray coated, either partially or completely, in the fibrosis-inhibiting agent polymer/solvent solution for a specific period of time. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. This process will result in the fibrosis-inhibiting agent/polymer being coated on the surface of the soft tissue implant.

Fibrosis-Inhibiting Agent/Polymer with a Swelling Solvent

In one embodiment, the solvent is one that will not dissolve the soft tissue implant but will be absorbed by it. These solvents can thus swell the implant to some extent. The soft tissue implant can be spray coated, either partially or completely, in the fibrosis-inhibiting agent/polymer/solvent solution. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. This process will result in the fibrosis-inhibiting agent/polymer being coated onto the surface of the soft tissue implant as well as the potential for the fibrosis-inhibiting agent being adsorbed into the soft tissue implant. The fibrosis-inhibiting agent may also be present on the surface of the implant. The amount of surface associated fibrosis-inhibiting agent may be reduced by dipping the coated implant into a solvent for the fibrosis-inhibiting agent or by spraying the coated implant with a solvent for the fibrosis-inhibiting agent.

Fibrosis-Inhibiting Agent/Polymer with a Solvent

In one embodiment, the solvent is one that will be absorbed by the soft tissue implant and that will dissolve it. The soft tissue implant can be spray coated, either partially or completely, in the fibrosis-inhibiting agent/solvent solution. The rate of spraying of the fibrosis-inhibiting agent/solvent solution can be altered (e.g., 0.001 ml per sec to 10 ml per sec) to ensure that a good coating of the fibrosis-inhibiting agent is obtained. The coated implant can be air-dried. The spray coating process can be repeated one or more times depending on the specific application. The implant can be dried under vacuum to reduce residual solvent levels. In the preferred embodiment, the exposure time of the implant to the solvent may not incur significant permanent dimensional changes to it (other than those associated with the coating itself. The fibrosis-inhibiting agent may also be present on the surface of the implant. The amount of surface associated fibrosis-inhibiting agent may be reduced by dipping the coated implant into a solvent for the fibrosis-inhibiting agent or by spraying the coated implant with a solvent for the fibrosis-inhibiting agent.

In the above description, the soft tissue implant can be one that has not been modified as well as one that has been further modified by coating with a polymer, surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting agent in a polymer solution can be prepared. The suspension can be prepared by choosing a solvent that can dissolve the polymer but not the fibrosis-inhibiting agent, or a solvent that can dissolve the polymer and in which the fibrosis-inhibiting agent is above its solubility limit. In similar processes described above, the suspension of the fibrosis-inhibiting and polymer solution can be sprayed onto the soft tissue implant such that it is coated with a polymer that has a fibrosis-inhibiting agent suspended within it.

The present invention in its various embodiments provides the following devices and methods for making and using the devices.

In one embodiment, the present invention provides a device comprising a soft tissue implant and either an anti-scarring agent or a composition comprising an anti-scarring agent, wherein the agent inhibits scarring between the device and the host into which the device is implanted.

In certain embodiments, the soft tissue implant is a cosmetic implant; the implant is a reconstructive implant; the implant is a breast implant; the implant is a facial implant; the implant is a chin implant; the implant is a mandibular implant; the implant is a lip implant; the implant is a nasal implant; the implant is a cheek implant; the implant is a pectoral implant; the implant is a buttocks implant; the implant is an autogenous tissue implant. In certain embodiments, the soft tissue implant is a breast implant, wherein the breast implant comprises saline. In another embodiment, the breast implant comprises silicone.

In one embodiment, the soft tissue implant is an autogenous tissue implant. In certain embodiments, the autogenous tissue implant is defined by one, two, or more of the following features: the autogenous tissue implant comprises adipose tissue; the implant comprises an autogenous fat implant; the implant comprises a dermal implant; the implant comprises a dermal plug; the implant comprises a tissue plug; the implant comprises a muscular tissue flap; the implant comprises a pedicle flap; the implant comprises a pedicle flap, wherein the pedicle flap is from the back, abdomen, buttocks, thigh, or groin; the implant comprises a cell extraction implant; the implant comprises a suspension of autologous dermal fibroblasts. In another embodiment, the device that comprises an autogenous tissue implant is a tissue filler, and in still another embodiment, the device is a fat graft.

In another embodiment the invention provides a device comprising a breast implant and either an anti-scarring agent or a composition comprising an anti-scarring agent, wherein the agent inhibits scarring between the device and the host into which the device is implanted. In one embodiment, a device comprises a facial implant and either an anti-scarring agent or a composition comprising an anti-scarring agent, wherein the agent inhibits scarring between the device and the host into which the device is implanted. In another embodiment, the device comprises a chin implant and either an anti-scarring agent or a composition comprising an anti-scarring agent, wherein the agent inhibits scarring between the device and the host into which the device is implanted. In one embodiment, a device is provided that comprises a mandibular implant and either an anti-scarring agent or a composition comprising an anti-scarring agent, wherein the agent inhibits scarring between the device and the host into which the device is implanted. In still another embodiment the invention provides a device comprising a lip implant and either an anti-scarring agent or a composition comprising an anti-scarring agent, wherein the agent inhibits scarring between the device and the host into which the device is implanted. In another embodiment, a device comprises a nasal implant and either an anti-scarring agent or a composition comprising an anti-scarring agent, wherein the agent inhibits scarring between the device and the host into which the device is implanted. In another embodiment, a device comprising a cheek implant and either an anti-scarring agent or a composition comprising an anti-scarring agent, wherein the agent inhibits scarring between the device and the host into which the device is implanted. In another embodiment, a device is provided that comprises a pectoral implant and either an anti-scarring agent or a composition comprising an anti-scarring agent, wherein the agent inhibits scarring between the device and the host into which the device is implanted. In still another embodiment of the invention, a device comprises a buttocks implant and either an anti-scarring agent or a composition comprising an anti-scarring agent, wherein the agent inhibits scarring between the device and the host into which the device is implanted. In one embodiment, the invention provides a device comprising an autogenous tissue implant and either an anti-scarring agent or a composition comprising an anti-scarring agent, wherein the agent inhibits scarring between the autogenous tissue implant and the host into which the device is implanted.

The invention also provides a method for inhibiting scarring between a soft tissue implant and a host comprising placing a device that comprises the soft tissue implant and either an anti-scarring agent or a composition comprising the anti-scarring agent into the host, wherein the agent inhibits scarring; a method for inhibiting scarring between a breast implant and a host comprising placing a device that comprises the breast implant and either an anti-scarring agent or a composition comprising the anti-scarring agent into the host, wherein the agent inhibits scarring; a method for inhibiting scarring between a facial implant and a host comprising placing a device that comprises the facial implant and either an anti-scarring agent or a composition comprising the anti-scarring agent into the host, wherein the agent inhibits scarring. The invention also provides a method for inhibiting scarring between a chin implant and a host comprising placing a device that comprises the chin implant and either an anti-scarring agent or a composition comprising the anti-scarring agent into the host, wherein the agent inhibits scarring; a method for inhibiting scarring between a mandibular implant and a host comprising placing a device that comprises the mandibular implant and either an anti-scarring agent or a composition comprising the anti-scarring agent into the host, wherein the agent inhibits scarring; a method for inhibiting scarring between a lip implant and a host comprising placing a device that comprises the lip implant and either an anti-scarring agent or a composition comprising the anti-scarring agent into the host, wherein the agent inhibits scarring; and a method for inhibiting scarring between a nasal implant and a host comprising placing a device that comprises the nasal implant and either an anti-scarring agent or a composition comprising the anti-scarring agent into the host, wherein the agent inhibits scarring. Also provided is a method for inhibiting scarring between a cheek implant and a host comprising placing a device that comprises the cheek implant and either an anti-scarring agent or a composition comprising the anti-scarring agent into the host, wherein the agent inhibits scarring; a method for inhibiting scarring between a pectoral implant and a host comprising placing a device that comprises the pectoral implant and either an anti-scarring agent or a composition comprising the anti-scarring agent into the host, wherein the agent inhibits scarring; a method for inhibiting scarring between a buttocks implant and a host comprising placing a device that comprises the buttocks implant and either an anti-scarring agent or a composition comprising the anti-scarring agent into the host, wherein the agent inhibits scarring; and a method for inhibiting scarring between an autogenous tissue implant and a host comprising placing a device that comprises the autogenous tissue implant and either an anti-scarring agent or a composition comprising the anti-scarring agent into the host, wherein the agent inhibits scarring.

The invention also provides methods for making devices described herein. Provided herein is a method for making a device comprising combining a soft tissue implant and either an anti-scarring agent or a composition comprising an anti-scarring agent, wherein the agent inhibits scarring between the device and a host into which the device is implanted; a method for making a device comprising combining a breast implant and either an anti-scarring agent or a composition comprising an anti-scarring agent, wherein the agent inhibits scarring between the device and a host into which the device is implanted; a method for making a device comprising combining a facial implant and either an anti-scarring agent or a composition comprising an anti-scarring agent, wherein the agent inhibits scarring between the device and a host into which the device is implanted; and a method for making a device comprising combining a chin implant and either an anti-scarring agent or a composition comprising an anti-scarring agent, wherein the agent inhibits scarring between the device and a host into which the device is implanted. Also provided is a method for making a device comprising combining a mandibular implant and either an anti-scarring agent or a composition comprising an anti-scarring agent, wherein the agent inhibits scarring between the device and a host into which the device is implanted; a method for making a device comprising combining a lip implant and either an anti-scarring agent or a composition comprising an anti-scarring agent, wherein the agent inhibits scarring between the device and a host into which the device is implanted; a method for making a device comprising combining a nasal implant and either an anti-scarring agent or a composition comprising an anti-scarring agent, wherein the agent inhibits scarring between the device and a host into which the device is implanted; a method for making a device comprising combining a cheek implant and either an anti-scarring agent or a composition comprising an anti-scarring agent, wherein the agent inhibits scarring between the device and a host into which the device is implanted. In another embodiment, the invention provides a method for making a device comprising combining a pectoral implant and either an anti-scarring agent or a composition comprising an anti-scarring agent, wherein the agent inhibits scarring between the device and a host into which the device is implanted. Also provided is a method for making a device comprising combining a buttocks implant and either an anti-scarring agent or a composition comprising an anti-scarring agent, wherein the agent inhibits scammg between the device and a host into which the device is implanted; a method for making a device comprising combining an autogenous tissue implant and either an anti-scarring agent or a composition comprising an anti-scarring agent, wherein the agent inhibits scarring between the device and a host into which the device is implanted.

The invention also provides a method for reconstructing a breast comprising placing into a host a device that comprises a breast implant and either an anti-scarring agent or a composition comprising an anti-scarring agent, wherein the agent inhibits scarring between the device and the host into which the device is implanted; and the invention provides a method for augmenting a breast comprising placing into a host a device that comprises a breast implant and either an anti-scarring agent or a composition comprising an anti-scarring agent, wherein the agent inhibits scarring between the device and the host into which the device is implanted. The invention also provides a method for augmenting the malar or submalar region comprising placing into a host a device that comprises a facial implant and either an anti-scarring agent or a composition comprising an anti-scarring agent, wherein the agent inhibits scarring between the device and the host into which the device is implanted. In another embodiment, a method is provided for reconstructing a jaw comprising placing into a host a device that comprises a mandibular implant and either an anti-scarring agent or a composition comprising an anti-scarring agent, wherein the agent inhibits scarring between the device and the host into which the device is implanted. In one embodiment, the invention provides a method for reconstructing a chin comprising placing into a host a device that comprises a chin implant and either an anti-scarring agent or a composition comprising an anti-scarring agent, wherein the agent inhibits scarring between the device and the host into which the device is implanted; a method for reconstructing a nose comprising placing into a host a device that comprises a nasal implant and either an anti-scarring agent or a composition comprising an anti-scarring agent, wherein the agent inhibits scarring between the device and the host into which the device is implanted; a method for reconstructing a lip comprising placing into a host a device that comprises a lip implant and either an anti-scarring agent or a composition comprising an anti-scarring agent, wherein the agent inhibits scarring between the device and the host into which the device is implanted; and a method for reconstructing a chest comprising placing into a host a device that comprises a pectoral implant and either an anti-scarring agent or a composition comprising an anti-scarring agent, wherein the agent inhibits scarring between the device and the host into which the device is implanted. Also provided herein is a method for augmenting soft tissue comprising placing into a host a device that comprises an autogenous tissue implant and either an anti-scarring agent or a composition comprising an anti-scarring agent, wherein the agent inhibits scarring between the device and the host into which the device is implanted.

In particular embodiments, the anti-scarring agent reduces tissue regeneration; the agent inhibits inflammation; the agent inhibits fibrosis; the agent inhibits adhesion between the device and the host into which the device is implanted; the agent inhibits angiogenesis; the agent inhibits migration of connective tissue cells; the agent inhibits proliferation of connective tissue cells; the agent inhibits fibroblast migration; the agent inhibits fibroblast proliferation; the agent inhibits extracellular matrix production; the agent enhances extracellular matrix breakdown; the agent inhibits deposition of extracellular matrix; the agent inhibits tissue remodeling; the agent inhibits formation of a fibrous connective tissue capsule enclosing the device.

In certain embodiments, the anti-scarring agent is an angiogenesis inhibitor; a 5-lipoxygenase inhibitor or antagonist; a chemokine receptor antagonist; a C—C chemokine receptor 1, C—C chemokine receptor 3, or C—C chemokine receptor 5; a cell cycle inhibitor; a taxane; an anti-microtubule agent; paclitaxel; docetaxel; an analogue or derivative of paclitaxel; a vinca alkaloid; a vincak alkaloid wherein the vinca alkaloid is vinblastine; camptothecin or an analogue or derivative thereof; a podophyllotoxin; a podophyllotoxin, wherein the podophyllotoxin is etoposide or an analogue or derivative thereof; an anthracycline; an anthracycline, wherein the anthracycline is doxorubicin or an analogue or derivative thereof; an anthracycline, wherein the anthracycline is mitoxantrone or an analogue or derivative thereof; a platinum compound; a nitrosourea; a nitroimidazole; a folic acid antagonist; a cytidine analogue; a pyrimidine analogue; a fluoropyrimidine analogue; a purine analogue; a purine analogue, wherein the purine analogue is tubercidin; nitrogen mustard or an analogue or derivative thereof; a hydroxyurea; a mytomicin or an analogue or derivative thereof; an alkyl sulfonate; a benzamide or an analogue or derivative thereof; a nicotinamide or an analogue or derivative thereof; a halogenated sugar or an analogue or derivative thereof; a DNA alkylating agent; an anti-microtubule agent; a topoisomerase inhibitor; a DNA cleaving agent; and/or an antimetabolite. In certain embodiments, the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; and/or the agent is a cyclin dependent protein kinase inhibitor. In certain embodiments, the anti-scarring agent is an epidermal growth factor kinase inhibitor; an elastase inhibitor; a factor Xa inhibitor; a farnesyltransferase inhibitor; a fibrinogen antagonist; a guanylate cyclase stimulant; a heat shock protein 90 antagonist; a heat shock protein 90 antagonist, wherein the heat shock protein 90 antagonist is geldanamycin or an analogue or derivative thereof; a guanylate cyclase stimulant; a hydroxymethylglutaryl coenzyme A reductase (HMGCoA reductase) inhibitor; a HMGCoA reductase inhibitor, wherein the HMGCoA reductase inhibitor is simvastatin or an analogue or derivative thereof; a hydroorotate dehydrogenase inhibitor; an IkappaB kinase 2 (IKK2) inhibitor; an IL-1 antagonist; an interleukin-1beta-converting enzyme (ICE) antagonist; an IL-1R-associated kinase (IRAK) antagonist; an IL-4 agonist; and/or an immunomodulatory agent. In other particular embodiments, the anti-scarring agent is sirolimus or an analogue or derivative thereof and in certain other embodiments, the agent is not sirolimus. In another embodiment, the agent is everolimus or an analogue or derivative thereof, or is tacrolimus or an analogue or derivative thereof, or is not tacrolimus. In another embodiment, the agent is biolmus or an analogue or derivative thereof; tresperimus or an analogue or derivative thereof; auranofin or an analogue or derivative thereof; 27-O-demethylrapamycin or an analogue or derivative thereof; gusperimus or an analogue or derivative thereof; pimecrolimus or an analogue or derivative thereof; ABT-578 or an analogue or derivative thereof; an inosine monophosphate dehydrogenase (IMPDH) inhibitor; an IMPDH inhibitor, wherein the IMPDH inhibitor is mycophenolic acid or an analogue or derivative thereof; an IMPDH inhibitor, wherein the IMPDH inhibitor is 1-alpha-25 dihydroxy vitamin D₃ or an analogue or derivative thereof; a leukotriene inhibitor; a monocyte chemoattractant protein-1 (MCP-1) antagonist; a matrix metalloproteinase (MMP) inhibitor; an NF kappa B inhibitor; an NF kappa B inhibitor, wherein the NF kappa B inhibitor is Bay 11-7082; a nitric oxide (NO) antagonist; a p38 mitogen-activated protein (MAP) kinase inhibitor; a p38 mitogen-activated protein (MAP) kinase inhibitor, wherein the p38 MAP kinase inhibitor is SB 202190; a phosphodiesterase inhibitor; a transforming growth factor (TGF) beta inhibitor; a thromboxane A2 antagonist; a tumor necrosis factor alpha (TNFα) antagonist; a TNF-alpha converting enzyme (TACE) inhibitor; a tyrosine kinase inhibitor; a vitronectin inhibitor; a fibroblast growth factor inhibitor; a protein kinase inhibitor; a platelet derived growth factor (PDGF) receptor kinase inhibitor; an endothelial growth factor receptor kinase inhibitor; a retinoic acid receptor antagonist; and/or a fibrinogin antagonist. In other embodiments, the anti-scarring agent is an antimycotic agent; an antimycotic agent, wherein the antimycotic agent is sulconizole; a bisphosphonate; a phospholipase A1 inhibitor; a histamine H1/H2/H3 receptor antagonist; a macrolide antibiotic; a GPIIb/IIa receptor antagonist; an endothelin receptor antagonist; a peroxisome proliferator-activated receptor agonist; an estrogen receptor agent; a somastostatin analogue; a neurokinin 1 antagonist; a neurokinin 3 antagonist; a neurokinin antagonist; a (very late antigen-4 (VLA-4) antagonist; an osteoclast inhibitor; a DNA topoisomerase ATP hydrolyzing inhibitor; an angiotensin I converting enzyme inhibitor; an angiotensin II antagonist; an enkephalinase inhibitor; a peroxisome proliferator-activated receptor gamma agonist insulin sensitizer; a protein kinase C inhibitor; a ROCK (rho-associated kinase) inhibitor; a CXCR3 inhibitor; an Itk inhibitor; a cytosolic phospholipase A₂-alpha inhibitor; a peroxisome proliferator activated receptor (PPAR) agonist; an immunosuppressant; an Erb inhibitor; an apoptosis agonist; a lipocortin agonist; a vascular cell adhesion molecule-1 (VCAM-1) antagonist; a collagen antagonist; an alpha 2 integrin antagonist; a TNF alpha inhibitor; a nitric oxide inhibitor; a cathepsin inhibitor; and/or epithilone B. In certain other particular embodiments, the anti-scarring agent is not an anti-inflammatory agent; is not paclitaxel; is not a steroid; is not a glucocorticosteroid; is not dexamethasone; is not an anti-infective agent; is not an antibiotic; and/or the agent is not an anti-fungal agent.

In particular embodiments, the devices described herein that comprise a soft tissue implant (breast, facial, chin, mandibular, lip, nasal, cheek, pectoral, buttocks, autogenous tissue) and the methods that use these devices (for inhibiting scarring between a soft tissue implant and the host; for reconstructing or augmenting), and/or methods for making these devices have one or more of the following features: the anti-scarring agent or the composition comprising the anti-scarring agent is incorporated into the capsule of the implant; the agent or the composition is coated onto the surface of the implant; the agent or the composition is incorporated into the filling material of the implant.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In certain embodiments, the device comprises a soft tissue implant that comprises a polymer; wherein the polymer is silicone; the implant comprises a polymer, wherein the polymer is poly(tetrafluorethylene) (PTFE); the implant comprises a polymer, wherein the polymer is expanded poly(tetrafluorethylene) (ePTFE); the implant comprises a polymer, wherein the polymer is polyethylene; the implant comprises a polymer, wherein the polymer is polyurethane; the implant comprises a polymer, wherein the polymer is polymethylmethacrylate; the implant comprises a polymer, wherein the polymer is polyester; the implant comprises a polymer, wherein the polymer is polyamide; the implant comprises a polymer, wherein the polymer is polypropylene. In certain other embodiments, the device comprises a polymer independent from a polymer with which the implant is constructed.

In still other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and that are used in the methods for inhibiting scarring between a soft tissue implant and the host; and/or for reconstructing or augmenting), and that are made by methods described herein further comprise a coating. In one embodiment, the coating is not formed by graft polymerization. In another embodiment, the coating comprises a polymer. In still another embodiment, the device further comprises a first coating and a second coating, wherein the first coating comprises a polymer, and wherein the second coating comprises the anti-scarring agent. In one embodiment, the device further comprises a coating, wherein the coating comprises the anti-scarring agent and a polymer. In another embodiment, the device further comprises one or more of the following features: a coating, wherein the coating comprises the anti-scarring agent; a coating, wherein the coating is disposed on a surface of the device; a coating, wherein the coating directly contacts the device; a coating, wherein the coating directly contacts the implant and wherein the coating is a parylene coating; a coating, wherein the coating indirectly contacts the device; a coating, wherein the coating partially covers the device; a coating, wherein the coating completely covers the device; a coating, wherein the coating is a uniform coating; a coating, wherein the coating is a non-uniform coating; a coating, wherein the coating is a discontinuous coating; a coating, wherein the coating is a patterned coating; comprising a coating, wherein the coating has a thickness of 100 μm or less; a coating, wherein the coating has a thickness of 10 μm or less; a coating, wherein the coating adheres to the surface of the device upon deployment of the device; a coating, wherein the coating is stable at room temperature for a period of 1 year; a coating, wherein the anti-scarring agent is present in the coating in an amount ranging between about 0.0001% to about 1% by weight; a coating, wherein the anti-scarring agent is present in the coating in an amount ranging between about 1% to about 10% by weight; a coating, wherein the anti-scarring agent is present in the coating in an amount ranging between about 10% to about 25% by weight; a coating, wherein the anti-scarring agent is present in the coating in an amount ranging between about 25% to about 70% by weight; a coating, wherein the coating further comprises a polymer; a first coating having a first composition and the second coating having a second composition; a first coating having a first composition and the second coating having a second composition, wherein the first composition and the second composition are different.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. The device further comprises a polymer; a polymeric carrier; a polymeric carrier wherein the carrier is a sprayable formulation comprising collagen; a polymeric carrier wherein the carrier is a sprayable formulation comprising PEG; a polymeric carrier wherein the carrier is a formulation comprising fibrinogen; a polymeric carrier wherein the carrier is a formulation comprising hyaluronic acid; a polymeric carrier wherein the carrier is comprises a polymeric gel; a polymeric carrier wherein the carrier comprises glycol (pentaerythritol poly(ethylene glycol)ether tetra-succinimidyl glutarate (4-armed NHS-PEG); a polymeric carrier wherein the carrier comprises an electrospun material; a polymeric carrier wherein the carrier comprises an electrospun material wherein the material is collagen or PLGA; a polymeric carrier wherein the carrier comprises a polysaccharide gel; a polymeric carrier wherein the carrier comprises an orthopedic cement; a polymeric carrier wherein the carrier comprises a surgical adhesive; a polymeric carrier wherein the carrier comprises a surgical adhesive, wherein the adhesive comprises a cyanoacrylate; a polymeric carrier wherein the carrier comprises a biocompatible tissue filler; a polymeric carrier wherein the carrier is a film; a polymeric carrier wherein the carrier is a mesh; a polymeric carrier wherein the carrier is a sponge.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In certain embodiments, the device further comprises a polymeric matrix. In one embodiment, the polymeric matrix is formed from either one or both of pentaerythritol poly(ethylene glycol)ether tetra-sulfhydryl (4-armed thiol PEG) and pentaerythritol poly(ethylene glycol)ether tetra-succinimidyl glutarate (4-armed NHS PEG), and in another embodiment, the matrix further comprises collagen or a derivative thereof. In another embodiment, a polymeric matrix is formed from either one or both of pentaerythritol poly(ethylene glycol)ether tetra-amino](4-armed amino PEG) and pentaerythritol poly(ethylene glycol)ether tetra-succinimidyl glutarate (4-armed NHS PEG), and in a certain embodiment further comprises collagen or a derivative thereof. In certain other embodiments, a polymeric matrix is formed by at least one of the following: reacting a first synthetic polymer comprising two or more nucleophilic groups with a second synthetic polymer comprising two or more electrophilic groups; reacting a first synthetic polymer comprising two or more nucleophilic groups with a hydrophilic polymer; reacting a synthetic polymer comprising two or more electrophilic groups with a hydrophilic polymer; reacting a first synthetic polymer comprising two or more nucleophilic groups and a second synthetic polymer comprising two or more electrophilic groups with a hydrophilic polymer; reacting a synthetic polymer comprising two or more nucleophilic groups with a composition comprising a protein; reacting a synthetic polymer comprising two or more nucleophilic groups with a composition comprising a protein, wherein the protein is collagen; reacting a synthetic polymer comprising two or more nucleophilic groups with a composition comprising a protein, wherein the protein is methylated collagen; reacting a synthetic polymer comprising two or more nucleophilic groups with a composition comprising a protein, wherein the protein is fibrinogen; reacting a synthetic polymer comprising two or more nucleophilic groups with a composition comprising a protein, wherein the protein is thrombin; reacting a synthetic polymer comprising two or more nucleophilic groups with a composition comprising a protein, wherein the protein is albumin; reacting a synthetic polymer comprising two or more nucleophilic groups with a composition comprising a polysaccharide; reacting a synthetic polymer comprising two or more nucleophilic groups with a composition comprising a polysaccharide, wherein the polysaccharide is glycosaminoglycan; reacting a synthetic polymer comprising two or more nucleophilic groups with a composition comprising a polysaccharide, wherein the polysaccharide is deacetylated glycosaminoglycan; reacting a synthetic polymer comprising two or more nucleophilic groups with a composition comprising a polysaccharide, wherein the polysaccharide is desulfated glycosaminoglycan; reacting a synthetic polymer comprising two or more electrophilic groups with a composition comprising a protein, wherein the protein is collagen; reacting a synthetic polymer comprising two or more electrophilic groups with a composition comprising a protein, wherein the protein is methylated collagen; reacting a synthetic polymer comprising two or more electrophilic groups with a composition comprising a protein, wherein the protein is fibrinogen; reacting a synthetic polymer comprising two or more electrophilic groups with a composition comprising a protein, wherein the protein is thrombin; reacting a synthetic polymer comprising two or more electrophilic groups with a composition comprising a protein, wherein the protein is albumin; reacting a synthetic polymer comprising two or more electrophilic groups with a composition comprising a polysaccharide; reacting a synthetic polymer comprising two or more electrophilic groups with a composition comprising a polysaccharide, wherein the polysaccharide is glycosaminoglycan; reacting a synthetic polymer comprising two or more electrophilic groups with a composition comprising a polysaccharide, wherein the polysaccharide is deacetylated glycosaminoglycan; reacting a synthetic polymer comprising two or more electrophilic groups with a composition comprising a polysaccharide, wherein the polysaccharide is desulfated glycosaminoglycan; and/or a polymeric matrix is formed by a self-reactive compound that comprises a core substituted with at least three reactive groups.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In certain embodiments, the device further comprises a polymer. In one embodiment, the device comprises a polymer, wherein the polymer permits sustained release of the anti-scarring agent. In other embodiments, the device comprises a polymeric carrier that comprises one or more of the following: a copolymer; a block copolymer; a random copolymer; a biodegradable polymer; a non-biodegradable polymer; a hydrophilic polymer; a hydrophobic polymer; a polymer having hydrophilic domains; a polymer having hydrophobic domains; a non-conductive polymer; an elastomer; a hydrogel; a silicone polymer; a hydrocarbon polymer; a styrene-derived polymer; a butadiene polymer; a macromer; a poly(ethylene glycol)polymer; poly(D,L-lactic acid); poly (glycolic acid); comprises a copolymer of lactic acid and glycolic acid; comprises poly(caprolactone); poly(valerolactone); a polyanhydride; a copolymer comprising either poly(caprolactone) or poly(lactic acid) with a polyethylene glycol; a silicone rubber; poly(styrene)block-poly(isobutylene)-block-poly(styrene); a poly(acrylate); collagen; a poly(alkylene oxide); a polysaccharide; a polysaccharide wherein the polysaccharide is hyaluronic acid; a polysaccharide wherein the polysaccharide is chitosan; and a polysaccharide wherein the polysaccharide is fucan. In a particular embodiment, the device further comprises a polymeric carrier, wherein the polymeric carrier is pH sensitive; wherein the polymeric carrier is temperature sensitive; wherein the polymeric carrier is a thermogelling polymer; wherein the polymeric carrier comprises an amorphous polymer; wherein the carrier is formed in situ in the host; wherein the carrier is formed by polymerization in situ in the host; and/or wherein the carrier is formed by cross-linking in situ in the host.

In certain embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and that are used in the methods for inhibiting scarring between a soft tissue implant and the host, for reconstructing or augmenting, and/or that are made according to the methods for making these devices further comprise a non-polymeric carrier. In certain embodiments, the non-polymeric carrier is a sucrose derivative; a sterol; a C₁₂-C₂₄ fatty acid; a C₁₈-C₃₆ mono-, di- or tri-glyceride; a sucrose fatty acid ester; a sorbitan fatty acid ester; a C₁₆-C₁₈ fatty alcohol; a phospholipid; an ester of a fatty alcohol; sphingosine or a derivative thereof; a spingomyelin; a ceramide; a lanolin or a lanolin alcohol; calcium phosphate; hydroxyapatite; and/or a zeolite. In another embodiment, the device further comprises a lubricious coating.

In other embodiments, the invention provides devices and methods that use these devices, wherein the anti-scarring agent is located within a reservoir or a plurality of reservoirs of the implant (soft tissue, breast, facial, chin, mandibular, lip, nasal cheek, pectoral, buttocks, or autogenous); is located within a cavity, pore, or hole of the implant; and/or is located within a channel, lumen, or divet of the implant.

In yet other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In yet other embodiments, the device further comprises one or more of the following: a second pharmaceutically active agent; an anti-inflammatory agent; an anti-microbial agent; an agent that inhibits infection; an agent that inhibits infection, wherein the agent is an anthracycline; an agent that inhibits infection, wherein the agent is doxorubicin; an agent that inhibits infection, wherein the agent is mitoxantrone; an agent that inhibits infection, wherein the agent is a fluoropyrimidine; an agent that inhibits infection, wherein the agent is 5-fluorouracil (5-FU); an agent that inhibits infection, wherein the agent is a folic acid antagonist; an agent that inhibits infection, wherein the agent is methotrexate; an agent that inhibits infection, wherein the agent is a podophylotoxin; an agent that inhibits infection, wherein the agent is etoposide; an agent that inhibits infection, wherein the agent is a camptothecin; an agent that inhibits infection, wherein the agent is a hydroxyurea; an agent that inhibits infection, wherein the agent is a platinum complex; an agent that inhibits infection, wherein the agent is cisplatin; an anti-thrombotic agent.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In still other embodiments, the invention provides devices and methods that use these devices, wherein the device further comprises a fibrosis-promoting agent. The fibrosis-promoting agent comprises one or more of the following: an irritant; silk; silica; bleomycin; neomycin; talcum powder; metallic beryllium; a retinoic acid compound; copper; vinyl chloride or a polymer of vinyl chloride; a growth factor; a growth factor selected from an epidermal growth factor, transforming growth factor-α, a transforming growth factor-β, platelet-derived growth factor, a fibroblast growth factor, fibroblast stimulating factor-1, an activin, a vascular endothelial growth factor, an angiopoietin, an insulin-like growth factor, hepatocyte growth factor, connective tissue growth factor, a myeloid colony-stimulating factor, monocyte chemotactic protein, a granulocyte-macrophage colony-stimulating factor, granulocyte colony-stimulating factor, macrophage colony-stimulating factor, nerve growth factor, and erythropoietin, tumor necrosis factors, nerve growth factor, interferon-α, interferon-β, histamine, endothelin-1, angiotensin II, growth hormone, an interleukin (IL), IL-1, IL-8, and IL-6, or a peptide, analogue, or derivative thereof; at least one of calcium phosphate, calcium sulfate, calcium carbonate, or hydroxyapatite; an inflammatory microcrystal; a tissue adhesive; at least one of bromocriptine, methylsergide, methotrexate, chitosan, N-carboxybutyl chitosan, carbon tetrachloride, thioacetamide, fibrosin, ethanol, or a naturally occurring or synthetic peptide containing the Arg-Gly-Asp peptide sequence; an inhibitor of a matrix metalloproteinase; a cytokine, wherein the cytokine is a bone morphogenic protein (BMP) or demineralized bone matrix; and a component of extracellular matrix. In certain embodiments, the fibrosis-promoting agent stimulates cell proliferation. In other embodiments, the fibrosis-promoting agent is selected from dexamethasone, isotretinoin, 17-β-estradiol, estradiol, 1-α-25 dihydroxyvitamin D₃, diethylstibesterol, cyclosporine A, N(omega-nitro-L-arginine methyl ester (L-NAME), and all-trans retinoic acid.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In other embodiments, the invention provides devices and methods that use these devices, wherein the device further comprises a visualization agent. In particular embodiments, the visualization agent is a radio-opaque material, wherein the radio-opaque material comprises a metal, a halogenated compound, or a barium-containing compound. In other embodiments, the Visualization agent is a radio-opaque material, wherein the radio-opaque material comprises barium, tantalum, or technetium; or a MRI responsive material. In one embodiment the visualization agent comprises a gadolinium chelate, and in another embodiment, the visualization agent comprises iron, magnesium, manganese, copper, or chromium. In other embodiments, the visualization agent comprises one or more of the following: an iron oxide compound; a dye, pigment, or colorant; an echogenic material; an echogenic material, wherein the echogenic material is in the form of a coating.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. The invention also provides devices and methods that use these devices, wherein the device further comprises a surfactant; a preservative; an anti-oxidant; and/or an anti-platelet agent. In a particular embodiment, the device is sterile.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In one embodiment, the anti-scarring agent inhibits adhesion between the device and a host into which the device is implanted. In another embodiment, the composition comprising the anti-scarring agent further comprises a secondary carrier. In a certain embodiment, the secondary carrier is a microsphere; the secondary carrier is a nanosphere; the secondary carrier is a liposome; the secondary carrier is an emulsion; the secondary carrier is a microemulsion; the secondary carrier is a micelle; the secondary carrier is a block polymer; the secondary carrier is a zeolite; the secondary carrier is a cyclodextrin. In still other embodiments, the composition comprising the anti-scarring agent further comprises an inert solvent; a swelling solvent; or a solvent, wherein the solvent dissolves the implant. In still other embodiments, the composition comprising the anti-scarring agent further comprises a polymer and a solvent, wherein the solvent is an inert solvent; the solvent is a swelling solvent; or the solvent dissolves the implant. In still other embodiments, the composition comprising the anti-scarring agent is in the form of a gel, paste, film, or spray.

In other embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In certain embodiments, the implant is partially constructed with the agent or the composition comprising the anti-scarring agent. In another embodiment, the implant is impregnated with the agent or the composition comprising the anti-scarring agent. In yet another embodiment, the device delivers the anti-scarring agent locally to tissue proximate to the device. In another embodiment, the anti-scarring agent is released into tissue in the vicinity of the device after deployment of the device. In another embodiment, the anti-scarring agent is released into tissue in the vicinity of the device after deployment of the device, wherein the tissue is connective tissue, muscle tissue, nerve tissue, or epithelium tissue.

In certain embodiments, the devices described herein that comprise a soft tissue implant (breast, facial, chin, mandibular, lip, nasal, cheek, pectoral, buttocks, autogenous tissue) and either an anti-scarring agent or a composition comprising an anti-scarring agent, and the methods that use these devices (for inhibiting scarring between a soft tissue implant and the host; for reconstructing or augmenting), and/or methods for making these devices have one or more of the following features. In particular embodiments, the anti-scarring agent is released in effective concentrations from the device over a period ranging from the time of deployment of the device to about 1 year; the anti-scarring agent is released in effective concentrations from the device over a period ranging from about 1 month to 6 months; or the anti-scarring agent is released in effective concentrations from the device over a period ranging from about 1-90 days. In other embodiments, the anti-scarring agent is released in effective concentrations from the device at a constant rate; the anti-scarring agent is released in effective concentrations from the device at an increasing rate; the anti-scarring agent is released in effective concentrations from the device at a decreasing rate; the anti-scarring agent is released in effective concentrations from the composition comprising the anti-scarring agent by diffusion over a period ranging from the time of deployment of the device to about 90 days; and/or the anti-scarring agent is released in effective concentrations from the composition comprising the anti-scarring agent by erosion of the composition over a period ranging from the time of deployment of the device to about 90 days. In certain embodiments, the device comprises about 0.01 μg to about 10 μg of the anti-scarring agent, about 10 μg to about 10 mg of the anti-scarring agent, about 10 mg to about 250 mg of the anti-scarring agent, about 250 mg to about 1000 mg of the anti-scarring agent, or about 1000 mg to about 2500 mg of the anti-scarring agent. In other particular embodiments, a surface of the device comprises less than 0.01 μg of the anti-scarring agent per mm² of device surface to which the anti-scarring agent is applied; comprises about 0.01 μg to about 1 μg of the anti-scarring agent per mm² of device surface to which the anti-scarring agent is applied; comprises about 1 μg to about 10 μg of the anti-scarring agent per mm² of device surface to which the anti-scarring agent is applied; comprises about 10 μg to about 250 μg of the anti-scarring agent per mm² of device surface to which the anti-scarring agent is applied; comprises about 250 μg to about 1000 μg of the anti-scarring agent of anti-scarring agent per mm² of device surface to which the anti-scarring agent is applied; or comprises about 1000 μg to about 2500 μg of the anti-scarring agent per mm² of device surface to which the anti-scarring agent is applied. In particular embodiments, the anti-scarring agent or the composition comprising the anti-scarring agent is affixed to the implant; covalently attached to the implant; or non-covalently attached to the implant. In a particular embodiment, the device further comprises a coating that absorbs the agent or the composition. In another particular embodiment, the implant is interweaved with a thread composed of, or coated with, the agent or the composition. In certain embodiments, the implant is covered with a sleeve that contains the agent or the composition. In a particular embodiment, the implant is completely covered with a sleeve that contains the agent or the composition, and in another embodiment a portion of the implant is covered with a mesh that contains the agent or the composition. In still another embodiment, the implant is completely covered with a mesh that contains the agent or the composition.

In certain embodiments, the invention provides a method for inhibiting scarring between an implant (a soft tissue, a breast, a facial, a chin, a mandibular, a lip, a nasal, a cheek, a pectoral, a buttocks, or an autogenous tissue implant) and either an anti-scarring agent or a composition comprising an anti-scarring agent into the host, wherein the agent inhibits scarring and wherein the agent is released in effective concentrations from the composition comprising the agent by erosion of the composition over a period ranging from the time of administration to about 90 days. In another embodiment, the agent is released in effective concentrations from the composition comprising the agent by diffusion over a period ranging from the time of administration to about 90 days. In certain other embodiments, the agent or the composition is applied to the implant surface prior to placing of the implant into the host, or the agent or the composition is applied to the implant surface during placing of the implant into the host, or the agent or the composition is applied to the implant surface after placing of the implant into the host. In another embodiment, the agent or the composition is applied to the surface of the host tissue that will surround the implant prior to placing the implant into the host, during placement of the implant into the host, or after placing the implant into the host. In yet another embodiment, the agent or the composition is sprayed onto the implant surface prior to placing of the implant into the host, during placing of the implant into the host, or after placing of the implant into the host. In yet another embodiment, the agent or the composition is sprayed onto the surface of the host tissue that will surround the implant prior to placing the implant into the host, during placement of the implant into the host, or after placing the implant into the host. In yet another embodiment, the agent or the composition is applied to the implant surface and to the surface of the host tissue prior to placing of the implant into the host, during placing of the implant into the host, or after placing of the implant into the host. In a particular embodiment, the agent or the composition is sprayed onto the implant surface and onto the surface of the host tissue prior to placing of the implant into the host, during placing of the implant into the host, or after placing of the implant into the host. In a certain embodiment, the agent or the composition is topically applied into the anatomical region where the implant is placed into the host. In another certain embodiment, the agent or the composition is percutaneously injected into the tissue surrounding the implant in the host. In still other embodiments, the method for inhibiting scarring comprises inserting the implant into a sleeve, wherein the sleeve comprises the anti-scarring agent, and wherein in certain embodiments, the sleeve comprises a mesh.

In other specific embodiments, a method is provided for reconstructing a breast or for augmenting a breast that comprises placing into a host a device that comprises a breast implant and either an anti-scarring agent or a composition comprising an anti-scarring agent, wherein the agent inhibits scarring between the device and the host into which the device is implanted and wherein the agent is released in effective concentrations from the composition comprising the agent by erosion of the composition over a period ranging from the time of administration to about 90 days. In another embodiment, the agent is released in effective concentrations from the composition comprising the agent by diffusion over a period ranging from the time of administration to about 90 days. In certain other embodiments, the agent or the composition is applied to the implant surface prior to placing of the implant into the host, or the agent or the composition is applied to the implant surface during placing of the implant into the host, or the agent or the composition is applied to the implant surface after placing of the implant into the host. In another embodiment, the agent or the composition is applied to the surface of the host tissue that will surround the implant prior to placing the implant into the host, during placement of the implant into the host, or after placing the implant into the host. In yet another embodiment, the agent or the composition is sprayed onto the implant surface prior to placing of the implant into the host, during placing of the implant into the host, or after placing of the implant into the host. In yet another embodiment, the agent or the composition is sprayed onto the surface of the host tissue that will surround the implant prior to placing the implant into the host, during placement of the implant into the host, or after placing the implant into the host. In yet another embodiment, the agent or the composition is applied to the implant surface and to the surface of the host tissue prior to placing of the implant into the host, during placing of the implant into the host, or after placing of the implant into the host. In a particular embodiment, the agent or the composition is sprayed onto the implant surface and onto the surface of the host tissue prior to placing of the implant into the host, during placing of the implant into the host, or after placing of the implant into the host. In a certain embodiment, the agent or the composition is topically applied into the anatomical region where the implant is placed into the host. In another certain embodiment, the agent or the composition is percutaneously injected into the tissue surrounding the implant in the host. In still other embodiments, the method for reconstructing or augmenting a breast comprises inserting the implant into a sleeve, wherein the sleeve comprises the anti-scarring agent, and wherein in certain embodiments, the sleeve comprises a mesh.

In other specific embodiments, a method is provided for augmenting a malar or submalar region that comprises placing into a host a device that comprises a facial implant and either an anti-scarring agent or a composition comprising an anti-scarring agent, wherein the agent inhibits scarring between the device and the host into which the device is implanted and wherein the agent is released in effective concentrations from the composition comprising the agent by erosion of the composition over a period ranging from the time of administration to about 90 days. In another embodiment, the agent is released in effective concentrations from the composition comprising the agent by diffusion over a period ranging from the time of administration to about 90 days. In certain other embodiments, the agent or the composition is applied to the implant surface prior to placing of the implant into the host, or the agent or the composition is applied to the implant surface during placing of the implant into the host, or the agent or the composition is applied to the implant surface after placing of the implant into the host. In another embodiment, the agent or the composition is applied to the surface of the host tissue that will surround the implant prior to placing the implant into the host, during placement of the implant into the host, or after placing the implant into the host. In yet another embodiment, the agent or the composition is sprayed onto the implant surface prior to placing of the implant into the host, during placing of the implant into the host, or after placing of the implant into the host. In yet another embodiment, the agent or the composition is sprayed onto the surface of the host tissue that will surround the implant prior to placing the implant into the host, during placement of the implant into the host, or after placing the implant into the host. In yet another embodiment, the agent or the composition is applied to the implant surface and to the surface of the host tissue prior to placing of the implant into the host, during placing of the implant into the host, or after placing of the implant into the host. In a particular embodiment, the agent or the composition is sprayed onto the implant surface and onto the surface of the host tissue prior to placing of the implant into the host, during placing of the implant into the host, or after placing of the implant into the host. In a certain embodiment, the agent or the composition is topically applied into the anatomical region where the implant is placed into the host. In another certain embodiment, the agent or the composition is percutaneously injected into the tissue surrounding the implant in the host. In still other embodiments, the method for augmenting a malar or submalar comprises inserting the implant into a sleeve, wherein the sleeve comprises the anti-scarring agent, and wherein in certain embodiments, the sleeve comprises a mesh.

In other specific embodiments, a method is provided for reconstructing a jaw, a chin, a nose, a lip, or a chest that comprises placing into a host a device that comprises a mandibular implant, chin implant, nasal implant, lip implant, or pectoral implant, respectively, and either an anti-scarring agent or a composition comprising an anti-scarring agent, wherein the agent inhibits scarring between the device and the host into which the device is implanted and wherein the agent is released in effective concentrations from the composition comprising the agent by erosion of the composition over a period ranging from the time of administration to about 90 days. In another embodiment, the agent is released in effective concentrations from the composition comprising the agent by diffusion over a period ranging from the time of administration to about 90 days. In certain other embodiments, the agent or the composition is applied to the implant surface prior to placing of the implant into the host, or the agent or the composition is applied to the implant surface during placing of the implant into the host, or the agent or the composition is applied to the implant surface after placing of the implant into the host. In another embodiment, the agent or the composition is applied to the surface of the host tissue that will surround the implant prior to placing the implant into the host, during placement of the implant into the host, or after placing the implant into the host. In yet another embodiment, the agent or the composition is sprayed onto the implant surface prior to placing of the implant into the host, during placing of the implant into the host, or after placing of the implant into the host. In yet another embodiment, the agent or the composition is sprayed onto the surface of the host tissue that will surround the implant prior to placing the implant into the host, during placement of the implant into the host, or after placing the implant into the host. In yet another embodiment, the agent or the composition is applied to the implant surface and to the surface of the host tissue prior to placing of the implant into the host, during placing of the implant into the host, or after placing of the implant into the host. In a particular embodiment, the agent or the composition is sprayed onto the implant surface and onto the surface of the host tissue prior to placing of the implant into the host, during placing of the implant into the host, or after placing of the implant into the host. In a certain embodiment, the agent or the composition is topically applied into the anatomical region where the implant is placed into the host. In another certain embodiment, the agent or the composition is percutaneously injected into the tissue surrounding the implant in the host. In still other embodiments, the method for reconstructing a jaw, chin, nose, lip, or chest comprises inserting the implant into a sleeve, wherein the sleeve comprises the anti-scarring agent, and wherein in certain embodiments, the sleeve comprises a mesh.

In other specific embodiments, a method is provided for augmenting soft tissue that comprises placing into a host a device that comprises an autogenous tissue implant and either an anti-scarring agent or a composition comprising an anti-scarring agent, wherein the agent inhibits scarring between the device and the host into which the device is implanted and wherein the agent is released in effective concentrations from the composition comprising the agent by erosion of the composition over a period ranging from the time of administration to about 90 days. In another embodiment, the agent is released in effective concentrations from the composition comprising the agent by diffusion over a period ranging from the time of administration to about 90 days. In certain other embodiments, the agent or the composition is applied to the implant surface prior to placing of the implant into the host, or the agent or the composition is applied to the implant surface during placing of the implant into the host, or the agent or the composition is applied to the implant surface after placing of the implant into the host. In another embodiment, the agent or the composition is applied to the surface of the host tissue that will surround the implant prior to placing the implant into the host, during placement of the implant into the host, or after placing the implant into the host. In yet another embodiment, the agent or the composition is sprayed onto the implant surface prior to placing of the implant into the host, during placing of the implant into the host, or after placing of the implant into the host. In yet another embodiment, the agent or the composition is sprayed onto the surface of the host tissue that will surround the implant prior to placing the implant into the host, during placement of the implant into the host, or after placing the implant into the host. In yet another embodiment, the agent or the composition is applied to the implant surface and to the surface of the host tissue prior to placing of the implant into the host, during placing of the implant into the host, or after placing of the implant into the host. In a particular embodiment, the agent or the composition is sprayed onto the implant surface and onto the surface of the host tissue prior to placing of the implant into the host, during placing of the implant into the host, or after placing of the implant into the host. In a certain embodiment, the agent or the composition is topically applied into the anatomical region where the implant is placed into the host. In another certain embodiment, the agent or the composition is percutaneously injected into the tissue surrounding the implant in the host. In still other embodiments, the method for augmenting soft tissue comprises inserting the implant into a sleeve, wherein the sleeve comprises the anti-scarring agent, and wherein in certain embodiments, the sleeve comprises a mesh.

In other embodiments, a method is provided for making a device comprising combining a soft tissue implant (a breast, a facial, a chin, a mandibular, a lip, a nasal, a cheek, a pectoral, a buttocks, or an autogenous tissue implant) and either an anti-scarring agent or a composition comprising an anti-scarring agent, wherein the agent inhibits scarring between the device and a host into which the device is implanted, wherein combining is performed by any one or more of the following: directly affixing the agent or composition to the implant; by spraying the agent or composition onto the implant; electrospraying the agent or composition onto the implant; dipping the implant into a solution comprising the agent or composition; coating the implant with a substance that comprises the agent or the composition; coating the implant with a substance that comprises the agent or the composition, wherein coating is not performed by graft polymerization; coating the implant with a substance that absorbs the agent or composition; coating the implant with a substance that absorbs the agent or composition, wherein the substance comprises a hydrogel; incorporating the agent or composition into a polymer that comprises an outer coating of the implant; covalently attaching the agent or the composition to the implant; by covalently binding the agent or composition to a linker, wherein the linker is coated or attached to the implant surface; noncovalently attaching the agent or the composition to the implant; and/or by interweaving a thread composed of, or coated with, the agent or the composition.

In a particular embodiment, combining is performed during construction of the implant. In other embodiments, combining is performed by coating a portion of the implant with the agent or the composition or by coating the entire implant with the agent or composition. In another embodiment, combining is performed by incorporating the agent or composition into the central core of the implant; performed by incorporating the agent or composition into the central core of the implant, wherein the agent or composition is combined with a filler material; performed by incorporating the agent or composition into the central core of the implant, wherein the agent or composition is combined with a filler material that is saline; performed by incorporating the agent or composition into the central core of the implant, wherein the agent or composition is combined with a filler material that is silicone; and/or performed by incorporating the agent or composition into the central core of the implant, wherein the agent or composition is combined with a filler material that is polysiloxane, polyethylene glycol, vegetable, oil, monofilament yam, keratin hydrogen, or chondroitin sulfate. In particular embodiments, the agent or composition is incorporated into the central core by dissolving the agent or composition into an aqueous core material, wherein the agent or the composition is water soluble; the agent or composition is incorporated into the central core by combining the agent or the composition with a solubilizing agent or carrier, wherein the agent or the composition is water insoluble and wherein the core material is aqueous; the agent or composition is incorporated into the central core by dissolving the agent or composition in an organic core material, wherein the agent or the composition is water insoluble; the agent or composition is incorporated into the central core by incorporating the agent or the composition into threads contained in the implant central core; the agent or composition is incorporated into the central core by incorporating the agent or the composition into a central core gel material; the agent or composition is incorporated into the central core by formulating the agent or the composition into a formulation comprising a solution, microsphere, gel, paste, film, or solid particle, and incorporating the formulation into an implant filler material; the agent or composition is incorporated into the central core by forming a suspension of the agent or the composition with an implant filler material, wherein the agent or the composition is insoluble and the filler material is aqueous; and/or the agent or composition is incorporated into the central core by forming a suspension of the agent or the composition with an implant filler material, wherein the agent or the composition is aqueous and the filler material is organic.

In other embodiments, the step of combining is performed by any one of the following: completely covering the implant with a sleeve that contains the agent or the composition; covering a portion of the implant with a sleeve that contains the agent or the composition; completely covering the implant with a cover that contains the agent or the composition; covering a portion of the implant with a cover that contains the agent or the composition; completely covering the implant with an electrospun fabric that contains the agent or the composition; covering a portion of the implant with an electrospun fabric that contains the agent or the composition; completely covering the implant with a mesh that contains the agent or the composition; covering a portion of the implant with a mesh that contains the agent or the composition; constructing a portion of the implant with the agent or the composition; impregnating the implant with the agent or the composition; by constructing a portion of the implant from a degradable polymer that releases the agent; dipping the implant into a solution that comprises either the agent or the composition and an inert solvent; dipping the implant into a solution that comprises either the agent or the composition and a solvent that will swell the implant; dipping the implant into a solution that comprises either the agent or the composition and a solvent that will dissolve the implant; spraying the implant with a solution that comprises either the agent or the composition and an inert solvent; spraying the implant with a solution that comprises either the agent or the composition and a solvent that will swell the implant; spraying the implant with a solution that comprises either the agent or the composition and a solvent that will dissolve the implant; spraying the implant with a solution that comprises the agent, a polymer, and an inert solvent; spraying the implant with a solution that comprises the agent, a polymer, and a solvent that will swell the implant; spraying the implant with a solution that comprises the agent, a polymer, and a solvent that will dissolve the implant. In another embodiment, such a method for making a device further comprises one or more of the following: incorporating a fibrosis-promoting agent wherein the fibrosis-promoting agent is applied to one portion of the implant and the anti-scarring agent or the composition comprising the anti-scarring agent is applied to a second portion of the implant; incorporating a fibrosis-promoting agent wherein the fibrosis-promoting agent is sprayed onto one portion of the implant and the anti-scarring agent or the composition comprising the anti-scarring agent is sprayed onto a second portion of the implant; constructing the implant to comprise a reservoir for containing at least one drug; constructing the implant to comprise a reservoir for containing at least one drug and a carrier; constructing the implant to comprise a reservoir for containing the anti-scarring agent or the composition comprising the anti-scarring agent; constructing the implant to comprise a reservoir for containing the anti-scarring agent or the composition comprising the anti-scarring agent and a carrier; constructing the implant to comprise a reservoir for containing a drug combined with a carrier, wherein the agent is released from the carrier; constructing the implant to comprise a reservoir for containing a drug, wherein the reservoir comprises a plurality of layers; constructing the implant to comprise a reservoir for containing at least one drug, wherein the reservoir comprises a plurality of layers wherein each layer permits release of a drug; constructing the implant to comprise a reservoir for containing at least one drug, wherein the reservoir comprises a plurality of layers, wherein each layer contains and permits release of a different drug; and constructing the implant to comprise a reservoir for containing at least one drug, wherein the reservoir comprises a plurality of layers wherein at least one layer is a barrier layer that prevents the release of a drug.

The following examples are offered by way of illustration, and not by way of limitation.

### EXAMPLES

### Example 1

### Drug-Loading a Porous Facial Implant—Paclitaxel Dipping

100 ml solutions of paclitaxel are prepared by weighing in 10 mg, 50 mg, 100 mg, 200 mg, 500 mg, 750 mg, 1000 mg, 2000 mg, and 5000 mg paclitaxel into a 250 ml glass jar with a TEFLON lined lid respectively and then adding 100 ml HPLC grade methanol. The solutions are gently shaken on an orbital shaker for 1 hour at room temperature. A porous high density poly(ethylene) facial implant (Design M Malar Implant, Cat # 9509, Porex Corporation) is placed into each of the paclitaxel solutions. After about 2 hours, the implant is removed from the solution, gently shaken and is allowed to air dry for 6 hours. The implant is further dried under vacuum for 24 hours. In additional examples, one of the following exemplary compounds may be used in lieu of paclitaxel: rapamycin, everolimus, pimecrolimus, mithramycin, and halifuginone.

### Example 2

### Drug-Loading a Porous Facial Implant—Paclitaxel/Water-Soluble Polymer: Dipping

Nine samples of a MePEG(2000)-PDLLA (60:40) diblock copolymer solution are prepared by dissolving 1 g MePEG(2000)-PDLLA (60:40) diblock copolymer in 0.100 ml HPLC grade acetonitrile in 250 ml glass jars that have TEFLON lined lids. The solutions are rolled on a roller mill until all the polymer is dissolved. 10 mg, 50 mg, 100 mg, 200 mg, 500 mg, 750 mg, 1000 mg, 2000 mg, and 5000 mg paclitaxel are weighed into each polymer solution respectively. A magnetic stir bar is added to each solution and the solutions are stirred for 1 hour at room temperature. A porous high density poly(ethylene) facial implant (Design M Malar Implant, Cat # 95,09, Porex Corporation) is placed into each of the paclitaxel solutions. After about 2 hours, the implant is removed from the solution, gently shaken and allowed to air dry for 6 hour. The implant is further dried under vacuum for 24 hours. In additional examples, one of the following exemplary compounds may be used in lieu of paclitaxel: rapamycin, everolimus, pimecrolimus, mithramycin and halifuginone in place of paclitaxel.

### Example 3

### Drug-Loading a Porous Facial Implant—Paclitaxel/Degradable Polymer: Dipping

Nine samples of a pply(D,L-lactide-co-glycolide) (PLG) polymer (50:50, IV=0.25, Birmingham Polymers, Inc) solution are prepared by dissolving 10 g PLG copolymer in 100 ml ethyl acetate in 250 ml glass jars that have TEFLON lined lids. The solutions are rolled on a roller mill until all the polymer is dissolved. 10 mg, 50 mg, 100 mg, 200 mg, 500 mg, 750 mg, 1000 mg, 2000 mg, and 5000 mg paclitaxel are weighed into each polymer solution, respectively. A magnetic stir bar is added to each solution and the solutions are stirred for 1 hour at room temperature. A porous high density poly(ethylene) facial implant (Design M Malar Implant, Cat # 9509, Porex Corporation) is placed into each of the paclitaxel solutions. After about 2 hours, the implant is removed from the solution, gently shaken and is allowed to air dry for 6 hour. The implant is further dried under vacuum for 24 hours. In additional examples, one of the following exemplary compounds may be used in lieu of paclitaxel: rapamycin, everolimus, pimecrolimus, mithramycin and halifuginone.

### Example 4

### Drug-Loading a Porous Facial Implant—Paclitaxel Spraying

Ten ml solutions of paclitaxel are prepared by weighing 1 mg, 5 mg, 10 mg, 20 mg, 50 mg, 75 mg, 100 mg, 200 mg, and 500 mg paclitaxel into a 20 ml glass scintillation vial respectively and then adding 100 ml HPLC grade methanol. The solutions are gently shaken on an orbital shaker for 1 hour at room temperature. A pin is pushed into a porous high density poly(ethylene) facial implant (Design M Malar Implant, Cat # 9509, Porex Corporation). Using a piece of stainless steel wire attached to the protruding pin, the implant is suspended in the air by attaching the wire to a clamp on a retort stand. The 0.1 mg/ml paclitaxel solution is placed in a TLC spray device (Aldrich), which is then coupled to a nitrogen gas line. The implant is then sprayed with the paclitaxel solution such that the surface of the implant is wetted by the solution. The implant is allowed to air dry for 1 hour. The pin is removed and the implant is further dried under vacuum for 24 hours. In additional examples, one of the following exemplary compounds may be used in lieu of paclitaxel: rapamycin, everolimus, pimecrolimus, mithramycin, and halifuginone.

### Example 5

### Drug-Loading a Porous Facial Implant—Paclitaxel/Water-Soluble Polymer: Spraying

Nine samples of a MePEG(2000)-PDLLA (60:40 w/w) diblock copolymer solution are prepared by dissolving 10 g MePEG(2000)-PDLLA. (60:40) diblock copolymer in 100 ml HPLC grade acetonitrile in 250 ml glass jars that have TEFLON lined lids. The solutions are rolled on a roller mill until all the polymer is dissolved. 10 mg, 50 mg, 100 mg, 200 mg, 500 mg, 750 mg, 1000 mg, 2000 mg, and 5000 mg paclitaxel are weighed into each polymer solution respectively. A magnetic stir bar is added to each solution and the solutions are stirred for 1 hour at room temperature. A pin is pushed into a porous high density poly(ethylene) facial implant (Design M Malar Implant, Cat # 9509, Porex Corporation). Using a piece of stainless steel wire attached to the protruding pin, the implant is suspended in the air by attaching the wire to a clamp on a retort stand. The 0.1 mg/ml paclitaxel solution is placed in a TLC spray device (Aldrich), which is then coupled to a nitrogen gas line. The implant is then sprayed with the paclitaxel solution such that the surface of the implant is wetted by the solution. The implant is allowed to air dry for 1 hour. The pin is removed and the implant is further dried under vacuum for 24 hours. In additional examples, one of the following exemplary compounds may be used in lieu of paditaxel: rapamycin, everolimus, pimecrolimus, mithramycin, and halifuginone.

### Example 6

### Drug-Loading a Porous Facial Implant—Paclitaxel/Degradable Polymer: Spraying

Nine samples of a poly(D,L-lactide-co-glycolide) (PLG) polymer (50:50, IV=0.25, Birmingham Polymers, Inc) solution are prepared by dissolving 10 g PLG copolymer in 100 ml ethyl acetate in 250 ml glass jars that have TEFLON lined lids. The solutions are rolled on a roller mill until all the polymer is dissolved. Ten mg, 50 mg, 100 mg, 200 mg, 500 mg, 750 mg, 1000 mg, 2000 mg, and 5000 mg paclitaxel are weighed into each polymer solution respectively. A magnetic stir bar is added to each solution and the solutions are stirred for 1 hour at room temperature. A pin is pushed into a porous high density poly(ethylene) facial implant (Design M Malar Implant, Cat # 9509, Porex Corporation). Using a piece of stainless steel wire attached to the protruding pin, the implant is suspended in the air by attaching the wire to a clamp on a retort stand. The 0.1 mg/ml paclitaxel solution is placed in a TLC spray device (Aldrich), which is then coupled to a nitrogen gas line. The implant is then sprayed with the paclitaxel solution such that the surface of the implant is wetted by the solution. The implant is allowed to air dry for 1 hour. The pin is removed and the implant is further dried under vacuum for 24 hours. In additional examples, one of the following exemplary compounds may be used in lieu of paclitaxel: rapamycin, everolimus, pimecrolimus, mithramycin, and halifuginone.

### Example 7

### Drug-Loading a Porous Facial Implant—Paclitaxel/Anti-Infective/Degradable Polymer: Dipping

Nine samples of a poly(D,L-lactide-co-glycolide) (PLG) polymer (50:50, IV=0.25, Birmingham Polymers, Inc) solution are prepared by dissolving 10 g PLG copolymer in 100 ml ethyl acetate in 250 ml glass jars that have TEFLON lined lids. The solutions are rolled on a roller mill until all the polymer is dissolved. One hundred mg 5-fluorouracil is added to each sample. Ten mg, 50 mg, 100 mg, 200 mg, 500 mg, 750 mg, 1000 mg, 2000 mg, and 5000 mg paclitaxel are weighed into each polymer solution, respectively. A magnetic stir bar is added to each solution and the solutions are stirred for 1 hour at room temperature. A porous high density poly(ethylene) facial implant (Design M Malar Implant, Cat # 9509, Porex Corporation) is placed into each of the paclitaxel solutions. After about 2 hours, the implant is removed from the solution, gently shaken and is allowed to air dry for 6 hour. The implant is further dried under vacuum for 24 hours. In additional examples, one of the following exemplary compounds may be used in lieu of paclitaxel: rapamycin, everolimus, pimecrolimus, mithramycin and halifuginone.

### Example 8

### Drug-Loading a Porous Facial Implant—Paclitaxel/Degradable Polymer: Dipping

Nine samples of a MePEG(750)-PDLLA (20:80 w/w) diblock copolymer solution are prepared by dissolving 10 g MePEG(750)-PDLLA copolymer in 100 ml acetone in 250 ml glass jars that have TEFLON lined lids. The solutions are rolled on a roller mill for until all the polymer is dissolved. Ten mg, 50 mg, 100 mg, 200 mg, 500 mg, 750 mg, 1000 mg, 2000 mg, and 5000 mg paclitaxel are weighed into each polymer solution, respectively. A magnetic stir bar is added to each solution and the solutions are stirred for 1 hour at room temperature. A porous ePTFE facial implant (Nasal Dorsum, Cat # 1 NS001, W. L. Gore) is placed into each of the paclitaxel solutions. The solutions are then sonicated in an ultrasonic bath for about 2 minutes. The implants are removed from the solution, gently shaken and allowed to air dry for 6 hours. The implants are further dried under vacuum for 24 hours. In additional examples, one of the following exemplary compounds may be used in lieu of paclitaxel: rapamycin, everolimus, pimecrolimus, mithramycin, and halifuginone.

### Example 9

### Drug-Loading a Porous Facial Implant—Paclitaxel/Degradable Polymer: Dipping

Nine samples of a MePEG(2000)-PDLLA (60:40) diblock copolymer solution are prepared by dissolving 10 g MePEG(2000)-PDLLA (60:40) diblock copolymer in 100 ml anhydrous methanol in 250 ml glass jars that have TEFLON lined lids. The solutions are rolled on a roller mill until all the polymer is dissolved. Five grams Tetra functional poly(ethylene glycol) succinimidyl glutarate (4-arm-NHS-PEG, Cat # P4SG-10, Sunbio Inc., Anyang City, Korea) is weighed into each solution. Ten mg, 50 mg, 100 mg, 200 mg, 500 mg, 750 mg, 1000 mg, 2000 mg, and 5000 mg paclitaxel are then weighed into each polymer solution, respectively. A magnetic stir bar is added to each solution and the solutions are stirred for 1 hour at room temperature. A porous ePTFE facial implant (Nasal Dorsum, Cat # 1 NS001, W. L. Gore) is placed into each of the paclitaxel solutions. The solutions are then sonicated in an ultrasonic bath for about 2 minutes. The implants are removed from the solution, gently shaken and allowed to dry for 10 minutes by passing a stream of dry nitrogen over the surface of the implant. The implants are further dried under vacuum for 24 hours. In additional examples, one of the following exemplary compounds may be used in lieu of paclitaxel: rapamycin, everolimus, pimecrolimus, mithramycin, and halifuginone.

### Example 10

### Drug-Loading a Porous Facial Implant—Paclitaxel/Peg Polymer: Dipping

Nine samples of a tetra functional poly(ethylene glycol) succinimidyl glutarate (4-arm-NHS-PEG, Cat # P4SG-10, Sunbio Inc., Anyang City, Korea) solution are prepared by dissolving 10 g 4-arm-NHS-PEG in 100 ml anhydrous methanol in 250 ml glass jars that has TEFLON lined lids. The solutions are rolled on a roller mill until all the polymer has dissolved. Ten mg, 50 mg, 100 mg, 200 mg, 500 mg, 750 mg, 1000 mg, 2000 mg, and 5000 mg paclitaxel are then weighed into each polymer solution, respectively. A magnetic stir bar is added to each solution and the solutions are stirred for 30 minutes at room temperature. A porous ePTFE facial implant (Nasal Dorsum, Cat # 1 NS001, W. L. Gore) is placed into each of the paclitaxel solutions. The solutions are then sonicated in an ultrasonic bath for about 2 minutes. The implants are removed from the solution, gently shaken and allowed to dry for 10 minutes by passing a stream of dry nitrogen over the surface of the implant. The implants are further dried under vacuum for 24 hours. In additional examples, one of the following exemplary compounds may be used in lieu of paclitaxel: rapamycin, everolimus, pimecrolimus, mithramycin, and halifuginone.

### Example 11

### Drug-Loading a Pectoral Implant —Paclitaxel Dipping

100 ml solutions of paclitaxel are prepared by weighing 10 mg, 50 mg, 100 mg, 200 mg, 500 mg, 750 mg, 1000 mg, 2000 mg, and 5000 mg paclitaxel into a 250 ml glass jar with a TEFLON lined lid, respectively, and then adding 100 ml HPLC grade methanol. The solutions are gently shaken on an orbital shaker for 1 hour at room temperature. A silicone pectoral implant (Pectoralis Implant, Cat # ACPI-1, Allied Biomedical) is placed into each of the paclitaxel solutions. After about 2 hours, the implants are removed from the solution, gently shaken and allowed to air dry for 6 hours. The implants are further dried under vacuum for 24 hours. In additional examples, one of the following exemplary compounds may be used in lieu of paclitaxel: rapamycin, everolimus, pimecrolimus, mithramycin, and halifuginone.

### Example 12

### Drug-Loading a Pectoral Implant —Paclitaxel/Non-Degradable Dipping

500 g Dimethylacetamide (DMAC) are added to a 2 L glass beaker. 330 g of a polyurethane solution (CHRONOFLEX AR, 25% solids in DMAC, CT Biomaterials, Inc) is added to the solution. The solution is stirred for 15 min using an overhead stirrer unit (Cole Parmer) with a TEFLON-coated paddle type stirrer blade. 31 g poly(vinylpyrrolidone) (PLASDONE K-90D) is added to the solution. The solution is covered with aluminum foil and is stirred for 6 hours until the polymers are all dissolved. 100 g of the polymer solution is transferred to a 250 ml glass jar with a TEFLON lined lid. This is repeated 4 times. To each of the polymer solutions, paclitaxel is added such that paclitaxel to polymer ratios (w/w) of 0.1%, 0.5%, 1%, 10%, and 20% are obtained, respectively. A magnetic stir bar is added to each solution and the solutions are stirred for 30 min at room temperature. Using a pair of large tweezers, a silicone pectoral implant (Pectoralis Implant, Cat # ACPI-1, Allied Biomedical) is dipped into the 0.1% paclitaxel solution. The implant is withdrawn and is dried using a gentle stream of nitrogen. The implant is then allowed to air dry for 6 hours. The dip coating process is repeated holding the implant with the tweezers at a different location compared to the first coat. This coating process is repeated for each of the paclitaxel containing solutions. In additional examples, one of the following exemplary compounds may be used in lieu of paclitaxel: rapamycin, everolimus, pimecrolimus, mithramycin, and halifuginone.

### Example 13

### Drug-Loading a Breast Implant—Paclitaxel/Non-Degradable Dipping

500 g dimethylacetamide (DMAC) is added to a 2 L glass beaker. 330 g of a polyurethane solution (CHRONOFLEX AR, 25% solids in DMAC, CardioTech Biomaterials, Inc) is added to the solution. The solution is stirred for 15 min using an overhead stirrer unit (Cole Parmer) with a TEFLON-coated paddle type stirrer blade. 31 g poly(vinylpyrrolidone) (PLASDONE K-90D) is added to the solution. The solution is covered with aluminum foil and is stirred for 6 hours until the polymers are all dissolved. 100 g of the polymer solution are transferred to a 500 ml glass jar with a TEFLON lined lid. This is repeated 4 times. To each of the polymer solutions, paclitaxel is added such that paclitaxel to polymer ratios (w/w) of 0.1%, 0.5%, 1%, 10% and 20% are obtained, respectively. A magnetic stir bar is added to each solution and the solutions are stirred for 30 min at room temperature. Using a pair of large tweezers, a silicone smooth-surfaced breast implant (Cat # 350-1610, Mentor Corporation) is dipped into the 0.1% paclitaxel solution. The implant is withdrawn and is dried using a gentle stream of nitrogen. The implant is then allowed to air dry for 6 hours. The dip coating process is repeated holding the implant with the tweezers at a different location compared to the first coat. This coating process is repeated for each of the paclitaxel containing solutions. In additional examples, one of the following exemplary compounds may be used in lieu of paclitaxel: rapamycin, everolimus, pimecrolimus, mithramycin, and halifuginone.

### Example 14

### Drug-Loading a Smooth Surfaced Breast Implant—Paclitaxel Spraying

Ten ml solutions of paclitaxel are prepared by weighing 1 mg, 5 mg, 10 mg, 20 mg, 50 mg, 75 mg, 100 mg, 200 mg, and 500 mg paclitaxel into a 20 ml glass scintillation vial respectively and then adding to 100 ml HPLC grade methanol. The solutions are gently shaken on an orbital shaker for 1 hour at room temperature. A smooth surfaced breast implant (Cat # 350-1610, Mentor Corporation) is placed on a flat sheet of glass. The 0.1 mg/ml paclitaxel solution is placed in a TLC spray device (Aldrich), which is then coupled to a nitrogen gas line. The exposed implant is then sprayed with the paclitaxel solution such that the surface of the implant is wetted by the solution. The implant is allowed to air dry for 1 hour. The implant is turned over and the process is repeated. The implant is allowed to air dry for 4 hours. In additional examples, one of the following exemplary compounds may be used in lieu of paclitaxel: rapamycin, everolimus, pimecrolimus, mithramycin, and halifuginone.

### Example 15

### Drug-Loading a Smooth Surfaced Breast Implant—Paclitaxel/Anti-Infective Spraying

Ten ml solutions of paclitaxel are prepared by weighing 1 mg, 5 mg, 10 mg, 20 mg, 50 mg, 75 mg, 100 mg, 200 mg, and 500 mg paclitaxel into a 20 ml glass scintillation vial respectively and then adding to 100 ml HPLC grade methanol. 50 ml minocycline is added to each sample vial. The solutions are gently shaken on an orbital shaker for 1 hour at room temperature. A smooth-surfaced breast implant (Cat # 350-1610, Mentor Corporation) is placed on a flat sheet of glass. The 0.1 mg/ml paclitaxel solution is placed in a TLC spray device (Aldrich), which is then coupled to a nitrogen gas line. The exposed implant is then sprayed with the paclitaxel solution such that the surface of the implant is wetted by the solution. The implant is allowed to air dry for 1 hour. The implant is turned over and the process is repeated. The implant is allowed to air dry for 4 hours. In additional examples, one of the following exemplary compounds may be used in lieu of paclitaxel: rapamycin, everolimus, pimecrolimus, mithramycin, and halifuginone.

### Example 16

### Drug-Loading a Surface Textured Breast Implant—Paclitaxel Spraying

Ten ml solutions of paclitaxel are prepared by weighing 1 mg, 0.5 mg, 10 mg, 20 mg, 50 mg, 75 mg, 100 mg, 200 mg, and 500 mg paclitaxel into a 20 ml glass scintillation vial respectively and then adding to 100 ml anhydrous methanol. The solutions are gently shaken on an orbital shaker for 1 hour at room temperature. One gram tetrafunctional poly(ethylene glycol) succinimidyl glutarate (4-arm-NHS-PEG, Cat # P4SG-10, Sunbio Inc., Anyang City, Korea) is added to each solution. A surface textured breast implant (Cat # 354-2610, Mentor Corporation) is placed on a flat sheet of glass. The 0.1 mg/ml paclitaxel solution is placed in a TLC spray device (Aldrich), which is then coupled to a nitrogen gas line. The exposed implant is then sprayed with the paclitaxel solution such that the surface of the implant is wetted by the solution. The implant is allowed to dry for 20 min by passing a stream of dry nitrogen over the surface of the implant. The implant is turned over and the process is repeated. The implant is allowed to dry for 4 hours in a dry atmosphere. In additional examples, one of the following exemplary compounds may be used in lieu of paclitaxel: rapamycin, everolimus, pimecrolimus, mithramycin, and halifuginone.

### Example 17

### Drug-Loading Silicone Oil Used to Manufacture a Breast Implant

200 g silicone gel is added to a 500 ml round bottom flask. 200 mg paclitaxel in 50 ml methanol is added to the silicone gel. The round bottom flask is then attached to a rotavap (Buchi) and is rotated for 2 hours at a speed setting of 3. A partial vacuum is then applied for 3 hours while stirring at a speed setting of 3. The resultant material is used as the filling in a silicone breast implant. The process is repeated using 400 mg, 1 g, 2 g, and 5 g paclitaxel, respectively. In additional examples, one of the following exemplary compounds may be used in lieu of paclitaxel: rapamycin, everolimus, pimecrolimus, mithramycin, and halifuginone.

### Example 18

### DRUG-Loading the Saline Used to Manufacture a Breast Implant

Samples of a MePEG(2000)-PDLLA (60:40) diblock copolymer/paclitaxel matrix are prepared by dissolving 10 g MePEG(2000)-PDLLA (60:40) diblock copolymer in 100 ml acetonitrile in 250 ml glass jars that have TEFLON lined lids. The solutions are rolled on a roller mill until all the polymer is dissolved. 0.5 g paclitaxel is added to the solution. The solvent is removed by placing the sample in a water bath (30° C.) and blowing a stream of dry nitrogen over the solution surface. The samples are then dried under vacuum for 24 hours at 30° C. 100 ml sterile saline in then added to the paclitaxel/polymer matrix and the material is dissolved by gentle swirling on an orbital shaker. Once the polymer matrix is dissolved, the material is ready for filling a breast implant to produce a drug-loaded saline-filled breast implant, or it can be used to modify the fill volume of an expandable breast implant (for example, Spectrum Expandables, Cat #350-1410, Mentor Corporation)).

### Example 19

### Screening Assay for Assessing the Effect of Various Compounds on Nitric Oxide Production by Macrophages

The murine macrophage cell line RAW 264.7 was trypsinized to remove cells from flasks and plated in individual wells of a 6-well plate. Approximately 2×10⁶ cells were plated in 2 ml of media containing 5% heat-inactivated fetal bovine serum (FBS). RAW 264.7 cells were incubated at 37° C. for 1.5 hours to allow adherence to plastic. Mitoxantrone was prepared in DMSO at a concentration of 10⁻² M and serially diluted 10-fold to give a range of stock concentrations (10⁻⁸ M to 10⁻² M). Media was then removed and cells were incubated in 1 ng/ml of recombinant murine IFNγ and 5 ng/ml of LPS with or without mitoxantrone in fresh media containing 5% FBS. Mitoxantrone was added to cells by directly adding mitoxantrone DMSO stock solutions, prepared earlier, at a dilution, to each well. Plates containing IFNγ, LPS plus or minus mitoxantrone were incubated at 37° C. for 24 hours (Chem. Ber. (1979) 12: 426; J. AOAC (1977) 60: 594; Annu. Rev. Biochem. (1994) 63:175).

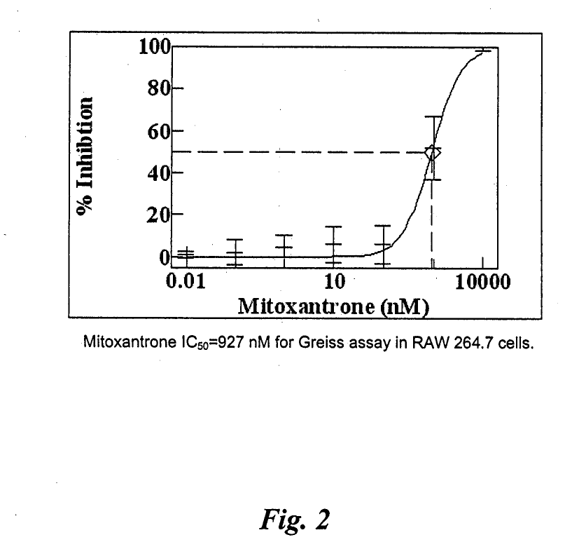

At the end of the 24 hour period, supernatants were collected from the cells and assayed for the production of nitrites. Each sample was tested in triplicate by aliquoting 50 μl of supernatant in a 96-well plate and adding 50 μl of Greiss Reagent A (0.5 g sulfanilamide, 1.5 ml H₃PO₄, 48.5 ml ddH₂O) and 50 μl of Greiss Reagent B (0.05 g N-(1-naphthyl)-ethylenediamine, 1.5 ml H₃PO₄, 48.5 ml ddH₂O). Optical density was read immediately on microplate spectrophotometer at 562 nm absorbance. Absorbance over triplicate wells was averaged after subtracting background and concentration values were obtained from the nitrite standard curve (1 μM to 2 mM). Inhibitory concentration of 50% (IC₅₀) was determined by comparing average nitrite concentration to the positive control (cell stimulated with IFNγ and LPS). An average of n=4 replicate experiments was used to determine IC₅₀ values for mitoxantrone (see FIG. 2 (IC₅₀=927 nM)). The IC₅₀ values for the following additional compounds were determined using this assay: IC₅₀ (nM): paclitaxel, 7; CNI-1493, 249; halofuginone, 12; geldanamycin, 51; anisomycin, 68; 17-AAG, 840; epirubicin hydrochloride, 769.

### Example 20

### Screening Assay for Assessing the Effect of Various Anti-Scarring Agents on TNF-Alpha Production by Macrophages

The human macrophage cell line, THP-1 was plated in a 12 well plate such that each well contained 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250×g, resuspended in 4 ml of human serum for a final concentration of 5 mg/ml, and incubated in a 37° C. water bath for 20 minutes to enable opsonization. Bay 11-7082 was prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 48044811; J. Immunol Meth. (2000) 235 (1-2): 33-40).

THP-1 cells were stimulated to produce TNFα by the addition of 1 mg/ml opsonized zymosan. Bay 11-7082 was added to THP-1 cells by directly adding DMSO stock solutions, prepared earlier, at a dilution, to each well. Each drug concentration was tested in triplicate wells. Plates were incubated at 37° C. for 24 hours.

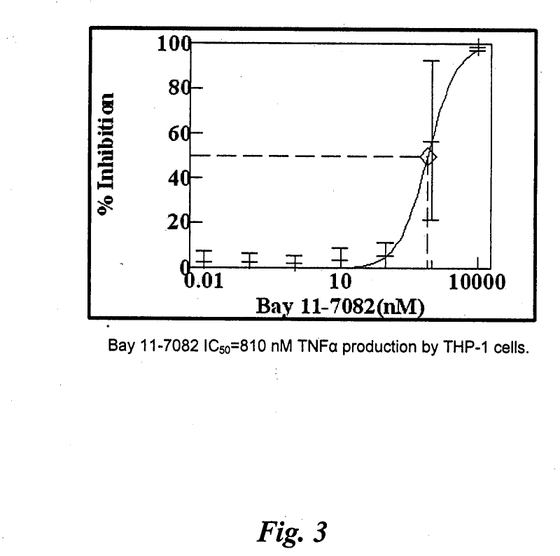

After a 24 hour stimulation, supernatants were collected to quantify TNFα production. TNFα concentrations in the supernatants were determined by ELISA using recombinant human TNFα to obtain a standard curve. A 96-well MaxiSorb plate was coated with 100 μl of anti-human TNFα Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/8 and 1/16; (b) recombinant human TNFα was prepared at 500 pg/ml and serially diluted to yield as standard curve of 7.8 μg/ml to 500 μg/ml. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human TNFα detection antibody+avidin-HRP) for 1 hour at room temperature. Following this incubation, the plates were washed 7 times and 100 μl of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. TNFα concentration values were obtained from the standard curve. Inhibitory concentration of 50% (IC₅₀) was determined by comparing average TNFα concentration to the positive control (THP-1 cells stimulated with opsonized zymosan). An average of n=4 replicate experiments was used to determine IC₅₀ values for Bay 11-7082 (FIG. 3; IC₅₀=810 nM)) and rapamycin (IC₅₀=51 nM; FIG. 4). The IC₅₀ values for the following additional compounds were determined using this assay: IC₅₀ (nM): geldanamycin, 14; mycophenolic acid, 756; mofetil, 792; chlorpromazine, 6; CNI-1493, 0.15; SKF 86002, 831; 15-deoxy prostaglandin J2, 742; fascaplycin, 701; podophyllotoxin, 75; mithramycin, 570; daunorubicin, 195; celastrol, 87; chromomycin A3, 394; vinorelbine, 605; vinblastine, 65.

### Example 21

### Surgical Adhesions Model to Assess Fibrosis Inhibiting Agents in Rats

The rat caecal sidewall model is used to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind tom fibres from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively.

### Example 22

### Surgical Adhesions Model to Assess Fibrosis Inhibiting Agents in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 23

### Screening Assay for Assessing the Effect of Various Compounds on Cell Proliferation

Fibroblasts at 70-90% confluency were trypsinized, replated at 600 cells/well in media in 96-well plates, and allowed to attach overnight. Mitoxantrone was prepared in DMSO at a concentration of 10⁻² M and diluted 10-fold to give a range of stock concentrations (10⁻⁸ M to 10⁻² M). Drug dilutions were diluted in media and added to cells to give a total volume of 200 μl/well. Each drug concentration was tested in triplicate wells. Plates containing fibroblasts and mitoxantrone were incubated at 37° C. for 72 hours (In Vitro Toxicol. (1990) 3: 219; Biotech. Histochem. (1993) 68: 29; Anal. Biochem. (1993) 213: 426).

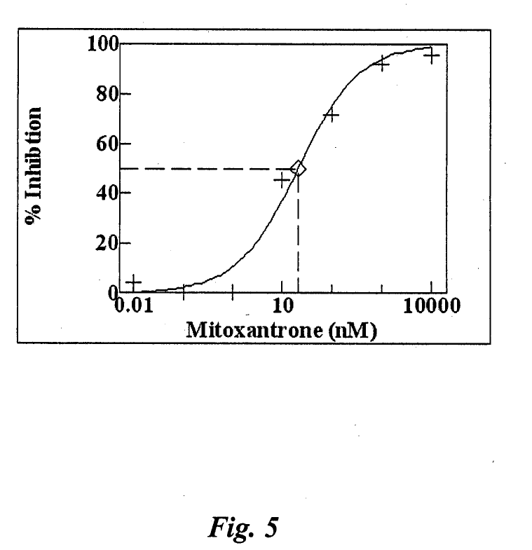

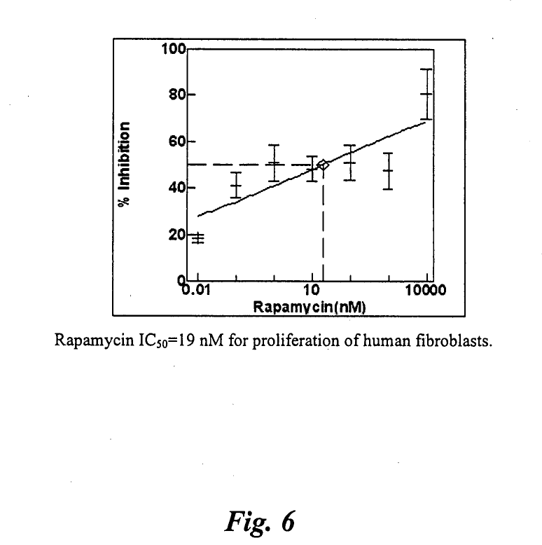

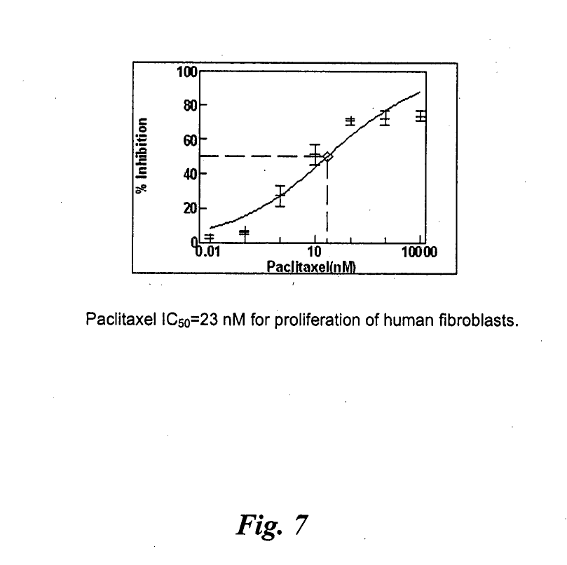

To terminate the assay, the media was removed by gentle aspiration. A 1/400 dilution of CYQUANT 400×GR dye indicator (Molecular Probes; Eugene, Oreg.) was added to 1× Cell Lysis buffer, and 200 μl of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=4 replicate experiments was used to determine IC₅₀ values. The IC₅₀ values for the following compounds were determined using this assay: IC₅₀ (nM): mitoxantrone, 20 (FIG. 5); rapamycin, 19 (FIG. 6); paclitaxel, 23 (FIG. 7); mycophenolic acid, 550; mofetil, 601; GW8510, 98; simvastatin, 885; doxorubicin, 84; geldanamycin, 11; anisomycin, 435; 17-MG, 106; bleomycin, 86; halofuginone, 36; gemfibrozil, 164; ciprofibrate, 503; bezafibrate, 184; epirubicin hydrochloride, 57; topotemay, 81; fascaplysin, 854; tamoxifen, 13; etanidazole, 55; gemcitabine, 7; purormiycin, 254; mithramycin, 156; daunorubicin, 51; L(−)-perillyl alcohol, 966; celastrol, 271; anacitabine, 225; oxalipatin, 380; chromomycin A3, 4; vinorelbine, 4; idarubicin, 34; nogalamycin, 5; 17-DMAG, 5; epothilone D, 2; vinblastine, 2; vincristine, 7; cytarabine, 137.

### Example 24

### Evaluation of Paclitaxel Containing Mesh on Intimal Hyperplasia Development in a Rat Balloon Injury Carotid Artery Model as an Example to Evaluate Fibrosis Inhibiting Agents

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French FOGARTY balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml), and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Paclitaxel Treatment

Immediately after injury of the left common carotid artery, a 1 cm long distal segment of the artery was exposed and treated with a 1×1 cm paclitaxel-containing mesh (345 μg paclitaxel in a 50:50 PLG coating on a 10:90 PLG mesh). The wound was then closed the animals were kept for 14 days.

Histology and Immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

Results

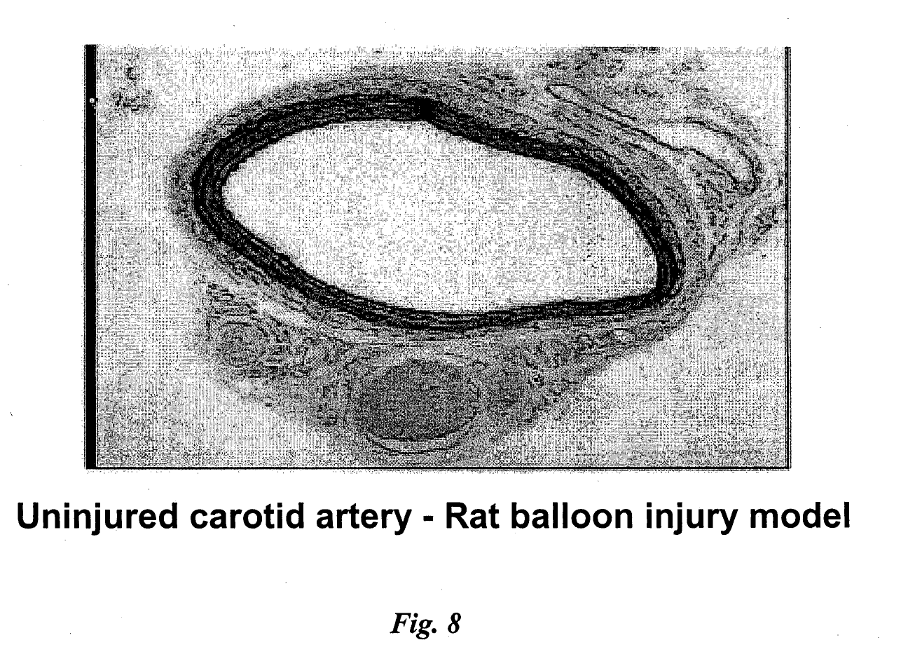

From FIGS. 8-10, it is evident that the perivascular delivery of paclitaxel using the paclitaxel mesh formulation resulted in a dramatic reduction in intimal hyperplasia.

### Example 25

### Effect of Paclitaxel and Other Anti-Microtubule Agents on Matrix Metalloproteinase Production

A. Materials and Methods

1. IL-1 Stimulated AP-1 Transcriptional Activity is Inhibited by Paclitaxel

Chondrocytes were transfected with constructs containing an AP-1 driven CAT reporter gene and stimulated with IL-1, IL-1 (50 ng/ml), which was added and incubated for 24 hours in the absence and presence of paclitaxel at various concentrations. Paclitaxel treatment decreased CAT activity in a concentration dependent manner (mean±SD). The data noted with an asterisk (*) have significance compared with IL-1-induced CAT activity according to a t-test, P<0.05. The results shown are representative of three independent experiments.

2. Effect of Paclitaxel on IL-1 Induced AP-1 DNA Binding Activity. AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3. Effect of Paclitaxel on IL-1 Induced MMP-1 and MMP-3 mRNA Expression

Cells were treated with paclitaxel at various concentrations (10⁻⁷- to 10⁻⁵ M) for 24 hours, then treated with IL-1β(20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels was conducted. The MMP-1 and MMP-3 expression levels were normalized with GAPDH.

4. Effect of Other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hybridized with the ³²P-labeled collagenase cDNA probe. ³²P labeled glyceraldehyde phosphate dehydrase (GAPDH) cDNA as an internal standard to ensure roughly equal loading. The exposed films were scanned and quantitatively analyzed with IMAGEQUANT.

B. Results

1. Promoters on the Family of Matrix Metalloproteinases

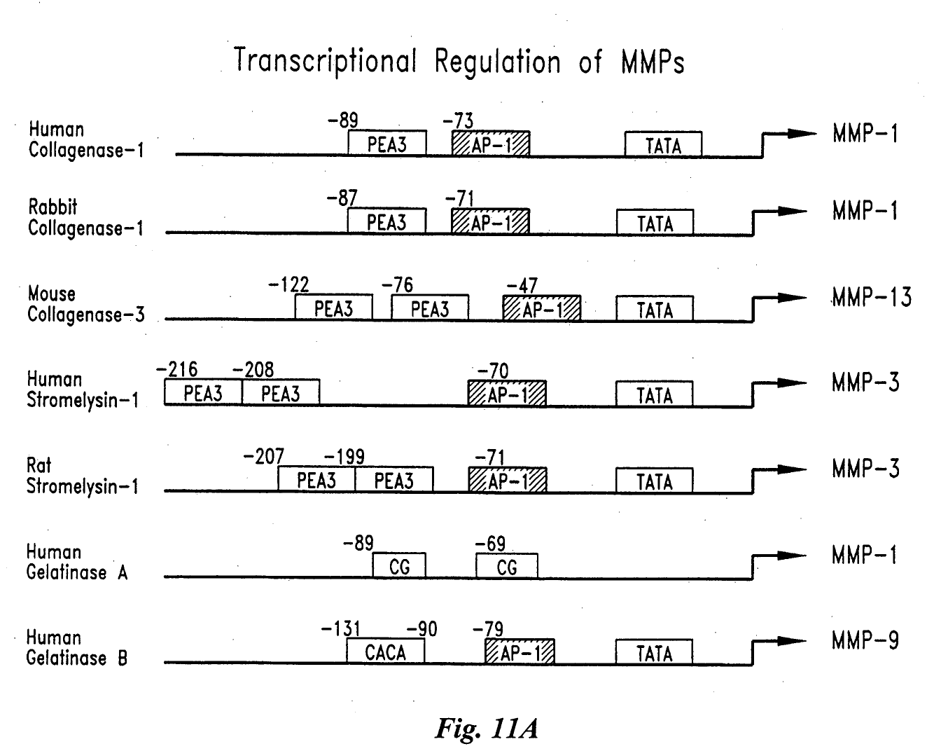

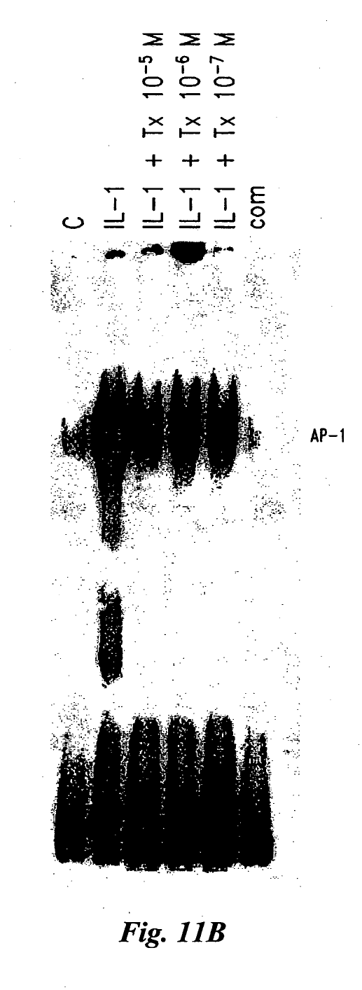

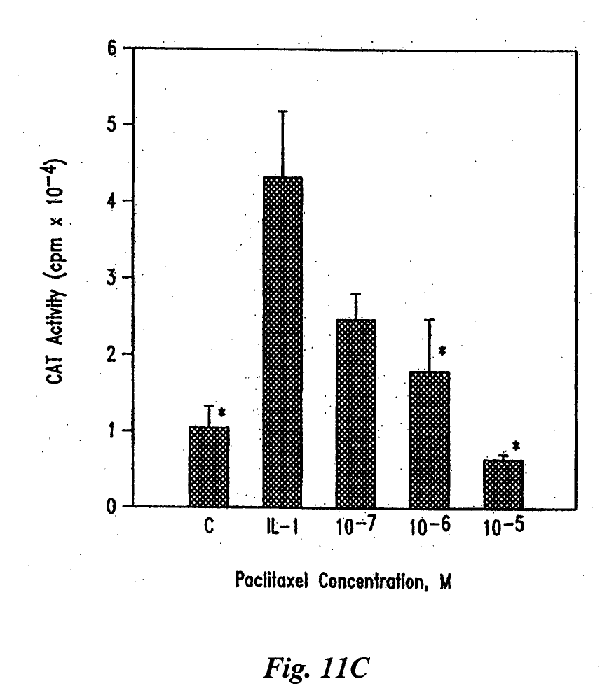

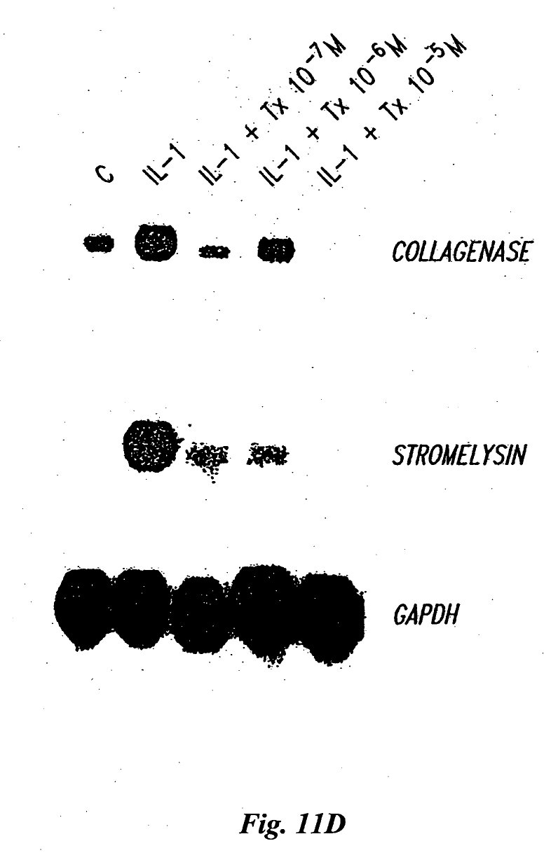

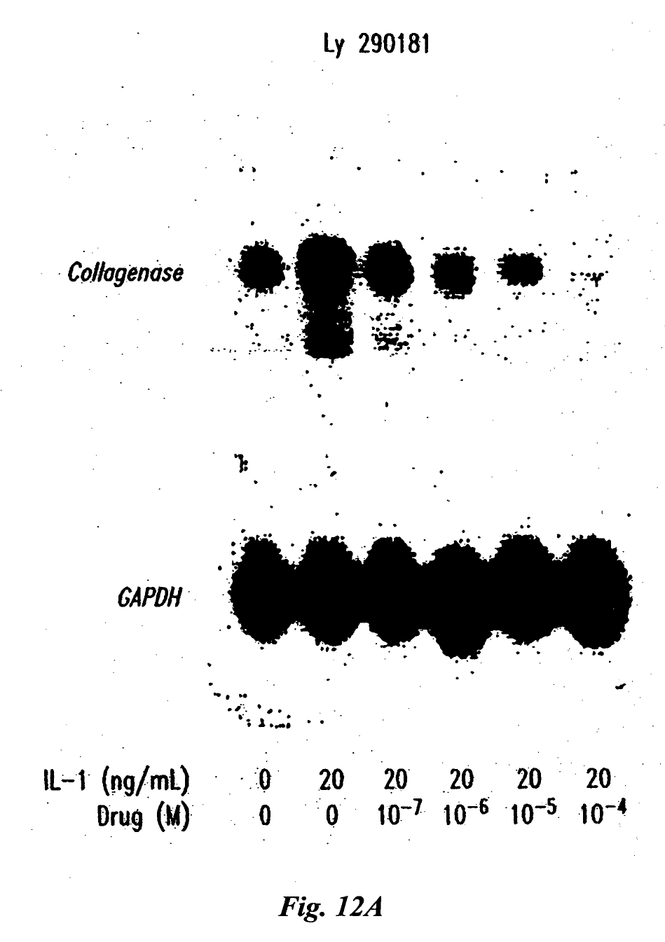

FIG. 11A shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2. Effect of Paclitaxel on AP-1 Transcriptional Activity

As demonstrated in FIG. 11B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3. Effect of Daclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 11C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4. Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 11D, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules on Collagenase Expression

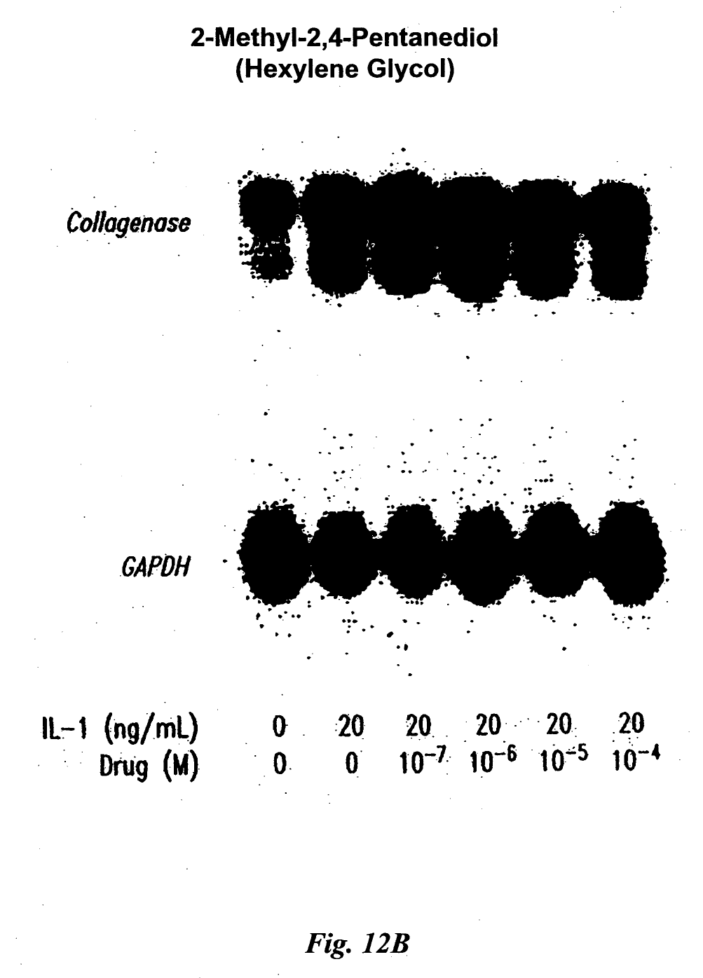

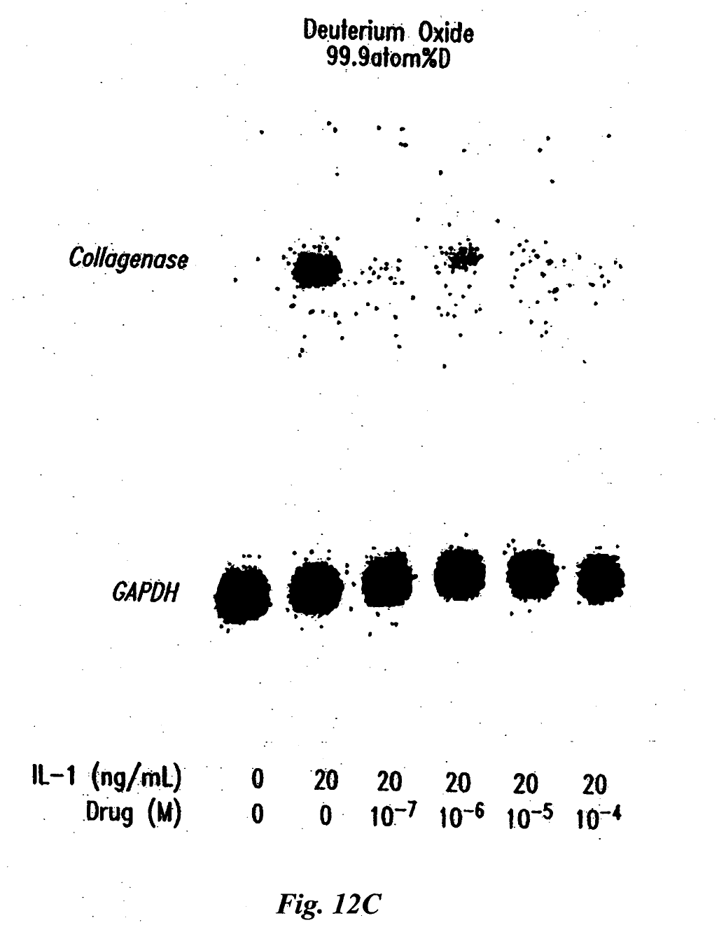

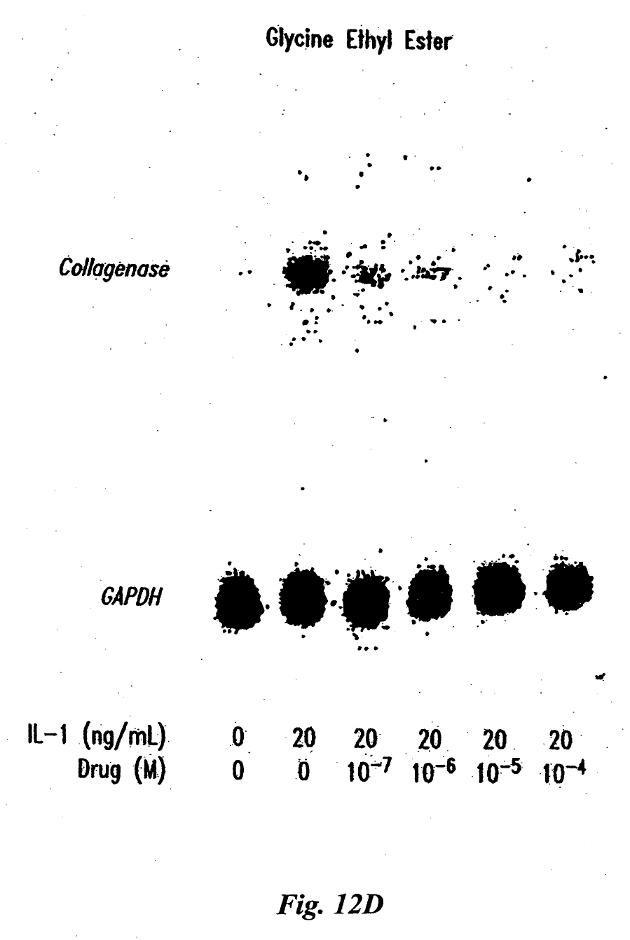

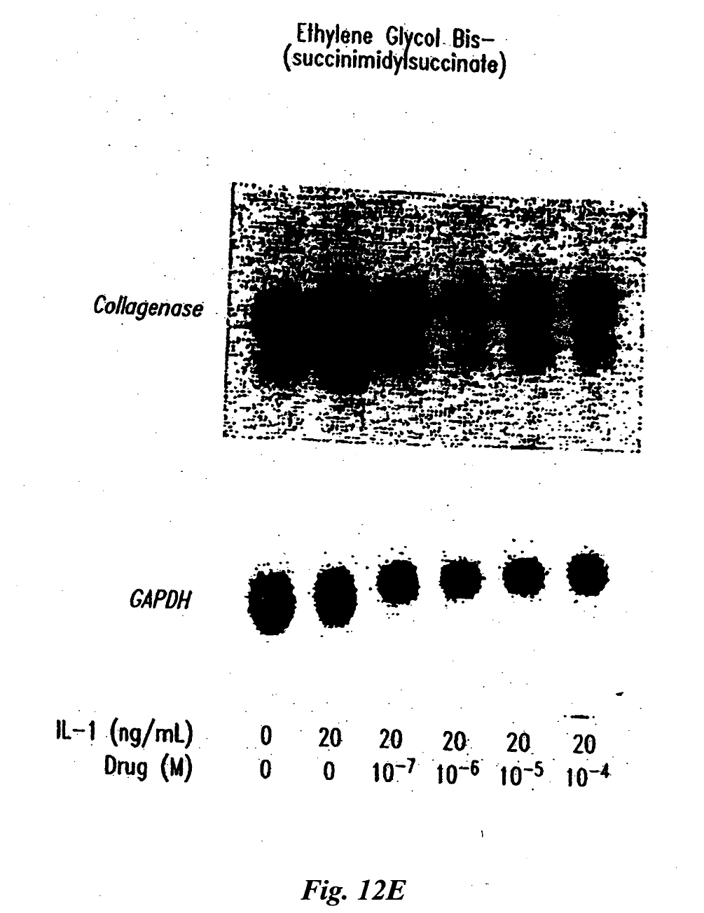

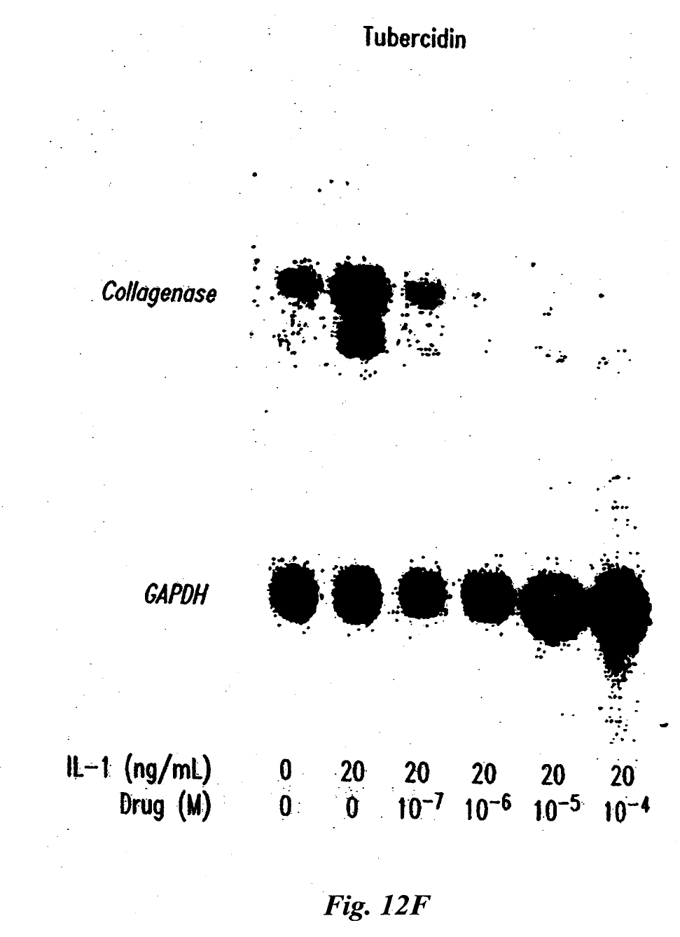

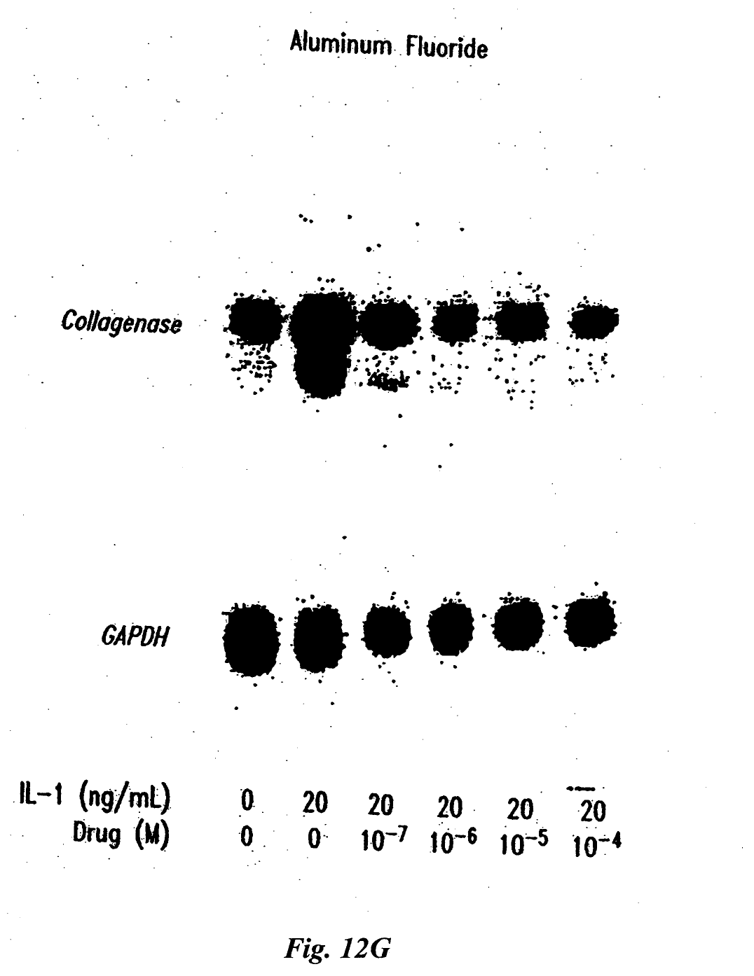

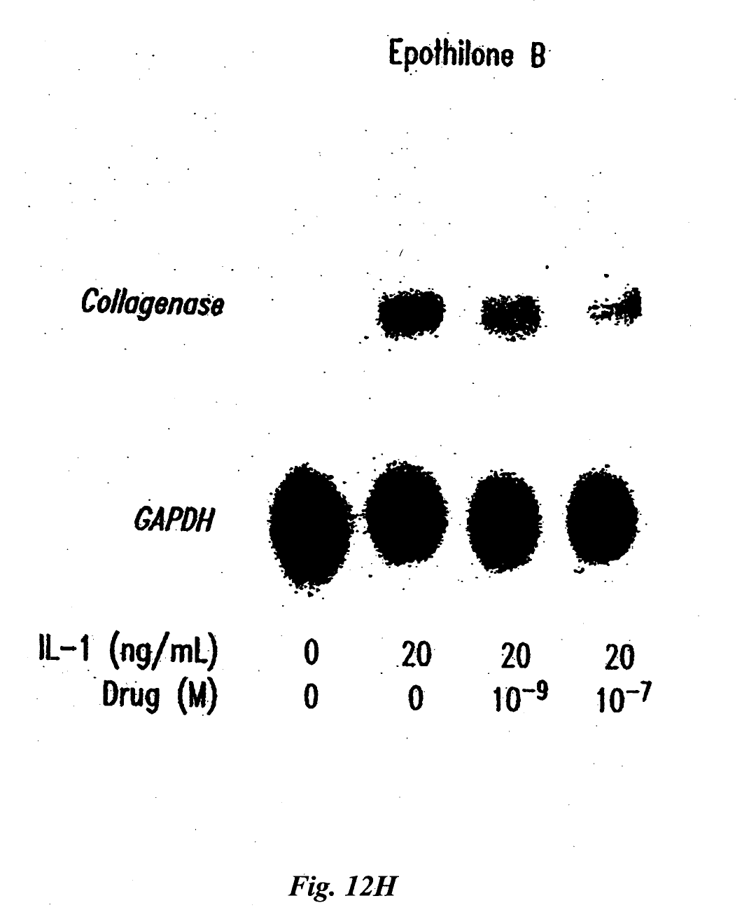

FIGS. 12A-H demonstrate that anti-microtubule agents inhibited collagenase expression. Expression of collagenase was stimulated by the addition of IL-1, which is a proinflammatory cytokine. Pre-incubation of chondrocytes with various anti-microtubule agents, specifically LY290181 (FIG. 12A); hexylene glycol (FIG. 12B); deuterium oxide (FIG. 12C); glycine ethyl ester (FIG. 12D); ethylene glycol bis-(succinimidylsuccinate) (FIG. 12E); tubercidin (FIG. 12F); AIF₃ (FIG. 12G): and epothilone (FIG. 12H), all prevented IL-1-induced collagenase expression at concentrations as low as 1×10⁻⁷ M.

C. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases that are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 26

### Inhibition of Angiogenesis by Paclitaxel

A. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 μl aliquot of 0.5% aqueous methylcellulose. Since paclitaxel is insoluble in water, glass beads were used to produce fine particles. Ten microliter aliquots of this solution were dried on paraflim for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1 M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160×, which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
AVASCULAR GRADIENT

| | |
|---|---|
| 0-- | normal vascularity |
| 1-- | lacking some microvascular movement |
| 2*-- | small avascular zone approximately 2 mm in diameter |
| 3*-- | avascularity extending beyond the disk (6 mm in diameter) |

*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 ul) | 0.25 ug | 2/11 |
| | methylcellulose (10 ul) | 0.5 ug | 6/11 |
| | methylcellulose (10 ul) | 1 ug | 6/15 |
| | methylcellulose (10 ul) | 5 ug | 20/27 |
| | methytcellulose (10 ul) | 10 ug | 16/21 |
| | methylcellulose (10 ul) | 30 ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone that was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 27

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media, and diluted to a concentration of 2-2.5×10⁵ cells/ml in migration media. Migration media consists of phenol red free Dulbecco's Modified Eagle Medium (DMEM) containing 0.35% human serum albumin. A 100 μl volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/ml in a total volume of 150 μl. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a dilution, to the cells in the top chamber. Plates were incubated for 4 hours to allow cell migration.

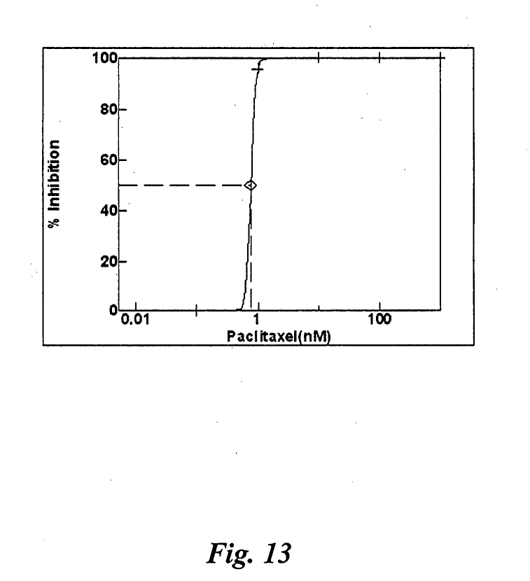

At the end of the 4 hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 13 (IC₅₀=0.76 nM). References: Biotechniques (2000) 29: 81; J. Immunol. Meth. (2001) 254: 85.

### Example 28

### Screening Assay for Assessing the Effect of Various Compounds on IL-1β Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250×g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1β by the addition of 1 mg/ml opsonized zymosan. Geldanamycin was added to THP-1 cells by directly adding DMSO stock solutions, prepared earlier, at a dilution, to each well. Each drug concentration was tested in triplicate wells. Plates were incubated at 37° C. for 24 hours.

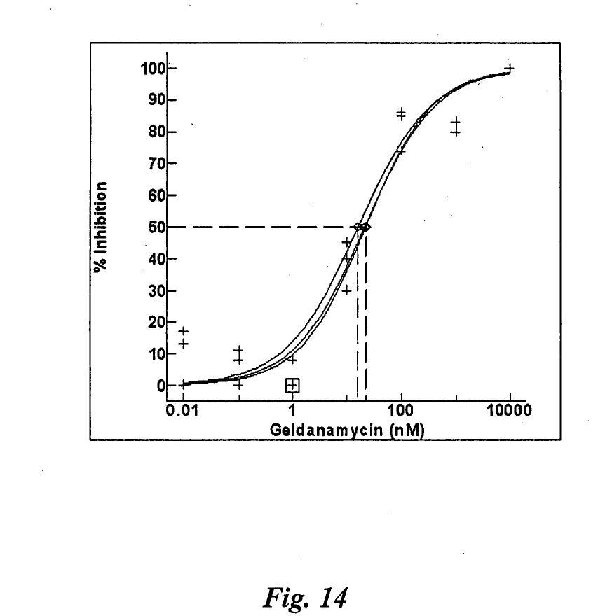

After a 24 hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μl of anti-human IL-1β Capture Antibody diluted in Coating Buffer (0.1 M sodium carbonate, pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/4 and 1/8; (b) recombinant human IL-1β was prepared at 1000 pg/ml and serially diluted to yield as standard curve of 15.6 pg/ml to 1000 pg/ml. Sample supernatants and standards were assayed in triplicate and were incubated at room temperature for 2 hours after addition to the plate coated with Capture Antibody. The plates were washed 5 times and incubated with 100 pI of Working Detector (biotinylated anti-human IL-1β detection antibody+avidin-HRP) for 1 hour at room temperature. Following this incubation, the plates were washed 7 times and 100 μl of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with λ correction at 570 nm. Mean absorbance was determined from triplicate data readings and the mean background was subtracted. IL-1β concentration values were obtained from the standard curve. Inhibitory concentration of 50% (IC₅₀) was determined by comparing average IL-1β concentration to the positive control (THP-1 cells stimulated with opsonized zymosan). An average of n=4 replicate experiments was used to determine IC₅₀ values for geldanamycin (IC₅₀=20 nM). See FIG. 14. The IC₅₀ values for the following additional compounds were determined using this assay: IC₅₀ (nM): mycophenolic acid, 2888 nM; anisomycin, 127; rapamycin, 0.48; halofuginone, 919; IDN-6556, 642; epirubicin hydrochloride, 774; topotemay, 509; fascaplycin, 425; daunorubicin, 517; celastrol, 23; oxalipatin, 107; chromomycin A3,148.

References: J. Immunol. (2000) 165: 411-418; J. Immunol. (2000) 164: 4804-11; J. Immunol Meth. (2000) 235 (1-2): 33-40.

### Example 29

### Screening Assay for Assessing the Effect of Various Compounds on IL-8 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250×g, resuspended in 4 ml of human serum for a final concentration of 5 mg/ml, and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-8 by the addition of 1 mg/ml opsonized zymosan. Geldanamycin was added to THP-1 cells by directly adding DMSO stock solutions, prepared earlier, at a 1/1000 dilution, to each well. Each drug concentration Was tested in triplicate wells. Plates were incubated at 37° C. for 24 hours.

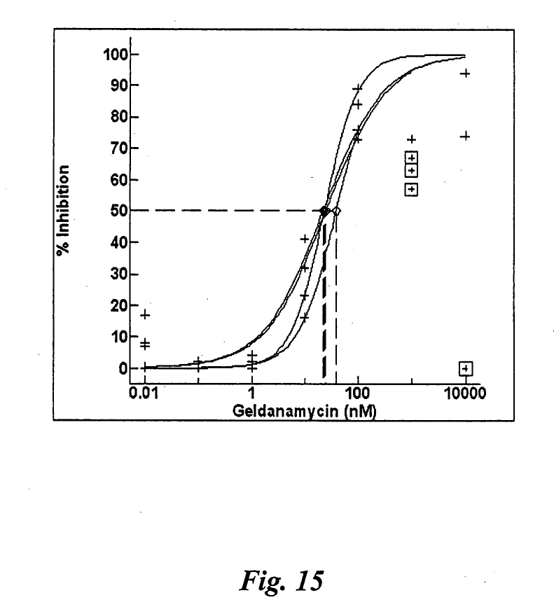

After a 24 hour stimulation, supernatants were collected to quantify IL-8 production. IL-8 concentrations in the supernatants Were determined by ELISA using recombinant human IL-8 to obtain a standard curve. A 96-well MAXISORB plate was coated with 100 μl of anti-human IL-8 Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human IL-8 was prepared at 200 pg/ml and serially diluted to yield as standard curve of 3.1 pg/ml to 200 pg/ml. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human IL-8 detection antibody+avidin-HRP) for 1 hour at room temperature. Following this incubation, the plates were washed 7 times and 100 μl of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with λ correction at 570 nm. Mean absorbance was determined from triplicate data readings and the mean background was subtracted. IL-8 concentration values were obtained from the standard curve. Inhibitory concentration of 50% (IC₅₀) was determined by comparing average IL-8 concentration to the positive control (THP-1 cells stimulated with opsonized zymosan). An average of n=4 replicate experiments was used to determine IC₅₀ values for geldanamycin (IC₅₀=27 nM). See FIG. 15. The IC₅₀ values for the following additional compounds were determined using this assay: IC₅₀ (nM): 17-AAG, 56; mycophenolic acid, 549; resveratrol, 507; rapamycin, 4; 41; SP600125, 344; halofuginone, 641; D-mannose-6-phosphate, 220; epirubicin hydrochloride, 654; topotemay, 257; mithramycin, 33; daunorubicin, 421; celastrol, 490; chromomycin A3, 36.

References: Sugawara et al., J. Immunol. (2000) 165: 411-418; Dankesreiter et al., J. Immunol. (2000) 164: 4804-4811; J. Immunol Meth. (2000) 235 (1-2): 33-40.

### Example 30

### Screening Assay for Assessing the Effect of Various Compounds on Mcp-1 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250×g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce MCP-1 by the addition of 1 mg/ml opsonized zymosan. Eldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

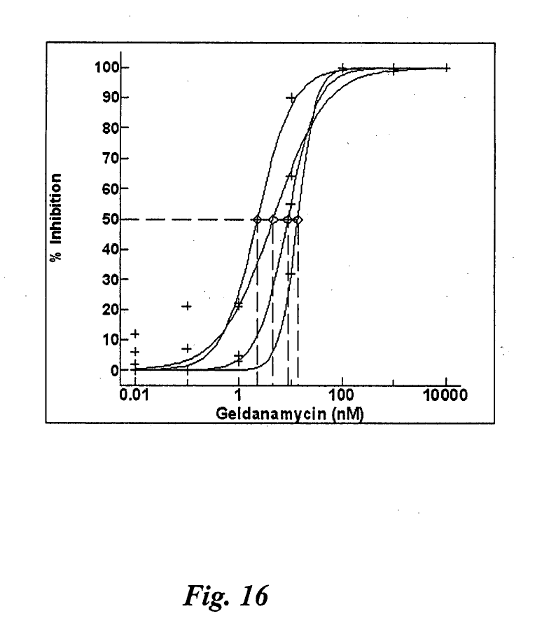

After a 24 hour stimulation, supernatants were collected to quantify MCP-1 production. MCP-1 concentrations in the supernatants were determined by ELISA using recombinant human MCP-1 to obtain a standard curve. A 96-well MaxiSorb plate was coated with 100 μl of anti-human MCP-1 Capture Antibody diluted in Coating Buffer (0.1 M Sodium carbonate, pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human MCP-1 was prepared at 500 pg/ml and serially diluted to yield as standard curve of 7.8 pg/ml to 500 pg/ml. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human MCP-1 detection antibody+avidin-HRP) for 1 hour at room temperature. Following this incubation, the plates were washed 7 times and 100 μl of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. MCP-1 concentration values were obtained from the standard curve. Inhibitory concentration of 50% (IC₅₀) was determined by comparing average MCP-1 concentration to the positive control (THP-1 cells stimulated with opsonized zymosan). An average of n=4 replicate experiments was used to determine IC₅₀ values for geldanamycin (IC₅₀=7 nM). See FIG. 16. The IC₅₀ values for the following additional compounds were determined using this assay: IC₅₀ (nM): 17-MG, 135; anisomycin, 71; mycophenolic acid, 764; mofetil, 217; mitoxantrone, 62; chlorpromazine, 0.011; 1-α-25 dihydroxy vitamin D₃, 1; Bay 58-2667, 216; 15-deoxy prostaglandin J2, 724; rapamycin, 0.05; CNI-1493, 0.02; BXT-51072, 683; halofuginone, 9; CYC 202, 306; topotemay, 514; fascaplycin, 215; podophyllotoxin, 28; gemcitabine, 50; puromycin, 161; mithramycin, 18; daunorubicin, 570; celastrol, 421; chromomycin A3, 37; vinorelbine, 69; tubercidin, 56; vinblastine, 19; vincristine, 16.

References: Sugawara et al., J. Immunol. (2000) 165: 411-18; J. Immunol. (2000) 164: 4804-11; J. Immunol Meth. (2000) 235 (1-2): 33-40.

### Example 31

### Screening Assay for Assessing the Effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μl/well. Each drug concentration was tested in triplicate wells. Plates containing cells and paclitaxel were incubated at 37° C. for 72 hours.

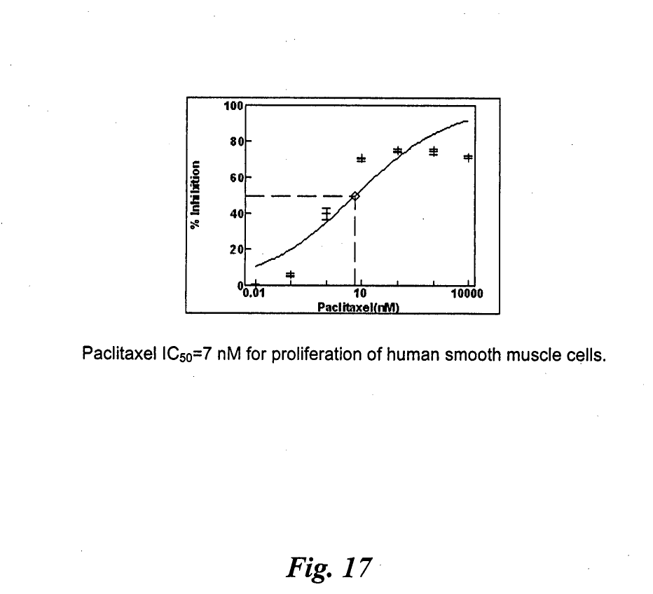

To terminate the assay, the media was removed by gentle aspiration. A 1/400 dilution of CYQUANT 400×GR dye indicator (Molecular Probes; Eugene, Oreg.) was added to 1× Cell Lysis buffer, and 200 μl of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values. See FIG. 17 (IC₅₀=7 nM). The IC₅₀ values for the following additional compounds were determined using this assay: IC₅₀ (nM): mycophenolic acid, 579; mofetil, 463; doxorubicin, 64; mitoxantrone, 1; geldanamycin, 5; anisomycin, 276; 17-MG, 47; cytarabine, 85; halofuginone, 81; mitomycin C, 53; etoposide, 320; cladribine, 137; lovastatin, 978; epirubicin hydrochloride, 19; topotemay, 51; fascaplysin, 510; podophyllotoxin, 21; cytochalasin A, 221; gemcitabine, 9; puromycin, 384; mithramycin, 19; daunorubicin, 50; celastrol, 493; chromomycin A3, 12; vinorelbine, 15; idarubicin, 38; nogalamycin, 49; itraconazole, 795; 17-DMAG, 17; epothilone D, 5; tubercidin, 30; vinblastine, 3; vincristine, 9.

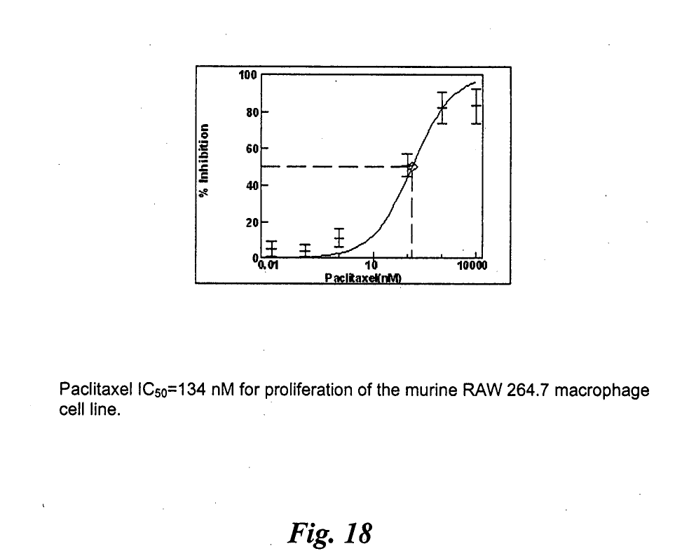

This assay also may be used assess the effect of compounds on proliferation of fibroblasts and murine macrophage cell line RAW 264.7. The results of the assay for assessing the effect of paclitaxel on proliferation of murine RAW 264.7 macrophage cell line were shown in FIG. 18 (IC₅₀=134 nM).

References: In Vitro Toxicol. (1990) 3: 219; Biotech. Histochem. (1993) 68: 29; Anal. Biochem. (1993) 213: 426.

### Example 32

### Perivascular Administration of Paclitaxel to Assess Inhibition of Fibrosis

WISTAR rats weighing 250-300 g are anesthetized by the intramuscular injection of Innovar (0.33 ml/kg). Once sedated, the rats are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Paclitaxel (33%) in ethelyne vinyl acetate (EVA) is then injected in a circumferential fashion around the common carotid artery in ten rats. EVA alone is injected around the common carotid artery in ten additional rats. (The paclitaxel may also be coated onto an EVA film that is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

### Example 33

### In Vivo Evaluation of Silk Coated Perivascular PU Films to Assess the Ability of an Agent to Induce Scarring

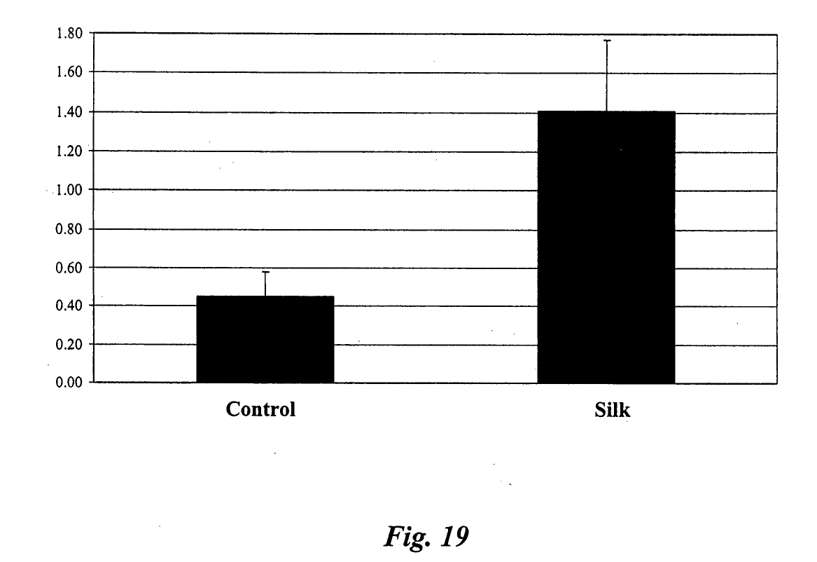

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk strands or a control uncoated PU film is wrapped around a distal segment of the common carotid artery. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area (mm²) of perivascular granulation tissue is quantified by computer-assisted morphometric analysis. Area of the granulation tissue is significantly higher in the silk coated group than in the control uncoated group. See FIG. 19.

### Example 34

### In Vivo Evaluation of Perivascular PU Films Coated with Different Silk Suture Material to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk sutures from one of three different manufacturers (3-0 Silk-Black Braided (Davis & Geck), 3-0 SOFSILK (U.S. Surgical/Davis & Geck), and 3-0 Silk-Black Braided (LIGAPAK) (Ethicon, Inc.) is wrapped around a distal segment of the common carotid artery. (The polyurethane film can also be coated with other agents to induce fibrosis.) The wound is closed and the animal is allowed to recover.

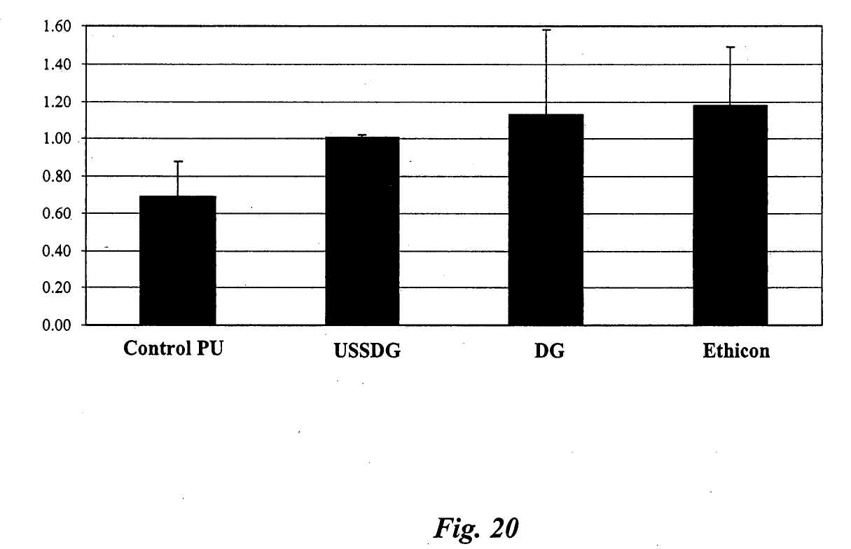

After 28 days, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Thickness of the granulation tissue is the same in the three groups showing that tissue proliferation around silk suture is independent of manufacturing processes. See FIG. 20.

### Example 35

### In Vivo Evaluation of Perivascular Silk Powder to Assess the Capacity of an Agent to Induce Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a PU film. Natural silk powder or purified silk powder (without contaminant proteins) is used in different groups of animals. Carotids wrapped with PU films only are used as a control group. The wound is closed and the animal is allowed to recover. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area of tunica intima, tunica media and perivascular granulation tissue is quantified by computer-assisted morphometric analysis.

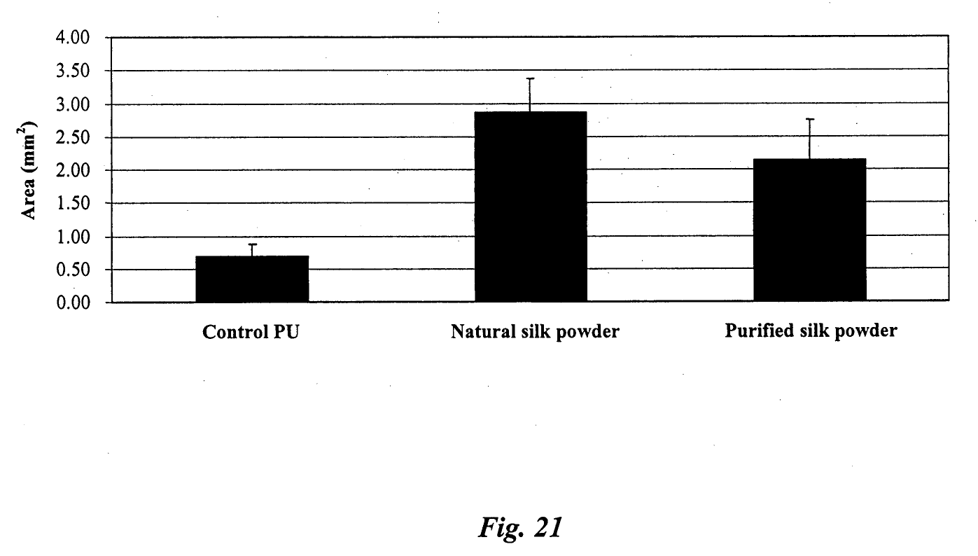

The natural silk caused a severe cellular inflammation consisting mainly of a neutrophil and lymphocyte infiltrate in a fibrin network without any extracellular matrix or blood vessels. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells, and blood vessels. The treated artery was intact. These results show that removing the coating proteins from natural silk prevents the immune response and promotes benign tissue growth. Degradation of the regenerated silk powder was underway in some histology sections indicating that the tissue response can likely mature and heal over time. See FIG. 21.

### Example 36

### In Vivo Evaluation of Perivascular Talcum Powder to Assess the Capacity of an Agent to Induce Scarring

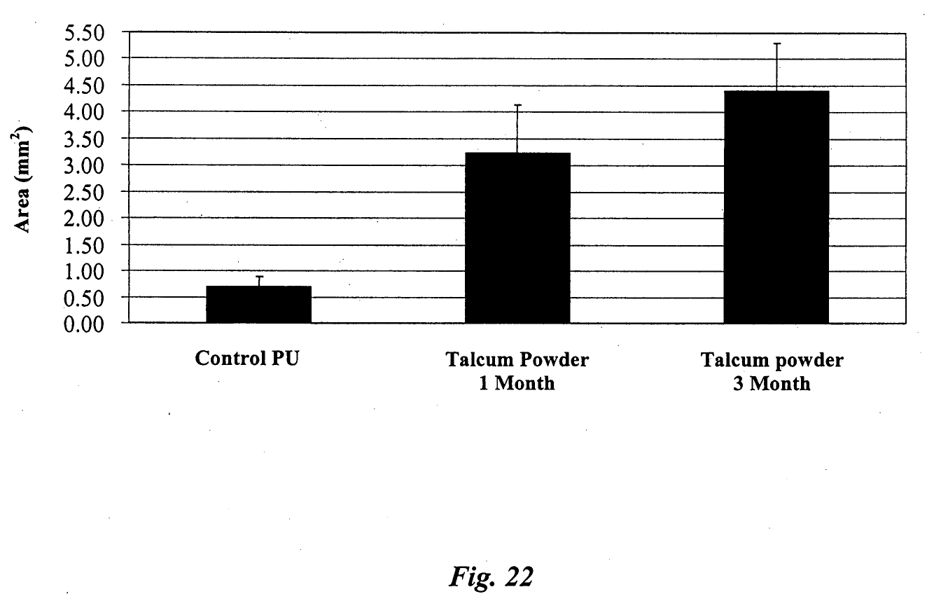

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Talcum powder is sprinkled on the exposed artery that is then wrapped with a PU film. Carotids wrapped with PU films only are used as a control group. The wound is closed and the animal is recovered. After 1 or 3 months, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Thickness of tunica intima, tunica media and perivascular granulation tissue is quantified by computer-assisted morphometric analysis. Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes and blood vessels. The treated blood vessel appeared intact and unaffected by the treatment. Overall, this result showed that talcum powder induced a mild long-lasting fibrotic reaction that was subclinical in nature and did not harm any adjacent tissue. See FIG. 22.

### Example 37

### Mic Determination by Microtitre Broth Dilution Method

A. MIC Assay of Various Gram Negative and Positive Bacteria

MIC assays were conducted essentially as described by Amsterdam, D. 1996, “Susceptibility testing of antimicrobials in liquid media”, p. 52-111, in Loman, V., ed. Antibiotics in Laboratory Medicine, 4th ed. Williams and Wilkins, Baltimore, Md. Briefly, a variety of compounds were tested for antibacterial activity against isolates of Pseudomonas aeruginosa, Klebsiella pneumoniae, E. col, Streptococcus pyogenes, S. epidermidis, and S. aureus in the MIC (minimum inhibitory concentration assay under aerobic conditions using 96 well polystyrene microtitre plates (Falcon 1177), and Mueller Hinton broth at 37° C. incubated for 24 hours. (MHB was used for most testing except C721 (S. pyogenes), which used Todd Hewitt broth, and Haemophilus influenzae, which used Haemophilus test medium (HTM)). Tests were conducted in triplicate. The results are provided below in Table 1.

TABLE 1
MINIMUM INHIBITORY CONCENTRATIONS OF THERAPEUTIC AGENTS
AGAINST VARIOUS GRAM NEGATIVE AND POSITIVE BACTERIA

| | P. aeruginosa | K. pneumoniae | | S. aureus | | |
|---|---|---|---|---|---|---|
| | PAE/ | ATCC | E. coli | ATCC | | |
| | K799 | 13883 | UB1005 | 25923 | S. epidermidis | S. pyogenes |
| Bacterial | H187 | C238 | C498 | C622 | C621 | C721 |
| Strain | Wt | wt | wt | wt | wt | wt |
| Drug | Gram − | Gram − | Gram − | Gram + | Gram + | Gram + |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5- | 10⁻⁵ | 10⁻⁶ | 10⁻⁶ | 10⁻⁷ | 10⁻⁷ | 10⁻⁴ |
| fluorouracil | | | | | | |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2- | N | N | N | N | N | N |
| mercaptopurine | | | | | | |
| 6- | N | N | N | N | N | N |
| mercaptopurine | | | | | | |
| Cytarabine | N | N | N | N | N | N |

Activities are in molar concentrations
Wt = wild type
N = No activity

B. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea, and cytarabine were tested for antibacterial activity against clinical isolates of a methicillin-resistant S. aureus and a vancomycin resistant pediocoocus clinical isolate in an MIC assay as described above. Compounds which showed inhibition of growth (MIC value of <1.0×10⁻³) included: mitoxantrone (both strains), methotrexate (vancomycin resistant pediococcus), 5-fluorouracil (both strains), etoposide (both strains), and 2-mercaptopurine (vancomycin resistant pediococcus).

### Example 38

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1 N NaOH or 0.1 N phosphoric acid.

### Example 39

### Release Study to Determine Release Profile of the Therapeutic Agent from a Coated Device

A sample of the therapeutic agent-loaded catheter is placed in a 15 ml culture tube. 15 ml release buffer (Example 38) is added to the culture tube. The tube is sealed with a TEFLON lined screw cap and is placed on a rotating wheel in a 37° C. oven. At various time points, the buffer is withdrawn from the culture tube and is replaced with fresh buffer. The withdrawn buffer is then analyzed for the amount of therapeutic agent contained in this buffer solution using HPLC.

From the foregoing, it is appreciated that, although specific embodiments of the invention have been described herein for purposes of illustration, various modifications may be made without deviating from the spirit and scope of the invention. Accordingly, the invention is not limited except as by the appended claims.

1.-878. (canceled)

879. A device comprising a facial implant and either an anti-scarring agent or a composition comprising an anti-scarring agent, wherein the agent inhibits scarring between the device and the host into which the device is implanted.

880. The device of claim 879 wherein the implant is a cosmetic implant.

881. The device of claim 879 wherein the implant is a reconstructive implant.

882. The device of claim 879 wherein the agent reduces tissue regeneration.

883. The device of claim 879 wherein the agent inhibits inflammation.

884. The device of claim 879 wherein the agent inhibits fibrosis.

885. The device of claim 879 wherein the agent inhibits adhesion between the device and the host into which the device is implanted.

886. The device of claim 879 wherein the agent inhibits angiogenesis.

887. The device of claim 879 wherein the agent inhibits migration of connective tissue cells.

888. The device of claim 879 wherein the agent inhibits proliferation of connective tissue cells.

889. The device of claim 879 wherein the agent inhibits fibroblast migration.

890. The device of claim 879 wherein the agent inhibits fibroblast proliferation.

891. The device of claim 879 wherein the agent inhibits extracellular matrix production.

892. The device of claim 879 wherein the agent enhances extracellular matrix breakdown.

893. The device of claim 879 wherein the agent inhibits deposition of extracellular matrix.

894. The device of claim 879 wherein the agent inhibits tissue remodeling.

895. The device of claim 879 wherein the agent inhibits formation of a fibrous connective tissue capsule enclosing the device.

896.-900. (canceled)

901. The device of claim 879 wherein the agent is a taxane.

902.-1200. (canceled)

1201. The device of claim 879, further comprising a second pharmaceutically active agent.

1202. (canceled)

1203. (canceled)

1204. The device of claim 879, further comprising an agent that inhibits infection.

1205.-5687. (canceled)

5688. A method for inhibiting scarring between a facial implant and a host comprising placing a device that comprises the facial implant and either an anti-scarring agent or a composition comprising the anti-scarring agent into the host, wherein the agent inhibits scarring.

5689. The method of claim 5688 wherein the implant is a cosmetic implant.

5690. The method of claim 5688 wherein the implant is a reconstructive implant.

5691. The method of claim 5688 wherein the agent reduces tissue regeneration.

5692. The method of claim 5688 wherein the agent inhibits inflammation.

5693. The method of claim 5688 wherein the agent inhibits fibrosis.

5694. The method of claim 5688 wherein the agent inhibits adhesion between the device and the host into which the device is implanted.

5695. The method of claim 5688 wherein the agent inhibits angiogenesis.

5696. The method of claim 5688 wherein the agent inhibits migration of connective tissue cells.

5697. The method of claim 5688 wherein the agent inhibits proliferation of connective tissue cells.

5698. The method of claim 5688 wherein the agent inhibits fibroblast migration.

5699. The method of claim 5688 wherein the agent inhibits fibroblast proliferation.

5700. The method of claim 5688 wherein the agent inhibits extracellular matrix production.

5701. The method of claim 5688 wherein the agent enhances extracellular matrix breakdown.

5702. The method of claim 5688 wherein the agent inhibits deposition of extracellular matrix.

5703. The method of claim 5688 wherein the agent inhibits tissue remodeling.

5704. The method of claim 5688 wherein the agent inhibits formation of a fibrous connective tissue capsule enclosing the device.

5705.-5709. (canceled)

5710. The method of claim 5688 wherein the agent is a taxane.

5711.-6009. (canceled)

6010. The method of claim 5688, wherein the device further comprises a second pharmaceutically active agent.

6011. (canceled)

6012. (canceled)

6013. The method of claim 5688, wherein the device further comprises an agent that inhibits infection.

6014.-10653. (canceled)

10654. A method for making a device comprising combining a facial implant and either an anti-scarring agent or a composition comprising an anti-scarring agent, wherein the agent inhibits scarring between the device and a host into which the device is implanted.

10655. The method of claim 10654 wherein the implant is a cosmetic implant.

10656. The method of claim 10654 wherein the implant is a reconstructive implant.

10657. The method of claim 10654 wherein the agent reduces tissue regeneration.

10658. The method of claim 10654 wherein the agent inhibits inflammation.

10659. The method of claim 10654 wherein the agent inhibits fibrosis.

10660. The method of claim 10654 wherein the agent inhibits adhesion between the device and the host into which the device is implanted.

10661. The method of claim 10654 wherein the agent inhibits angiogenesis.

10662. The method of claim 10654 wherein the agent inhibits migration of connective tissue cells.

10663. The method of claim 10654 wherein the agent inhibits proliferation of connective tissue cells.

10664. The method of claim 10654 wherein the agent inhibits fibroblast migration.

10665. The method of claim 10654 wherein the agent inhibits fibroblast proliferation.

10666. The method of claim 10654 wherein the agent inhibits extracellular matrix production.

10667. The method of claim 10654 wherein the agent enhances extracellular matrix breakdown.

10668. The method of claim 10654 wherein the agent inhibits deposition of extracellular matrix.

10669. The method of claim 10654 wherein the agent inhibits tissue remodeling.

10670. The method of claim 10654 wherein the agent inhibits formation of a fibrous connective tissue capsule enclosing the device.

10671.-10675. (canceled)

10676. The method of claim 10654 wherein the agent is a taxane.

10677.-10975. (canceled)

10976. The method of claim 10654, wherein the device further comprises a second pharmaceutically active agent.

10977. (canceled)

10978. (canceled)

10979. The method of claim 10654, wherein the device further comprises an agent that inhibits infection.

10980.-15455. (canceled)

15456. A method for augmenting the malar or submalar region comprising placing into a host a device that comprises a facial implant and either an anti-scarring agent or a composition comprising an anti-scarring agent, wherein the agent inhibits scarring between the device and the host into which the device is implanted.

15457. The method of claim 15456 wherein the implant is a cosmetic implant.

15458. The method of claim 15456 wherein the implant is a reconstructive implant.

15459. The method of claim 15456 wherein the agent reduces tissue regeneration.

15460. The method of claim 15456 wherein the agent inhibits inflammation.

15461. The method of claim 15456 wherein the agent inhibits fibrosis.

15462. The method of claim 15456 wherein the agent inhibits adhesion between the device and the host into which the device is implanted.

15463. The method of claim 15456 wherein the agent inhibits angiogenesis.

15464. The method of claim 15456 wherein the agent inhibits migration of connective tissue cells.

15465. The method of claim 15456 wherein the agent inhibits proliferation of connective tissue cells.

15466. The method of claim 15456 wherein the agent inhibits fibroblast migration.

15467. The method of claim 15456 wherein the agent inhibits fibroblast proliferation.

15468. The method of claim 15456 wherein the agent inhibits extracellular matrix production.

15469. The method of claim 15456 wherein the agent enhances extracellular matrix breakdown.

15470. The method of claim 15456 wherein the agent inhibits deposition of extracellular matrix.

15471. The method of claim 15456 wherein the agent inhibits tissue remodeling.

15472. The method of claim 15456 wherein the agent inhibits formation of a fibrous connective tissue capsule enclosing the device.

15473.-15477. (canceled)

15478. The method of claim 15456 wherein the agent is a taxane.

15479.-19368. (canceled)
